(19) United States
(12) Patent Application Publication
(10) Pub. No.: US 2007/0281306 A1
(43) Pub. Date: Dec. 6, 2007

(54) Novel 15571, 2465, 14266, 2882, 52871, 8203 and 16852 molecules and uses therefor

(75) Inventors: Martin R. Hodge, Lexington, MA (US); Clare M. Lloyd, London (GB); Nadine S. Weich, Brookline, MA (US); Jose M. Lora, Arlington, MA (US); David White, Braintree, MA (US); Maria A. Glucksmann, Lexington, MA (US); Keith E. Robison, Wilmington, MA (US); Inmaculada Silos-Santiago, Del Mar, CA (US); Andrew D.J. Goodearl, Natick, MA (US); Rory A. J. Curtis, Ashland, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/524,793

(22) Filed: Sep. 21, 2006

Related U.S. Application Data
(63) Continuation of application No. 10/404,618, filed on Apr. 1, 2003, now abandoned.
Application No. 10/404,618 is a continuation-in-part of application No. 09/631,603, filed on Aug. 3, 2000, now Pat. No. 6,733,990.
Application No. 09/631,603 is a continuation-in-part of application No. 09/515,781, filed on Feb. 29, 2000, now abandoned.
Application No. 10/404,618 is a continuation-in-part of application No. 09/794,763, filed on Feb. 26, 2001, now abandoned.
Application No. 10/404,618 is a continuation-in-part of application No. 09/634,392, filed on Aug. 9, 2000, now abandoned.
Application No. 10/404,618 is a continuation-in-part of application No. 09/176,075, filed on Oct. 20, 1998, now abandoned.
Application No. 09/176,075 is a division of application No. 09/013,634, filed on Jan. 26, 1998, now Pat. No. 5,945,307.
Application No. 10/404,618 is a continuation-in-part of application No. 09/884,430, filed on Jun. 18, 2001, now abandoned.
Application No. 10/404,618 is a continuation-in-part of application No. 10/282,958, filed on Oct. 28, 2002, now abandoned.
Application No. 10/282,958 is a continuation of application No. 09/349,755, filed on Jul. 8, 1999, now abandoned.
Application No. 09/349,755 is a division of application No. 09/042,780, filed on Mar. 17, 1998, now abandoned.
Application No. 09/042,780 is a continuation-in-part of application No. 08/985,090, filed on Dec. 4, 1997, now Pat. No. 5,882,893.
Application No. 10/404,618 is a continuation-in-part of application No. 09/707,235, filed on Nov. 6, 2000, now abandoned.
Application No. 09/707,235 is a continuation-in-part of application No. 09/361,883, filed on Jul. 27, 1999, now abandoned.
Application No. 09/361,883 is a continuation-in-part of application No. 09/123,020, filed on Jul. 27, 1998, now abandoned.
(60) Provisional application No. 60/146,916, filed on Aug. 3, 1999.
(60) Provisional application No. 60/185,942, filed on Feb. 29, 2000.
(60) Provisional application No. 60/269,758, filed on Feb. 16, 2001.
(60) Provisional application No. 60/212,331, filed on Jun. 16, 2000.

(51) Int. Cl. C12Q 1/68; G01N 33/567; C07H 21/04; C12P 21/06; A61K 48/00; C07K 16/28; C07K 14/705

(52) U.S. Cl. 435/6; 435/69.1; 435/320.1; 435/325; 530/350; 536/23.5; 530/388.22; 435/7.2; 514/44

(57) ABSTRACT

The invention provides isolated nucleic acids molecules, designated 15571, 2465, 14266, 2882, 52871, 8203 or 16852 nucleic acid molecules. The invention also provides antisense nucleic acid molecules, recombinant expression vectors containing 15571, 2465, 14266, 2882, 52871, 8203 or 16852 nucleic acid molecules, host cells into which the expression vectors have been introduced, and nonhuman transgenic animals in which a 15571, 2465, 14266, 2882, 52871, 8203 or 16852 gene has been introduced or disrupted. The invention still further provides isolated 15571, 2465, 14266, 2882, 52871, 8203 or 16852 proteins, fusion proteins, antigenic peptides and anti-15571, 2465, 14266, 2882, 52871, 8203 or 16852 antibodies. Diagnostic and therapeutic methods utilizing compositions of the invention are also provided.

### RELATED APPLICATIONS

The present application is a continuation of U.S. patent application Ser. No. 10/404,618 (pending), filed Apr. 1, 2003, which is a continuation-in-part of U.S. patent application Ser. No. 09/631,603, filed Aug. 3, 2000, now U.S. Pat. No. 6,733,990, which is a continuation-in-part of U.S. patent application Ser. No. 09/515,781, filed Feb. 29, 2000 (abandoned), which claims the benefit of U.S. Provisional Application Ser. No. 60/146,916, filed Aug. 3, 1999 (abandoned). U.S. patent application Ser. No. 10/404,618 is also a continuation-in-part of U.S. patent application Ser. No. 09/794,763, filed Feb. 26, 2001 (abandoned), which claims the benefit of U.S. Provisional Application Ser. No. 60/185,942, filed Feb. 29, 2000 (abandoned). U.S. patent application Ser. No. 10/404,618 is also a continuation-in-part of U.S. patent application Ser. No. 09/634,392, filed Aug. 9, 2000 (abandoned). U.S. patent application Ser. No. 10/404,618 is also a continuation-in-part of U.S. patent application Ser. No. 09/176,075, filed Oct. 20, 1998 (abandoned), which is a divisional of U.S. patent application Ser. No. 09/013,634, filed Jan. 26, 1998, now U.S. Pat. No. 5,945,307. U.S. patent application Ser. No. 10/404,618 is also a continuation-in-part of U.S. patent application Ser. No. 09/884,430, filed Jun. 18, 2001 (abandoned), which claims the benefit of U.S. Provisional Application Ser. No. 60/269,758, filed Feb. 16, 2001 (abandoned) and of U.S. Provisional Application Ser. No. 60/212,331, filed Jun. 16, 2000 (abandoned). U.S. patent application Ser. No. 10/404,618 is also a continuation-in-part of U.S. patent application Ser. No. 10/282,958, filed Oct. 28, 2002 (pending), which is a continuation of U.S. patent application Ser. No. 09/349,755, filed Jul. 8, 1999 (abandoned), which is a divisional of U.S. patent application Ser. No. 09/042,780, filed Mar. 17, 1998 (abandoned), which is a continuation-in-part of U.S. patent application Ser. No. 08/985,090, filed Dec. 4, 1997, now U.S. Pat. No. 5,882,893. U.S. patent application Ser. No. 10/404,618 is also a continuation-in-part of U.S. patent application Ser. No. 09/707,235, filed Nov. 6, 2000 (abandoned), which is a continuation-in-part of U.S. patent application Ser. No. 09/361,883, filed Jul. 27, 1999 (abandoned), which is a continuation-in-part of U.S. patent application Ser. No. 09/123,020, filed Jul. 27, 1998 (abandoned). The entire contents of each of the above-referenced patent applications are incorporated herein by this reference.

INDEX

| Chapter | Page | Title |
|---|---|---|
| I. | 2 | 15571, A NOVEL GPCR-LIKE MOLECULE OF THE |
| | | SECRETIN-LIKE FAMILY AND USES THEREOF |
| II. | 78 | METHODS AND COMPOSITIONS FOR THE |
| | | DIAGNOSIS AND TREATMENT OF |
| | | CARDIOVASCULAR, HEPATIC, |
| | | AND BONE DISEASE |
| III. | 179 | METHODS FOR USING 14266, A HUMAN G |
| | | PROTEIN-COUPLED RECEPTOR |
| IV. | 255 | LIGAND RECEPTORS AND USES THIEREFOR |
| V. | 310 | 52871, A NOVEL HUMAN G PROTEIN |
| | | COUPLED RECEPTOR AND USES THEREOF |
| VI. | 409 | MUSCARINIC RECEPTORS AND USES' THEREFOR |
| VII. | 488 | C7F2- A NOVEL POTASSIUM CHANNEL |
| | | β-SUBUNIT |

### I. 15571, A NOVEL GPCR-LIKE MOLECULE OF THE SECRETIN-LIKE FAMILY AND USES THEREOF

### Background of the Invention

G-protein coupled receptors (GPCRs) constitute a major class of proteins responsible for transducing a signal within a cell (Strosberg (1991) Eur. J. Biochem. 196:1-10; Kerlavage (1991) Curr. Opin. Struct. Biol. 1:394-401; Probst et al. (1992) DNA Cell Biol. 11:1-20; Savarese et al. (1992) Biochem 283:1-9). GPCRs have three structural domains: an amino terminal extracellular domain; a transmembrane domain containing seven transmembrane segments, three extracellular loops, and three intracellular loops; and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

GPCR genes and gene-products are potential causative agents of disease (Spiegel et al. (1993) J. Clin. Invest. 92:1119-1125; McKusick et al. (1993) J. Med. Genet. 30:1-26). Specific defects in the rhodopsin gene and the V2 vasopressin receptor gene have been shown to cause various forms of retinitis pigmentosum (Nathans et al. (1992) Annu. Rev. Genet. 26:403-424) and nephrogenic diabetes insipidus (Holtzman et al. (1993) Hum. Mol. Genet. 2:1201-1204). These receptors are of critical importance to both the central nervous system and peripheral physiological processes. Evolutionary analyses suggest that the ancestor of these proteins originally developed in concert with complex body plans and nervous systems.

In addition to variability among individuals in their responses to drugs, several definable diseases arise from disorders of receptor function or receptor-effector systems. The loss of a receptor in a highly specialized signaling system may cause a relatively limited phenotypic disorder, such as the genetic deficiency of the androgen receptor in the testicular feminization syndrome (Griffin et al. (1995) The Metabolic and Molecular Bases of Inherited Diseases 7:2967-2998). Deficiencies of more widely used signaling systems have a broader spectrum of effects, as are seen in myasthenia gravis or some forms of insulin-resistant diabetes mellitus, which result from autoimmune depletion of nicotinic cholinergic receptors or insulin receptors, respectively. A lesion in a component of a signaling pathway that is used by many receptors can cause a generalized endocrinopathy. Heterozygous deficiency in G5, the G protein that activates adenylyl cyclase in all cells, causes multiple endocrine disorders; the disease is termed pseudohpoparathyroidism type 1a (Spiegel et al. (1995) The Metabolic and Molecular Bases of Inherited Diseases 7:3073-3089). Homozygous deficiency in G5 would presumably be lethal.

The expression of aberrant or ectopic receptors, effectors, or coupling proteins potentially can lead to supersensitivity, subsensitivity, or other untoward responses. Among the most interesting and significant events is the appearance of aberrant receptors as products of oncogenes, which transform otherwise normal cells into malignant cells. Virtually any type of signaling system may have oncogenic potential. G proteins can themselves be oncogenic when either overexpressed or constitutively activated by mutation (Lyons et al (1990) Science 249:655-659). In particular, the calcitonin receptor is a target for treatment of Paget's disease of the bone; the receptor for glucagon-like peptide 1 is a target for non-insulin dependent diabetes mellitus; parathyroid hormone is involved in calcium homeostasis. Antagonists of the parathyroid hormone receptor are of potential clinical use in the treatment of hyperparathyroidism and short-term hypercalcemic states.

The GPCR protein superfamily can be divided into five families: Family I, receptors typified by rhodopsin and the β2-adrenergic receptor and currently represented by over 200 unique members (Dohlman et al. (1991) Annu. Rev. Biochem. 60:653-688); Family II, the parathyroid hormone/calcitonin/secretin receptor family/Class B Secretin-like Family (Juppner et al. (1991) Science 254:1024-1026; Lin et al. (1991) Science 254:1022-1024); Family III, the metabotropic glutamate receptor family (Nakanishi (1992) Science 258 597:603); Family IV, the cAMP receptor family, important in the chemotaxis and development of D. discoideum (Klein et al. (1988) Science 241:1467-1472); and Family V, the fungal mating pheromone receptors such as STE2 (Kurjan (1992) Annu. Rev. Biochem. 61:1097-1129).

G proteins represent a family of heterotrimeric proteins composed of α, β, and γ subunits that bind guanine nucleotides. These proteins are usually linked to cell surface receptors, e.g., receptors containing seven transmembrane segments. Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cAMP (e.g., by activation of adenyl cyclase), diacylglycerol or inositol phosphates. Greater than 20 different types of α-subunits are known in humans. These subunits associate with a smaller pool of β and γ subunits. Examples of mammalian G proteins include Gi, Go, Gq, Gs, and Gt. G proteins are described extensively in Lodish et al. (1995) Molecular Cell Biology (Scientific American Books Inc., New York, N.Y.), the contents of which are incorporated herein by reference. GPCRs, G proteins and G protein-linked effector and second messenger systems have been reviewed in Watson et al., eds. (1994) The G-Protein Linked Receptor Fact Book (Academic Press, NY).

GPCRs are a major target for drug action and development. Accordingly, it is valuable to the field of pharmaceutical development to identify and characterize previously unknown GPCRs. The present invention advances the state of the art by providing previously unidentified human GPCR-like sequences.

### Summary of the Invention

Isolated nucleic acid molecules corresponding to GPCR-like nucleic acid sequences are provided. Additionally, amino acid sequences corresponding to the polynucleotides are encompassed. In particular, the present invention provides for isolated nucleic acid molecules comprising nucleotide sequences encoding the amino acid sequence shown in SEQ ID NO:2. Further provided are GPCR-like polypeptides having an amino acid sequence encoded by a nucleic acid molecule described herein, such as the sequence shown in SEQ ID NO:1.

The present invention also provides vectors and host cells for recombinant expression of the nucleic acid molecules described herein, as well as methods of making such vectors and host cells and for using them for production of the polypeptides or peptides of the invention by recombinant techniques.

The GPCR-like molecules of the present invention find use in identifying compounds that act as agonists and antagonists and modulate the expression of the novel receptors. Furthermore, compounds that modulate expression of the receptors for treatment and diagnosis of GPCR-related disorders are also encompassed. The molecules are useful for the treatment of immune, hematologic, fibrotic, hepatic, and respiratory disorders, including, but not limited to, atopic conditions, such as asthma and allergy, including allergic rhinitis, psoriasis, the effects of pathogen infection, chronic inflammatory diseases, organ-specific autoimmunity, graft rejection, graft versus host disease, cystic fibrosis, and liver fibrosis. Disorders associated with the following cells or tissues are also encompassed: lymph node; spleen; thymus; brain; lung; skeletal muscle; fetal liver; tonsil; colon; heart; liver; peripheral blood mononuclear cells (PBMC); CD34+; bone marrow cells; neonatal umbilical cord blood (CB CD34+); leukocytes from G-CSF treated patients (mPB leukocytes); CD14+ cells; monocytes; hepatic stellate cells; fibrotic liver; kidney; spinal cord; and dermal and lung fibroblasts.

Accordingly, in one aspect, this invention provides isolated nucleic acid molecules encoding GPCR-like proteins or biologically active portions thereof, as well as nucleic acid fragments suitable as primers or hybridization probes for the detection of GPCR-like-encoding nucleic acids. The invention also features isolated or recombinant GPCR-like proteins and polypeptides. Preferred GPCR-like proteins and polypeptides possess at least one biological activity possessed by naturally occurring GPCR-like proteins.

Variant nucleic acid molecules and polypeptides substantially homologous to the nucleotide and amino acid sequence set forth in the Sequence Listing are encompassed by the present invention. Additionally, fragments and substantially homologous fragments of the nucleotide and amino acid sequence are provided.

Antibodies and antibody fragments that selectively bind the GPCR-like polypeptides and fragments are provided. Such antibodies are useful for detecting the presence of receptor protein in cells or tissues. Antibodies can also be used to assess receptor expression in disease states, to assess normal and aberrant subcellular localization of cells in the various tissues in an organism. Antibodies are also useful as diagnostic tools as an immunological marker for aberrant receptor protein.

In one embodiment, the uses can be applied in a therapeutic context in which treatment involves modulating receptor function. An antibody can be used, for example, to block ligand binding. Antibodies can be prepared against specific fragments containing sites required for function or against intact receptor associated with a cell. The GPCR-like modulators include GPCR-like proteins, nucleic acid molecules, peptides, or other small molecules.

The present invention also provides a diagnostic assay for identifying the presence or absence of a genetic lesion or mutation characterized by at least one of the following: (1) aberrant modification or mutation of a gene encoding a GPCR-like protein; (2) misregulation of a gene encoding a GPCR-like protein; and (3) aberrant post-translational modification of a GPCR-like protein, wherein a wild-type form of the gene encodes a protein with a GPCR-like activity.

In another aspect, the invention provides a method for identifying a compound that binds to or modulates the activity of a GPCR-like protein. In general, such methods entail measuring a biological activity of a GPCR-like protein in the presence and absence of a test compound and identifying those compounds that alter the activity of the GPCR-like protein.

The invention also features methods for identifying a compound that modulates the expression of GPCR-like genes by measuring the expression of the GPCR-like sequences in the presence and absence of the compound.

Other features and advantages of the invention will be apparent from the following detailed description and claims.

### DETAILED DESCRIPTION OF THE INVENTION

The present invention provides GPCR-like molecules. By “GPCR-like molecules” is intended a novel human sequence referred to as h15571, and variants and fragments thereof. These full-length gene sequences or fragments thereof are referred to as “GPCR-like” sequences, indicating they share sequence similarity with GPCR genes. Isolated nucleic acid molecules comprising nucleotide sequences encoding the h15571 polypeptide whose amino acid sequence is given in SEQ ID NO:2, or a variant or fragment thereof, are provided. A nucleotide sequence encoding the h15571 polypeptide is set forth in SEQ ID NO:1. The sequences are members of the secretin-like family of G-protein coupled receptors.

The secretin/VIP (vasoactive intestinal polypeptide) family includes receptors for peptides such as secretin, glucagon, glucagon-like peptide 1 (GLP-1), gastric inhibitory peptide, parathyroid hormone, VIP, pituitary adenylate cyclase activating polypeptide (PACAP), calcitonin, and growth hormone releasing hormone. VIP has a wide profile of physiological actions. In the periphery, VIP induces relaxation in smooth muscle, inhibits secretion in certain tissues such as the stomach, stimulates secretion in tissues such as the intestinal epithelium, pancreas, and gall bladder, and modulates activity of cells in the immune system. In the central nervous system, VIP has a wide range of excitatory and inhibitory actions.

Members of the Class B Secretin-like family of GPCRs (Juppner et al. (1991) Science 254:1024-1026; Hamann et al. (1996) Genomics 32:144-147) include: calcitonin receptor, calcitonin gene-related peptide receptor, corticotropin releasing factor receptor types 1 and 2, gastric inhibitory polypeptide receptor, glucagon receptor, glucagon-like peptide 1 receptor, growth hormone-releasing hormone receptor, parathyroid hormone/parathyroid home-related peptide types 1 and 2, pituitary adenylate cyclase activating polypeptide receptor, secretin receptor, vasoactive intestinal peptide receptor types 1 and 2, insects diuretic hormone receptor, Caenorhabditis elegans putative receptor C13B9.4 (Swiss-Prot accession number Q09460), Caenorhabditis elegans putative receptor ZK64.3 (Swiss-Prot accession numbers P30650 and P30649), human leucocyte antigen CD97 (a protein that contains, in its N-terminal section, 3 EGF-like domains) (Swiss-Prot accession number P48960), and mouse cell surface glycoprotein F4/80 (murine EMR1 hormone receptor that contains, in its N-terminal section, 7 EGF-like domains) (GenBank accession number X93328), human EMR1 (EMR1 hormone receptor containing 6 EGF-like domains) (GenBank accession number X81479), BAI1 (a brain-specific p53-target gene containing thrombospondin type 1 repeats) (GenBank accession number AB005297), GPR56 (GenBank accession number AF106858), HE6 (a human receptor having an amino terminal region with identity to highly glycosylated mucin-like cell surface molecules) (GenBank accession number X81892), alpha-latrotxin receptors, and MEGF2 (a human protein containing EGF-like motifs) (GenBank accession number AB011536).

The receptor-like proteins of the invention function as GPCR-like proteins that participate in signaling pathways. As used herein, a “signaling pathway” refers to the modulation (e.g., stimulation or inhibition) of a cellular function/activity upon the binding of a ligand to the GPCR-like protein. Examples of such functions include mobilization of intracellular molecules that participate in a signal transduction pathway, e.g., phosphatidylinositol 4,5-bisphosphate (PIP2), inositol 1,4,5-triphosphate (IP3), and adenylate cyclase; polarization of the plasma membrane; production or secretion of molecules; alteration in the structure of a cellular component; cell proliferation, e.g., synthesis of DNA; cell migration; cell differentiation; and cell survival.

The response mediated by the receptor-like proteins of the invention depends on the type of cell. For example, in some cells, binding of a ligand to the receptor-like protein may stimulate an activity such as release of compounds, gating of a channel, cellular adhesion, migration, differentiation, etc., through phosphatidylinositol or cyclic AMP (cAMP) metabolism and turnover while in other cells, the binding of the ligand will produce a different result. Regardless of the cellular activity/response modulated by the receptor-like protein, it is universal that the protein is a GPCR-like protein and interacts with G proteins to produce one or more secondary signals, in a variety of intracellular signal transduction pathways, e.g., through phosphatidylinositol or cyclic AMP metabolism and turnover, in a cell.

As used herein, “phosphatidylinositol turnover and metabolism” refers to the molecules involved in the turnover and metabolism of phosphatidylinositol 4,5-bisphosphate (PIP2) as well as to the activities of these molecules. PIP2 is a phospholipid found in the cytosolic leaflet of the plasma membrane. Binding of ligand to the receptor activates, in some cells, the plasma-membrane enzyme phospholipase C that in turn can hydrolyze PIP2 to produce 1,2-diacylglycerol (DAG) and inositol 1,4,5-triphosphate (IP3). Once formed, IP3 can diffuse to the endoplasmic reticulum surface where it can bind an IP3 receptor, e.g., a calcium channel protein containing an IP3 binding site. IP3 binding can induce opening of the channel, allowing calcium ions to be released into the cytoplasm. IP3 can also be phosphorylated by a specific kinase to form inositol 1,3,4,5-tetraphosphate (IP4), a molecule that can cause calcium entry into the cytoplasm from the extracellular medium. IP3 and IP4 can subsequently be hydrolyzed very rapidly to the inactive products inositol 1,4-biphosphate (IP2) and inositol 1,3,4-triphosphate, respectively. These inactive products can be recycled by the cell to synthesize PIP2. The other second messenger produced by the hydrolysis of PIP2, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-κB. The language “phosphatidylinositol activity”, as used herein, refers to an activity of PIP2 or one of its metabolites.

Another signaling pathway in which the receptor-like proteins may participate is the cyclic AMP (cAMP) turnover pathway. As used herein, “cAMP turnover and metabolism” refers to the molecules involved in the turnover and metabolism of cAMP as well as to the activities of these molecules. Cyclic AMP is a second messenger produced in response to ligand-induced stimulation of certain G-protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

The disclosed invention relates to methods and compositions for the modulation, diagnosis, and treatment of immune, hematologic, fibrotic, inflammatory, liver, and respiratory disorders. Such immune disorders include, but are not limited to, chronic inflammatory diseases and disorders, inflammatory bowel disease, such as Crohn's disease and ulcerative colitis, rheumatoid arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis), certain viral infections, including HIV, HBV, HCV, and bacterial infections, including tuberculosis and lepromatous leprosy.

Respiratory disorders include, but are not limited to, apnea, asthma, particularly bronchial asthma, berillium disease, bronchiectasis, bronchitis, bronchopneumonia, cystic fibrosis, diphtheria, dyspnea, emphysema, chronic obstructive pulmonary disease, allergic bronchopulmonary aspergillosis, pneumonia, acute pulmonary edema, pertussis, pharyngitis, atelectasis, Wegener's granulomatosis, Legionnaires disease, pleurisy, rheumatic fever, and sinusitis.

Fibrotic disorders or diseases include fibrosis in general, e.g., chronic pulmonary obstructive disease; ideopathic pulmonary fibrosis; crescentic glomerulofibrosis; sarcoidosis; cystic fibrosis; fibrosis/cirrhosis, including cirrhosis secondary to chronic alcoholism, cirrhosis secondary to hepatitis type B or hepatitis type C, and primary biliary cirrhosis; liver disorders disclosed below, particularly liver fibrosis; and other fibrotic diseases; as well as in the treatment of burns and scarring.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, a1-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Hematologic disorders include but are not limited to anemias including chemotherapy-induced anemia, sickle cell and hemolytic anemia, hemophilias including types A and B, leukemias, thalassemias, spherocytosis, Von Willebrand disease, chronic granulomatous disease, glucose-6-phosphate dehydrogenase deficiency, thrombosis, clotting factor abnormalities and deficiencies including factor VIII and IX deficiencies, hemarthrosis, hematemesis, hematomas, hematuria, hemochromatosis, hemoglobinuria, hemolytic-uremic syndrome, thrombocytopenias including chemotherapy-induced thrombocytopenia, HIV-associated thrombocytopenia, hemorrhagic telangiectasia, idiopathic thrombocytopenic purpura, thrombotic microangiopathy, hemosiderosis, chemotherapy induced neutropenias. Other disorders include polycythemias, including polycythemia vera, secondary polycythemia, and relative polycythemia, neutropenias, including chemotherapy-induced neutropenia, chronic idiopathic neutropenia, Felty's syndrome, neutropenias resulting from acute infectious diseases, lymphoma or aleukemic lymphocytic leukemia with neutropenia, myelodysplastic syndrome, rheumatic disease induced neutropenias such as systemic lupus, erythematosus, rheumatoid arthritis, and polymyositis.

A novel human GPCR-like gene sequence, referred to as h15571, is provided. This gene sequence and variants and fragments thereof are encompassed by the term “GPCR-like” molecules or sequences as used herein. The GPCR-like sequences find use in modulating a GPCR-like function. By “modulating” is intended the upregulating or downregulating of a response. That is, the compositions of the invention affect the targeted activity in either a positive or negative fashion.

The GPCR-like gene, designated clone h15571, was identified in human thymus and spleen cDNA libraries. Clone h15571 encodes an approximately 6.09 Kb mRNA transcript having the corresponding cDNA set forth in SEQ ID NO:1. This transcript has a 4014-nucleotide open reading frame (nucleotides 366-4379 of SEQ ID NO:1), which encodes a 1338 amino acid polypeptide (SEQ ID NO:2).

An analysis of the full-length h15571 polypeptide (SEQ ID NO:2) predicts that the N-terminal 33 amino acids represent a signal peptide. Thus, the mature polypeptide is predicted to be 1305 amino acids in length (aa 34-1338 of SEQ ID NO:2). Transmembrane domains (TM) at the following positions of the sequence set forth in SEQ ID NO:2 were predicted by MEMSAT as well as by alignment with members of the secretin-like family of GPCRs and visual inspection; TM I, 772-793 of SEQ ID NO:2, TM II, 807-826 of SEQ ID NO:2; TM III, 836-855 of SEQ ID NO:2; TM IV, 887-904 of SEQ ID NO:2; TM V, 925-947 of SEQ ID NO:2; TM VI 1021-1040 of SEQ ID NO:2; and TM VII, 1048-1066 of SEQ ID NO:2. Based on the predicted positions of TM I-VII, the predicted positions of the N-terminus extracellular domain (EC), the extracellular loops (EL) I-III, the intracellular loops (IL) I-III, and the C-terminus intracellular domain (IC) are as follows as shown in the sequence in SEQ ID NO:2: EC, about aa 34-771; EL I, about aa 827-835; EL II, about aa 905-924; EL III, about aa 1041-1048; IL I, about aa 794-806; IL II, about aa 856-886; IL III, about aa 948-1020; and IC, about aa 1067-1338. Prosite program analysis was used to predict various sites within the h15571 protein. N-glycosylation sites were predicted at aa 84-87, 101-104, 162-165, 207-210, 275-278, 336-339, 436-439, 602-605, 659-662, 690-693, 737-740, and 794-797 of SEQ ID NO:2. A glycosaminoglycan attachment site was predicted at aa 684-687 of SEQ ID NO:2. Protein Kinase C phosphorylation sites were predicted at aa 40-42, 43-45, 253-255, 338-340, 400-402, 598-600, 660-662, 698-700, 797-799, 801-803, 865-867, 976-978, 997-999, 1041-1043, 1079-1081, 1116-1118, 1233-1235, 1279-1281, and 1290-1292 of SEQ ID NO:2. Casein Kinase II phosphorylation sites were predicted at aa 69-72, 108-111, 231-234, 456-459, 1225-1228, and 1251-1254 of SEQ ID NO:2. N-myristoylation sites were predicted at aa 36-41, 53-58, 80-85, 98-103, 126-131, 145-150, 165-170, 295-300, 319-324, 392-397, 555-560, 566-571, 682-687, 722-727, 763-768, 825-830, 900-905, 961-966, 990-995, 1016-1021, 1055-1060, 1150-1155, 1163-1168, 1206-1211, 1220-1225, 1232-1237, 1255-1260, 1270-1275, 1304-1309, 1318-1323, and 1325-1330 of SEQ ID NO:2. Amidation sites were predicted at aa 4-7, 668-671, and 1178-1181 of SEQ ID NO:2. A prokaryotic membrane lipoproptein lipid attachment site was predicted at aa 676-686 of SEQ ID NO:2. An RGD cell attachment sequence was predicted at aa 362-364 of SEQ ID NO:2.

Domain matches using HMMER 2.1.1 (Washington University School of Medicine) indicated the presence of several key protein domains. A search of the HMM database using Pfam (Protein Family) indicated the presence of five leucine rich repeat domains, residing at aa 85-108, 109-132, 133-156, 157-180, and 604-630 of SEQ ID NO:2. A leucine rich repeat C-terminal domain was identified at aa 190-240 of SEQ ID NO:2. An immunoglobulin domain was identified at aa 261-330 of SEQ ID NO:2. A latrophilin/CL-1-like GPS domain was identified at aa 706-758 of SEQ ID NO:2. A search of the HMM database using SMART (Simple Modular Architecture Research Tool) revealed the following domain matches: four leucine rich repeat typical-2 subfamily domains were identified, residing at aa 82-106, 107-130, 131-154, and 155-178 of SEQ ID NO:2. Two leucine rich repeat SDS22-like subfamily domains were identified, residing at aa 107-128 and 131-157 of SEQ ID NO:2. A leucine rich repeat ribonuclease inhibitor type domain was identified at aa 131-157 of SEQ ID NO:2. A leucine rich repeat C-terminal domain was identified at aa 190-240 of SEQ ID NO:2. An immunoglobulin C-2 type domain was identified at aa 259-335 of SEQ ID NO:2. An immunoglobulin 3-C domain was identified at aa 253-346 of SEQ ID NO:2. A hormone receptor domain was identified at aa 349-426 of SEQ ID NO:2. A G-protein coupled receptor proteolytic site domain was identified at aa 706-758 of SEQ ID NO:2.

ProDom analysis indicates that the h15571 polypeptide has regions sharing similarity with other GPCRs. Amino acid residues 367-1077 of SEQ ID NO:2 share approximately 33% identity with portions of a consensus sequence for Family II GPCRs including calcitonin receptor (CALR), corticotrophin releasing factor receptor (CRFR), and parathyroid hormone/parathyroid hormone related receptor (PTRR). ProDom analysis also indicates that the h15571 polypeptide has regions sharing similarity with several other proteins. Amino acid residues 84-131, 85-155, 110-179, and 134-187 of SEQ ID NO:2 share approximately 43%, 36%, 34%, and 24% identity with amino acid residues 26-73, 3-73, 4-73, and 4-57, respectively, of a consensus sequence for the rat MEGF5 glycoprotein EGF-like domain. Amino acid residues 89-237 of SEQ ID NO:2 share approximately 30% identity with a consensus sequence for a family that groups together the CYAA, ESA8, and CD14 proteins. Amino acid residues 182-356 of SEQ ID NO:2 share approximately 21% identity with a protein encoded by the C. elegans YK6G3.3, which also has multiple leucine-rich repeats. Amino acid residues 88-221 of SEQ ID NO:2 share approximately 32% identity with a leucine-rich repeat protein. Amino acid residues 37-176 of SEQ ID NO:2 share approximately 23% identity with the C. elegans C44H4.1 protein (Accession No. CABD1867). Amino acid residues 180-237 and 860-883 of SEQ ID NO:2 share an identity of approximately 37% and 45%, respectively, with aa residues 4-64 and 166-187 of the human KIAA0644 protein.

An alignment of the seven transmembrane (7 TM) domains of h15571 with several members of the Class B secretin-like family of GPCRs is described herein. Based on sequence homology of the 7 TM domains, h15571 appears to be related to a subfamily of the Class B Secretin-like Family of GPCRs. The members of this subfamily share similar sequences in the 7 TM domains that are distinct from other members of the secretin-like family. This subfamily includes CD97, EMR1, BAI1, GPR56, HE6, alpha-latrotoxin receptors, MEGF2, and two putative GPCRs identified by sequencing the C. elegans genome (GenBank™ accession numbers Z54306 and U39848). The members of this subfamily are further characterized by the presence of an extremely large N-terminal extracellular region (containing, for example, several hundred amino acid residues, e.g., at least 200, 250, 300, 350, 400, 450, 500, 550, 600, 650, 700, 750, 800, 850, 900, 950, or 1000, or more amino acid residues). The members of this family of molecules also share a box of four conserved cysteine residues in the N-terminus of TM I, which is the purported area of proteolytic cleavage for at least two members, CD97 and the latrotoxin receptor. Further, there is a cellular adhesion domain (e.g., mucin-like, thrombospondin-like, EGF-like, or lectin-like) seen in the N-terminus of members of this subfamily. See Liu et al. (1999) Genomics 55:296-305. h15571 shares with other members of this subfamily a large N-terminal extracellular region (approximately 738 aa residues), but differs by the presence of two of the four conserved cysteine residues in the N-terminus of TM I. Further, no known cellular adhesion domain has been identified in the N-terminus of h15571. The 7 TM region of h15571 (from about aa 772 to about 1066 of SEQ ID NO:2) shows the highest homology (approximately 19.4%) with the CD97 7 TM region.

The GPCR-like sequences of the invention are members of a family of molecules (the “secretin-like receptor family”) having conserved functional features. The term “family” or “subfamily” when referring to the proteins and nucleic acid molecules of the invention is intended to mean two or more proteins or nucleic acid molecules having sufficient amino acid or nucleotide sequence identity as defined herein. Such family members can be naturally occurring and can be from either the same or different species. For example, a family can contain a first protein of murine origin and a homologue of that protein of human origin, as well as a second, distinct protein of human origin and a murine homologue of that protein. Members of a family may also have common functional characteristics.

Preferred GPCR-like polypeptides of the present invention have an amino acid sequence sufficiently identical to the amino acid sequence of SEQ ID NO:2. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain (e.g., leucine rich repeat domain, immunoglobulin domain, transmembrane receptor domain, G-protein receptor domain, etc.) and/or common functional activity. For example, amino acid or nucleotide sequences that contain a common structural domain having at least about 45%, 55%, 60% or 65% identity, preferably at least about 70%, 75%, 80%, identity, more preferably at least about 85%, 90%, 91%, 92%, 93%, 94%, 95%, 96%, 97%, 98%, or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to GPCR-like nucleic acid molecules of the invention. BLAST protein searches can be performed with the XBLAST program, score=50, wordlength=3, to obtain amino acid sequences homologous to GPCR-like protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. An additional preferred program is the Pairwise Alignment Program (Sequence Explorer), using default parameters.

Accordingly, another embodiment of the invention features isolated GPCR-like proteins and polypeptides having a GPCR-like protein activity. As used interchangeably herein, a “GPCR-like protein activity”, “biological activity of a GPCR-like protein”, or “functional activity of a GPCR-like protein” refers to an activity exerted by a GPCR-like protein, polypeptide, or nucleic acid molecule on a GPCR-like responsive cell as determined in vivo, or in vitro, according to standard assay techniques. A GPCR-like activity can be a direct activity, such as an association with or an enzymatic activity on a second protein, or an indirect activity, such as a cellular signaling activity mediated by interaction of the GPCR-like protein with a second protein. In a preferred embodiment, a GPCR-like activity includes at least one or more of the following activities: (1) modulating (i.e., stimulating and/or enhancing or inhibiting) cellular proliferation, differentiation, and/or function (in the cells and organs in which it is expressed, for example, lymph node; spleen; thymus; brain; lung; skeletal muscle; fetal liver; tonsil; colon; heart; liver; peripheral blood mononuclear cells (PBMC); CD34+; bone marrow cells; neonatal umbilical cord blood (CB CD34+); leukocytes from G-CSF treated patients (mPB leukocytes); CD14+ cells; monocytes; hepatic stellate cells; fibrotic liver; kidney; spinal cord; dermal and lung fibroblasts; and the K562, HEK 293, Jurkat, and HL60 cell lines; (2) modulating a GPCR-like response; (3) modulating an inflammatory or immune response; (4) modulating a respiratory response; and (5) binding a GPCR-like receptor ligand.

An “isolated” or “purified” GPCR-like nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated GPCR-like nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A GPCR-like protein that is substantially free of cellular material includes preparations of GPCR-like protein having less than about 30%, 20%, 10%, or 5% (by dry weight) of non-GPCR-like protein (also referred to herein as a “contaminating protein”). When the GPCR-like protein or biologically active portion thereof is recombinantly produced, preferably, culture medium represents less than about 30%, 20%, 10%, or 5% of the volume of the protein preparation. When GPCR-like protein is produced by chemical synthesis, preferably the protein preparations have less than about 30%, 20%, 10%, or 5% (by dry weight) of chemical precursors or non-GPCR-like chemicals.

Various aspects of the invention are described in further detail in the following subsections.

I. Isolated Nucleic Acid Molecules

One aspect of the invention pertains to isolated nucleic acid molecules comprising nucleotide sequences encoding GPCR-like proteins and polypeptides or biologically active portions thereof, as well as nucleic acid molecules sufficient for use as hybridization probes to identify GPCR-like-encoding nucleic acids (e.g., GPCR-like mRNA) and fragments for use as PCR primers for the amplification or mutation of GPCR-like nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences encoding the GPCR-like proteins of the present invention include the sequence set forth in SEQ ID NO:1. By “complement” is intended a nucleotide sequence that is sufficiently complementary to a given nucleotide sequence such that it can hybridize to the given nucleotide sequence to thereby form a stable duplex. The corresponding amino acid sequence for the polypeptide encoded by these nucleotide sequences is set forth in SEQ ID NO:2.

Nucleic acid molecules that are fragments of these GPCR-like nucleotide sequences are also encompassed by the present invention. By “fragment” is intended a portion of the nucleotide sequence encoding a GPCR-like protein. A fragment of a GPCR-like nucleotide sequence may encode a biologically active portion of a GPCR-like protein, or it may be a fragment that can be used as a hybridization probe or PCR primer using methods disclosed below. A biologically active portion of a GPCR-like protein can be prepared by isolating a portion of one of the GPCR-like nucleotide sequences of the invention, expressing the encoded portion of the GPCR-like protein (e.g., by recombinant expression in vitro), and assessing the activity of the encoded portion of the GPCR-like protein. Nucleic acid molecules that are fragments of a GPCR-like nucleotide sequence comprise at least about 15, 20, 50, 75, 100, 200, 300, 350, 400, 450, 500, 550, 600, 650, 700, 750, 800, 850, 900, 950, 1000, 1250, 1500, 1750, 2000, 2500, 3000, 3500, 4000, 4500, 5000, 5250, 5500, 5750, or 6000 nucleotides, or up to the number of nucleotides present in a full-length GPCR-like nucleotide sequence disclosed herein (6090 nucleotides for the h15571 sequence set forth in SEQ ID NO:1) depending upon the intended use.

It is understood that isolated fragments include any contiguous sequence not disclosed prior to the invention as well as sequences that are substantially the same and which are not disclosed. Accordingly, if an isolated fragment is disclosed prior to the present invention, that fragment is not intended to be encompassed by the invention. When a sequence is not disclosed prior to the present invention, an isolated nucleic acid fragment is at least about 12, 15, 20, 25, or 30 contiguous nucleotides. Other regions of the nucleotide sequence may comprise fragments of various sizes, depending upon potential homology with previously disclosed sequences.

A fragment of a GPCR-like nucleotide sequence that encodes a biologically active portion of a GPCR-like protein of the invention will encode at least about 15, 25, 30, 50, 75, 100, 125, 150, 175, 200, 300, 400, 500, 600, 700, 800, 900, 1000, 1050, 1100, 1150, 1200, 1250, 1300 contiguous amino acids, or up to the total number of amino acids present in a full-length GPCR-like polypeptide of the invention (1338 amino acids for the full-length h15571 protein set forth in SEQ ID NO:2). Fragments of a GPCR-like nucleotide sequence that are useful as hybridization probes for PCR primers generally need not encode a biologically active portion of a GPCR-like protein.

Nucleic acid molecules that are variants of the GPCR-like nucleotide sequences disclosed herein are also encompassed by the present invention. “Variants” of the GPCR-like nucleotide sequences include those sequences that encode the GPCR-like proteins disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the GPCR-like proteins disclosed in the present invention as discussed below. Generally, nucleotide sequence variants of the invention will have at least about 45%, 55%, 60%, 65%, 70%, 75%, 80%, 85%, 90%, 91%, 92%, 93%, 94%, 95%, 96%, 97%, 98%, or 99% identity to a particular nucleotide sequence disclosed herein. A variant GPCR-like nucleotide sequence will encode a GPCR-like protein that has an amino acid sequence having at least about 45%, 55%, 60%, 65%, 70%, 75%, 80%, 85%, 90%, 91%, 92%, 93%, 94%, 95%, 96%, 97%, 98%, or 99% identity to the amino acid sequence of a GPCR-like protein disclosed herein.

In addition to the GPCR-like nucleotide sequence shown in SEQ ID NO:1 and the nucleotide sequence of the cDNA of ATCC PTA-1660, it will be appreciated by those skilled in the art that DNA sequence polymorphisms that lead to changes in the amino acid sequences of GPCR-like proteins may exist within a population (e.g., the human population). Such genetic polymorphism in a GPCR-like gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a GPCR-like protein, preferably a mammalian GPCR-like protein. As used herein, the phrase “allelic variant” refers to a nucleotide sequence that occurs at a GPCR-like locus or to a polypeptide encoded by the nucleotide sequence. Such natural allelic variations can typically result in 1-5% variance in the nucleotide sequence of the GPCR-like gene. Any and all such nucleotide variations and resulting amino acid polymorphisms or variations in a GPCR-like sequence that are the result of natural allelic variation and that do not alter the functional activity of GPCR-like proteins are intended to be within the scope of the invention.

Moreover, nucleic acid molecules encoding GPCR-like proteins from other species (GPCR-like homologues), that have a nucleotide sequence differing from that of the GPCR-like sequences disclosed herein, are intended to be within the scope of the invention. For example, nucleic acid molecules corresponding to natural allelic variants and homologues of the human GPCR-like cDNA of the invention can be isolated based on their identity to the human GPCR-like nucleic acid disclosed herein using the human cDNA, or a portion thereof, as a hybridization probe according to standard hybridization techniques under stringent hybridization conditions as disclosed below.

In addition to naturally-occurring allelic variants of the GPCR-like sequences that may exist in the population, the skilled artisan will further appreciate that changes can be introduced by mutation into the nucleotide sequences of the invention thereby leading to changes in the amino acid sequence of the encoded GPCR-like proteins, without altering the biological activity of the GPCR-like proteins. Thus, an isolated nucleic acid molecule encoding a GPCR-like protein having a sequence that differs from that of SEQ ID NO:2 can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of a GPCR-like protein (e.g., the sequence of SEQ ID NO:2) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, such as the 7 transmembrane receptor domains (i.e., TM I, 772-793, TM II, 807-826; TM III, 836-855; TM IV, 887-904; TM V, 925-947; TM VI 1021-1040; and TM VII, 1048-1066 of SEQ ID NO:2), where such residues are essential for protein activity.

Alternatively, variant GPCR-like nucleotide sequences can be made by introducing mutations randomly along all or part of a GPCR-like coding sequence, such as by saturation mutagenesis, and the resultant mutants can be screened for GPCR-like biological activity to identify mutants that retain activity. Following mutagenesis, the encoded protein can be expressed recombinantly, and the activity of the protein can be determined using standard assay techniques.

Thus the nucleotide sequences of the invention include the sequences disclosed herein as well as fragments and variants thereof. The GPCR-like nucleotide sequences of the invention, and fragments and variants thereof, can be used as probes and/or primers to identify and/or clone GPCR-like homologues in other cell types, e.g., from other tissues, as well as GPCR-like homologues from other mammals. Such probes can be used to detect transcripts or genomic sequences encoding the same or identical proteins. These probes can be used as part of a diagnostic test kit for identifying cells or tissues that misexpress a GPCR-like protein, such as by measuring levels of a GPCR-like-encoding nucleic acid in a sample of cells from a subject, e.g., detecting GPCR-like mRNA levels or determining whether a genomic GPCR-like gene has been mutated or deleted.

In this manner, methods such as PCR, hybridization, and the like can be used to identify such sequences having substantial identity to the sequences of the invention. See, for example, Sambrook et al. (1989) Molecular Cloning: Laboratory Manual (2d ed., Cold Spring Harbor Laboratory Press, Plainview, N.Y.) and Innis, et al. (1990) PCR Protocols: A Guide to Methods and Applications (Academic Press, NY). GPCR-like nucleotide sequences isolated based on their sequence identity to the GPCR-like nucleotide sequences set forth herein or to fragments and variants thereof are encompassed by the present invention.

In a hybridization method, all or part of a known GPCR-like nucleotide sequence can be used to screen cDNA or genomic libraries. Methods for construction of such cDNA and genomic libraries are generally known in the art and are disclosed in Sambrook et al. (1989) Molecular Cloning: A Laboratory Manual (2d ed., Cold Spring Harbor Laboratory Press, Plainview, N.Y.). The so-called hybridization probes may be genomic DNA fragments, cDNA fragments, RNA fragments, or other oligonucleotides, and may be labeled with a detectable group such as 32P, or any other detectable marker, such as other radioisotopes, a fluorescent compound, an enzyme, or an enzyme co-factor. Probes for hybridization can be made by labeling synthetic oligonucleotides based on the known GPCR-like nucleotide sequence disclosed herein. Degenerate primers designed on the basis of conserved nucleotides or amino acid residues in a known GPCR-like nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, or 400 consecutive nucleotides of a GPCR-like nucleotide sequence of the invention or a fragment or variant thereof. Preparation of probes for hybridization is generally known in the art and is disclosed in Sambrook et al. (1989) Molecular Cloning: A Laboratory Manual (2d ed., Cold Spring Harbor Laboratory Press, Plainview, N.Y.), herein incorporated by reference.

For example, in one embodiment, a previously unidentified GPCR-like nucleic acid molecule hybridizes under stringent conditions to a probe that is a nucleic acid molecule comprising one of the GPCR-like nucleotide sequences of the invention or a fragment thereof. In another embodiment, the previously unknown GPCR-like nucleic acid molecule is at least about 300, 325, 350, 375, 400, 425, 450, 500, 550, 600, 650, 700, 800, 900, 1000, 2000, 3000, 4000, 5000, or 6000 nucleotides in length and hybridizes under stringent conditions to a probe that is a nucleic acid molecule comprising one of the GPCR-like nucleotide sequences disclosed herein or a fragment thereof.

Accordingly, in another embodiment, an isolated previously unknown GPCR-like nucleic acid molecule of the invention is at least about 300, 325, 350, 375, 400, 425, 450, 500, 550, 600, 650, 700, 800, 900, 1000, 1,100, 1,200, 1,300, or 1,400 nucleotides in length and hybridizes under stringent conditions to a probe that is a nucleic acid molecule comprising one of the nucleotide sequences of the invention, preferably the coding sequence set forth in SEQ ID NO:1, the cDNA of ATCC PTA-1660, or a complement, fragment, or variant thereof.

As used herein, the term “hybridizes under stringent conditions” is intended to describe conditions for hybridization and washing under which nucleotide sequences having at least about 60%, 65%, 70%, preferably 75% identity to each other typically remain hybridized to each other. Such stringent conditions are known to those skilled in the art and can be found in Current Protocols in Molecular Biology (John Wiley & Sons, New York (1989)), 6.3.1-6.3.6. A preferred, non-limiting example of stringent hybridization conditions is hybridization in 6× sodium chloride/sodium citrate (SSC) at about 45 C, followed by one or more washes in 0.2×SSC, 0.1% SDS at 50-65 C. In another preferred embodiment, stringent conditions comprise hybridization in 6×SSC at 42 C, followed by washing with 1×SSC at 55 C. Preferably, an isolated nucleic acid molecule that hybridizes under stringent conditions to a GPCR-like sequence of the invention corresponds to a naturally occurring nucleic acid molecule. As used herein, a “naturally occurring” nucleic acid molecule refers to an RNA or DNA molecule having a nucleotide sequence that occurs in nature (e.g., encodes a natural protein).

Thus, in addition to the GPCR-like nucleotide sequences disclosed herein and fragments and variants thereof, the isolated nucleic acid molecules of the invention also encompass homologous DNA sequences identified and isolated from other cells and/or organisms by hybridization with entire or partial sequences obtained from the GPCR-like nucleotide sequences disclosed herein or variants and fragments thereof.

The present invention also encompasses antisense nucleic acid molecules, i.e., molecules that are complementary to a sense nucleic acid encoding a protein, e.g., complementary to the coding strand of a double-stranded cDNA molecule, or complementary to an mRNA sequence. Accordingly, an antisense nucleic acid can hydrogen bond to a sense nucleic acid. The antisense nucleic acid can be complementary to an entire GPCR-like coding strand, or to only a portion thereof, e.g., all or part of the protein coding region (or open reading frame). An antisense nucleic acid molecule can be antisense to a noncoding region of the coding strand of a nucleotide sequence encoding a GPCR-like protein. The noncoding regions are the 5′ and 3′ sequences that flank the coding region and are not translated into amino acids.

Given the coding-strand sequence encoding a GPCR-like protein disclosed herein (e.g., the coding-strand sequence of SEQ ID NO:1), antisense nucleic acids of the invention can be designed according to the rules of Watson and Crick base pairing. The antisense nucleic acid molecule can be complementary to the entire coding region of GPCR-like mRNA, but more preferably is an oligonucleotide that is antisense to only a portion of the coding or noncoding region of GPCR-like mRNA. For example, the antisense oligonucleotide can be complementary to the region surrounding the translation start site of GPCR-like mRNA. An antisense oligonucleotide can be, for example, about 5, 10, 15, 20, 25, 30, 35, 40, 45, or 50 nucleotides in length. An antisense nucleic acid of the invention can be constructed using chemical synthesis and enzymatic ligation procedures known in the art.

For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, including, but not limited to, for example e.g., phosphorothioate derivatives and acridine substituted nucleotides. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When used therapeutically, the antisense nucleic acid molecules of the invention are typically administered to a subject or generated in situ such that they hybridize with or bind to cellular mRNA and/or genomic DNA encoding a GPCR-like protein to thereby inhibit expression of the protein, e.g., by inhibiting transcription and/or translation. An example of a route of administration of antisense nucleic acid molecules of the invention includes direct injection at a tissue site. Alternatively, antisense nucleic acid molecules can be modified to target selected cells and then administered systemically. For example, antisense molecules can be linked to peptides or antibodies to form a complex that specifically binds to receptors or antigens expressed on a selected cell surface. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes, which are catalytic RNA molecules with ribonuclease activity that are capable of cleaving a single-stranded nucleic acid, such as an mRNA, to which they have a complementary region. Ribozymes (e.g., hammerhead ribozymes (described in Haselhoff and Gerlach (1988) Nature 334:585-591)) can be used to catalytically cleave GPCR-like mRNA transcripts to thereby inhibit translation of GPCR-like mRNA. A ribozyme having specificity for a GPCR-like-encoding nucleic acid can be designed based upon the nucleotide sequence of a GPCR-like cDNA disclosed herein (e.g., SEQ ID NO:1). See, e.g., Cech et al., U.S. Pat. No. 4,987,071; and Cech et al., U.S. Pat. No. 5,116,742. Alternatively, GPCR-like mRNA can be used to select a catalytic RNA having a specific ribonuclease activity from a pool of RNA molecules. See, e.g., Bartel and Szostak (1993) Science 261:1411-1418.

The invention also encompasses nucleic acid molecules that form triple helical structures. For example, GPCR-like gene expression can be inhibited by targeting nucleotide sequences complementary to the regulatory region of the GPCR-like protein (e.g., the GPCR-like promoter and/or enhancers) to form triple helical structures that prevent transcription of the GPCR-like gene in target cells. See generally Helene (1991) Anticancer Drug Des. 6(6):569; Helene (1992) Ann. N.Y. Acad. Sci. 660:27; and Maher (1992) Bioassays 14(12):807.

In preferred embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid-phase peptide synthesis protocols as described, for example, in Hyrup et al. (1996), supra; Perry-O'Keefe et al. (1996) Proc. Natl. Acad. Sci. USA 93:14670.

PNAs of a GPCR-like molecule can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA-directed PCR clamping; as artificial restriction enzymes when used in combination with other enzymes, e.g., S1 nucleases (Hyrup (1996), supra); or as probes or primers for DNA sequence and hybridization (Hyrup (1996), supra; Perry-O'Keefe et al. (1996), supra).

In another embodiment, PNAs of a GPCR-like molecule can be modified, e.g., to enhance their stability, specificity, or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra; Finn et al. (1996) Nucleic Acids Res. 24(17):3357-63; Mag et al. (1989) Nucleic Acids Res. 17:5973; and Peterson et al. (1975) Bioorganic Med. Chem. Lett. 5:1119.

II. Isolated GPCR-Like Proteins and Anti-GPCR-Like Antibodies

GPCR-like proteins are also encompassed within the present invention. By “GPCR-like protein” is intended a protein comprising the amino acid sequence set forth in SEQ ID NO:2, as well as fragments, biologically active portions, and variants thereof.

“Fragments” or “biologically active portions” include polypeptide fragments suitable for use as immunogens to raise anti-GPCR-like antibodies. Fragments include peptides comprising amino acid sequences sufficiently identical to or derived from the amino acid sequence of a GPCR-like protein, or partial-length protein, of the invention and exhibiting at least one activity of a GPCR-like protein, but which include fewer amino acids than the full-length GPCR-like protein (SEQ ID NO:2) disclosed herein. Typically, biologically active portions comprise a domain or motif with at least one activity of the GPCR-like protein. A biologically active portion of a GPCR-like protein can be a polypeptide which is, for example, 10, 25, 50, 100 or more amino acids in length. Such biologically active portions can be prepared by recombinant techniques and evaluated for one or more of the functional activities of a native GPCR-like protein. As used here, a fragment comprises at least 7 contiguous amino acids of SEQ ID NO:2. The invention encompasses other fragments, however, such as any fragment in the protein greater than 8, 9, 10, or 11 amino acids.

Biologically active fragments (peptides which are, for example, 5, 7, 10, 12, 15, 20, 30, 35, 36, 37, 38, 39, 40, 50, 100 or more amino acids in length) can comprise, for example, a domain or motif, e.g., leucine rich repeats and leucine rich repeat C-terminal domains, latrophilin/CL-1-like GPS domain, immunoglobulin domain, 7 transmembrane receptor domain, and sites for glycosylation, protein kinase C phosphorylation, casein kinase II phosphorylation, glycosaminoglycan attachment, amidation, N-myristoylation, prokaryotic membrane lipoprotein lipid attachment, and RGD cell attachment. Further possible fragments include sites important for cellular and subcellular targeting. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. Such domains or motifs and their sub-fragments can be identified by means of routine computerized homology searching procedures.

The invention also provides fragments with immunogenic properties. These contain an epitope-bearing portion of the GPCR-like polypeptides of the invention. These epitope-bearing peptides are useful to raise antibodies that bind specifically to a GPCR-like polypeptide or region or fragment. These peptides can contain at least 10, 12, at least 14, or between at least about 15 to about 30 amino acids. Non-limiting examples of antigenic polypeptides that can be used to generate antibodies include but are not limited to peptides derived from an extracellular site. However, intracellularly-made antibodies (“intrabodies”) are also encompassed, which would recognize intracellular peptide regions. The epitope-bearing GPCR-like polypeptides may be produced by any conventional means (Houghten, R. A. (1985) Proc. Natl. Acad. Sci. USA 82:5131-5135). Simultaneous multiple peptide synthesis is described in U.S. Pat. No. 4,631,211.

By “variants” is intended proteins or polypeptides having an amino acid sequence that is at least about 45%, 55%, 60%, 65%, preferably about 70%, 75%, 80%, 85%, 90%, 91%, 92%, 93%, 94%, 95%, 96%, 97%, 98%, or 99% identical to the amino acid sequence of SEQ ID NO:2. Variants also include polypeptides encoded by the cDNA insert of the plasmid deposited with ATCC as Patent Deposit No. PTA-1660, or polypeptides encoded by a nucleic acid molecule that hybridizes to the nucleic acid molecule of SEQ ID NO:1, or a complement thereof, under stringent conditions. Such variants generally retain the functional activity of the GPCR-like proteins of the invention. Variants include polypeptides that differ in amino acid sequence due to natural allelic variation or mutagenesis.

The invention also provides GPCR-like chimeric or fusion proteins. As used herein, a GPCR-like “chimeric protein” or “fusion protein” comprises a GPCR-like polypeptide operably linked to a non-GPCR-like polypeptide. A “GPCR-like polypeptide” refers to a polypeptide having an amino acid sequence corresponding to a GPCR-like protein, whereas a “non-GPCR-like polypeptide” refers to a polypeptide having an amino acid sequence corresponding to a protein that is not substantially identical to the GPCR-like protein, e.g., a protein that is different from the GPCR-like protein and which is derived from the same or a different organism. Within a GPCR-like fusion protein, the GPCR-like polypeptide can correspond to all or a portion of a GPCR-like protein, preferably at least one biologically active portion of a GPCR-like protein. Within the fusion protein, the term “operably linked” is intended to indicate that the GPCR-like polypeptide and the non-GPCR-like polypeptide are fused in-frame to each other. The non-GPCR-like polypeptide can be fused to the N-terminus or C-terminus of the GPCR-like polypeptide.

One useful fusion protein is a GST-GPCR-like fusion protein in which the GPCR-like sequences are fused to the C-terminus of the GST sequences. Such fusion proteins can facilitate the purification of recombinant GPCR-like proteins.

In yet another embodiment, the fusion protein is a GPCR-like-immunoglobulin fusion protein in which all or part of a GPCR-like protein is fused to sequences derived from a member of the immunoglobulin protein family. The GPCR-like-immunoglobulin fusion proteins of the invention can be incorporated into pharmaceutical compositions and administered to a subject to inhibit an interaction between a GPCR-like ligand and a GPCR protein on the surface of a cell, thereby suppressing GPCR-like-mediated signal transduction in vivo. The GPCR-immunoglobulin fusion proteins can be used to affect the bioavailability of a GPCR-like cognate ligand. Inhibition of the GPCR-like ligand/GPCR-like interaction may be useful therapeutically, both for treating proliferative and differentiative disorders and for modulating (e.g., promoting or inhibiting) cell survival. Moreover, the GPCR-like-immunoglobulin fusion proteins of the invention can be used as immunogens to produce anti-GPCR-like antibodies in a subject, to purify GPCR-like ligands, and in screening assays to identify molecules that inhibit the interaction of a GPCR-like protein with a GPCR-like ligand.

Preferably, a GPCR-like chimeric or fusion protein of the invention is produced by standard recombinant DNA techniques. For example, DNA fragments coding for the different polypeptide sequences may be ligated together in-frame, or the fusion gene can be synthesized, such as with automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, e.g., Ausubel et al., eds. (1995) Current Protocols in Molecular Biology) (Greene Publishing and Wiley-Interscience, NY). Moreover, a GPCR-like-encoding nucleic acid can be cloned into a commercially available expression vector such that it is linked in-frame to an existing fusion moiety.

Variants of the GPCR-like proteins can function as either GPCR-like agonists (mimetics) or as GPCR-like antagonists. Variants of the GPCR-like protein can be generated by mutagenesis, e.g., discrete point mutation or truncation of the GPCR-like protein. An agonist of the GPCR-like protein can retain substantially the same, or a subset, of the biological activities of the naturally occurring form of the GPCR-like protein. An antagonist of the GPCR-like protein can inhibit one or more of the activities of the naturally occurring form of the GPCR-like protein by, for example, competitively binding to a downstream or upstream member of a cellular signaling cascade that includes the GPCR-like protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the GPCR-like proteins.

Variants of a GPCR-like protein that function as either GPCR-like agonists or as GPCR-like antagonists can be identified by screening combinatorial libraries of mutants, e.g., truncation mutants, of a GPCR-like protein for GPCR-like protein agonist or antagonist activity. In one embodiment, a variegated library of GPCR-like variants is generated by combinatorial mutagenesis at the nucleic acid level and is encoded by a variegated gene library. A variegated library of GPCR-like variants can be produced by, for example, enzymatically ligating a mixture of synthetic oligonucleotides into gene sequences such that a degenerate set of potential GPCR-like sequences is expressible as individual polypeptides, or alternatively, as a set of larger fusion proteins (e.g., for phage display) containing the set of GPCR-like sequences therein. There are a variety of methods that can be used to produce libraries of potential GPCR-like variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GPCR-like sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a GPCR-like protein coding sequence can be used to generate a variegated population of GPCR-like fragments for screening and subsequent selection of variants of a GPCR-like protein. In one embodiment, a library of coding sequence fragments can be generated by treating a double-stranded PCR fragment of a GPCR-like coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the GPCR-like protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of GPCR-like proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify GPCR-like variants (Arkin and Yourvan (1992) Proc. Natl. Acad. Sci. USA 89:7811-7815; Delgrave et al. (1993) Protein Engineering 6(3):327-331).

An isolated GPCR-like polypeptide or fragments thereof of the invention can be used as an immunogen to generate antibodies that bind GPCR-like proteins using standard techniques for polyclonal and monoclonal antibody preparation. The full-length GPCR-like protein can be used or, alternatively, the invention provides antigenic peptide fragments of GPCR proteins for use as immunogens. The antigenic peptide of a GPCR-like protein comprises at least 8, preferably 10, 15, 20, or 30 amino acid residues of the amino acid sequence shown in SEQ ID NO:2 and encompasses an epitope of a GPCR-like protein such that an antibody raised against the peptide forms a specific immune complex with the GPCR-like protein. Preferred epitopes encompassed by the antigenic peptide are regions of a GPCR-like protein that are located on the surface of the protein, e.g., hydrophilic regions.

Accordingly, another aspect of the invention pertains to anti-GPCR-like polyclonal and monoclonal antibodies that bind a GPCR-like protein. Polyclonal anti-GPCR-like antibodies can be prepared by immunizing a suitable subject (e.g., rabbit, goat, mouse, or other mammal) with a GPCR-like immunogen. The anti-GPCR-like antibody titer in the immunized subject can be monitored over time by standard techniques, such as with an enzyme linked immunosorbent assay (ELISA) using immobilized GPCR-like protein. At an appropriate time after immunization, e.g., when the anti-GPCR-like antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal anti-GPCR-like antibody can be identified and isolated by screening a recombinant combinatorial immunoglobulin library (e.g., an antibody phage display library) with a GPCR-like protein to thereby isolate immunoglobulin library members that bind the GPCR-like protein. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-GPCR-like antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in PCT Publication Nos. WO 86/101533 and WO 87/02671; European Patent Application Nos. 184,187, 171, 496, 125,023, and 173,494; U.S. Pat. Nos. 4,816,567 and 5,225,539; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) Bio/Techniques 4:214; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

An anti-GPCR-like antibody (e.g., a monoclonal antibody) can be used to isolate GPCR-like proteins by standard techniques, such as affinity chromatography or immunoprecipitation. An anti-GPCR-like antibody can facilitate the purification of natural GPCR-like protein from cells and of recombinantly produced GPCR-like protein expressed in host cells. Moreover, an anti-GPCR-like antibody can be used to detect GPCR-like protein (e.g., in a cellular lysate or cell supernatant) in order to evaluate the abundance and pattern of expression of the GPCR-like protein. Anti-GPCR-like antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 35S, or 3H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor-alpha, tumor necrosis factor-beta, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84:Biological And clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

III. Recombinant Expression Vectors and Host Cells

Another aspect of the invention pertains to vectors, preferably expression vectors, containing a nucleic acid encoding a GPCR-like protein (or a portion thereof). “Vector” refers to a nucleic acid molecule capable of transporting another nucleic acid to which it has been linked, such as a “plasmid”, a circular double-stranded DNA loop into which additional DNA segments can be ligated, or a viral vector, where additional DNA segments can be ligated into the viral genome. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses), that serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, operably linked to the nucleic acid sequence to be expressed. “Operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GPCR-like proteins, mutant forms of GPCR-like proteins, fusion proteins, etc.).

The recombinant expression vectors of the invention can be designed for expression of GPCR-like protein in prokaryotic or eukaryotic host cells. Expression of proteins in prokaryotes is most often carried out in E. coli with vectors containing constitutive or inducible promoters directing the expression of either fusion or nonfusion proteins. Fusion vectors add a number of amino acids to a protein encoded therein, usually to the amino terminus of the recombinant protein. Typical fusion expression vectors include pGEX (Pharmacia Biotech Inc; Smith and Johnson (1988) Gene 67:31-40), pMAL (New England Biolabs, Beverly, Mass.), and pRIT5 (Pharmacia, Piscataway, N.J.) which fuse glutathione S-transferase (GST), maltose E binding protein, or protein A, respectively, to the target recombinant protein. Examples of suitable inducible nonfusion E. coli expression vectors include pTrc (Amann et al. (1988) Gene 69:301-315) and pET 11d (Studier et al. (1990) in Gene Expression Technology Methods in Enzymology 185 (Academic Press, San Diego, Calif.), pp. 60-89). Strategies to maximize recombinant protein expression in E. coli can be found in Gottesman (1990) in Gene Expression Technology: Methods in Enzymology 185 (Academic Press, CA), pp. 119-128 and Wada et al. (1992) Nucleic Acids Res. 20:2111-2118. Target gene expression from the pTrc vector relies on host RNA polymerase transcription from a hybrid trp-lac fusion promoter.

Suitable eukaryotic host cells include insect cells (examples of Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cereivisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or SV40 transformed simian kidney cells (COS). In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook et al. (1989) Molecular cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.). Alternatively, the recombinant expression vector can be transcribed and translated in vitro, for example using T7 promoter regulatory sequences and T7 polymerase.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell but are still included within the scope of the term as used herein.

In one embodiment, the expression vector is a recombinant mammalian expression vector that comprises tissue-specific regulatory elements that direct expression of the nucleic acid preferentially in a particular cell type. Suitable tissue-specific promoters include the albumin promoter (e.g., liver-specific promoter; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Patent Publication No. 264,166). Developmentally-regulated promoters are also encompassed, for example the murine homeobox (Hox) promoter (Kessel and Gruss (1990) Science 249:374-379), the α-fetoprotein promoter (Campes and Tilghman (1989) Genes Dev. 3:537-546), and the like.

The invention further provides a recombinant expression vector comprising a DNA molecule of the invention cloned into the expression vector in an antisense orientation. That is, the DNA molecule is operably linked to a regulatory sequence in a manner that allows for expression (by transcription of the DNA molecule) of an RNA molecule that is antisense to GPCR-like mRNA. Regulatory sequences operably linked to a nucleic acid cloned in the antisense orientation can be chosen to direct the continuous expression of the antisense RNA molecule in a variety of cell types, for instance viral promoters and/or enhancers, or regulatory sequences can be chosen to direct constitutive, tissue-specific, or cell-type-specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub et al. (1986) Reviews—Trends in Genetics, Vol. 1(1).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin, and methotrexate. Nucleic acid encoding a selectable marker can be introduced into a host cell on the same vector as that encoding a GPCR-like protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GPCR-like protein. Accordingly, the invention further provides methods for producing GPCR-like protein using the host cells of the invention. In one embodiment, the method comprises culturing the host cell of the invention, into which a recombinant expression vector encoding a GPCR-like protein has been introduced, in a suitable medium such that GPCR-like protein is produced. In another embodiment, the method further comprises isolating GPCR-like protein from the medium or the host cell.

The host cells of the invention can also be used to produce nonhuman transgenic animals. For example, in one embodiment, a host cell of the invention is a fertilized oocyte or an embryonic stem cell into which GPCR-like-coding sequences have been introduced. Such host cells can then be used to create nonhuman transgenic animals in which exogenous GPCR-like sequences have been introduced into their genome or homologous recombinant animals in which endogenous GPCR-like sequences have been altered. Such animals are useful for studying the function and/or activity of GPCR-like genes and proteins and for identifying and/or evaluating modulators of GPCR-like activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA that is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous GPCR-like gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GPCR-like-encoding nucleic acid into the male pronuclei of a fertilized oocyte, e.g., by microinjection, retroviral infection, and allowing the oocyte to develop in a pseudopregnant female foster animal. The GPCR-like cDNA sequence can be introduced as a transgene into the genome of a nonhuman animal. Alternatively, a homologue of the mouse GPCR-like gene can be isolated based on hybridization and used as a transgene. Intronic sequences and polyadenylation signals can also be included in the transgene to increase the efficiency of expression of the transgene. A tissue-specific regulatory sequence(s) can be operably linked to the GPCR-like transgene to direct expression of GPCR-like protein to particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan (1986) Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the GPCR-like transgene in its genome and/or expression of GPCR-like mRNA in tissues or cells of the animals. A transgenic founder animal can then be used to breed additional animals carrying the transgene. Moreover, transgenic animals carrying a transgene encoding GPCR-like gene can further be bred to other transgenic animals carrying other transgenes.

To create a homologous recombinant animal, one prepares a vector containing at least a portion of a GPCR-like gene or a homolog of the gene into which a deletion, addition, or substitution has been introduced to thereby alter, e.g., functionally disrupt, the GPCR-like gene. In a preferred embodiment, the vector is designed such that, upon homologous recombination, the endogenous GPCR-like gene is functionally disrupted (i.e., no longer encodes a functional protein; also referred to as a “knock out” vector). Alternatively, the vector can be designed such that, upon homologous recombination, the endogenous GPCR-like gene is mutated or otherwise altered but still encodes functional protein (e.g., the upstream regulatory region can be altered to thereby alter the expression of the endogenous GPCR-like protein). In the homologous recombination vector, the altered portion of the GPCR-like gene is flanked at its 5′ and 3′ ends by additional nucleic acid of the GPCR-like gene to allow for homologous recombination to occur between the exogenous GPCR-like gene carried by the vector and an endogenous GPCR-like gene in an embryonic stem cell. The additional flanking GPCR-like nucleic acid is of sufficient length for successful homologous recombination with the endogenous gene. Typically, several kilobases of flanking DNA (at both the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation), and cells in which the introduced GPCR-like gene has homologously recombined with the endogenous GPCR-like gene are selected (see, e.g., Li et al. (1992) Cell 69:915). The selected cells are then injected into a blastocyst of an animal (e.g., a mouse) to form aggregation chimeras (see, e.g., Bradley (1987) in Teratocarcinomas and Embryonic Stem Cells: A Practical Approach, ed. Robertson (IRL, Oxford pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic nonhuman animals containing selected systems that allow for regulated expression of the transgene can be produced. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut et al. (1997) Nature 385:810-813 and PCT Publication Nos. WO 97/07668 and WO 97/07669.

IV. Pharmaceutical Compositions

The GPCR-like nucleic acid molecules, GPCR-like proteins, and modulators thereof (e.g., anti-GPCR-like antibodies) (also referred to herein as “active compounds”) of the invention can be incorporated into pharmaceutical compositions suitable for administration. Such compositions typically comprise the nucleic acid molecule, protein, or modulators thereof (e.g., antibody or small molecule) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions of the invention are useful to treat any of the disorders discussed herein. The compositions are provided in therapeutically effective amounts. By “therapeutically effective amounts” is intended an amount sufficient to modulate the desired response. As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELD (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a GPCR-like protein or anti-GPCR-like antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. Depending on the type and severity of the disease, about 1 μg/kg to about 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: (a) screening assays; (b) detection assays (e.g., chromosomal mapping, tissue typing, forensic biology); (c) predictive medicine (e.g., diagnostic assays, prognostic assays, monitoring clinical trials, and pharmacogenomics); and (d) methods of treatment (e.g., therapeutic and prophylactic). The isolated nucleic acid molecules of the invention can be used to express GPCR-like protein (e.g., via a recombinant expression vector in a host cell in gene therapy applications), to detect GPCR-like mRNA (e.g., in a biological sample) or a genetic lesion in a GPCR-like gene, and to modulate GPCR-like activity. In addition, the GPCR-like proteins can be used to screen drugs or compounds that modulate the immune response as well as to treat disorders characterized by insufficient or excessive production of GPCR-like protein or production of GPCR-like protein forms that have decreased or aberrant activity compared to GPCR-like wild type protein. In addition, the anti-GPCR-like antibodies of the invention can be used to detect and isolate GPCR-like proteins and modulate GPCR-like activity.

A. Screening Assays

The invention provides a method (also referred to herein as a “screening assay”) for identifying modulators, i.e., candidate or test compounds or agents (e.g., peptides, peptidomimetics, small molecules, or other drugs) that bind to GPCR-like proteins or have a stimulatory or inhibitory effect on, for example, GPCR-like expression or GPCR-like activity.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Determining the ability of the test compound to bind to the GPCR-like protein can be accomplished, for example, by coupling the test compound with a radioisotope or enzymatic label such that binding of the test compound to the GPCR-like protein or biologically active portion thereof can be determined by detecting the labeled compound in a complex. For example, test compounds can be labeled with 125I, 35S, 14C, or 3H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In a similar manner, one may determine the ability of the GPCR-like protein to bind to or interact with a GPCR-like target molecule. By “target molecule” is intended a molecule with which a GPCR-like protein binds or interacts in nature. In a preferred embodiment, the ability of the GPCR-like protein to bind to or interact with a GPCR-like target molecule can be determined by monitoring the activity of the target molecule. For example, the activity of the target molecule can be monitored by detecting induction of a cellular second messenger of the target (e.g., intracellular Ca2+, diacylglycerol, IP3, etc.), detecting catalytic/enzymatic activity of the target on an appropriate substrate, detecting the induction of a reporter gene (e.g., a GPCR-like-responsive regulatory element operably linked to a nucleic acid encoding a detectable marker, e.g. luciferase), or detecting a cellular response, for example, cellular differentiation or cell proliferation.

In yet another embodiment, an assay of the present invention is a cell-free assay comprising contacting a GPCR-like protein or biologically active portion thereof with a test compound and determining the ability of the test compound to bind to the GPCR-like protein or biologically active portion thereof. Binding of the test compound to the GPCR-like protein can be determined either directly or indirectly as described above. In a preferred embodiment, the assay includes contacting the GPCR-like protein or biologically active portion thereof with a known compound that binds GPCR-like protein to form an assay mixture, contacting the assay mixture with a test compound, and determining the ability of the test compound to preferentially bind to GPCR-like protein or biologically active portion thereof as compared to the known compound.

In another embodiment, an assay is a cell-free assay comprising contacting GPCR-like protein or biologically active portion thereof with a test compound and determining the ability of the test compound to modulate (e.g., stimulate or inhibit) the activity of the GPCR-like protein or biologically active portion thereof. Determining the ability of the test compound to modulate the activity of a GPCR-like protein can be accomplished, for example, by determining the ability of the GPCR-like protein to bind to a GPCR-like target molecule as described above for determining direct binding. In an alternative embodiment, determining the ability of the test compound to modulate the activity of a GPCR-like protein can be accomplished by determining the ability of the GPCR-like protein to further modulate a GPCR-like target molecule. For example, the catalytic/enzymatic activity of the target molecule on an appropriate substrate can be determined as previously described.

In yet another embodiment, the cell-free assay comprises contacting the GPCR-like protein or biologically active portion thereof with a known compound that binds a GPCR-like protein to form an assay mixture, contacting the assay mixture with a test compound, and determining the ability of the test compound to preferentially bind to or modulate the activity of a GPCR-like target molecule.

In the above-mentioned assays, it may be desirable to immobilize either a GPCR-like protein or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, glutathione-S-transferase/GPCR-like fusion proteins or glutathione-S-transferase/target fusion proteins can be adsorbed onto glutathione sepharose beads (Sigma Chemical, St. Louis, Mo.) or glutathione-derivatized microtitre plates, which are then combined with the test compound or the test compound and either the nonadsorbed target protein or GPCR-like protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of GPCR-like binding or activity determined using standard techniques.

Other techniques for immobilizing proteins on matrices can also be used in the screening assays of the invention. For example, either GPCR-like protein or its target molecule can be immobilized utilizing conjugation of biotin and streptavidin. Biotinylated GPCR-like molecules or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with a GPCR-like protein or target molecules but which do not interfere with binding of the GPCR-like protein to its target molecule can be derivatized to the wells of the plate, and unbound target or GPCR-like protein trapped in the wells by antibody conjugation. Methods for detecting such complexes, in addition to those described above for the GST-immobilized complexes, include immunodetection of complexes using antibodies reactive with the GPCR-like protein or target molecule, as well as enzyme-linked assays that rely on detecting an enzymatic activity associated with the GPCR-like protein or target molecule.

In another embodiment, modulators of GPCR-like expression are identified in a method in which a cell is contacted with a candidate compound and the expression of GPCR-like mRNA or protein in the cell is determined relative to expression of GPCR-like mRNA or protein in a cell in the absence of the candidate compound. When expression is greater (statistically significantly greater) in the presence of the candidate compound than in its absence, the candidate compound is identified as a stimulator of GPCR-like mRNA or protein expression. Alternatively, when expression is less (statistically significantly less) in the presence of the candidate compound than in its absence, the candidate compound is identified as an inhibitor of GPCR-like mRNA or protein expression. The level of GPCR-like mRNA or protein expression in the cells can be determined by methods described herein for detecting GPCR-like mRNA or protein.

In yet another aspect of the invention, the GPCR-like proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with GPCR-like protein (“GPCR-like-binding proteins” or “GPCR-like-bp”) and modulate GPCR-like activity. Such GPCR-like-binding proteins are also likely to be involved in the propagation of signals by the GPCR-like proteins as, for example, upstream or downstream elements of the GPCR-like pathway.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (1) map their respective genes on a chromosome; (2) identify an individual from a minute biological sample (tissue typing); and (3) aid in forensic identification of a biological sample. These applications are described in the subsections below.

1. Chromosome Mapping

The isolated complete or partial GPCR-like gene sequences of the invention can be used to map their respective GPCR-like genes on a chromosome, thereby facilitating the location of gene regions associated with genetic disease. Computer analysis of GPCR-like sequences can be used to rapidly select PCR primers (preferably 15-25 bp in length) that do not span more than one exon in the genomic DNA, thereby simplifying the amplification process. These primers can then be used for PCR screening of somatic cell hybrids containing individual human chromosomes. Only those hybrids containing the human gene corresponding to the GPCR-like sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio et al. (1983) Science 220:919-924). Somatic cell hybrids containing only fragments of human chromosomes can also be produced by using human chromosomes with translocations and deletions.

Other mapping strategies that can similarly be used to map a GPCR-like sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Furthermore, fluorescence in situ hybridization (FISH) of a DNA sequence to a metaphase chromosomal spread can be used to provide a precise chromosomal location in one step. For a review of this technique, see Verma et al. (1988) Human Chromosomes: A Manual of Basic Techniques (Pergamon Press, NY). The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results in a reasonable amount of time.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Another strategy to map the chromosomal location of GPCR-like genes uses GPCR-like polypeptides and fragments and sequences of the present invention and antibodies specific thereto. This mapping can be carried out by specifically detecting the presence of a GPCR-like polypeptide in members of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al. (1988) Cytogenet. Cell Genet. 47:37-41 and Van Keuren et al. (1986) Hum. Genet. 74:34-40. Alternatively, the presence of a GPCR-like polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with the GPCR-like gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The GPCR-like sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes and probed on a Southern blot to yield unique bands for identification. The sequences of the present invention are useful as additional DNA markers for RFLP (described, e.g., in U.S. Pat. No. 5,272,057).

Furthermore, the sequences of the present invention can be used to provide an alternative technique for determining the actual base-by-base DNA sequence of selected portions of an individual's genome. Thus, the GPCR-like sequences of the invention can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The GPCR-like sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. The noncoding sequences of a nucleotide sequence comprising the sequence shown SEQ ID NO:1 can comfortably provide positive individual identification with a panel of perhaps 10 to 1,000 primers that each yield a noncoding amplified sequence of 100 bases. If a predicted coding sequence, such as that in SEQ ID NO:1, is used, a more appropriate number of primers for positive individual identification would be 500 to 2,000.

3. Use of Partial GPCR-Like Sequences in Forensic Biology

DNA-based identification techniques can also be used in forensic biology. In this manner, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” that is unique to a particular individual. As mentioned above, actual base sequence information can be used for identification as an accurate alternative to patterns formed by restriction enzyme generated fragments. Sequences targeted to noncoding regions of a sequence comprising the sequence shown in SEQ ID NO:1 are particularly appropriate for this use as greater numbers of polymorphisms occur in the noncoding regions, making it easier to differentiate individuals using this technique. Examples of polynucleotide reagents include the GPCR-like sequences or portions thereof, e.g., fragments derived from the noncoding regions of sequences comprising the sequence shown in SEQ ID NO:1 having a length of at least 20 or 30 bases.

The GPCR-like sequences described herein can further be used to provide polynucleotide reagents, e.g., labeled or labelable probes that can be used in, for example, an in situ hybridization technique, to identify a specific tissue. This can be very useful in cases where a forensic pathologist is presented with a tissue of unknown origin. Panels of such GPCR-like probes, can be used to identify tissue by species and/or by organ type.

In a similar fashion, these reagents, e.g., GPCR-like primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. These applications are described in the subsections below.

1. Diagnostic Assays

One aspect of the present invention relates to diagnostic assays for detecting GPCR-like protein and/or nucleic acid expression as well as GPCR-like activity, in the context of a biological sample. An exemplary method for detecting the presence or absence of GPCR-like proteins in a biological sample involves obtaining a biological sample from a test subject and contacting the biological sample with a compound or an agent capable of detecting GPCR-like protein or nucleic acid (e.g., mRNA, genomic DNA) that encodes GPCR-like protein such that the presence of GPCR-like protein is detected in the biological sample. Results obtained with a biological sample from the test subject may be compared to results obtained with a biological sample from a control subject.

A preferred agent for detecting GPCR-like mRNA or genomic DNA is a labeled nucleic acid probe capable of hybridizing to GPCR-like mRNA or genomic DNA. The nucleic acid probe can be, for example, a full-length GPCR-like nucleic acid, such as the full-length sequence shown in SEQ ID NO:1, or a portion thereof, such as a nucleic acid molecule of at least 15, 30, 50, 100, 250, or 500 nucleotides in length and sufficient to specifically hybridize under stringent conditions to GPCR-like mRNA or genomic DNA. Other suitable probes for use in the diagnostic assays of the invention are described herein.

A preferred agent for detecting GPCR-like protein is an antibody capable of binding to GPCR-like protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect GPCR-like mRNA, protein, or genomic DNA in a biological sample in vitro as well as in vivo. For example, in vitro techniques for detection of GPCR-like mRNA include Northern hybridizations and in situ hybridizations. In vitro techniques for detection of GPCR-like protein include enzyme linked immunosorbent assays (ELISAs), Western blots, immunoprecipitations, and immunofluorescence. In vitro techniques for detection of GPCR-like genomic DNA include Southern hybridizations. Furthermore, in vivo techniques for detection of GPCR-like protein include introducing into a subject a labeled anti-GPCR-like antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. Preferred biological samples are fibroblast samples, particularly dermal and lung fibroblasts, fibrotic samples, particularly liver fibrotic samples, and hepatic stellate cells isolated by conventional means from a subject.

The invention also encompasses kits for detecting the presence of GPCR-like proteins in a biological sample (a test sample). Such kits can be used to determine if a subject is suffering from or is at increased risk of developing a disorder associated with aberrant expression of GPCR-like protein (e.g., an immunological disorder). For example, the kit can comprise a labeled compound or agent capable of detecting GPCR-like protein or mRNA in a biological sample and means for determining the amount of a GPCR-like protein in the sample (e.g., an anti-GPCR-like antibody or an oligonucleotide probe that binds to DNA encoding a GPCR-like protein, e.g., SEQ ID NO:1). Kits can also include instructions for observing that the tested subject is suffering from or is at risk of developing a disorder associated with aberrant expression of GPCR-like sequences if the amount of GPCR-like protein or mRNA is above or below a normal level.

For antibody-based kits, the kit can comprise, for example: (1) a first antibody (e.g., attached to a solid support) that binds to GPCR-like protein; and, optionally, (2) a second, different antibody that binds to GPCR-like protein or the first antibody and is conjugated to a detectable agent. For oligonucleotide-based kits, the kit can comprise, for example: (1) an oligonucleotide, e.g., a detectably labeled oligonucleotide, that hybridizes to a GPCR-like nucleic acid sequence or (2) a pair of primers useful for amplifying a GPCR-like nucleic acid molecule.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for observing whether the tested subject is suffering from or is at risk of developing a disorder associated with aberrant expression of GPCR-like proteins.

2. Other Diagnostic Assays

In another aspect, the invention features a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a GPCR-like nucleic acid, preferably purified, polypeptide, preferably purified, or antibody, and thereby evaluating the plurality of capture probes. Binding (e.g., in the case of a nucleic acid, hybridization) with a capture probe at an address of the plurality, is detected, e.g., by a signal generated from a label attached to the GPCR-like nucleic acid, polypeptide, or antibody. The capture probes can be a set of nucleic acids from a selected sample, e.g., a sample of nucleic acids derived from a control or non-stimulated tissue or cell.

The method can include contacting the GPCR-like nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of a GPCR-like sequence of the invention. Such methods can be used to diagnose a subject, e.g., to evaluate risk for a disease or disorder, to evaluate suitability of a selected treatment for a subject, to evaluate whether a subject has a disease or disorder. Thus, for example, the h15571 sequence set forth in SEQ ID NO:1 encodes a GPCR-like polypeptide that is associated with liver function, thus it is useful for evaluating liver disorders.

The method can be used to detect single nucleotide polymorphisms (SNPs), as described below.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express a GPCR-like polypeptide of the invention or from a cell or subject in which a GPCR-like-mediated response has been elicited, e.g., by contact of the cell with a GPCR-like nucleic acid or protein of the invention, or administration to the cell or subject a GPCR-like nucleic acid or protein of the invention; contacting the array with one or more inquiry probes, wherein an inquiry probe can be a nucleic acid, polypeptide, or antibody (which is preferably other than a GPCR-like nucleic acid, polypeptide, or antibody of the invention); providing a two dimensional array having a plurality of addresses, each address of the plurality being positionally distinguishable from each other address of the plurality, and each address of the plurality having a unique capture probe, e.g., wherein the capture probes are from a cell or subject which does not express a GPCR-like sequence of the invention (or does not express as highly as in the case of the GPCR-like positive plurality of capture probes) or from a cell or subject in which a GPCR-like-mediated response has not been elicited (or has been elicited to a lesser extent than in the first sample); contacting the array with one or more inquiry probes (which is preferably other than a GPCR-like nucleic acid, polypeptide, or antibody of the invention), and thereby evaluating the plurality of capture probes. Binding, e.g., in the case of a nucleic acid, hybridization, with a capture probe at an address of the plurality, is detected, e.g., by signal generated from a label attached to the nucleic acid, polypeptide, or antibody.

In another aspect, the invention features a method of analyzing a GPCR-like sequence of the invention, e.g., analyzing structure, function, or relatedness to other nucleic acid or amino acid sequences. The method includes: providing a GPCR-like nucleic acid or amino acid sequence, e.g., the h15571 sequence set forth in SEQ ID NO:1 or SEQ ID NO:2 or a portion thereof; comparing the GPCR-like sequence with one or more preferably a plurality of sequences from a collection of sequences, e.g., a nucleic acid or protein sequence database; to thereby analyze the GPCR-like sequence of the invention.

The method can include evaluating the sequence identity between a GPCR-like sequence of the invention, e.g., the h15571 sequence, and a database sequence. The method can be performed by accessing the database at a second site, e.g., over the internet.

In another aspect, the invention features, a set of oligonucleotides, useful, e.g., for identifying SNP's, or identifying specific alleles of a GPCR-like sequence of the invention, e.g., the h15571 sequence. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotides which hybridizes to one allele provides a signal that is distinguishable from an oligonucleotides which hybridizes to a second allele.

3. Prognostic Assays

The methods described herein can furthermore be utilized as diagnostic or prognostic assays to identify subjects having or at risk of developing a disease or disorder associated with GPCR-like protein, GPCR-like nucleic acid expression, or GPCR-like activity. Prognostic assays can be used for prognostic or predictive purposes to thereby prophylactically treat an individual prior to the onset of a disorder characterized by or associated with GPCR-like protein, GPCR-like nucleic acid expression, or GPCR-like activity.

Thus, the present invention provides a method in which a test sample is obtained from a subject, and GPCR-like protein or nucleic acid (e.g., mRNA, genomic DNA) is detected, wherein the presence of GPCR-like protein or nucleic acid is diagnostic for a subject having or at risk of developing a disease or disorder associated with aberrant GPCR-like expression or activity. As used herein, a “test sample” refers to a biological sample obtained from a subject of interest. For example, a test sample can be a biological fluid (e.g., serum), cell sample, or tissue.

Furthermore, using the prognostic assays described herein, the present invention provides methods for determining whether a subject can be administered a specific agent (e.g., an agonist, antagonist, peptidomimetic, protein, peptide, nucleic acid, small molecule, or other drug candidate) or class of agents (e.g., agents of a type that decrease GPCR-like activity) to effectively treat a disease or disorder associated with aberrant GPCR-like expression or activity. In this manner, a test sample is obtained and GPCR-like protein or nucleic acid is detected. The presence of GPCR-like protein or nucleic acid is diagnostic for a subject that can be administered the agent to treat a disorder associated with aberrant GPCR-like expression or activity.

The methods of the invention can also be used to detect genetic lesions or mutations in a GPCR-like gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a GPCR-like protein, or the misexpression of the GPCR-like gene. For example, such genetic lesions or mutations can be detected by ascertaining the existence of at least one of: (1) a deletion of one or more nucleotides from a GPCR-like gene; (2) an addition of one or more nucleotides to a GPCR-like gene; (3) a substitution of one or more nucleotides of a GPCR-like gene; (4) a chromosomal rearrangement of a GPCR-like gene; (5) an alteration in the level of a messenger RNA transcript of a GPCR-like gene; (6) an aberrant modification of a GPCR-like gene, such as of the methylation pattern of the genomic DNA; (7) the presence of a non-wild-type splicing pattern of a messenger RNA transcript of a GPCR-like gene; (8) a non-wild-type level of a GPCR-like protein; (9) an allelic loss of a GPCR-like gene; and (10) an inappropriate post-translational modification of a GPCR-like protein. As described herein, there are a large number of assay techniques known in the art that can be used for detecting lesions in a GPCR-like gene. Any cell type or tissue, for example, hepatic stellate cells, dermal and lung fibroblasts, fibrotic tissues, particularly fibrotic liver tissues, in which the GPCR-like proteins are expressed may be utilized in the prognostic assays described herein.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the GPCR-like gene (see, e.g., Abravaya et al. (1995) Nucleic Acids Res. 23:675-682). It is anticipated that PCR and/or LCR may be desirable to use as a preliminary amplification step in conjunction with any of the techniques used for detecting mutations described herein.

Alternative amplification methods include self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a GPCR-like gene from a sample cell can be identified by alterations in restriction enzyme cleavage patterns of isolated test sample and control DNA digested with one or more restriction endonucleases. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in a GPCR-like molecule can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). In yet another embodiment, any of a variety of sequencing reactions known in the art can be used to directly sequence the GPCR-like gene and detect mutations by comparing the sequence of the sample GPCR-like gene with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the GPCR-like gene include methods in which protection from cleavage agents is used to detect mismatched bases in RNA/RNA or RNA/DNA heteroduplexes (Myers et al. (1985) Science 230:1242). See, also Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more “DNA mismatch repair” enzymes that recognize mismatched base pairs in double-stranded DNA in defined systems for detecting and mapping point mutations in GPCR-like cDNAs obtained from samples of cells. See, e.g., Hsu et al. (1994) Carcinogenesis 15:1657-1662. According to an exemplary embodiment, a probe based on a GPCR-like sequence, e.g., a wild-type GPCR-like sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in GPCR-like genes. For example, single-strand conformation polymorphism (SSCP) may be used to detect differences in electrophoretic mobility between mutant and wild-type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc. Natl. Acad. Sci. USA 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology, which depends on selective PCR amplification, may be used in conjunction with the instant invention. Oligonucleotides used as primers for specific amplification may carry the mutation of interest in the center of the molecule so that amplification depends on differential hybridization (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnosed patients exhibiting symptoms or family history of a disease or illness involving a GPCR-like gene.

4. Pharmacogenomics

Agents, or modulators that have a stimulatory or inhibitory effect on GPCR-like activity (e.g., GPCR-like gene expression) as identified by a screening assay described herein, can be administered to individuals to treat (prophylactically or therapeutically) disorders associated with aberrant GPCR-like activity as well as to modulate the phenotype of an immune response. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of GPCR-like protein, expression of GPCR-like nucleic acid, or mutation content of GPCR-like genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (antimalarials, sulfonamides, analgesics, nitrofurans) and consumption of fava beans.

Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a GPCR-like molecule or GPCR-like modulator of the invention as well as tailoring the dosage and/or therapeutic regimen of treatment with a GPCR-like molecule or GPCR-like modulator of the invention.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, an “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a GPCR-like protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a GPCR-like molecule or GPCR-like modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a GPCR-like molecule or GPCR-like modulator of the invention, such as a modulator identified by one of the exemplary screening assays described herein.

The present invention further provides methods for identifying new agents, or combinations, that are based on identifying agents that modulate the activity of one or more of the gene products encoded by one or more of the GPCR-like genes of the present invention, wherein these products may be associated with resistance of the cells to a therapeutic agent. Specifically, the activity of the proteins encoded by the GPCR-like genes of the present invention can be used as a basis for identifying agents for overcoming agent resistance. By blocking the activity of one or more of the resistance proteins, target cells, e.g., hepatic stellate cells, will become sensitive to treatment with an agent that the unmodified target cells were resistant to.

Monitoring the influence of agents (e.g., drugs) on the expression or activity of a GPCR-like protein can be applied in clinical trials. For example, the effectiveness of an agent determined by a screening assay as described herein to increase GPCR-like gene expression, protein levels, or upregulate GPCR-like activity, can be monitored in clinical trials of subjects exhibiting decreased GPCR-like gene expression, protein levels, or downregulated GPCR-like activity. Alternatively, the effectiveness of an agent determined by a screening assay to decrease GPCR-like gene expression, protein levels, or downregulate GPCR-like activity, can be monitored in clinical trials of subjects exhibiting increased GPCR-like gene expression, protein levels, or upregulated GPCR-like activity. In such clinical trials, the expression or activity of a GPCR-like gene, and preferably, other genes that have been implicated in, for example, a GPCR-like-associated disorder can be used as a “read out” or markers of the phenotype of a particular cell.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of GPCR-like protein, expression of GPCR-like nucleic acid, or mutation content of GPCR-like genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a GPCR-like modulator, such as a modulator identified by one of the exemplary screening assays described herein.

5. Monitoring of Effects During Clinical Trials

Monitoring the influence of agents (e.g., drugs, compounds) on the expression or activity of GPCR-like genes (e.g., the ability to modulate aberrant cell proliferation and/or differentiation) can be applied not only in basic drug screening but also in clinical trials. For example, the effectiveness of an agent, as determined by a screening assay as described herein, to increase or decrease GPCR-like gene expression, protein levels, or protein activity, can be monitored in clinical trials of subjects exhibiting decreased or increased GPCR-like gene expression, protein levels, or protein activity. In such clinical trials, GPCR-like expression or activity and preferably that of other genes that have been implicated in for example, a cellular proliferation disorder, can be used as a marker of the immune responsiveness of a particular cell.

For example, and not by way of limitation, genes that are modulated in cells by treatment with an agent (e.g., compound, drug, or small molecule) that modulates GPCR-like activity (e.g., as identified in a screening assay described herein) can be identified. Thus, to study the effect of agents on cellular proliferation disorders, for example, in a clinical trial, cells can be isolated and RNA prepared and analyzed for the levels of expression of GPCR-like genes and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of GPCR-like genes or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (1) obtaining a preadministration sample from a subject prior to administration of the agent; (2) detecting the level of expression of a GPCR-like protein, mRNA, or genomic DNA in the preadministration sample; (3) obtaining one or more postadministration samples from the subject; (4) detecting the level of expression or activity of the GPCR-like protein, mRNA, or genomic DNA in the postadministration samples; (5) comparing the level of expression or activity of the GPCR-like protein, mRNA, or genomic DNA in the preadministration sample with the GPC GPCR-like R protein, mRNA, or genomic DNA in the postadministration sample or samples; and (vi) altering the administration of the agent to the subject accordingly to bring about the desired effect, i.e., for example, an increase or a decrease in the expression or activity of a GPCR-like protein.

C. Methods of Treatment

The present invention provides for both prophylactic and therapeutic methods of treating a subject at risk of (or susceptible to) a disorder or having a disorder associated with aberrant GPCR-like expression or activity. Additionally, the compositions of the invention find use in modulating the treatment of disorders described herein. Thus, therapies for immune, inflammatory, hematologic, fibrotic, hepatic, and respiratory disorders; disorders associated with the following cells or tissues: lymph node; spleen; thymus; brain; lung; skeletal muscle; fetal liver; tonsil; colon; heart; liver; peripheral blood mononuclear cells (PBMC); CD34+; bone marrow cells; neonatal umbilical cord blood (CB CD34+); leukocytes from G-CSF treated patients (mPB leukocytes); CD14+ cells; monocytes; hepatic stellate cells; fibrotic liver; kidney; spinal cord; and dermal and lung fibroblasts; are encompassed herein.

1. Prophylactic Methods

In one aspect, the invention provides a method for preventing in a subject a disease or condition associated with an aberrant GPCR-like expression or activity by administering to the subject an agent that modulates GPCR-like expression or at least one GPCR-like gene activity. Subjects at risk for a disease that is caused, or contributed to, by aberrant GPCR-like expression or activity can be identified by, for example, any or a combination of diagnostic or prognostic assays as described herein. Administration of a prophylactic agent can occur prior to the manifestation of symptoms characteristic of the GPCR-like aberrancy, such that a disease or disorder is prevented or, alternatively, delayed in its progression. Depending on the type of GPCR-like aberrancy, for example, a GPCR-like agonist or GPCR-like antagonist agent can be used for treating the subject. The appropriate agent can be determined based on screening assays described herein.

2. Therapeutic Methods

Another aspect of the invention pertains to methods of modulating GPCR-like expression or activity for therapeutic purposes. The modulatory method of the invention involves contacting a cell with an agent that modulates one or more of the activities of GPCR-like protein activity associated with the cell. An agent that modulates GPCR-like protein activity can be an agent as described herein, such as a nucleic acid or a protein, a naturally-occurring cognate ligand of a GPCR-like protein, a peptide, a GPCR peptidomimetic, or other small molecule. In one embodiment, the agent stimulates one or more of the biological activities of GPCR-like protein. Examples of such stimulatory agents include active GPCR-like protein and a nucleic acid molecule encoding a GPCR-like protein that has been introduced into the cell. In another embodiment, the agent inhibits one or more of the biological activities of GPCR-like protein. Examples of such inhibitory agents include antisense GPCR-like nucleic acid molecules and anti-GPCR-like antibodies.

These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a GPCR-like protein or nucleic acid molecule. In one embodiment, the method involves administering an agent (e.g., an agent identified by a screening assay described herein), or a combination of agents, that modulates (e.g., upregulates or downregulates) GPCR-like expression or activity. In another embodiment, the method involves administering a GPCR-like protein or nucleic acid molecule as therapy to compensate for reduced or aberrant GPCR-like expression or activity.

Stimulation of GPCR activity is desirable in situations in which a GPCR-like protein is abnormally downregulated and/or in which increased GPCR-like activity is likely to have a beneficial effect. Conversely, inhibition of GPCR-like activity is desirable in situations in which GPCR-like activity is abnormally upregulated and/or in which decreased GPCR-like activity is likely to have a beneficial effect.

This invention is further illustrated by the following examples, which should not be construed as limiting.

### EXPERIMENTAL

### Example 1

### Isolation of h15571

The clone h15571 was isolated from human thymus and spleen cDNA libraries. The identified clone h15571 encodes a transcript of approximately 6.09 Kb (corresponding cDNA set forth in SEQ ID NO:1). Nucleotides 366-4014 of this transcript represent an open reading frame that encodes a predicted 1338 amino acid polypeptide (SEQ ID NO:2).

An analysis of the h15571 GPCR-like amino acid sequence for physico-chemical characteristics, such as αβ turn and coil regions, hydrophilicity, amphipathic regions, flexible regions, antigenic index, and surface probability plot is described herein.

A search of the nucleotide and protein databases revealed that h15571 shares similarity with other sequences, primarily in the C-terminal portion. The closest similarity resides with human cDNA DKFZp434C211 (GenBank Accession No. AL110244). Nucleotides 2986-5685 of SEQ ID NO:1 share approximately 99.4% sequence identity with this cDNA, as determined by global pairwise alignment. This cDNA encodes a hypothetical uncharacterized protein (GenBank Accession No. CAB53694, having 100% identity with amino acid residues 999-1338 of SEQ ID NO:2, the protein encoded by h15571, as determined by local pairwise alignment (BESTFIT). Local pairwise alignment (using BESTFIT) of the h15571 polypeptide indicates this protein shares sequence similarity to other GPCR proteins. Specifically, amino acid residues 695-944 of SEQ ID NO:2 share approximately 41.6% similarity and 30.5% identity with amino acid residues 2411-2646 of a mouse seven-pass transmembrane receptor precursor (GenBank Accession No. AAC68836); amino acid residues 689-946 of SEQ ID NO:2 share approximately 37.7% similarity and 30.5% identity with human MEGF2, a seven-pass transmembrane protein (GenBank Accession No. BAA32464); and amino acid residues 703-946 of SEQ ID NO:2 share approximately 37.8% similarity and 25.2% identity with amino acid residues 703-946 of rat MEGF2, a seven-pass transmembrane protein (GenBank Accession No. ABB32459).

An alignment of the sequence encompassing the region of the seven transmembrane domain (7tm) of h15571 (SEQ ID NO:2) and the following human GPCRs of the Class B secretin-like family demonstrates the close relation of this GPCR to other known seven transmembrane molecules: CD97R (leukocyte antigen CD97, Swiss-Prot accession number P48960) (SEQ ID NO:75); CGRR (a calcitonin gene-related peptide type 1 receptor; Swiss-Prot accession number Q16602) (SEQ ID NO:76); CRF1 (corticotropin releasing factor receptor 1; Swiss-Prot accession numbers P34998 and Q13008) (SEQ ID NO:77); CRF2 (corticotropin releasing factor receptor 2; Swiss-Prot accession numbers Q13324, Q99431, and O43461) (SEQ ID NO:78); CTR (calcitonin receptor; Swiss-Prot accession number P30988) (SEQ ID NO:79); EMR1 (cell surface glycoprotein EMR1; Swiss-Prot accession number Q14246) (SEQ ID NO:80); GIPR (glucose-dependent insulinotropic polypeptide receptor; Swiss-Prot accession numbers P48546, Q16400, and Q14401) (SEQ ID NO:81); GLRP (glucagon-like peptide 1 receptor; Swiss-Prot accession numbers P43220 and Q99669) (SEQ ID NO:82); GLR (glucagon receptor; Swiss-Prot accession number P47871) (SEQ ID NO:83); GRFR (growth hormone-releasing hormone receptor; Swiss-Prot accession numbers Q02643 and Q99863) (SEQ ID NO:84); PACR (pituitary adenylate cyclase activating polypeptide type I receptor; Swiss-Prot accession number P41586) (SEQ ID NO:85); PTR2 (parathyroid hormone receptor; Swiss-Prot accession number P49190) (SEQ ID NO:86); PTRR (parathyroid hormone/parathyroid hormone-related peptide receptor; Swiss-Prot accession number Q03431) (SEQ ID NO:87) SCRC (secretin receptor; Swiss-Prot accession numbers P47872, Q13213, and Q12961) (SEQ ID NO:88); VIPR (pituitary adenylate cyclase activating polypeptide type II receptor; Swiss-Prot accession numbers P32241 and Q15871) (SEQ ID NO:89); and, VIPS (pituitary adenylate cyclase activating polypeptide type III receptor; Swiss-Prot accession numbers P41587, Q15870, and Q13053) (SEQ ID NO:90).

### Example 2

### h15571 Expression Analysis

Total RNA was prepared from various human tissues by a single step extraction method using RNA STAT-60 according to the manufacturer's instructions (TelTest, Inc). Each RNA preparation was treated with DNase I (Ambion) at 37° C. for 1 hour. DNAse I treatment was determined to be complete if the sample required at least 38 PCR amplification cycles to reach a threshold level of fluorescence using β-2 microglobulin as an internal amplicon reference. The integrity of the RNA samples following DNase I treatment was confirmed by agarose gel electrophoresis and ethidium bromide staining.

After phenol extraction, cDNA was prepared from the sample using the SuperScript™ Choice System following the manufacturer's instructions (GibcoBRL). A negative control of RNA without reverse transcriptase was mock reverse transcribed for each RNA sample.

Expression of the novel h15571 GPCR-like gene sequence was measured by TaqMan® quantitative PCR (Perkin Elmer Applied Biosystems) in cDNA prepared from the following normal human tissues: lymph node, spleen, thymus, brain, lung, skeletal muscle, fetal liver, tonsil, colon, heart, and normal and fibrotic liver; the following primary cells: resting and phytohemaglutinin (PHA) activated peripheral blood mononuclear cells (PBMC); resting and PHA activated CD3+ cells, CD4+ and CD8+ T cells; Th1 and Th2 cells stimulated for six or 48 hours with anti-CD3 antibody; resting and lipopolysaccharide (LPS) activated CD19+B cells; resting and LPS activated CD19+ cells from tonsil; CD34+ cells from mobilized peripheral blood (mPB CD34+), adult resting bone marrow (ABM CD34+), G-CSF mobilized bone marrow (mBM CD34+), and neonatal umbilical cord blood (CB CD34+); G-CSF mobilized peripheral blood leukocytes (mPB leukocytes) and CD34− cells purified from mPB leukocytes (mPB CD34−); CD14+ cells; granulocytes; hepatic stellate cells maintained in serum-free or fetal bovine serum (FBS) containing medium; resting and activated (phorbol 12-myristate 13-acetate (TPA) and ionomycin) normal human liver hepatocytes (NHLH); and fibroblasts (NHDF, normal human dermal fibroblasts; NHLF, normal human lung fibroblasts) mock stimulated or stimulated with transforming growth factor β (TGF-β). Transformed human cell lines included K526, an erythroleukemia; HL60, an acute promyelocytic leukemia; Jurkat, a T cell leukemia; HEK 293, epithelial cells from embryonic kidney transformed with adenovirus 5 DNA; and Hep3B hepatocellular liver carcinoma cells cultured in normal (HepB normoxia) or reduced oxygen tension (Hep3B hypoxia), or mock stimulated or stimulated with TGF-Probes were designed by PrimerExpress software (PE Biosystems) based on the h15571 sequence. The primers and probes for expression analysis of h15571 and β-2 microglobulin were as follows:

h15571 Forward Primer

(SEQ ID NO:3)

GCATCACAGCTGCAGTCAACA
h15571 Reverse Primer

(SEQ ID NO:4)

GCCACACCAGCCAGCAGTA
h15571 TaqMan Probe

(SEQ ID NO:5)

CCACAACTACCGGGACCACAGCCC
.β-2 microglobulin Forward Primer

(SEQ ID NO:6)

CACCCCCACTGAAAAAGATGA
.β-2 microglobulin Reverse Primer

(SEQ ID NO:7)

CTTAACTATCTTGGGCTGTGACAAAG
.β-2 microglobulin TaqMan Probe

(SEQ ID NO:8)

TATGCCTGCCGTGTGAACCACGTG

The h15571 sequence probe was labeled using FAM (6-carboxyfluorescein), and the β2-microglobulin reference probe was labeled with a different fluorescent dye, VIC. The differential labeling of the target GPCR-like sequence and internal reference gene thus enabled measurement in the same well. Forward and reverse primers and the probes for both β2-microglobulin and the target h15571 sequence were added to the TaqMan® Universal PCR Master Mix (PE Applied Biosystems). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target h15571 sequence. TaqMan matrix experiments were carried out on an ABI PRISM 7700 Sequence Detection System (PE Applied Biosystems). The thermal cycler conditions were as follows: hold for 2 min at 50° C. and 10 min at 95° C., followed by two-step PCR for 40 cycles of 95° C. for 15 sec followed by 60° C. for 1 min.

The following method was used to quantitatively calculate h15571 expression in the various tissues relative to β-2 microglobulin expression in the same tissue. The threshold cycle (Ct) value is defined as the cycle at which a statistically significant increase in fluorescence is detected. A lower Ct value is indicative of a higher mRNA concentration. The Ct value of the h15571 sequence is normalized by subtracting the Ct value of the β-2 microglobulin gene to obtain a ΔCt value using the following formula: ΔCt=Cth15571−Ct β-2 microglobulin. Expression is then calibrated against a cDNA sample showing a comparatively low level of expression of the h15571 sequence. The ΔCt value for the calibrator sample is then subtracted from ΔCt for each tissue sample according to the following formula: AΔCt=ΔCt-sample−ΔCt-calibrator. Relative expression is then calculated using the arithmetic formula given by 2−ΔΔCt. Expression of the target h15571 sequence in each of the tissues tested was then graphically represented as discussed in more detail below.

Expression of h15571 was determined in a broad panel of tissues and cell lines as described above, relative to expression in CD3+ T cells. The results indicate significant expression in lung, skeletal muscle, colon, fibrotic liver, and the K562 cell line; moderate expression in brain, and in the HEK 293 and Jurkat cell lines; and low level expression in lymph node, spleen, thymus, fetal liver, tonsil, heart, normal liver, and CB CD34+ cells.

Expression of h15571 in various tissues and cell lines as described above, relative to expression in CD3+ resting cells. The results indicate significant expression in normal human dermal and lung fibroblasts, and in hepatic stellate cells, which are involved in liver fibrosis.

The high expression observed in fibrotic liver samples was reexamined in a comparison of h15571 expression in thirteen fibrotic liver samples against six normal liver samples. The six samples taken from patients with no histological or clinical evidence of liver disease showed minimal expression of h15571. The thirteen samples from patients with histologically defined liver fibrosis, of mixed aetologies including chronic alcohol induced fibrosis, cryptogenic cirrhosis and primary biliary disease, showed upregulation of h15571 to differing degrees.

Isolated cells from this study were used to localize the expression of h15571 to the component cells of the liver or infiltrating inflammatory cells. h15571 expression was seen to be restricted to stellate cells and fibroblasts (NHDF=normal human dermal fibroblasts; NHLF=normal human lung fibroblasts). Activation with either transforming growth factor β (TGF-β) or fetal bovine serum (FBS) was seen to further increase the expression of h15571 in these cells.

The upregulation of h15571 in fibrotic liver samples, and the apparent localization of h15571 expression to activated stellate cells was examined further using similar TaqMan® PCR assays. Expression of h15571 as determined in several tissue and hepatic stellate cell samples relative to expression in hepatocytes 24 hours post-treatment with TGF (Hep-3 cells) is described herein. Expression is clearly elevated in the human liver fibrotic samples, with low-level expression seen in human heart tissue, and nondetectable expression in normal human liver, brain, and kidney tissues. Furthermore, h15571 is not expressed in normal hepatocytes and those treated with PMA or TGF-β. Relative expression within hepatic stellate cells depends upon their physiological state. Thus, quiescent stellate cells show background levels of expression, while passaged stellate (fully activated stellate cells that have been exposed to prolonged culture), resting stellate, and stellate cells reactivated from their resting state with fetal bovine serum (FBS) have high levels of expression.

Elevated expression levels in human liver fibrotic samples and in activated stellate cells indicates a potential role for h15571 in liver fibrosis. This potential role was examined further using rats and three models of liver fibrosis: bile duct ligation (see Kossakowska et al. (1998) Amer. J. Pathol. 153 (6): 1895), a surgical-base model; porcine serum injection (Paronetto and Popper (1966) Amer. J. Pathol. 49:1087, an immunological-based model; and carbon tetrachloride (CCL4) treatment, a toxicity-based model. Significant expression is seen in brain and lung samples, and moderate expression in spinal cord samples. However, expression in normal liver, spleen, kidney, small intestine, and muscle samples is low or even nondetectable. Relative to normal liver, h15571 expression is elevated in rats that have undergone sham operation (i.e., control rats that have been exposed to surgical procedures such as anesthesia, but without bile duct ligation), and markedly elevated in livers of rats having their bile duct ligated for 14 days. Also, expression is elevated in fibrotic livers from rats treated with porcine serum for 7 weeks at 24 hours following the last injection of serum, though the effect is less dramatic than that seen with bile duct ligation.

Expression of rat 15571 in rat liver samples from rats treated with CCL4 is described herein. This toxicity-based model indicates variable expression, but no clear demonstration of upregulation of the h15571 gene.

In summary, these TaqMan® assays reveal significant expression of h15571 in human lung, brain, skeletal muscle, colon, heart, and more particularly in liver fibrosis biopsies. Expression is high in activated hepatic stellate cells, TGF-beta-treated normal human lung fibroblasts, and TGF-beta-treated normal human dermal fibroblasts. Of particular significance is the low expression in normal human liver and nondetectable expression in normal human hepatocytes. Two rat models of liver fibrosis confirm that expression of this gene is elevated in the fibrotic liver tissues from treated animals relative to untreated control animals.

The h15571 protein, a secretin-like/GPCR-like protein, has restricted expression so that high levels of mRNA are detected only in activated hepatic stellate cells, not quiescent cells. Expression in fibrotic livers is elevated as compared to normal livers, and is undetectable in normal human hepatocytes and activated hepatocytes. These data indicate a role for h15571 in the process of fibrosis of the liver.

### Example 3

### In Situ Expression of h15571

Expression of h15571 was also examined by in situ hybridization of riboprobes to cellular mRNAs in the following human tissues: normal liver, fibrotic liver, normal fetal liver, kidney, colon adenocarcinoma, lung, and skeletal muscle. Sense and antisense riboprobes (RNA transcripts) of cDNA encoding h15571 were generated using 35S-dUTP, T3 polymerase, and T7 polymerase, and standard in vitro transcription reaction reagents.

Six um sections of cryopreserved human tissue were prepared using a cryostat and annealed to glass slides, pre-hybed and hybridized to sense and antisense h15771 riboprobes according to standard protocols. Slides containing hybridized tissues and riboprobes were washed extensively (according to standard procedures), dipped in NTB-2 photoemulsion, and were allowed to expose for two weeks. Slides were developed and counterstained with hematoxylin to assist in identifying different subtypes of leukocytes. Data were recorded as pictures of these tissue sections as visualized under a microscope using bright and dark fields. The data from two separate experiments are summarized in Table 1 below.

High levels of h15571 expression were detected in some fibrotic adult livers and in skeletal muscle in two separate experiments. In those fibrotic liver samples exhibiting h15571 expression, activity was consistently detected in mesenchymal cells bordering fibrotic septae.

More specifically, expression of h15571 appears to be localized within activated stellate cells. These stellate cells are a type of myofibroblast believed to mediate the architectural changes that cause liver fibrosis. Thus activated stellate cells cause liver fibrosis, and it is these cells that express high levels of h15571 in liver fibrotic samples. No expression of h15571 was detected in tissue from: normal liver, normal fetal liver, kidney, colon adenocarcinoma, and lung.

The significant and remarkably consistent expression of h15571 in skeletal muscle is an indication of the relatedness of skeletal muscle cells and stellate cells. Myofibroblasts represent a cell type that shares properties with smooth muscle, such as contractability. Both types of cells/tissues express the protein alpha-actin, a mediator of contractability. Changes in this property may contribute to liver fibrosis.

TABLE 1
Expression Analysis of Human 15571 by In Situ Hybridization

| Tissue | h15571 | Comments |
|---|---|---|
| Normal Liver (NDR45) | − | |
| Normal Liver (NDR154) | − | |
| Fibrotic Liver (NDR112) | − | |
| Fibrotic Liver (NDR113) | − | |
| Fibrotic Liver (NDR126) | − | |
| Fibrotic Liver (NDR141) | − | |
| Fibrotic Liver (NDR190) | − | |
| Fibrotic Liver (NDR191) | + | Specific hybridization observed on mesenchymal |
| | | cells bordering fibrotic septae. |
| Fibrotic Liver (NDR192) | + | Specific hybridization observed on mesenchymal |
| | | cells bordering fibrotic septae. |
| Fibrotic Liver (NDR193) | + | Specific hybridization observed on mesenchymal |
| | | cells bordering fibrotic septae. |
| Fibrotic Liver (NDR194) | − | |
| Fibrotic Liver (NDR195) | − | |
| Fibrotic Liver (NDR204) | + | Specific hybridization observed on mesenchymal |
| | | cells bordering fibrotic septae. |
| Fibrotic Liver (NDR225) | − | |
| Normal Fetal Liver (BWH54) | − | |
| Normal Skeletal Muscle | + | |
| (PIT201) | | |
| Normal Kidney (NDR169) | − | |
| Normal Lung (NDR44) | − | |
| Colon adenocarcinoma | − | |
| (NDR99) | | |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

### II. METHODS AND COMPOSITIONS FOR THE DIAGNOSIS AND TREATMENT OF CARDIOVASCULAR, HEPATIC, AND BONE DISEASE

### Background of the Invention

Liver Disorders

One of the most important organs in the body, the liver is specially designed to perform many essential functions, such as the excretion of harmful substances from the body. However, its distinctive characteristics and activities render it susceptible to damage from a variety of sources, and such damage can have enormous impact on a person's health. Typical liver disorders include those related to viral infection (hepatitis), cancer, cirrhosis in response to toxins (e.g., alcohol), parasites, autoimmune conditions, and genetic deficiencies in one or more enzymes critical to liver function leading to, for example, biliary atresia or hemochromatosis.

In response to damage or insult to any of its cell populations, the liver will trigger an immediate response to re-establish tissue integrity. Although different mechanisms may be used, one of two possible responses are generally observed. There is either a re-generation of tissue with complete restoration of tissue architecture and function, or there is a sustained scarring of the tissue, marked by an overproduction of matrix components. This scarring, known as fibrosis, causes deterioration of liver function and can ultimately result in liver failure.

Hepatic stellate cells are the major connective tissue-producing cells in both normal and fibrotic livers. In the normal situation, stellate cells serve as vitamin A storage sites. These cells are quiescent, show little proliferative activity, and express a limited spectrum of connective tissue proteins. In injured or fibrotic livers, however, stellate cells lose their fat-droplets and change their phenotype into myofibroblast-like cells. These myofibroblast-like cells are “activated” cells, show high proliferative activity, and produce large amounts of collagens and other extracellular matrix proteins.

A compound with an anti-fibrotic effect on stellate cells or other hepatic cells will be a promising candidate molecule for the treatment of liver disorders, such as liver fibrosis and cirrhosis. However, to date, there are no truly effective therapeutic drugs for the treatment of the fibrotic condition brought about through liver injury.

Bone Disorders

Human bone is subject to constant breakdown and re-synthesis in a complex process mediated by two cell types: osteoblasts, which produce new bone, and osteoclasts, which destroy bone. The activities of these two cell types are kept under control and in proper balance by a complex network of cytokines, growth factors and other cellular signals. It is understood that a number of known bone disorders may have their genesis in aberrant control of these cells. Likewise, a considerable amount of medical research has focused on identifying the aspects of this control network which can be exploited to re-generate bone in patients with bone diseases.

Osteoporosis is one of several known degenerative bone disorders which can cause significant risk and hardship to those affected. It is generally defined as the gradual decrease in bone strength and density that occurs with advancing age, particularly among post-menopausal women. The clinical manifestations of osteoporosis include fractures of the vertebral bodies, the neck, and intertrochanteric regions of the femur, and the distal radius. Osteoporotic individuals may fracture any bone more easily than their non-osteoporotic counterparts. As many as many as 15-20 million individuals in the United States are afflicted with osteoporosis. About 1.3 million fractures attributable to osteoporosis occur annually in people age 45 and older. Among those who live to be age 90, 32 percent of women and 17 percent of men will suffer a hip fracture, primarily due to osteoporosis.

In addition to osteoporosis, there is a plethora of other conditions which are characterized by the need to enhance bone formation. Perhaps the most obvious is in the case of bone fractures, where it would be desirable to stimulate bone growth and to hasten and complete bone repair. Agents that enhance bone formation would also be useful in certain surgical procedures (e.g., facial reconstruction). Other conditions which result in a deficit or abnormal formation of bone include osteogenesis imperfecta (brittle bone disease), hypophosphatasia, Paget's disease, fibrous dysplasia, osteopetrosis, myeloma bone disease, and the depletion of calcium in bone which is related to primary hyperparathyroidism.

There are currently no pharmaceutical approaches to managing any of these conditions that is completely satisfactory. Bone deterioration associated with osteoporosis and other bone conditions may be treated with estrogens or bisphosphonates, which have known side effects, or with further invasive surgical procedures. Bone fractures are still treated exclusively using casts, braces, anchoring devices and other strictly mechanical means. More recently, surgical approaches to these types of injury utilize bovine or human cadaver bone which is chemically treated (to remove proteins) in order to prevent rejection. However, such bone implants, while mechanically important, are biologically dead (they do not contain bone-forming cells, growth factors, or other regulatory proteins). Thus, they do not greatly modulate the repair process. All of these concerns demonstrate a great need for new or novel forms of bone therapy.

Vascular Disorders

Cardiovascular disease is a major health risk throughout the industrialized world. Atherosclerosis, the most prevalent of cardiovascular diseases, is the principal cause of heart attack, stroke, and gangrene of the extremities, and thereby the principle cause of death in the United States. Atherosclerosis is a complex disease involving many cell types and molecular factors (described in, for example, Ross, 1993, Nature 362: 801-809). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. Injury or dysfunction of the vascular endothelium is a common feature of many conditions that predispose an individual to accelerated development of atherosclerotic cardiovascular disease. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDLs are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

These lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque.

Such plaques occlude the blood vessel concerned and, thus, restrict the flow of blood, resulting in ischemia. Ischemia is a condition characterized by a lack of oxygen supply in tissues of organs due to inadequate perfusion. Such inadequate perfusion can have a number of natural causes, including atherosclerotic or restenotic lesions, anemia, or stroke. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease. Ischemia may occur in any organ, however, that is suffering a lack of oxygen supply.

The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented. Coronary blood flow can also be limited by arterial thrombi, spasm, and, rarely, coronary emboli, as well as by ostial narrowing due to luetic aortitis. Congenital abnormalities, such as anomalous origin of the left anterior descending coronary artery from the pulmonary artery, may cause myocardial ischemia and infarction in infancy, but this cause is very rare in adults.

Myocardial ischemia can also occur if myocardial oxygen demands are abnormally increased, as in severe ventricular hypertrophy due to hypertension or aortic stenosis. The latter can be present with angina that is indistinguishable from that caused by coronary atherosclerosis. A reduction in the oxygen-carrying capacity of the blood, as in extremely severe anemia or in the presence of carboxy-hemoglobin, is a rare cause of myocardial ischemia. Not infrequently, two or more causes of ischemia will coexist, such as an increase in oxygen demand due to left ventricular hypertrophy and a reduction in oxygen supply secondary to coronary atherosclerosis.

The principal surgical approaches to the treatment of ischemic atherosclerosis are bypass grafting, endarterectomy, and percutaneous translumenal angioplasty (PCTA). The failure rate after these approaches due to restenosis, in which the occlusions recur and often become even worse, is extraordinarily high (30-50%). It appears that much of the restenosis is due to further inflammation, smooth muscle accumulation, and thrombosis. Additional therapeutic approaches to cardiovascular disease have included treatments that encouraged angiogenesis in such conditions as ischemic heart and limb disease.

Angiogenesis is a fundamental process by which new blood vessels are formed, as reviewed, for example, by Folkman and Shing, J. Biol. Chem. 267 (16), 10931-10934 (1992). Capillary blood vessels consist of endothelial cells and pericytes. These two cell types carry all of the genetic information to form tubes, branches and whole capillary networks. Specific angiogenic molecules and growth factors can initiate this process, while specific inhibitory molecules can stop it. These molecules with opposing function appear to be continuously acting in concert to maintain a stable microvasculature in which endothelial cell turnover is thousands of days. However, the same endothelial cells can undergo rapid proliferation, i.e. less than five days, during burst of angiogenesis, for example, during wound healing.

Key components of the angiogenic process are the degradation of the basement membrane, the migration and proliferation of capillary endothelial cell (EC) and the formation of three dimensional capillary tubes. The normal vascular turnover is rather low: the doubling time for capillary endothelium is from 50-20,000 days, but it is 2-13 days for tumor capillary endothelium. The current understanding of the sequence of events leading to angiogenesis is that a cytokine capable of stimulating endothelial cell proliferation, such as fibroblast growth factor (FGF), causes release of collagenase or plasminogen activator which, in turn, degrade the basement membrane of the parent venule to facilitate the migration of the endothelial cells. These capillary cells, having sprouted from the parent vessel, proliferate in response to growth factors and angiogenic agents in the surrounding environment to form lumen and eventually new blood vessels.

The development of a vascular blood supply is essential in reproduction, development and wound repair (Folkman, et al., Science 43, 1490-1493 (1989)). Under these conditions, angiogenesis is highly regulated, so that it is turned on only as necessary, usually for brief periods of days, then completely inhibited. However, a number of serious diseases are also dominated by persistent unregulated angiogenesis and/or abnormal neovascularization including solid tumor growth and metastasis, psoriasis, endometriosis, Grave's disease, ischemic disease (e.g., atherosclerosis), and chronic inflammatory diseases (e.g., rheumatoid arthritis), and some types of eye disorders, (reviewed by Auerbach, et al., J. Microvasc. Res. 29, 401-411 (1985); Folkman, Advances in Cancer Research, eds. Klein and Weinhouse, pp. 175-203 (Academic Press, New York 1985); Patz, Am. J. Opthalmol. 94, 715-743 (1982); and Folkman, et al., Science 221, 719-725 (1983)). For example, there are a number of eye diseases, many of which lead to blindness, in which ocular neovascularization occurs in response to the diseased state. These ocular disorders include diabetic retinopathy, macular degeneration, neovascular glaucoma, inflammatory diseases and ocular tumors (e.g., retinoblastoma). There are a number of other eye diseases which are also associated with neovascularization, including retrolental fibroplasia, uveitis, eye diseases associated with choroidal neovascularization and eye diseases which are associated with iris neovascularization.

Vascular tone refers to the degree of constriction experienced by a blood vessel relative to its maximal dilated state. All vessels under basal conditions exhibit some degree of smooth muscle contraction that determines the diameter, and hence tone, of the vessel. Basal vascular tone differs among organs wherein organs with a large vasodilatory capacity have high vascular tone (e.g., myocardium, skeletal muscle, skin), and organs with low vasodilatory capacity have low vascular tone (e.g., cerebral and renal circulatory systems).

Vascular tone is determined by many different competing vasoconstrictor and vasodilator influences acting upon the blood vessel. These influences can be separated into extrinsic factors that originate from outside of the organ or tissue where the blood vessel is located, and intrinsic factors that originate from the vessel itself or the surrounding tissue. Extrinsic factors primarily serve the function of regulating arterial blood pressure, while intrinsic mechanisms are concerned with local blood flow regulation within an organ. Vascular tone at any given instant is determined by the balance of competing vasoconstrictor and vasodilator influences.

### Summary of the Invention

The present invention provides methods and compositions for the diagnosis and treatment of hepatic disease and bone associated disease, including but not limited to, liver fibrosis, hepatitis, liver tumors, cirrhosis of the liver, hemochromatosis, liver parasite induced disorders, alpha-1 antitrypsin deficiency, autoimmune hepatitis, biliary atresia osteogenesis imperfecta (brittle bone disease), osteoporosis, Paget's disease (enlarged bones), fibrous dysplasia (uneven bone growth), hypophosphatasia, osteopetrosis, primary gyperthyroidism, or myeloma bone disease. The present invention is based, at least in part, on the discovery that the 2465 gene is up-regulated in stellate cells (the main effectors of liver fibrosis) as compared to its expression in hepatic cells, and, thus, may be associated with a hepatic disorder. The present invention is further based, at least in part, on the discovery that the 2465 gene is up-regulated during osteoblast differentiation, and, thus, may be associated with a bone disorder.

The present invention is also based, at least in part, on the discovery that the 2465 gene is expressed in isolated human blood vessels (e.g., in isolated endothelial vasculature cells and smooth muscle vasculature cells), and is upregulated in response to laminar shear stress, under proliferating conditions, and during treatment with IL-1β. Accordingly, the present invention also provides methods and compositions for the diagnosis and treatment of cardiovascular disease, including but not limited to, atherosclerosis, ischemia/reperfusion injury, hypertension, restenosis, arterial inflammation, and endothelial cell disorders, such as disorders associated with aberrant endothelial cell growth, angiogenesis and/or vascularization.

In one aspect, the invention provides a method for identifying the presence of a nucleic acid molecule associated with a hepatic, bone, cardiovascular, or endothelial cell disorder in a sample by contacting a sample comprising nucleic acid molecules with a hybridization probe comprising at least 25 contiguous nucleotides of SEQ ID NO:9, and detecting the presence of a nucleic acid molecule associated with a hepatic, bone, cardiovascular, or endothelial cell disorder when the sample contains a nucleic acid molecule that hybridizes to the nucleic acid probe. In one embodiment, the hybridization probe is detectably labeled. In another embodiment the sample comprising nucleic acid molecules is subjected to agarose gel electrophoresis and southern blotting prior to contacting with the hybridization probe. In a further embodiment, the sample comprising nucleic acid molecules is subjected to agarose gel electrophoresis and northern blotting prior to contacting with the hybridization probe. In yet another embodiment, the detecting is by in situ hybridization. In other embodiments, the method is used to detect mRNA or genomic DNA in the sample.

The invention also provides a method for identifying a nucleic acid associated with a hepatic, bone, cardiovascular, or endothelial cell disorder in a sample, by contacting a sample comprising nucleic acid molecules with a first and a second amplification primer, the first primer comprising at least 25 contiguous nucleotides of SEQ ID NO:9 and the second primer comprising at least 25 contiguous nucleotides from the complement of SEQ ID NO:9, incubating the sample under conditions that allow for nucleic acid amplification, and detecting the presence of a nucleic acid molecule associated with a hepatic, bone, cardiovascular, or endothelial cell disorder when the sample contains a nucleic acid molecule that is amplified. In one embodiment, the sample comprising nucleic acid molecules is subjected to agarose gel electrophoresis after the incubation step.

In addition, the invention provides a method for identifying a polypeptide associated with a hepatic, bone, cardiovascular, or endothelial cell disorder in a sample by contacting a sample comprising polypeptide molecules with a binding substance specific for a 2465 polypeptide, and detecting the presence of a polypeptide associated with a hepatic, bone, cardiovascular, or endothelial cell disorder when the sample contains a polypeptide molecule that binds to the binding substance. In one embodiment the binding substance is an antibody. In another embodiment, the binding substance is a 2465 ligand. In a further embodiment, the binding substance is detectably labeled.

In another aspect, the invention provides a method of identifying a subject at risk for a hepatic, bone, cardiovascular, or endothelial cell disorder by contacting a sample obtained from the subject comprising nucleic acid molecules with a hybridization probe comprising at least 25 contiguous nucleotides of SEQ ID NO:9, and detecting the presence of a nucleic acid molecule which identifies a subject a risk for a hepatic, bone, cardiovascular, or endothelial cell disorder when the sample contains a nucleic acid molecule that hybridizes to the nucleic acid probe.

In a further aspect, the invention provides a method for identifying a subject at risk for a hepatic, bone, cardiovascular, or endothelial cell disorder by contacting a sample obtained from a subject comprising nucleic acid molecules with a first and a second amplification primer, the first primer comprising at least 25 contiguous nucleotides of SEQ ID NO:9 and the second primer comprising at least 25 contiguous nucleotides from the complement of SEQ ID NO:9, incubating the sample under conditions that allow for nucleic acid amplification, and detecting a nucleic acid molecule which identifies a subject at risk for a hepatic, bone, cardiovascular, or endothelial cell disorder when the sample contains a nucleic acid molecule that is amplified.

In yet another aspect, the invention provides a method of identifying a subject at risk for a hepatic, bone, cardiovascular, or endothelial cell disorder by contacting a sample obtained from the subject comprising polypeptide molecules with a binding substance specific for a 2465 polypeptide, and identifying a subject at risk for a hepatic, bone, cardiovascular, or endothelial cell disorder by detecting the presence of a polypeptide molecule in the sample that binds to the binding substance.

In another aspect, the invention provides a method for identifying a compound capable of treating a hepatic, bone, cardiovascular, or endothelial cell disorder characterized by aberrant 2465 nucleic acid expression or 2465 protein activity by assaying the ability of the compound to modulate the expression of a 2465 nucleic acid or the activity of a 2465 protein. In one embodiment, the disorder is liver fibrosis. In another embodiment, the disorder is osteoporosis. In another embodiment, the disorder is cardiovascular. In a further embodiment, the ability of the compound to modulate the activity of the 2465 protein is determined by detecting the induction of an intracellular second messenger.

In addition, the invention provides a method for treating a subject having a hepatic, bone, cardiovascular, or endothelial cell disorder characterized by aberrant 2465 protein activity or aberrant 2465 nucleic acid expression by administering to the subject a 2465 modulator. In one embodiment, the 2465 modulator is administered in a pharmaceutically acceptable formulation. In another embodiment the 2465 modulator is administered using a gene therapy vector. In a further embodiment, the 2465 modulator is a small molecule.

In one embodiment, a modulator is capable of modulating 2465 polypeptide activity. In another embodiment, the 2465 modulator is an anti-2465 antibody. In a further embodiment, the 2465 modulator is a 2465 polypeptide comprising the amino acid sequence of SEQ ID NO:10, or a fragment thereof. In yet another embodiment, the 2465 modulator is a 2465 polypeptide comprising an amino acid sequence which is at least 90 percent identical to the amino acid sequence of SEQ ID NO:10, wherein the percent identity is calculated using the ALIGN program for comparing amino acid sequences, a PAM120 weight residue table, a gap length penalty of 12, and a gap penalty of 4. In a further embodiment, the 2465 modulator is an isolated naturally occurring allelic variant of a polypeptide consisting of the amino acid sequence of SEQ ID NO:10, wherein the polypeptide is encoded by a nucleic acid molecule which hybridizes to a complement of a nucleic acid molecule consisting of SEQ ID NO:9 at 6×SSC at 45° C., followed by one or more washes in 0.2×SSC, 0.1% SDS at 50-65° C.

In one embodiment, the 2465 modulator is capable of modulating 2465 nucleic acid expression. In another embodiment, the 2465 modulator is an antisense 2465 nucleic acid molecule. In yet another embodiment, the 2465 modulator is a ribozyme. In a further embodiment, the 2465 modulator comprises the nucleotide sequence of SEQ ID NO:9, or a fragment thereof. In another embodiment, the 2465 modulator comprises a nucleic acid molecule encoding a polypeptide comprising an amino acid sequence which is at least 90 percent identical to the amino acid sequence of SEQ ID NO:10, wherein the percent identity is calculated using the ALIGN program for comparing amino acid sequences, a PAM120 weight residue table, a gap length penalty of 12, and a gap penalty of 4. In yet another embodiment, the 2465 modulator comprises a nucleic acid molecule encoding a naturally occurring allelic variant of a polypeptide comprising the amino acid sequence of SEQ ID NO:10, wherein the nucleic acid molecule which hybridizes to a complement of a nucleic acid molecule consisting of SEQ ID NO:9 at 6×SSC at 45° C., followed by one or more washes in 0.2×SSC, 0.1% SDS at 50-65° C.

In another aspect, the invention provides a method for identifying a compound capable of modulating a hepatocyte, osteocyte, or endothelial cell activity by contacting a hepatocyte, osteocyte, or endothelial cell with a test compound and assaying the ability of the test compound to modulate the expression of a 2465 nucleic acid or the activity of a 2465 protein. In certain embodiments, a compound that modulates the expression of a 2465 nucleic acid or the activity of a 2465 protein modulates hepatocyte, osteocyte, or endothelial cell proliferation, migration, or the expression of cell surface adhesion molecules.

Furthermore, the invention provides a method for modulating a hepatocyte, osteocyte, or endothelial cell activity comprising contacting a hepatocyte, osteocyte, or endothelial cell with a 2465 modulator.

Other features and advantages of the invention will be apparent from the following detailed description and claims.

### Detailed Description of the Invention

The present invention provides methods and compositions for the diagnosis and treatment of cardiovascular, hepatic disease, and bone associated disease, including but not limited to, atherosclerosis, ischemia/reperfusion injury, hypertension, restenosis, arterial inflammation, liver fibrosis, hepatitis, liver tumors, cirrhosis of the liver, hemochromatosis, liver parasite induced disorders, alpha-1 antitrypsin deficiency, autoimmune hepatitis, biliary atresia osteogenesis imperfecta (brittle bone disease), osteoporosis, Paget's disease (enlarged bones), fibrous dysplasia (uneven bone growth), hypophosphatasia, osteopetrosis, primary gyperthyroidism, or myeloma bone disease. The present invention is based, at least in part, on the discovery that G protein-coupled receptor genes, referred to herein as “G protein-coupled receptor 2465” or “2465” nucleic acid and protein molecules, are up-regulated in stellate cells (the main effectors of liver fibrosis) as compared to their expression in hepatic cells, and, thus, may be associated with a hepatic disorder. The present invention is further based, at least in part, on the discovery that the 2465 molecules are up-regulated during osteoblast differentiation, and, thus, may be associated with a bone disorder.

The present invention is also based, at least in part, on the discovery that the 2465 gene is expressed in isolated human blood vessels (e.g., in isolated endothelial vasculature cells and smooth muscle vasculature cells), and is upregulated in response to laminar shear stress, under proliferating conditions, and during treatment with IL-1β.

As used herein, “differential expression” includes both quantitative as well as qualitative differences in the temporal and/or tissue expression pattern of a gene. Thus, a differentially expressed gene may have its expression activated or inactivated in normal versus hepatic, bone, or cardiovascular conditions (for example, in an experimental liver fibrosis disease system or a laminar shear stress system). The degree to which expression differs in normal versus hepatic, bone, or cardiovascular disorder or control versus experimental states need only be large enough to be visualized via standard characterization techniques, e.g., quantitative PCR, Northern analysis, or subtractive hybridization. The expression pattern of a differentially expressed gene may be used as part of a prognostic or diagnostic hepatic, bone, or cardiovascular disorder evaluation, or may be used in methods for identifying compounds useful for the treatment of hepatic, bone, or cardiovascular disorder. In addition, a differentially expressed gene involved in hepatic, bone, or cardiovascular disorders may represent a target gene such that modulation of the level of target gene expression or of target gene product activity may act to ameliorate a hepatic, bone, or cardiovascular disorder condition. Compounds that modulate target gene expression or activity of the target gene product can be used in the treatment of hepatic, bone, or cardiovascular disorders. Although the 2465 genes described herein may be differentially expressed with respect to hepatic, bone, or cardiovascular disorders, and/or their products may interact with gene products important to hepatic, bone, or cardiovascular disorders, the genes may also be involved in mechanisms important to additional hepatic, bone, or cardiovascular processes.

The 2465 molecules of the present invention may be involved in signal transduction and, thus, may function to modulate cell proliferation, differentiation, and motility. Thus, the 2465 molecules of the present invention may play a role in cellular growth signaling mechanisms. As used herein, the term “cellular growth signaling mechanisms” includes signal transmission from cell receptors, e.g., G protein coupled receptors, which regulates 1) cell transversal through the cell cycle, 2) cell differentiation, 3) cell survival, 4) cell migration and patterning, and/or 5) cell proliferation (e.g., endothelial cell proliferation).

Accordingly, the 2465 molecules of the present invention may be involved in cellular signal transduction pathways that modulate hepatic, bone, or cardiovascular cell activity. As used herein, a “hepatic cell activity”, “hepatocyte activity”, or “hepatic cell function” includes cell proliferation, differentiation, migration, and expression of cell surface adhesion molecules, as well as cellular process that contribute to the physiological role of hepatic cells (e.g., the regulation of bile secretion). As used herein, a “bone cell activity”, “osteocyte activity”, or “bone cell function” includes cell proliferation, differentiation, migration, and expression of cell surface adhesion molecules, as well as cellular process that contribute to the physiological role of bone cells (e.g., the regulation of calcium secretion). As used herein, a “cardiovascular cell activity”, “cardiovascular activity”, or “cardiovascular function” includes cell proliferation, differentiation, migration, and expression of cell surface adhesion molecules, as well as cellular process that contribute to the physiological role of cardiovascular cells such as endothelial cells (e.g., the regulation of angiogenesis and/or vascular tone).

The 2465 molecules of the present invention may act as novel diagnostic targets and therapeutic agents for hepatic diseases or disorders. As used herein, a “hepatic disorder” includes a disease or disorder which affects the liver. The term hepatic disorder includes a disorder caused by the over- or under-production of hepatic enzymes, e.g., alanine aminotransferase, aspartate aminotransferase, or γ-glutammyl transferase, in the liver. For example, a hepatic disorder includes hepatic fibrosis, hepatic cirrhosis, a hepatic disorder caused by a drug, a hepatic disorder caused by prolonged ethanol uptake, a hepatic injury caused by carbon tetrachloride exposure, hepatitis, liver tumors, cirrhosis of the liver, hemochromatosis, liver parasite induced disorders, alpha-1 antitrypsin deficiency, or autoimmune hepatitis. Hepatic disorders are disclosed at, for example, the American Liver Foundation website (on the world wide web at: gi.ucsf.edu/alf.html).

The 2465 molecules of the present invention may also act as novel diagnostic targets and therapeutic agents for bone associated diseases or disorders. As used herein, a “bone associated disease or disorder” includes a disease or disorder which affects bones. The term bone associated disorder includes a disorder affecting the normal function of the bones. For example, a bone associated disorder includes biliary atresia osteogenesis imperfecta (brittle bone disease), osteoporosis, Paget's disease (enlarged bones), fibrous dysplasia (uneven bone growth), hypophosphatasia, osteopetrosis, primary gyperthyroidism, or myeloma bone disease. Bone associated disorders are described in, for example, Lamber et al. (2000) Pharmacotherapy 20:34-51; Eisman et al. (1999) Endocrine Reviews 20:788-804; Byers et al. (1992) Annual Rev. Med., 43:269-282.

A hepatic, bone, or cardiovascular disorder also includes a hepatic cell or bone cell disorder. As used herein a “hepatic cell disorder” includes a disorder characterized by aberrant or unwanted hepatic cell activity, e.g., proliferation, migration, angiogenesis, or aberrant expression of cell surface adhesion molecules. As used herein a “bone cell disorder” includes a disorder characterized by aberrant or unwanted bone cell activity, e.g., proliferation, migration, angiogenesis, or aberrant expression of cell surface adhesion molecules.

The 2465 molecules of the present invention may also act as novel diagnostic targets and therapeutic agents for cardiovascular diseases or disorders. As used herein, “cardiovascular disease” or a “cardiovascular disorder” includes a disease or disorder which affects the cardiovascular system, e.g., the heart or the blood vessels. A cardiovascular disorder includes disorders such as arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrillation, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, ischemic disease, arrhythmia, and cardiovascular developmental disorders (e.g., arteriovenous malformations, arteriovenous fistulae, Raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects).

A cardiovasular disease or disorder also includes an endothelial cell and/or smooth muscle cell disorder. As used herein, an “endothelial cell disorder” and/or a “smooth muscle cell disorder” includes a disorder characterized by aberrant, unregulated, or unwanted endothelial cell activity, e.g., vascular tone, vasodilation, vasoconstriction, proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), chronic inflammatory diseases (e.g., rheumatoid arthritis), arterial hypertension, pulmonary hypertension, primary pulmonary hypertension (PPH), Raynaud's phenomenon (RP), migraine headache, chronic heart failure, erythromelalgia, familial dysautonomia, hemolytic uremic syndrome, preeclampsia, reperfusion injury, postangioplasty enothelial regeneration, degeneration of venous bypass grafts, angina, pure spastic angina, diabetes, reflex sympathetic dystrophy syndrome, and vasculitis.

The present invention provides methods for identifying the presence of a 2465 nucleic acid or polypeptide molecule associated with a hepatic, bone, or cardiovascular disorder. In addition, the invention provides methods for identifying a subject at risk for a hepatic, bone, or cardiovascular disorder by detecting the presence of a 2465 nucleic acid or polypeptide molecule.

The invention also provides a method for identifying a compound capable of treating a hepatic, bone, or cardiovascular disorder characterized by aberrant 2465 nucleic acid expression or 2465 protein activity by assaying the ability of the compound to modulate the expression of a 2465 nucleic acid or the activity of a 2465 protein. Furthermore, the invention provides a method for treating a subject having a hepatic, bone, or cardiovascular disorder characterized by aberrant 2465 protein activity or aberrant 2465 nucleic acid expression by administering to the subject a 2465 modulator which is capable of modulating 2465 protein activity or 2465 nucleic acid expression.

Moreover, the invention provides a method for identifying a compound capable of modulating an endothelial cell activity by modulating the expression of a 2465 nucleic acid or the activity of a 2465 protein. The invention provides a method for modulating an endothelial cell activity comprising contacting an endothelial cell with a 2465 modulator.

Various aspects of the invention are described in further detail in the following subsections.

1. Screening Assays

The invention provides a method (also referred to herein as a “screening assay”) for identifying modulators, i.e., candidate or test compounds or agents (e.g., peptides, peptidomimetics, small molecules (organic or inorganic) or other drugs) which bind to 2465 proteins, have a stimulatory or inhibitory effect on, for example, 2465 expression or 2465 activity, or have a stimulatory or inhibitory effect on, for example, the expression or activity of a 2465 substrate.

These assays are designed to identify compounds that bind to a 2465 protein, bind to other cellular or extracellular proteins that interact with a 2465 protein, and interfere with the interaction of the 2465 protein with other cellular or extracellular proteins. For example, in the case of the 2465 protein, which is a transmembrane receptor-type protein, such techniques can identify ligands for such a receptor. A 2465 protein ligand can, for example, act as the basis for amelioration of hepatic, bone, or cardiovascular disorders, such as, for example, atherosclerosis, hypertension, liver fibrosis or osteoporosis. Such compounds may include, but are not limited to peptides, antibodies, or small organic or inorganic compounds. Such compounds may also include other cellular proteins.

Compounds identified via assays such as those described herein may be useful, for example, for ameliorating hepatic, bone, or cardiovascular disorders. In instances whereby a hepatic, bone, or cardiovascular disorder condition results from an overall lower level of 2465 gene expression and/or 2465 protein in a cell or tissue, compounds that interact with the 2465 protein may include compounds which accentuate or amplify the activity of the bound 2465 protein. Such compounds would bring about an effective increase in the level of 2465 protein activity, thus ameliorating symptoms.

In other instances mutations within the 2465 gene may cause aberrant types or excessive amounts of 2465 proteins to be made which have a deleterious effect that leads to hepatic, bone, or cardiovascular disorders. Similarly, physiological conditions may cause an excessive increase in 2465 gene expression leading to hepatic, bone, or cardiovascular disorders. In such cases, compounds that bind to a 2465 protein may be identified that inhibit the activity of the 2465 protein. Assays for testing the effectiveness of compounds identified by techniques such as those described in this section are discussed herein.

In one embodiment, the invention provides assays for screening candidate or test compounds which are substrates of a 2465 protein or polypeptide or biologically active portion thereof. In another embodiment, the invention provides assays for screening candidate or test compounds which bind to or modulate the activity of a 2465 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 2465 protein or biologically active portion thereof is contacted with a test compound and the ability of the test compound to modulate 2465 activity is determined. Determining the ability of the test compound to modulate 2465 activity can be accomplished by monitoring, for example, intracellular calcium, IP3, cAMP, or diacylglycerol concentration, the phosphorylation profile of intracellular proteins, cell proliferation and/or migration, the expression of cell surface adhesion molecules, or the activity of a 2465-regulated transcription factor or gene expression of, for example, cell surface adhesion molecules or genes associated with angiogenesis. The cell can be of mammalian origin, e.g., a hepatic, bone, or endothelial cell. In one embodiment, compounds that interact with a 2465 receptor domain can be screened for their ability to function as ligands, i.e., to bind to the 2465 receptor and modulate a signal transduction pathway. Identification of 2465 ligands, and measuring the activity of the ligand-receptor complex, leads to the identification of modulators (e.g., antagonists) of this interaction. Such modulators may be useful in the treatment of hepatic, bone, or cardiovascular disorders.

The ability of the test compound to modulate 2465 binding to a substrate or to bind to 2465 can also be determined. Determining the ability of the test compound to modulate 2465 binding to a substrate can be accomplished, for example, by coupling the 2465 substrate with a radioisotope or enzymatic label such that binding of the 2465 substrate to 2465 can be determined by detecting the labeled 2465 substrate in a complex. 2465 could also be coupled with a radioisotope or enzymatic label to monitor the ability of a test compound to modulate 2465 binding to a 2465 substrate in a complex. Determining the ability of the test compound to bind 2465 can be accomplished, for example, by coupling the compound with a radioisotope or enzymatic label such that binding of the compound to 2465 can be determined by detecting the labeled 2465 compound in a complex. For example, compounds (e.g., 2465 ligands or substrates) can be labeled with 125I, 35S, 14C, or 3H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 2465 ligand or substrate) to interact with 2465 without the labeling of any of the interactants. For example, a microphysiometer can be used to detect the interaction of a compound with 2465 without the labeling of either the compound or the 2465 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 2465.

In another embodiment, an assay is a cell-based assay comprising contacting a cell expressing a 2465 target molecule (e.g., a 2465 substrate) with a test compound and determining the ability of the test compound to modulate (e.g., stimulate or inhibit) the activity of the 2465 target molecule. Determining the ability of the test compound to modulate the activity of a 2465 target molecule can be accomplished, for example, by determining the ability of the 2465 protein to bind to or interact with the 2465 target molecule.

Determining the ability of the 2465 protein or a biologically active fragment thereof, to bind to or interact with a 2465 target molecule can be accomplished by one of the methods described above for determining direct binding. In a preferred embodiment, determining the ability of the 2465 protein to bind to or interact with a 2465 target molecule can be accomplished by determining the activity of the target molecule. For example, the activity of the target molecule can be determined by detecting induction of a cellular second messenger of the target (i.e., intracellular Ca2+, diacylglycerol, IP3, cAMP), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response (e.g., cell proliferation or migration).

In yet another embodiment, an assay of the present invention is a cell-free assay in which a 2465 protein or biologically active portion thereof, is contacted with a test compound and the ability of the test compound to bind to the 2465 protein or biologically active portion thereof is determined. Preferred biologically active portions of the 2465 proteins to be used in assays of the present invention include fragments which participate in interactions with non-2465 molecules, e.g., fragments with high surface probability scores. Binding of the test compound to the 2465 protein can be determined either directly or indirectly as described above. In a preferred embodiment, the assay includes contacting the 2465 protein or biologically active portion thereof with a known compound which binds 2465 to form an assay mixture, contacting the assay mixture with a test compound, and determining the ability of the test compound to interact with a 2465 protein, wherein determining the ability of the test compound to interact with a 2465 protein comprises determining the ability of the test compound to preferentially bind to 2465 or biologically active portion thereof as compared to the known compound. Compounds that modulate the interaction of 2465 with a known target protein may be useful in regulating the activity of a 2465 protein, especially a mutant 2465 protein.

In another embodiment, the assay is a cell-free assay in which a 2465 protein or biologically active portion thereof is contacted with a test compound and the ability of the test compound to modulate (e.g., stimulate or inhibit) the activity of the 2465 protein or biologically active portion thereof is determined. Determining the ability of the test compound to modulate the activity of a 2465 protein can be accomplished, for example, by determining the ability of the 2465 protein to bind to a 2465 target molecule by one of the methods described above for determining direct binding. Determining the ability of the 2465 protein to bind to a 2465 target molecule can also be accomplished using a technology such as real-time Biomolecular Interaction Analysis (BIA) (Sjolander, S, and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 2465 protein can be accomplished by determining the ability of the 2465 protein to further modulate the activity of a downstream effector of a 2465 target molecule. For example, the activity of the effector molecule on an appropriate target can be determined or the binding of the effector to an appropriate target can be determined as previously described.

In yet another embodiment, the cell-free assay involves contacting a 2465 protein or biologically active portion thereof with a known compound which binds the 2465 protein to form an assay mixture, contacting the assay mixture with a test compound, and determining the ability of the test compound to interact with the 2465 protein, wherein determining the ability of the test compound to interact with the 2465 protein comprises determining the ability of the 2465 protein to preferentially bind to or modulate the activity of a 2465 target molecule.

In more than one embodiment of the above assay methods of the present invention, it may be desirable to immobilize either 2465 or its target molecule to facilitate separation of complexed from uncomplexed forms of one or both of the proteins, as well as to accommodate automation of the assay. Binding of a test compound to a 2465 protein, or interaction of a 2465 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/2465 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 2465 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 2465 binding or activity determined using standard techniques.

Other techniques for immobilizing proteins on matrices can also be used in the screening assays of the invention. For example, either a 2465 protein or a 2465 target molecule can be immobilized utilizing conjugation of biotin and streptavidin. Biotinylated 2465 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 2465 protein or target molecules but which do not interfere with binding of the 2465 protein to its target molecule can be derivatized to the wells of the plate, and unbound target or 2465 protein trapped in the wells by antibody conjugation. Methods for detecting such complexes, in addition to those described above for the GST-immobilized complexes, include immunodetection of complexes using antibodies reactive with the 2465 protein or target molecule, as well as enzyme-linked assays which rely on detecting an enzymatic activity associated with the 2465 protein or target molecule.

In another embodiment, modulators of 2465 expression are identified in a method wherein a cell is contacted with a candidate compound and the expression of 2465 mRNA or protein in the cell is determined. The level of expression of 2465 mRNA or protein in the presence of the candidate compound is compared to the level of expression of 2465 mRNA or protein in the absence of the candidate compound. The candidate compound can then be identified as a modulator of 2465 expression based on this comparison. For example, when expression of 2465 mRNA or protein is greater (statistically significantly greater) in the presence of the candidate compound than in its absence, the candidate compound is identified as a stimulator of 2465 mRNA or protein expression. Alternatively, when expression of 2465 mRNA or protein is less (statistically significantly less) in the presence of the candidate compound than in its absence, the candidate compound is identified as an inhibitor of 2465 mRNA or protein expression. The level of 2465 mRNA or protein expression in the cells can be determined by methods described herein for detecting 2465 mRNA or protein.

In yet another aspect of the invention, the 2465 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 2465 (“2465-binding proteins” or “2465-bp”) and are involved in 2465 activity. Such 2465-binding proteins are also likely to be involved in the propagation of signals by the 2465 proteins or 2465 targets as, for example, downstream elements of a 2465-mediated signaling pathway. Alternatively, such 2465-binding proteins are likely to be 2465 inhibitors.

The two-hybrid system is based on the modular nature of most transcription factors, which consist of separable DNA-binding and activation domains. Briefly, the assay utilizes two different DNA constructs. In one construct, the gene that codes for a 2465 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 2465-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 2465 protein.

In another aspect, the invention pertains to a combination of two or more of the assays described herein. For example, a modulating agent can be identified using a cell-based or a cell free assay, and the ability of the agent to modulate the activity of a 2465 protein can be confirmed in vivo, e.g., in an animal such as an animal model for hepatic, bone, or cardiovascular disorders, as described herein.

Examples of animal models of hepatic fibrosis include animal models suffering from carbon tetrachloride intoxication, iron and alcohol intoxication, streptococcal cell wall administration, and bile duct ligation, e.g., in rats, as well as mice suffering from schistosomiasis. These animal models are known in the art and are described in, for example, Czaja et al. (1989) J. Cell. Biol. 108:2477-2482; Manthey et al. (1990) Growth Factors 4:17-26; Bissell et al. (1995) J. Clin. Invest. 96:447-455; Tsukamoto et al. (1995) J. Clin. Invest. 96:620-630; Alcolado et al. (1997) Clin. Sci. 92:103-112; Cales (1998) Biomed. and Pharmacother. 52:259-263.

Animal-based model systems of cardiovascular disease may include, but are not limited to, non-recombinant and engineered transgenic animals.

Non-recombinant animal models for cardiovascular disease may include, for example, genetic models. Such genetic cardiovascular disease models may include, for example, apoB or apoR deficient pigs (Rapacz, et al., 1986, Science 234:1573-1577) and Watanabe heritable hyperlipidemic (WHHL) rabbits (Kita et al., 1987, Proc. Natl. Acad. Sci. USA 84: 5928-5931). Transgenic mouse models in cardiovascular disease and angiogenesis are reviewed in Carmeliet, P. and Collen, D. (2000) J. Pathol. 190:387-405.

Non-recombinant, non-genetic animal models of atherosclerosis may include, for example, pig, rabbit, or rat models in which the animal has been exposed to either chemical wounding through dietary supplementation of LDL, or mechanical wounding through balloon catheter angioplasty. Animal models of cardiovascular disease also include rat myocardial infarction models (described in, for example, Schwarz, E R et al. (2000) J. Am. Coll. Cardiol. 35:1323-1330) and models of chromic cardiac ischemia in rabbits (described in, for example, Operschall, C et al. (2000) J. Appl. Physiol. 88:1438-1445).

Models for studying angiogenesis in vivo include tumor cell-induced angiogenesis and tumor metastasis (Hoffman, R M (1998-99) Cancer Metastasis Rev. 17:271-277; Holash, J et al. (1999) Oncogene 18:5356-5362; Li, C Y et al. (2000) J. Natl Cancer Inst. 92:143-147), matrix induced angiogenesis (U.S. Pat. No. 5,382,514), the disc angiogenesis system (Kowalski, J. et al. (1992) Exp. Mol. Pathol. 56:1-19), the rodent mesenteric-window angiogenesis assay (Norrby, K (1992) EXS 61:282-286), experimental choroidal neovascularization in the rat (Shen, W Y et al. (1998) Br. J. Opthalmol. 82:1063-1071), and the chick embryo development (Brooks, P C et al. Methods Mol. Biol. (1999) 129:257-269) and chick embryo chorioallantoic membrane (CAM) models (McNatt L G et al. (1999) J. Ocul. Pharmacol. Ther. 15:413-423; Ribatti, D et al. (1996) Int. J. Dev. Biol. 40:1189-1197), and are reviewed in Ribatti, D and Vacca, A (1999) Int. J. Biol. Markers 14:207-213.

Models for studying vascular tone in vivo include the rabbit femoral artery model (Luo et al. (2000) J. Clin. Invest. 106:493-499), eNOS knockout mice (Hannan et al. (2000) J. Surg. Res. 93:127-132), rat models of cerebral ischemia (Cipolla et al. (2000) Stroke 31:940-945), the renin-angiotensin mouse system (Cvetkovik et al. (2000) Kidney Int. 57:863-874), the rat lung transplant model (Suda et al. (2000) J. Thorac. Cardiovasc. Surg. 119:297-304), the New Zealand White rabbit model of intracranial hypertension (Richards et al. (1999) Acta Neurochir. 141:1221-1227), the spontaneously hypertensive (SH) rat neurogenic model of chronic hypertension (Stekiel et al. (1999) Anesthesiology 91:207-214), the Prague hypertensive rat (PHR) (Vogel et al. (1999) Clin. Sci. 97:91-98), chronically angiotensin II (Ang II)-infused rats (Pasquie et al. (1999) Hypertension 33:830-834), Dahl-salt-sensitive rats (Boulanger (1999) J. Mol. Cell. Cardiol. 31:39-49), the mouse model of arterial remodeling (Bryant et al. (1999) Circ. Res. 84:323-328), and the obese Zucker (fa/fa) rat (Golub et al. (1998) Hypertens. Res. 21:283-288).

Cells that contain and express 2465 gene sequences which encode a 2465 protein, and, further, exhibit cellular phenotypes associated with cardiovascular disease, may be used to identify compounds that exhibit anti-cardiovascular disease activity. Such cells may include non-recombinant monocyte cell lines, such as U937 (ATCC # CRL-1593), THP-1 (ATCC #TIB-202), and P388D1 (ATCC # TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC # CRL-1651). Further, such cells may include recombinant, transgenic cell lines. For example, the cardiovascular disease animal models of the invention, discussed above, may be used to generate cell lines, containing one or more cell types involved in cardiovascular disease, that can be used as cell culture models for this disorder. While primary cultures derived from the cardiovascular disease transgenic animals of the invention may be utilized, the generation of continuous cell lines is preferred. For examples of techniques which may be used to derive a continuous cell line from the transgenic animals, see Small et al., (1985) Mol. Cell. Biol. 5:642-648.

Alternatively, cells of a cell type known to be involved in cardiovascular disease may be transfected with sequences capable of increasing or decreasing the amount of 2465 gene expression within the cell. For example, 2465 gene sequences may be introduced into, and overexpressed in, the genome of the cell of interest, or, if endogenous 2465 gene sequences are present, they may be either overexpressed or, alternatively disrupted in order to underexpress or inactivate 2465 gene expression.

This invention further pertains to novel agents identified by the above-described screening assays. Accordingly, it is within the scope of this invention to further use an agent identified as described herein in an appropriate animal model. For example, an agent identified as described herein (e.g., a 2465 modulating agent, an antisense 2465 nucleic acid molecule, a 2465-specific antibody, or a 2465-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate hepatic, bone, or cardiovascular disorder symptoms. Cell-based and animal model-based assays for the identification of compounds exhibiting such an ability to ameliorate hepatic, bone, or cardiovascular disorder systems are described herein.

In one aspect, cell-based systems, as described herein, may be used to identify compounds which may act to ameliorate hepatic, bone, or cardiovascular disorder symptoms. For example, such cell systems may be exposed to a compound, suspected of exhibiting an ability to ameliorate hepatic, bone, or cardiovascular disorder symptoms, at a sufficient concentration and for a time sufficient to elicit such an amelioration of hepatic, bone, or cardiovascular disorder symptoms in the exposed cells. After exposure, the cells are examined to determine whether one or more of the hepatic, bone, or cardiovascular disorder cellular phenotypes has been altered to resemble a more normal or more wild type, non-hepatic or non-bone associated disease phenotype. Cellular phenotypes that are associated with hepatic, bone, or cardiovascular disorder states include aberrant proliferation and migration, deposition of extracellular matrix components, and expression of growth factors, cytokines, and other inflammatory mediators.

In addition, animal-based hepatic, bone, or cardiovascular disorder or disease systems, such as those described herein, may be used to identify compounds capable of ameliorating hepatic, bone, or cardiovascular disorder symptoms. Such animal models may be used as test substrates for the identification of drugs, pharmaceuticals, therapies, and interventions which may be effective in treating hepatic, bone, or cardiovascular disorders. For example, animal models may be exposed to a compound, suspected of exhibiting an ability to ameliorate hepatic, bone, or cardiovascular disorder symptoms, at a sufficient concentration and for a time sufficient to elicit such an amelioration of hepatic, bone, or cardiovascular disorder symptoms in the exposed animals. The response of the animals to the exposure may be monitored by assessing the reversal of disorders associated with hepatic, bone, or cardiovascular disorders, for example, by measuring liver loss and/or measuring bone loss before and after treatment.

With regard to intervention, any treatments which reverse any aspect of hepatic, bone, or cardiovascular disorder symptoms should be considered as candidates for human hepatic, bone, or cardiovascular disorder therapeutic intervention. Dosages of test agents may be determined by deriving dose-response curves.

Additionally, gene expression patterns may be utilized to assess the ability of a compound to ameliorate hepatic, bone, or cardiovascular disorder symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, atherosclerosis, ischemia/reperfusion, hypertension, restenosis, arterial inflammation, and liver fibrosis including any of the control or experimental conditions described herein. Gene expression profiles may be generated, for example, by utilizing a differential display procedure, Northern analysis and/or RT-PCR. In one embodiment, 2465 gene sequences may be used as probes and/or PCR primers for the generation and corroboration of such gene expression profiles.

Gene expression profiles may be characterized for known states, either hepatic, bone, or cardiovascular disorders or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a hepatic, bone, or cardiovascular disorder model system to more closely resemble the control system. Administration of a compound may, alternatively, cause the gene expression profile of a control system to begin to mimic a hepatic, bone, or cardiovascular disorder state. Such a compound may, for example, be used in further characterizing the compound of interest, or may be used in the generation of additional animal models.

2. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 2465 protein and/or nucleic acid expression as well as 2465 activity, in the context of a biological sample (e.g., blood, serum, cells, tissue) to thereby determine whether an individual is afflicted with a disease or disorder, or is at risk of developing a hepatic, bone, or cardiovascular disorder, associated with aberrant or unwanted 2465 expression or activity. The invention also provides for prognostic (or predictive) assays for determining whether an individual is at risk of developing a disorder associated with 2465 protein, nucleic acid expression or activity. For example, mutations in a 2465 gene can be assayed in a biological sample. Such assays can be used for prognostic or predictive purpose to thereby prophylactically treat an individual prior to the onset of a disorder characterized by or associated with 2465 protein, nucleic acid expression or activity.

Another aspect of the invention pertains to monitoring the influence of agents (e.g., drugs, compounds) on the expression or activity of 2465 in clinical trials.

These and other agents are described in further detail in the following sections.

A. Diagnostic Assays

The present invention encompasses methods for diagnostic and prognostic evaluation of hepatic, bone, or cardiovascular disorder conditions, and for the identification of subjects exhibiting a predisposition to such conditions.

An exemplary method for detecting the presence or absence of 2465 protein or nucleic acid in a biological sample involves obtaining a biological sample from a test subject and contacting the biological sample with a compound or an agent capable of detecting 2465 protein or nucleic acid (e.g., mRNA, or genomic DNA) that encodes 2465 protein such that the presence of 2465 protein or nucleic acid is detected in the biological sample. A preferred agent for detecting 2465 mRNA or genomic DNA is a labeled nucleic acid probe capable of hybridizing to 2465 mRNA or genomic DNA. The nucleic acid probe can be, for example, the 2465 nucleic acid set forth in SEQ ID NO:9, or a portion thereof, such as an oligonucleotide of at least 15, 20, 25, 30, 35, 40, 45, 50, 100, 250 or 500 nucleotides in length and sufficient to specifically hybridize under stringent conditions to 2465 mRNA or genomic DNA. Other suitable probes for use in the diagnostic assays of the invention are described herein.

A preferred agent for detecting 2465 protein is an antibody capable of binding to 2465 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect 2465 mRNA, protein, or genomic DNA in a biological sample in vitro as well as in vivo. For example, in vitro techniques for detection of 2465 mRNA include Northern hybridizations and in situ hybridizations. In vitro techniques for detection of 2465 protein include enzyme linked immunosorbent assays (ELISAs), Western blots, immunoprecipitations and immunofluorescence. In vitro techniques for detection of 2465 genomic DNA include Southern hybridizations. Furthermore, in vivo techniques for detection of 2465 protein include introducing into a subject a labeled anti-2465 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting 2465 protein, mRNA, or genomic DNA, such that the presence of 2465 protein, mRNA or genomic DNA is detected in the biological sample, and comparing the presence of 2465 protein, mRNA or genomic DNA in the control sample with the presence of 2465 protein, mRNA or genomic DNA in the test sample.

The invention also encompasses kits for detecting the presence of 2465 in a biological sample. For example, the kit can comprise a labeled compound or agent capable of detecting 2465 protein or mRNA in a biological sample; means for determining the amount of 2465 in the sample; and means for comparing the amount of 2465 in the sample with a standard. The compound or agent can be packaged in a suitable container. The kit can further comprise instructions for using the kit to detect 2465 protein or nucleic acid.

B. Prognostic Assays

The diagnostic methods described herein can furthermore be utilized to identify subjects having or at risk of developing a hepatic, bone, or cardiovascular disease or disorder associated with aberrant or unwanted 2465 expression or activity. As used herein, the term “aberrant” includes a 2465 expression or activity which deviates from the wild type 2465 expression or activity. Aberrant expression or activity includes increased or decreased expression or activity, as well as expression or activity which does not follow the wild type developmental pattern of expression or the subcellular pattern of expression. For example, aberrant 2465 expression or activity is intended to include the cases in which a mutation in the 2465 gene causes the 2465 gene to be under-expressed or over-expressed and situations in which such mutations result in a non-functional 2465 protein or a protein which does not function in a wild-type fashion, e.g., a protein which does not interact with a 2465 ligand or substrate, or one which interacts with a non-2465 ligand or substrate. As used herein, the term “unwanted” includes an unwanted phenomenon involved in a biological response such as cellular proliferation. For example, the term unwanted includes a 2465 expression pattern or a 2465 protein activity which is undesirable in a subject.

The assays described herein, such as the preceding diagnostic assays or the following assays, can be utilized to identify a subject having or at risk of developing a disorder associated with a misregulation in 2465 protein activity or nucleic acid expression, such as a hepatic, bone, or cardiovascular disorder. Alternatively, the prognostic assays can be utilized to identify a subject having or at risk for developing a hepatic, bone, or cardiovascular disorder associated with a misregulation in 2465 protein activity or nucleic acid expression. Thus, the present invention provides a method for identifying a disease or disorder associated with aberrant or unwanted 2465 expression or activity in which a test sample is obtained from a subject and 2465 protein or nucleic acid (e.g., mRNA or genomic DNA) is detected, wherein the presence of 2465 protein or nucleic acid is diagnostic for a subject having or at risk of developing a disease or disorder associated with aberrant or unwanted 2465 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 2465 expression or activity. For example, such methods can be used to determine whether a subject can be effectively treated with an agent for a hepatic, bone, or cardiovascular disorder. Thus, the present invention provides methods for determining whether a subject can be effectively treated with an agent for a hepatic, bone, or cardiovascular disorder associated with aberrant or unwanted 2465 expression or activity in which a test sample is obtained and 2465 protein or nucleic acid expression or activity is detected (e.g., wherein the abundance of 2465 protein or nucleic acid expression or activity is diagnostic for a subject that can be administered the agent to treat a disorder associated with aberrant or unwanted 2465 expression or activity).

The methods of the invention can also be used to detect genetic alterations in a 2465 gene, thereby determining if a subject with the altered gene is at risk for a disorder characterized by misregulation in 2465 protein activity or nucleic acid expression, such as a proliferative disorder. In preferred embodiments, the methods include detecting, in a sample of cells from the subject, the presence or absence of a genetic alteration characterized by at least one of an alteration affecting the integrity of a gene encoding a 2465-protein, or the mis-expression of the 2465 gene. For example, such genetic alterations can be detected by ascertaining the existence of at least one of 1) a deletion of one or more nucleotides from a 2465 gene; 2) an addition of one or more nucleotides to a 2465 gene; 3) a substitution of one or more nucleotides of a 2465 gene, 4) a chromosomal rearrangement of a 2465 gene; 5) an alteration in the level of a messenger RNA transcript of a 2465 gene, 6) aberrant modification of a 2465 gene, such as of the methylation pattern of the genomic DNA, 7) the presence of a non-wild type splicing pattern of a messenger RNA transcript of a 2465 gene, 8) a non-wild type level of a 2465-protein, 9) allelic loss of a 2465 gene, and 10) inappropriate post-translational modification of a 2465-protein. As described herein, there are a large number of assays known in the art which can be used for detecting alterations in a 2465 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the 2465-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a 2465 gene under conditions such that hybridization and amplification of the 2465-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Other amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 2465 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 2465 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 2465 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 2465 gene and detect mutations by comparing the sequence of the sample 2465 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 2465 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type 2465 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 2465 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 2465 sequence, e.g., a wild-type 2465 sequence, is hybridized to a cDNA or other DNA product from a test cell(s). The duplex is treated with a DNA mismatch repair enzyme, and the cleavage products, if any, can be detected from electrophoresis protocols or the like (described in, for example, U.S. Pat. No. 5,459,039).

In other embodiments, alterations in electrophoretic mobility will be used to identify mutations in 2465 genes. For example, single strand conformation polymorphism (SSCP) may be used to detect differences in electrophoretic mobility between mutant and wild type nucleic acids (Orita et al. (1989) Proc Natl Acad. Sci. USA: 86:2766, see also Cotton (1993) Mutat. Res. 285:125-144; and Hayashi (1992) Genet. Anal. Tech. Appl. 9:73-79). Single-stranded DNA fragments of sample and control 2465 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 2465 gene.

Furthermore, any cell type or tissue in which 2465 is expressed may be utilized in the prognostic assays described herein.

C. Monitoring of Effects During Clinical Trials

The present invention provides methods for evaluating the efficacy of drugs and monitoring the progress of patients involved in clinical trials for the treatment of hepatic, bone, or cardiovascular disorders.

Monitoring the influence of agents (e.g., drugs) on the expression or activity of a 2465 protein (e.g., the modulation of cell proliferation and/or migration) can be applied not only in basic drug screening, but also in clinical trials. For example, the effectiveness of an agent determined by a screening assay as described herein to increase 2465 gene expression, protein levels, or upregulate 2465 activity, can be monitored in clinical trials of subjects exhibiting decreased 2465 gene expression, protein levels, or downregulated 2465 activity. Alternatively, the effectiveness of an agent determined by a screening assay to decrease 2465 gene expression, protein levels, or downregulate 2465 activity, can be monitored in clinical trials of subjects exhibiting increased 2465 gene expression, protein levels, or upregulated 2465 activity. In such clinical trials, the expression or activity of a 2465 gene, and preferably, other genes that have been implicated in, for example, a 2465-associated disorder can be used as a “read out” or markers of the phenotype a particular cell, e.g., an endothelial cell. In addition, the expression of a 2465 gene, or the level of 2465 protein activity may be used as a read out of a particular drug or agent's effect on a hepatic, bone, or cardiovascular disorders state.

For example, and not by way of limitation, genes, including 2465, that are modulated in cells by treatment with an agent (e.g., compound, drug or small molecule) which modulates 2465 activity (e.g., identified in a screening assay as described herein) can be identified. Thus, to study the effect of agents on 2465-associated disorders (e.g., hepatic, bone, or cardiovascular disorders characterized by deregulated endothelial cell activity), for example, in a clinical trial, cells can be isolated and RNA prepared and analyzed for the levels of expression of 2465 and other genes implicated in the 2465-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of 2465 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a 2465 protein, mRNA, or genomic DNA in the preadministration sample; (iii) obtaining one or more post-administration samples from the subject; (iv) detecting the level of expression or activity of the 2465 protein, mRNA, or genomic DNA in the post-administration samples; (v) comparing the level of expression or activity of the 2465 protein, mRNA, or genomic DNA in the pre-administration sample with the 2465 protein, mRNA, or genomic DNA in the post administration sample or samples; and (vi) altering the administration of the agent to the subject accordingly. For example, increased administration of the agent may be desirable to increase the expression or activity of 2465 to higher levels than detected, i.e., to increase the effectiveness of the agent. Alternatively, decreased administration of the agent may be desirable to decrease expression or activity of 2465 to lower levels than detected, i.e. to decrease the effectiveness of the agent. According to such an embodiment, 2465 expression or activity may be used as an indicator of the effectiveness of an agent, even in the absence of an observable phenotypic response.

3. Methods of Treatment:

The present invention provides for both prophylactic and therapeutic methods of treating a subject at risk of (or susceptible to) a disorder or having a disorder associated with aberrant or unwanted 2465 expression or activity, e.g. a hepatic, bone, or cardiovascular disorder. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 2465 molecules of the present invention or 2465 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Treatment is defined as the application or administration of a therapeutic agent to a patient, or the application or administration of a therapeutic agent to an isolated tissue or cell line from a patient, who has a disease, a symptom of disease or a predisposition toward a disease, with the purpose of curing, healing, alleviating, relieving, altering, remedying, ameliorating, improving or affecting the disease, the symptoms of disease or the predisposition toward disease as described herein.

A therapeutic agent includes, but is not limited to, small molecules, peptides, antibodies, ribozymes and antisense oligonucleotides.

A. Prophylactic Methods

In one aspect, the invention provides a method for preventing in a subject, a hepatic, bone, or cardiovascular disorder or condition associated with an aberrant or unwanted 2465 expression or activity, by administering to the subject a 2465 or an agent which modulates 2465 expression or at least one 2465 activity. Subjects at risk for a hepatic, bone, or cardiovascular disorder which is caused or contributed to by aberrant or unwanted 2465 expression or activity can be identified by, for example, any or a combination of diagnostic or prognostic assays as described herein. Administration of a prophylactic agent can occur prior to the manifestation of symptoms characteristic of the 2465 aberrancy, such that a disease or disorder is prevented or, alternatively, delayed in its progression. Depending on the type of 2465 aberrancy, for example, a 2465, 2465 agonist or 2465 antagonist agent can be used for treating the subject. The appropriate agent can be determined based on screening assays described herein.

B. Therapeutic Methods

Described herein are methods and compositions whereby hepatic, bone, or cardiovascular disorder symptoms may be ameliorated. Certain hepatic, bone, or cardiovascular disorders are brought about, at least in part, by an excessive level of a gene product, or by the presence of a gene product exhibiting an abnormal or excessive activity. As such, the reduction in the level and/or activity of such gene products would bring about the amelioration of hepatic, bone, or cardiovascular disorder symptoms. Techniques for the reduction of gene expression levels or the activity of a protein are discussed below.

Alternatively, certain other hepatic, bone, or cardiovascular disorders are brought about, at least in part, by the absence or reduction of the level of gene expression, or a reduction in the level of a protein's activity. As such, an increase in the level of gene expression and/or the activity of such proteins would bring about the amelioration of hepatic, bone, or cardiovascular disorder symptoms.

In some cases, the up-regulation of a gene in a disease state reflects a protective role for that gene product in responding to the disease condition. Enhancement of such a gene's expression, or the activity of the gene product, will reinforce the protective effect it exerts. Some hepatic, bone, or cardiovascular disorder states may result from an abnormally low level of activity of such a protective gene. In these cases also, an increase in the level of gene expression and/or the activity of such gene products would bring about the amelioration of hepatic, bone, or cardiovascular disorder symptoms. Techniques for increasing target gene expression levels or target gene product activity levels are discussed herein.

Accordingly, another aspect of the invention pertains to methods of modulating 2465 expression or activity for therapeutic purposes. Accordingly, in an exemplary embodiment, the modulatory method of the invention involves contacting a cell with a 2465 or agent that modulates one or more of the activities of 2465 protein activity associated with the cell (e.g., a hepatic cell). An agent that modulates 2465 protein activity can be an agent as described herein, such as a nucleic acid or a protein, a naturally-occurring target molecule of a 2465 protein (e.g., a 2465 ligand or substrate), a 2465 antibody, a 2465 agonist or antagonist, a peptidomimetic of a 2465 agonist or antagonist, or other small molecule. In one embodiment, the agent stimulates one or more 2465 activities. Examples of such stimulatory agents include active 2465 protein and a nucleic acid molecule encoding 2465 that has been introduced into the cell. In another embodiment, the agent inhibits one or more 2465 activities. Examples of such inhibitory agents include antisense 2465 nucleic acid molecules, anti-2465 antibodies, and 2465 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 2465 protein or nucleic acid molecule. In one embodiment, the method involves administering an agent (e.g., an agent identified by a screening assay described herein), or combination of agents that modulates (e.g., upregulates or downregulates) 2465 expression or activity. In another embodiment, the method involves administering a 2465 protein or nucleic acid molecule as therapy to compensate for reduced, aberrant, or unwanted 2465 expression or activity.

Stimulation of 2465 activity is desirable in situations in which 2465 is abnormally downregulated and/or in which increased 2465 activity is likely to have a beneficial effect. Likewise, inhibition of 2465 activity is desirable in situations in which 2465 is abnormally upregulated and/or in which decreased 2465 activity is likely to have a beneficial effect.

(i) Methods for Inhibiting Target Gene Expression, Synthesis, or Activity

As discussed above, genes involved in hepatic, bone, or cardiovascular disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate hepatic, bone, or cardiovascular disorder symptoms. Such molecules may include, but are not limited to, small organic molecules, peptides, antibodies, and the like.

For example, compounds can be administered that compete with endogenous ligand for the 2465 protein. The resulting reduction in the amount of ligand-bound 2465 protein will modulate endothelial cell physiology. Compounds that can be particularly useful for this purpose include, for example, soluble proteins or peptides, such as peptides comprising one or more of the extracellular domains, or portions and/or analogs thereof, of the 2465 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 2465 receptor site, but do not activate the protein, (e.g., receptor-ligand antagonists) can be effective in inhibiting 2465 protein activity.

Further, antisense and ribozyme molecules which inhibit expression of the 2465 gene may also be used in accordance with the invention to inhibit aberrant 2465 gene activity. Still further, triple helix molecules may be utilized in inhibiting aberrant 2465 gene activity.

The antisense nucleic acid molecules of the invention are typically administered to a subject or generated in situ such that they hybridize with or bind to cellular mRNA and/or genomic DNA encoding a 2465 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 2465 mRNA transcripts to thereby inhibit translation of 2465 mRNA. A ribozyme having specificity for a 2465-encoding nucleic acid can be designed based upon the nucleotide sequence of a 2465 cDNA disclosed herein (i.e., SEQ ID NO:9). For example, a derivative of a Tetrahymena L-19 IVS RNA can be constructed in which the nucleotide sequence of the active site is complementary to the nucleotide sequence to be cleaved in a 2465-encoding mRNA (see, for example, Cech et al. U.S. Pat. No. 4,987,071; and Cech et al. U.S. Pat. No. 5,116,742). Alternatively, 2465 mRNA can be used to select a catalytic RNA having a specific ribonuclease activity from a pool of RNA molecules (see, for example, Bartel, D. and Szostak, J. W. (1993) Science 261:1411-1418).

2465 gene expression can also be inhibited by targeting nucleotide sequences complementary to the regulatory region of the 2465 (e.g., the 2465 promoter and/or enhancers) to form triple helical structures that prevent transcription of the 2465 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 2465 protein and interfere with its activity may also be used to modulate or inhibit 2465 protein function. Such antibodies may be generated using standard techniques described herein, against the 2465 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 2465 protein. Antibodies that are specific for one or more extracellular domains of the 2465 protein, for example, and that interfere with its activity, are particularly useful in treating hepatic, bone, or cardiovascular disorders. Such antibodies are especially efficient because they can access the target domains directly from the bloodstream. Any of the administration techniques described below which are appropriate for peptide administration may be utilized to effectively administer inhibitory target gene antibodies to their site of action.

(ii) Methods for Restoring or Enhancing Target Gene Activity

Genes that cause hepatic, bone, or cardiovascular disorders may be underexpressed within hepatic, bone, or cardiovascular disorder situations. Alternatively, the activity of the protein products of such genes may be decreased, leading to the development of hepatic, bone, or cardiovascular disorder symptoms. Such down-regulation of gene expression or decrease of protein activity might have a causative or exacerbating effect on the disease state.

In some cases, genes that are up-regulated in the disease state might be exerting a protective effect. Specifically, 2465 is up-regulated in stellate cells (the main effectors of liver fibrosis), Furthermore, 2465 is up-regulated during osteoblast differentiation. 2465 is also up-regulated during laminar shear stress, proliferation, and in the presence of IL-1 [symbol] (stimuli relevant to angiogenesis, atherosclerosis, and vascular tone). A variety of techniques may be used to decrease the expression, synthesis, or activity of 2465 genes and/or proteins that exert a causatory effect on hepatic, bone, or cardiovascular disorder conditions.

Described in this section are methods whereby the level 2465 activity may be modulated to levels wherein hepatic, bone, or cardiovascular disorder symptoms are ameliorated. The level of 2465 activity may be modulated, for example, by either modulating the level of 2465 gene expression or by modulating the level of active 2465 protein which is present. For example, an inhibitor of a 2465 protein, at a level sufficient to ameliorate hepatic, bone, or cardiovascular disorder symptoms may be administered to a patient exhibiting such symptoms. Any of the techniques discussed below may be used for such administration. One of skill in the art will readily know how to determine the concentration of effective, non-toxic doses of an inhibitor of the 2465 protein, utilizing techniques such as those described below.

Additionally, antisense 2465 DNA sequences may be directly administered to a patient exhibiting hepatic, bone, or cardiovascular disorder symptoms, at a concentration sufficient to reduce the level of 2465 protein such that hepatic, bone, or cardiovascular disorder symptoms are ameliorated. Any of the techniques discussed below, which achieve intracellular administration of compounds, such as, for example, liposome administration, may be used for the administration of such antisense DNA molecules. The DNA molecules may be produced, for example, by recombinant techniques such as those described herein.

Further, subjects may be treated by gene replacement therapy. One or more copies of an antagonist of the 2465 molecule, e.g., a portion of the 2465 gene, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 2465 gene sequences into human cells.

Cells, preferably, autologous cells, containing 2465 antagonist expressing gene sequences may then be introduced or reintroduced into the subject at positions which allow for the amelioration of hepatic, bone, or cardiovascular disorder symptoms. Such cell replacement techniques may be preferred, for example, when the gene product is a secreted, extracellular gene product.

C. Pharmacogenomics

The 2465 molecules of the present invention, as well as agents, or modulators which have a stimulatory or inhibitory effect on 2465 activity (e.g., 2465 gene expression) as identified by a screening assay described herein can be administered to individuals to treat (prophylactically or therapeutically) 2465-associated disorders (e.g., hepatic, bone, or cardiovascular disorders) associated with aberrant or unwanted 2465 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 2465 molecule or a 2465 modulator as well as tailoring the dosage and/or therapeutic regimen of treatment with a 2465 molecule or 2465 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a 2465 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a 2465 molecule or 2465 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a 2465 molecule or 2465 modulator, such as a modulator identified by one of the exemplary screening assays described herein.

4. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

A. Chromosome Mapping

Once the sequence (or a portion of the sequence) of a gene has been isolated, this sequence can be used to map the location of the gene on a chromosome. This process is called chromosome mapping. Accordingly, portions or fragments of the 2465 nucleotide sequences, described herein, can be used to map the location of the 2465 genes on a chromosome. The mapping of the 2465 sequences to chromosomes is an important first step in correlating these sequences with genes associated with disease. The 2465 gene has been mapped to human chromosome position 15q14-15.

Briefly, 2465 genes can be mapped to chromosomes by preparing PCR primers (preferably 15-25 bp in length) from the 2465 nucleotide sequences. Computer analysis of the 2465 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the 2465 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the 2465 nucleotide sequences to design oligonucleotide primers, sublocalization can be achieved with panels of fragments from specific chromosomes. Other mapping strategies which can similarly be used to map a 2465 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 2465 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

B. Tissue Typing

The 2465 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the 2465 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The 2465 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of 2465 gene sequences can comfortably provide positive individual identification with a panel of perhaps 10 to 1,000 primers which each yield a noncoding amplified sequence of 100 bases. If predicted coding sequences, such as those in SEQ ID NO:9 are used, a more appropriate number of primers for positive individual identification would be 500-2,000.

If a panel of reagents from 2465 nucleotide sequences described herein is used to generate a unique identification database for an individual, those same reagents can later be used to identify tissue from that individual. Using the unique identification database, positive identification of the individual, living or dead, can be made from extremely small tissue samples.

C. Use of Partial 2465 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of 2465 gene sequences are particularly appropriate for this use as greater numbers of polymorphisms occur in the noncoding regions, making it easier to differentiate individuals using this technique. Examples of polynucleotide reagents include the 2465 nucleotide sequences or portions thereof, e.g., fragments derived from the noncoding regions having a length of at least 20 bases, preferably at least 30 bases.

The 2465 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such 2465 probes can be used to identify tissue by species and/or by organ type.

In a similar fashion, these reagents, e.g., 2465 primers or probes can be used to screen tissue culture for contamination (i.e. screen for the presence of a mixture of different types of cells in a culture).

D. Electronic Apparatus Readable Media and Arrays

Electronic apparatus readable media comprising 2465 sequence information is also provided. As used herein, “2465 sequence information” refers to any nucleotide and/or amino acid sequence information particular to the 2465 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said 2465 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon 2465 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 2465 sequence information.

A variety of software programs and formats can be used to store the sequence information on the electronic apparatus readable medium. For example, the sequence information can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the 2465 sequence information.

By providing 2465 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a 2465-associated disease or disorder or a pre-disposition to a 2465-associated disease or disorder, wherein the method comprises the steps of determining 2465 sequence information associated with the subject and based on the 2465 sequence information, determining whether the subject has a 2465-associated disease or disorder or a pre-disposition to a 2465-associated disease or disorder and/or recommending a particular treatment for the disease, disorder or pre-disease condition.

The present invention further provides in an electronic system and/or in a network, a method for determining whether a subject has a 2465-associated disease or disorder or a pre-disposition to a disease associated with a 2465 wherein the method comprises the steps of determining 2465 sequence information associated with the subject, and based on the 2465 sequence information, determining whether the subject has a 2465-associated disease or disorder or a pre-disposition to a 2465-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a 2465-associated disease or disorder or a pre-disposition to a 2465-associated disease or disorder associated with 2465, said method comprising the steps of receiving 2465 sequence information from the subject and/or information related thereto, receiving phenotypic information associated with the subject, acquiring information from the network corresponding to 2465 and/or a 2465-associated disease or disorder, and based on one or more of the phenotypic information, the 2465 information (e.g., sequence information and/or information related thereto), and the acquired information, determining whether the subject has a 2465-associated disease or disorder or a pre-disposition to a 2465-associated disease or disorder. The method may further comprise the step of recommending a particular treatment for the disease, disorder or pre-disease condition.

The present invention also provides a business method for determining whether a subject has a 2465-associated disease or disorder or a pre-disposition to a 2465-associated disease or disorder, said method comprising the steps of receiving information related to 2465 (e.g., sequence information and/or information related thereto), receiving phenotypic information associated with the subject, acquiring information from the network related to 2465 and/or related to a 2465-associated disease or disorder, and based on one or more of the phenotypic information, the 2465 information, and the acquired information, determining whether the subject has a 2465-associated disease or disorder or a pre-disposition to a 2465-associated disease or disorder. The method may further comprise the step of recommending a particular treatment for the disease, disorder or pre-disease condition.

The invention also includes an array comprising a 2465 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be 2465. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a 2465-associated disease or disorder, progression of 2465-associated disease or disorder, and processes, such a cellular transformation associated with the 2465-associated disease or disorder.

The array is also useful for ascertaining the effect of the expression of a gene on the expression of other genes in the same cell or in different cells (e.g., ascertaining the effect of 2465 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including 2465) that could serve as a molecular target for diagnosis or therapeutic intervention.

5. Recombinant Expression Vectors and Host Cells

The methods of the invention include the use of vectors, preferably expression vectors, containing a nucleic acid encoding a 2465 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the methods of the invention may include other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 2465 proteins, mutant forms of 2465 proteins, fusion proteins, and the like).

The recombinant expression vectors of the invention can be designed for expression of 2465 proteins in prokaryotic or eukaryotic cells, e.g., for use in the cell-based assays of the invention. For example, 2465 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 2465 activity assays, (e.g., direct assays or competitive assays described in detail below), or to generate antibodies specific for 2465 proteins, for example. In a preferred embodiment, a 2465 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the 2465 expression vector is a yeast expression vector. Examples of vectors for expression in yeast S. cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, 2465 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the □-fetoprotein promoter (Campes and Tilghman (1989) Genes Dev. 3:537-546).

The expression characteristics of an endogenous 2465 gene within a cell line or microorganism may be modified by inserting a heterologous DNA regulatory element into the genome of a stable cell line or cloned microorganism such that the inserted regulatory element is operatively linked with the endogenous 2465 gene. For example, an endogenous 2465 gene which is normally “transcriptionally silent”, i.e., a 2465 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous 2465 gene may be activated by insertion of a promiscuous regulatory element that works across cell types.

A heterologous regulatory element may be inserted into a stable cell line or cloned microorganism, such that it is operatively linked with an endogenous 2465 gene, using techniques, such as targeted homologous recombination, which are well known to those of skill in the art, and described, e.g., in Chappel, U.S. Pat. No. 5,272,071; PCT publication No. WO 91/06667, published May 16, 1991.

The methods of the invention further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 2465 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to the use of host cells into which a 2465 nucleic acid molecule of the invention is introduced, e.g., a 2465 nucleic acid molecule within a recombinant expression vector or a 2465 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 2465 protein can be expressed in bacterial cells such as E. coli, insect cells, yeast or mammalian cells (such as Chinese hamster ovary cells (CHO), COS cells, or human umbilical vein endothelial cells (HUVEC)). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a 2465 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a 2465 protein. Accordingly, the invention further provides methods for producing a 2465 protein using the host cells of the invention. In one embodiment, the method comprises culturing the host cell of the invention (into which a recombinant expression vector encoding a 2465 protein has been introduced) in a suitable medium such that a 2465 protein is produced. In another embodiment, the method further comprises isolating a 2465 protein from the medium or the host cell.

6. Cell- and Animal-Based Model Systems

Described herein are cell- and animal-based systems which act as models for hepatic, bone, or cardiovascular disorders. These systems may be used in a variety of applications. For example, the cell- and animal-based model systems may be used to further characterize differentially expressed genes associated with hepatic, bone, or cardiovascular disorders, e.g., 2465. In addition, animal- and cell-based assays may be used as part of screening strategies designed to identify compounds which are capable of ameliorating hepatic, bone, or cardiovascular disorder symptoms, as described, below. Thus, the animal- and cell-based models may be used to identify drugs, pharmaceuticals, therapies and interventions which may be effective in treating hepatic, bone, or cardiovascular disorders. Furthermore, such animal models may be used to determine the LD50 and the ED50 in animal subjects, and such data can be used to determine the in vivo efficacy of potential hepatic, bone, or cardiovascular disorder treatments.

A. Animal-Based Systems

Animal-based model systems of hepatic, bone, or cardiovascular disorders may include, but are not limited to, non-recombinant and engineered transgenic animals.

Non-recombinant animal models for hepatic, bone, or cardiovascular disorders may include, for example, genetic models.

Additionally, animal models exhibiting hepatic, bone, or cardiovascular disorders symptoms may be engineered by using, for example, 2465 gene sequences described above, in conjunction with techniques for producing transgenic animals that are well known to those of skill in the art. For example, 2465 gene sequences may be introduced into, and overexpressed in, the genome of the animal of interest, or, if endogenous 2465 gene sequences are present, they may either be overexpressed or, alternatively, be disrupted in order to underexpress or inactivate 2465 gene expression.

Non-recombinant animal models for cardiovascular disorders are described supra.

The host cells of the invention can also be used to produce non-human transgenic animals. For example, in one embodiment, a host cell of the invention is a fertilized oocyte or an embryonic stem cell into which 2465-coding sequences have been introduced. Such host cells can then be used to create non-human transgenic animals in which exogenous 2465 sequences have been introduced into their genome or homologous recombinant animals in which endogenous 2465 sequences have been altered. Such animals are useful for studying the function and/or activity of a 2465 and for identifying and/or evaluating modulators of 2465 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 2465 gene has been altered by homologous recombination between the endogenous gene and an exogenous DNA molecule introduced into a cell of the animal, e.g., an embryonic cell of the animal, prior to development of the animal.

A transgenic animal for use in the methods of the invention can be created by introducing a 2465-encoding nucleic acid into the male pronuclei of a fertilized oocyte, e.g., by microinjection, retroviral infection, and allowing the oocyte to develop in a pseudopregnant female foster animal. The 2465 cDNA sequence of SEQ ID NO:9 can be introduced as a transgene into the genome of a non-human animal. Alternatively, a nonhuman homologue of a human 2465 gene, such as a mouse or rat 2465 gene, can be used as a transgene. Alternatively, a 2465 gene homologue, such as another 2465 family member, can be isolated based on hybridization to the 2465 cDNA sequences of SEQ ID NO:9 and used as a transgene. Intronic sequences and polyadenylation signals can also be included in the transgene to increase the efficiency of expression of the transgene. A tissue-specific regulatory sequence(s) can be operably linked to a 2465 transgene to direct expression of a 2465 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 2465 transgene in its genome and/or expression of 2465 mRNA in tissues or cells of the animals. A transgenic founder animal can then be used to breed additional animals carrying the transgene. Moreover, transgenic animals carrying a transgene encoding a 2465 protein can further be bred to other transgenic animals carrying other transgenes.

To create a homologous recombinant animal, a vector is prepared which contains at least a portion of a 2465 gene into which a deletion, addition or substitution has been introduced to thereby alter, e.g., functionally disrupt, the 2465 gene. The 2465 gene can be a human gene (e.g., the cDNA of SEQ ID NO:9), but more preferably, is a non-human homologue of a human 2465 gene (e.g., a cDNA isolated by stringent hybridization with the nucleotide sequence of SEQ ID NO:9). For example, a mouse 2465 gene can be used to construct a homologous recombination nucleic acid molecule, e.g., a vector, suitable for altering an endogenous 2465 gene in the mouse genome. In a preferred embodiment, the homologous recombination nucleic acid molecule is designed such that, upon homologous recombination, the endogenous 2465 gene is functionally disrupted (i.e., no longer encodes a functional protein; also referred to as a “knock out” vector). Alternatively, the homologous recombination nucleic acid molecule can be designed such that, upon homologous recombination, the endogenous 2465 gene is mutated or otherwise altered but still encodes functional protein (e.g., the upstream regulatory region can be altered to thereby alter the expression of the endogenous 2465 protein). In the homologous recombination nucleic acid molecule, the altered portion of the 2465 gene is flanked at its 5′ and 3′ ends by additional nucleic acid sequence of the 2465 gene to allow for homologous recombination to occur between the exogenous 2465 gene carried by the homologous recombination nucleic acid molecule and an endogenous 2465 gene in a cell, e.g., an embryonic stem cell. The additional flanking 2465 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 2465 gene has homologously recombined with the endogenous 2465 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/oxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 2465 transgenic animals that express 2465 mRNA or a 2465 peptide (detected immunocytochemically, using antibodies directed against 2465 epitopes) at easily detectable levels should then be further evaluated to identify those animals which display characteristic hepatic, bone, or cardiovascular disorder symptoms. Such symptoms may include, for example, increased prevalence and size of fatty streaks and/or hepatic, bone, or cardiovascular disorder plaques.

Additionally, specific cell types within the transgenic animals may be analyzed and assayed for cellular phenotypes characteristic of hepatic, bone, or cardiovascular disorders. In the case of monocytes, such phenotypes may include but are not limited to increases in rates of LDL uptake, adhesion to endothelial cells, transmigration, foam cell formation, fatty streak formation, and production of foam cell specific products. Cellular phenotypes may include a particular cell type's pattern of expression of genes associated with hepatic, bone, or cardiovascular disorders as compared to known expression profiles of the particular cell type in animals exhibiting hepatic, bone, or cardiovascular disorder symptoms.

B. Cell-Based Systems

Cells that contain and express 2465 gene sequences which encode a 2465 protein, and, further, exhibit cellular phenotypes associated with hepatic, bone, or cardiovascular disorders, may be used to identify compounds that exhibit anti-hepatic, bone, or cardiovascular disorder activity. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); hepatic cells such as human Hepa; as well as generic mammalian cell lines such as HeLa cells and COS cells, e.g., COS-7 (ATCC# CRL-1651). Further, such cells may include recombinant, transgenic cell lines. For example, the hepatic, bone, or cardiovascular disorder animal models of the invention, discussed above, may be used to generate cell lines, containing one or more cell types involved in hepatic, bone, or cardiovascular disorders, that can be used as cell culture models for this disorder. While primary cultures derived from the hepatic, bone, or cardiovascular disorder transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in hepatic, bone, or cardiovascular disorders may be transfected with sequences capable of increasing or decreasing the amount of 2465 gene expression within the cell. For example, 2465 gene sequences may be introduced into, and overexpressed in, the genome of the cell of interest, or, if endogenous 2465 gene sequences are present, they may be either overexpressed or, alternatively disrupted in order to underexpress or inactivate 2465 gene expression.

In order to overexpress a 2465 gene, the coding portion of the 2465 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 2465 gene sequence, such a sequence may be isolated and engineered such that when reintroduced into the genome of the cell type of interest, the endogenous 2465 alleles will be inactivated. Preferably, the engineered 2465 sequence is introduced via gene targeting such that the endogenous 2465 sequence is disrupted upon integration of the engineered 2465 sequence into the cell's genome. Transfection of host cells with 2465 genes is discussed, above.

Cells treated with compounds or transfected with 2465 genes can be examined for phenotypes associated with hepatic, bone, or cardiovascular disorders. In the case of hepatocytes, such phenotypes include but are not limited to overproduction of matrix components. In the case of osteocytes, such phenotypes include but are not limited to expression of cytokines or growth factors. Expression of cytokines or growth factors may be measured using any of the assays described herein.

Similarly, hepatic, bone, or cardiovascular cells can be treated with test compounds or transfected with genetically engineered 2465 genes. The hepatic, bone, or cardiovascular cells can then be examined for phenotypes associated with hepatic, bone, or cardiovascular disorders, including, but not limited to changes in cellular morphology, cell proliferation, and cell migration; or for the effects on production of other proteins involved in hepatic, bone, or cardiovascular disorders such as adhesion molecules (e.g., ICAM, VCAM), PDGF, and E-selectin.

Transfection of 2465 nucleic acid may be accomplished by using standard techniques (described in, for example, Ausubel (1989) supra). Transfected cells should be evaluated for the presence of the recombinant 2465 gene sequences, for expression and accumulation of 2465 mRNA, and for the presence of recombinant 2465 protein production. In instances wherein a decrease in 2465 gene expression is desired, standard techniques may be used to demonstrate whether a decrease in endogenous 2465 gene expression and/or in 2465 protein production is achieved.

7. Pharmaceutical Compositions

Active compounds for use in the methods of the invention can be incorporated into pharmaceutical compositions suitable for administration. As used herein, the language “active compounds” includes 2465 nucleic acid molecules, fragments of 2465 proteins, and anti-2465 antibodies, as well as identified compounds that modulate 2465 gene expression, synthesis, and/or activity. Such compositions typically comprise the compound, nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a 2465 protein or a 2465 ligand) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. In one embodiment, a therapeutically effective dose refers to that amount of an active compound sufficient to result in amelioration of symptoms of hepatic, bone, or cardiovascular disorders.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses the identification and/or use of agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

8. Isolated Nucleic Acid Molecules

The nucleotide sequence of the isolated human 2465 cDNA and the predicted amino acid sequence of the human 2465 polypeptide are shown in SEQ ID NOs:9 and 10, respectively. The nucleotide sequence encoding human 2465 is identical to the nucleic acid molecule with GenBank Accession Number D38449 (Hata et al. BBA (1995) 1261:121-125).

The human 2465 gene, which is approximately 2816 nucleotides in length, encodes a protein having a molecular weight of approximately 59.34 kD and which is approximately 516 amino acid residues in length.

The methods of the invention include the use of isolated nucleic acid molecules that encode 2465 proteins or biologically active portions thereof, as well as nucleic acid fragments sufficient for use as hybridization probes to identify 2465-encoding nucleic acid molecules (e.g., 2465 mRNA) and fragments for use as PCR primers for the amplification or mutation of 2465 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated 2465 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:9, or a portion thereof, can be isolated using standard molecular biology techniques and the sequence information provided herein. Using all or portion of the nucleic acid sequence of SEQ ID NO:9, as a hybridization probe, 2465 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:9 can be isolated by the polymerase chain reaction (PCR) using synthetic oligonucleotide primers designed based upon the sequence of SEQ ID NO:9.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to 2465 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:9. The sequence of SEQ ID NO:9 corresponds to the human 2465 cDNA. This cDNA comprises sequences encoding the human 2465 protein (i.e., “the coding region”).

In another preferred embodiment, an isolated nucleic acid molecule of the invention comprises a nucleic acid molecule which is a complement of the nucleotide sequence shown in SEQ ID NO:9, or a portion of any of this nucleotide sequence. A nucleic acid molecule which is complementary to the nucleotide sequence shown in SEQ ID NO:9 is one which is sufficiently complementary to the nucleotide sequence shown in SEQ ID NO:9 such that it can hybridize to the nucleotide sequence shown in SEQ ID NO:9, thereby forming a stable duplex.

In still another preferred embodiment, the methods of the invention include the use of an isolated nucleic acid molecule that comprises a nucleotide sequence which is at least about 30%, 35%, 40%, 45%, 50%, 55%, 60%, 65%, 70%, 75%, 80%, 85%, 90%, 95%, 98% or more identical to the entire length of the nucleotide sequence shown in SEQ ID NO:9, or a portion of any of this nucleotide sequence.

Moreover, the methods of the invention include the use of a nucleic acid molecule that comprises only a portion of the nucleic acid sequence of SEQ ID NO:9, for example, a fragment which can be used as a probe or primer or a fragment encoding a portion of a 2465 protein, e.g., a biologically active portion of a 2465 protein. The nucleotide sequence determined from the cloning of the 2465 gene allows for the generation of probes and primers designed for use in identifying and/or cloning other 2465 family members, as well as 2465 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:9, of an anti-sense sequence of SEQ ID NO:9, or of a naturally occurring allelic variant or mutant of SEQ ID NO:9. In one embodiment, a nucleic acid molecule of the present invention comprises a nucleotide sequence which is greater than 100, 100-200, 200-300, 300-400, 400-500, 500-600, 600-700, 700-800, or more nucleotides in length and hybridizes under stringent hybridization conditions to a nucleic acid molecule of SEQ ID NO:9.

Probes based on the 2465 nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 2465 protein, such as by measuring a level of a 2465-encoding nucleic acid in a sample of cells from a subject e.g., detecting 2465 mRNA levels or determining whether a genomic 2465 gene has been mutated or deleted.

A nucleic acid fragment encoding a “biologically active portion of a 2465 protein” can be prepared by isolating a portion of the nucleotide sequence of SEQ ID NO:9 which encodes a polypeptide having a 2465 biological activity (the biological activities of the 2465 protein is described herein), expressing the encoded portion of the 2465 protein (e.g., by recombinant expression in vitro) and assessing the activity of the encoded portion of the 2465 protein.

The methods of the invention further encompass nucleic acid molecules that differ from the nucleotide sequence shown in SEQ ID NO:9, due to degeneracy of the genetic code and thus encode the same 2465 protein as those encoded by the nucleotide sequence shown in SEQ ID NO:9. In another embodiment, an isolated nucleic acid molecule of the invention has a nucleotide sequence encoding a protein having an amino acid sequence shown in SEQ ID NO:10.

In addition to the 2465 nucleotide sequence shown in SEQ ID NO:9, it will be appreciated by those skilled in the art that DNA sequence polymorphisms that lead to changes in the amino acid sequences of the 2465 protein may exist within a population (e.g., the human population). Such genetic polymorphism in the 2465 gene may exist among individuals within a population due to natural allelic variation. As used herein, the terms “gene” and “recombinant gene” refer to nucleic acid molecules which include an open reading frame encoding a 2465 protein, preferably a mammalian 2465 protein, and can further include non-coding regulatory sequences, and introns.

Allelic variants of human 2465 include both functional and non-functional 2465 proteins. Functional allelic variants are naturally occurring amino acid sequence variants of the human 2465 protein that maintain the ability to bind a 2465 ligand or substrate and/or modulate cell proliferation and/or migration mechanisms. Functional allelic variants will typically contain only conservative substitution of one or more amino acids of SEQ ID NO:10, or substitution, deletion or insertion of non-critical residues in non-critical regions of the protein.

Non-functional allelic variants are naturally occurring amino acid sequence variants of the human 2465 protein that do not have the ability to either bind a 2465 ligand or substrate and/or modulate cell proliferation and/or migration mechanisms. Non-functional allelic variants will typically contain a non-conservative substitution, a deletion, or insertion or premature truncation of the amino acid sequence of SEQ ID NO:10, or a substitution, insertion or deletion in critical residues or critical regions.

The methods of the present invention may further use non-human orthologues of the human 2465 protein. Orthologues of the human 2465 protein are proteins that are isolated from non-human organisms and possess the same 2465 ligand binding and/or modulation of cell proliferation and/or migration mechanisms of the human 2465 protein. Orthologues of the human 2465 protein can readily be identified as comprising an amino acid sequence that is substantially identical to SEQ ID NO:10.

Moreover, nucleic acid molecules encoding other 2465 family members and, thus, which have a nucleotide sequence which differs from the 2465 sequence of SEQ ID NO:9 are intended to be within the scope of the invention. For example, another 2465 cDNA can be identified based on the nucleotide sequence of human 2465. Moreover, nucleic acid molecules encoding 2465 proteins from different species, and which, thus, have a nucleotide sequence which differs from the 2465 sequence of SEQ ID NO:9 are intended to be within the scope of the invention. For example, a mouse 2465 cDNA can be identified based on the nucleotide sequence of human 2465.

Nucleic acid molecules corresponding to natural allelic variants and homologues of the 2465 cDNA of the invention can be isolated based on their homology to the 2465 nucleic acid disclosed herein using the cDNAs disclosed herein, or a portion thereof, as a hybridization probe according to standard hybridization techniques under stringent hybridization conditions. Nucleic acid molecules corresponding to natural allelic variants and homologues of the 2465 cDNA of the invention can further be isolated by mapping to the same chromosome or locus as the 2465 gene.

Accordingly, in another embodiment, the methods of the invention include the use of an isolated nucleic acid molecule that is at least 15, 20, 25, 30 or more nucleotides in length and hybridizes under stringent conditions to the nucleic acid molecule comprising the nucleotide sequence of SEQ ID NO:9. In other embodiment, the nucleic acid is at least 30, 50, 100, 150, 200, 250, 300, 350, 400, 450, 500, 550, 600, 650, 700, 750, 800, 1000, 1200, or more nucleotides in length. As used herein, the term “hybridizes under stringent conditions” is intended to describe conditions for hybridization and washing under which nucleotide sequences at least 60% identical to each other typically remain hybridized to each other. Preferably, the conditions are such that sequences at least about 70%, more preferably at least about 80%, even more preferably at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C. Ranges intermediate to the above-recited values, e.g., at 60-65° C. or at 55-60° C. are also intended to be encompassed by the present invention. Preferably, an isolated nucleic acid molecule of the invention that hybridizes under stringent conditions to the sequence of SEQ ID NO:9 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the 2465 sequences that may exist in the population, the skilled artisan will further appreciate that changes can be introduced by mutation into the nucleotide sequence of SEQ ID NO:9, thereby leading to changes in the amino acid sequence of the encoded 2465 protein, without altering the functional ability of the 2465 protein. For example, nucleotide substitutions leading to amino acid substitutions at “non-essential” amino acid residues can be made in the sequence of SEQ ID NO:9. A “non-essential” amino acid residue is a residue that can be altered from the wild-type sequence of 2465 (e.g., the sequence of SEQ ID NO:10) without altering the biological activity, whereas an “essential” amino acid residue is required for biological activity. For example, amino acid residues that are conserved among the 2465 proteins of the present invention are predicted to be particularly unamenable to alteration. Furthermore, additional amino acid residues that are conserved between the 2465 proteins of the present invention and other members of the G protein-coupled receptor family are not likely to be amenable to alteration.

Accordingly, the methods of the invention may include the use of nucleic acid molecules encoding 2465 proteins that contain changes in amino acid residues that are not essential for activity. Such 2465 proteins differ in amino acid sequence from SEQ ID NO:10, yet retain biological activity. In one embodiment, the isolated nucleic acid molecule comprises a nucleotide sequence encoding a protein, wherein the protein comprises an amino acid sequence at least about 30%, 35%, 40%, 45%, 50%, 55%, 60%, 65%, 70%, 75%, 80%, 85%, 90%, 95%, 98% or more identical to SEQ ID NO:10.

An isolated nucleic acid molecule encoding a 2465 protein identical to the protein of SEQ ID NO:10 can be created by introducing one or more nucleotide substitutions, additions or deletions into the nucleotide sequence of SEQ ID NO:9 such that one or more amino acid substitutions, additions or deletions are introduced into the encoded protein. Mutations can be introduced into SEQ ID NO:10 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 2465 protein is preferably replaced with another amino acid residue from the same side chain family. Alternatively, in another embodiment, mutations can be introduced randomly along all or part of a 2465 coding sequence, such as by saturation mutagenesis, and the resultant mutants can be screened for 2465 biological activity to identify mutants that retain activity. Following mutagenesis of SEQ ID NO:9, the encoded protein can be expressed recombinantly and the activity of the protein can be determined.

In a preferred embodiment, a mutant 2465 protein can be assayed for the ability to (1) interact with a non-2465 protein molecule, e.g., a 2465 ligand or substrate; (2) activate a 2465-dependent signal transduction pathway; or (3) modulate cell proliferation and/or migration mechanisms, or modulate the expression of cell surface adhesion molecules. In addition to the nucleic acid molecules encoding 2465 proteins described herein, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 2465 coding strand, or to only a portion thereof. In one embodiment, an antisense nucleic acid molecule is antisense to a “coding region” of the coding strand of a nucleotide sequence encoding 2465. The term “coding region” refers to the region of the nucleotide sequence comprising codons which are translated into amino acid residues (e.g., the coding region of human 2465 corresponds to SEQ ID NO:9). In another embodiment, the antisense nucleic acid molecule is antisense to a “noncoding region” of the coding strand of a nucleotide sequence encoding 2465. The term “noncoding region” refers to 5′ and 3′ sequences which flank the coding region that are not translated into amino acids (i.e., also referred to as 5′ and 3′ untranslated regions).

Given the coding strand sequences encoding 2465 disclosed herein (e.g., SEQ ID NO:9), antisense nucleic acids of the invention can be designed according to the rules of Watson and Crick base pairing. The antisense nucleic acid molecule can be complementary to the entire coding region of 2465 mRNA, but more preferably is an oligonucleotide which is antisense to only a portion of the coding or noncoding region of 2465 mRNA. For example, the antisense oligonucleotide can be complementary to the region surrounding the translation start site of 2465 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

In yet another embodiment, the 2465 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of 2465 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 2465 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of 2465 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 2465 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

9. Isolated 2465 Proteins and Anti-2465 Antibodies

The methods of the invention include the use of isolated 2465 proteins, and biologically active portions thereof, as well as polypeptide fragments suitable for use as immunogens to raise anti-2465 antibodies.

Isolated proteins of the present invention, preferably 2465 proteins, have an amino acid sequence sufficiently identical to the amino acid sequence of SEQ ID NO:10, or are encoded by a nucleotide sequence sufficiently identical to SEQ ID NO:9. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, a “2465 activity”, “biological activity of 2465” or “functional activity of 2465”, refers to an activity exerted by a 2465 protein, polypeptide or nucleic acid molecule on a 2465 responsive cell (e.g., an endothelial cell) or tissue, or on a 2465 protein substrate, as determined in vivo, or in vitro, according to standard techniques. In one embodiment, a 2465 activity is a direct activity, such as an association with a 2465 target molecule. As used herein, a “target molecule” or “binding partner” is a molecule with which a 2465 protein binds or interacts in nature, such that 2465-mediated function is achieved. A 2465 target molecule can be a non-2465 molecule or a 2465 protein or polypeptide of the present invention. In an exemplary embodiment, a 2465 target molecule is a 2465 ligand. Alternatively, a 2465 activity is an indirect activity, such as a cellular signaling activity mediated by interaction of the 2465 protein with a 2465 ligand. Preferably, a 2465 activity is the ability to act as a signal transduction molecule and to modulate endothelial cell proliferation, differentiation, and/or migration. Accordingly, another embodiment of the invention features isolated 2465 proteins and polypeptides having a 2465 activity.

In one embodiment, native 2465 proteins can be isolated from cells or tissue sources by an appropriate purification scheme using standard protein purification techniques. In another embodiment, 2465 proteins are produced by recombinant DNA techniques. Alternative to recombinant expression, a 2465 protein or polypeptide can be synthesized chemically using standard peptide synthesis techniques.

An “isolated” or “purified” protein or biologically active portion thereof is substantially free of cellular material or other contaminating proteins from the cell or tissue source from which the 2465 protein is derived, or substantially free from chemical precursors or other chemicals when chemically synthesized. The language “substantially free of cellular material” includes preparations of 2465 protein in which the protein is separated from cellular components of the cells from which it is isolated or recombinantly produced. In one embodiment, the language “substantially free of cellular material” includes preparations of 2465 protein having less than about 30% (by dry weight) of non-2465 protein (also referred to herein as a “contaminating protein”), more preferably less than about 20% of non-2465 protein, still more preferably less than about 10% of non-2465 protein, and most preferably less than about 5% non-2465 protein. When the 2465 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of 2465 protein in which the protein is separated from chemical precursors or other chemicals which are involved in the synthesis of the protein. In one embodiment, the language “substantially free of chemical precursors or other chemicals” includes preparations of 2465 protein having less than about 30% (by dry weight) of chemical precursors or non-2465 chemicals, more preferably less than about 20% chemical precursors or non-2465 chemicals, still more preferably less than about 10% chemical precursors or non-2465 chemicals, and most preferably less than about 5% chemical precursors or non-2465 chemicals.

As used herein, a “biologically active portion” of a 2465 protein includes a fragment of a 2465 protein which participates in an interaction between a 2465 molecule and a non-2465 molecule. Biologically active portions of a 2465 protein include peptides comprising amino acid sequences sufficiently identical to or derived from the amino acid sequence of the 2465 protein, e.g., the amino acid sequence shown in SEQ ID NO:10, which include less amino acids than the full length 2465 protein, and exhibit at least one activity of a 2465 protein. Typically, biologically active portions comprise a domain or motif with at least one activity of the 2465 protein, e.g., modulating cell proliferation mechanisms. A biologically active portion of a 2465 protein can be a polypeptide which is, for example, 10, 25, 50, 100, 200, or more amino acids in length. Biologically active portions of a 2465 protein can be used as targets for developing agents which modulate a 2465 mediated activity, e.g., a cell proliferation mechanism. A biologically active portion of a 2465 protein comprises a protein in which regions of the protein are deleted, can be prepared by recombinant techniques and evaluated for one or more of the functional activities of a native 2465 protein.

In a preferred embodiment, the 2465 protein has an amino acid sequence shown in SEQ ID NO:10. In other embodiments, the 2465 protein is substantially identical to SEQ ID NO:10, and retains the functional activity of the protein of SEQ ID NO:10, yet differs in amino acid sequence due to natural allelic variation or mutagenesis, as described in detail in subsection I above. Accordingly, in another embodiment, the 2465 protein is a protein which comprises an amino acid sequence at least about 30%, 35%, 40%, 45%, 50%, 55%, 60%, 65%, 70%, 75%, 80%, 85%, 90%, 95%, 98% or more identical to SEQ ID NO:10.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 2465 amino acid sequence of SEQ ID NO:10 having 516 amino acid residues, at least 136, preferably at least 181, more preferably at least 227, even more preferably at least 272, and even more preferably at least 317, 362 or 408 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Myers and Miller, Comput. Appl. Biosci. 4:11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 2465 nucleic acid molecules of the invention. BLAST protein searches can be performed with the XBLAST program, score=100, wordlength=3 to obtain amino acid sequences homologous to 2465 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The methods of the invention may also use 2465 chimeric or fusion proteins. As used herein, a 2465 “chimeric protein” or “fusion protein” comprises a 2465 polypeptide operatively linked to a non-2465 polypeptide. A “2465 polypeptide” refers to a polypeptide having an amino acid sequence corresponding to 2465, whereas a “non-2465 polypeptide” refers to a polypeptide having an amino acid sequence corresponding to a protein which is not substantially homologous to the 2465 protein, e.g., a protein which is different from the 2465 protein and which is derived from the same or a different organism. Within a 2465 fusion protein the 2465 polypeptide can correspond to all or a portion of a 2465 protein. In a preferred embodiment, a 2465 fusion protein comprises at least one biologically active portion of a 2465 protein. In another preferred embodiment, a 2465 fusion protein comprises at least two biologically active portions of a 2465 protein. Within the fusion protein, the term “operatively linked” is intended to indicate that the 2465 polypeptide and the non-2465 polypeptide are fused in-frame to each other. The non-2465 polypeptide can be fused to the N-terminus or C-terminus of the 2465 polypeptide.

For example, in one embodiment, the fusion protein is a GST-2465 fusion protein in which the 2465 sequences are fused to the C-terminus of the GST sequences. Such fusion proteins can facilitate the purification of recombinant 2465. In another embodiment, the fusion protein is a 2465 protein containing a heterologous signal sequence at its N-terminus. In certain host cells (e.g., mammalian host cells), expression and/or secretion of 2465 can be increased through use of a heterologous signal sequence.

The 2465 fusion proteins of the invention can be incorporated into pharmaceutical compositions and administered to a subject in vivo. The 2465 fusion proteins can be used to affect the bioavailability of a 2465 ligand. Use of 2465 fusion proteins may be useful therapeutically for the treatment of disorders caused by, for example, (i) aberrant modification or mutation of a gene encoding a 2465 protein; (ii) mis-regulation of the 2465 gene; and (iii) aberrant post-translational modification of a 2465 protein. In one embodiment, a 2465 fusion protein may be used to treat a hepatic, bone, or cardiovascular disorder. In another embodiment, a 2465 fusion protein may be used to treat an endothelial cell disorder.

Moreover, the 2465-fusion proteins of the invention can be used as immunogens to produce anti-2465 antibodies in a subject, to purify 2465 ligands and in screening assays to identify molecules which inhibit the interaction of 2465 with a 2465 substrate.

Preferably, a 2465 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 2465-encoding nucleic acid can be cloned into such an expression vector such that the fusion moiety is linked in-frame to the 2465 protein.

The methods of the present invention may also include the use of variants of the 2465 protein which function as either 2465 agonists (mimetics) or as 2465 antagonists. Variants of the 2465 protein can be generated by mutagenesis, e.g., discrete point mutation or truncation of a 2465 protein. An agonist of the 2465 protein can retain substantially the same, or a subset, of the biological activities of the naturally occurring form of a 2465 protein. An antagonist of a 2465 protein can inhibit one or more of the activities of the naturally occurring form of the 2465 protein by, for example, competitively modulating a 2465-mediated activity of a 2465 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 2465 protein.

In one embodiment, variants of a 2465 protein which function as either 2465 agonists (mimetics) or as 2465 antagonists can be identified by screening combinatorial libraries of mutants, e.g., truncation mutants, of a 2465 protein for 2465 protein agonist or antagonist activity. In one embodiment, a variegated library of 2465 variants is generated by combinatorial mutagenesis at the nucleic acid level and is encoded by a variegated gene library. A variegated library of 2465 variants can be produced by, for example, enzymatically ligating a mixture of synthetic oligonucleotides into gene sequences such that a degenerate set of potential 2465 sequences is expressible as individual polypeptides, or alternatively, as a set of larger fusion proteins (e.g., for phage display) containing the set of 2465 sequences therein. There are a variety of methods which can be used to produce libraries of potential 2465 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 2465 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a 2465 protein coding sequence can be used to generate a variegated population of 2465 fragments for screening and subsequent selection of variants of a 2465 protein. In one embodiment, a library of coding sequence fragments can be generated by treating a double stranded PCR fragment of a 2465 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 2465 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 2465 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify 2465 variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated 2465 library. For example, a library of expression vectors can be transfected into a cell line, e.g., an endothelial cell line, which ordinarily responds to a 2465 ligand in a particular 2465-dependent manner. The transfected cells are then contacted with a 2465 ligand and the effect of expression of the mutant on signaling by the 2465 receptor can be detected, e.g., by monitoring the generation of an intracellular second messenger (e.g., calcium, cAMP, IP3, or diacylglycerol), the phosphorylation profile of intracellular proteins, cell proliferation and/or migration, the expression profile of cell surface adhesion molecules, or the activity of a 2465-regulated transcription factor. Plasmid DNA can then be recovered from the cells which score for inhibition, or alternatively, potentiation of signaling by the 2465 receptor, and the individual clones further characterized.

An isolated 2465 protein, or a portion or fragment thereof, can be used as an immunogen to generate antibodies that bind 2465 using standard techniques for polyclonal and monoclonal antibody preparation. A full-length 2465 protein can be used or, alternatively, the invention provides antigenic peptide fragments of 2465 for use as immunogens. The antigenic peptide of 2465 comprises at least 8 amino acid residues of the amino acid sequence shown in SEQ ID NO:10 and encompasses an epitope of 2465 such that an antibody raised against the peptide forms a specific immune complex with 2465. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of 2465 that are located on the surface of the protein, e.g., hydrophilic regions, as well as regions with high antigenicity.

A 2465 immunogen typically is used to prepare antibodies by immunizing a suitable subject, (e.g., rabbit, goat, mouse or other mammal) with the immunogen. An appropriate immunogenic preparation can contain, for example, recombinantly expressed 2465 protein or a chemically synthesized 2465 polypeptide. The preparation can further include an adjuvant, such as Freund's complete or incomplete adjuvant, or similar immunostimulatory agent. Immunization of a suitable subject with an immunogenic 2465 preparation induces a polyclonal anti-2465 antibody response.

Accordingly, another aspect of the invention pertains to anti-2465 antibodies. The term “antibody” as used herein refers to immunoglobulin molecules and immunologically active portions of immunoglobulin molecules, i.e., molecules that contain an antigen binding site which specifically binds (immunoreacts with) an antigen, such as 2465. Examples of immunologically active portions of immunoglobulin molecules include F(ab) and F(ab′)2 fragments which can be generated by treating the antibody with an enzyme such as pepsin. The invention provides polyclonal and monoclonal antibodies that bind 2465. The term “monoclonal antibody” or “monoclonal antibody composition”, as used herein, refers to a population of antibody molecules that contain only one species of an antigen binding site capable of immunoreacting with a particular epitope of 2465. A monoclonal antibody composition thus typically displays a single binding affinity for a particular 2465 protein with which it immunoreacts.

Polyclonal anti-2465 antibodies can be prepared as described above by immunizing a suitable subject with a 2465 immunogen. The anti-2465 antibody titer in the immunized subject can be monitored over time by standard techniques, such as with an enzyme linked immunosorbent assay (ELISA) using immobilized 2465. If desired, the antibody molecules directed against 2465 can be isolated from the mammal (e.g., from the blood) and further purified by well known techniques, such as protein A chromatography to obtain the IgG fraction. At an appropriate time after immunization, e.g., when the anti-2465 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a 2465 immunogen as described above, and the culture supernatants of the resulting hybridoma cells are screened to identify a hybridoma producing a monoclonal antibody that binds 2465.

Any of the many well known protocols used for fusing lymphocytes and immortalized cell lines can be applied for the purpose of generating an anti-2465 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 2465, e.g., using a standard ELISA assay.

Alternative to preparing monoclonal antibody-secreting hybridomas, a monoclonal anti-2465 antibody can be identified and isolated by screening a recombinant combinatorial immunoglobulin library (e.g., an antibody phage display library) with 2465 to thereby isolate immunoglobulin library members that bind 2465. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-2465 antibodies, such as chimeric and humanized monoclonal antibodies, comprising both human and non-human portions, which can be made using standard recombinant DNA techniques, can also be used in the methods of the present invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-2465 antibody (e.g., monoclonal antibody) can be used to isolate 2465 by standard techniques, such as affinity chromatography or immunoprecipitation. An anti-2465 antibody can facilitate the purification of natural 2465 from cells and of recombinantly produced 2465 expressed in host cells. Moreover, an anti-2465 antibody can be used to detect 2465 protein (e.g., in a cellular lysate or cell supernatant) in order to evaluate the abundance and pattern of expression of the 2465 protein. Anti-2465 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include

125I, 131I, 35S or 3H.

This invention is further illustrated by the following examples which should not be construed as limiting. The contents of all references, patents and published patent applications cited throughout this application and the Sequence Listing are incorporated herein by reference.

### EXAMPLES

### Example 1

### Regulation of 2465 Expression in Fibrotic Liver Cells

Transcriptional profiling was used to detect the presence of RNA transcript corresponding to human 2465 in several tissues. It was found that the corresponding orthologs of 2465 are expressed in a variety of tissues. The results of this screening are shown in Tables 2 and 3 below.

TABLE 2

| | Relative |
|---|---|
| Tissue | Expression |
| Heart | 1 |
| Lung | 2 |
| Liver Pool | 0 |
| Brain | 18 |
| Fetal Liver | 2 |
| Spleen | 3 |
| Grans. | 0 |
| NHDF | 9 |
| mock | |
| NHLF | 23 |
| mock | |
| NHLF TGF | 16 |
| Hep4 | 12 |
| Pass Stell | 33 |
| Control | 0 |
| liver | |
| LF/NDR | 6 |
| 191 | |
| LF/NDR | 1 |
| 193 | |
| LF/NDR | 6 |
| 079 | |
| Tonsil | 1 |
| LN NDR | 2 |
| 173 | |
| Th1 MP39 | 0 |
| Th2 MP39 | 0 |
| Th1 MP21 | 0 |
| Th2 MP21 | 0 |
| CD4 | 0 |
| CD4 | 0 |
| CD19 | 0 |

TABLE 3

| | | | Relative |
|---|---|---|---|
| | | Delta | Expression |
| Tissue | Delta | Delta | LevelEL |
| Prostate | 9.0 | 9.0 | 1.9 |
| Osteoclasts | 1.6 | 1.6 | 335.0 |
| Liver | 9.4 | 9.4 | 1.5 |
| Liver | 10.0 | 10.0 | 1.0 |
| Breast | 12.2 | 12.2 | 0.2 |
| Sk. Muscle | 6.8 | 6.8 | 9.2 |
| Sk. Muscle | 6.7 | 6.7 | 9.8 |
| Brain | 5.2 | 5.2 | 28.2 |
| Colon | 9.3 | 9.3 | 1.6 |
| Colon | 6.6 | 6.6 | 10.3 |
| Heart | 4.7 | 4.7 | 39.8 |
| Heart | 7.5 | 7.5 | 5.5 |
| Ovary | 12.0 | 12.0 | 0.2 |
| Ovary | 13.0 | 13.0 | 0.1 |
| Kidney | 7.6 | 7.6 | 5.3 |
| Kidney | 5.7 | 5.7 | 19.3 |
| Lung | 8.3 | 8.3 | 3.2 |
| Lung | 7.5 | 7.5 | 5.4 |
| Vein | 8.3 | 8.3 | 3.3 |
| Vein | 6.0 | 6.0 | 15.4 |
| Adipose | 10.4 | 10.4 | 0.8 |
| Adipose | 5.5 | 5.5 | 22.3 |
| Sm Intestine | 7.4 | 7.4 | 6.0 |
| Thyroid | 5.2 | 5.2 | 27.0 |
| Bone Marrow | 14.8 | 14.8 | 0.0 |
| Skin | 3.6 | 3.6 | 80.0 |
| Testis | 3.7 | 3.7 | 78.8 |
| Placenta | 8.5 | 8.5 | 2.7 |
| Fetal liver | 10.4 | 10.4 | 0.7 |
| Fetal Liver | 7.7 | 7.7 | 5.0 |
| Fetal Heart | 6.4 | 6.4 | 11.9 |
| Fetal Heart | 10.1 | 10.1 | 0.9 |
| Undif | 3.7 | 3.7 | 76.9 |
| Osteoblasts | | | |
| Dif Osteoblasts | 3.6 | 3.6 | 81.9 |
| Prim Cult | 6.8 | 6.8 | 8.7 |
| Osteoblasts | | | |
| Spinal Cord | 10.3 | 10.3 | 0.8 |
| Cervix | 11.8 | 11.8 | 0.3 |
| Spleen | 9.9 | 9.9 | 1.0 |
| Spinal Cord | 9.3 | 9.3 | 1.5 |
| Thymus | 9.6 | 9.6 | 1.3 |
| Tonsil | 6.5 | 6.5 | 11.4 |
| Lymph Node | 6.2 | 6.2 | 13.2 |

Reverse Transcriptase PCR (RT-PCR) was used to detect the presence of RNA transcript corresponding to human 2465 in RNA prepared from cells and tissues related to liver fibrosis. The highest expression of the gene was noted in dividing liver stellate cells, which are known to contribute to fibrosis. Quiescent stellate cells and other liver cells showed much lowered levels of expression, as shown in table 4 below.

TABLE 4

| | Relative |
|---|---|
| | Expression |
| Tissue | LevelEL |
| Brain | 511.0 |
| Heart | 71.0 |
| Lung | 34.0 |
| Liver Pool | 9.0 |
| Passaged Stellate | 908.0 |
| Quiescant Stellate | 14.0 |
| Stellate | 529.0 |
| Stellate/FBS | 1338.0 |
| NHDF Moch 48 hrs | 262.0 |
| NHDF TGF 48 hrs | 779.0 |
| NHLF Mock 48 hrs | 889.0 |
| NHLF TGF 48 hrs | 901.0 |
| HepG2 Mock 48 hrs | 3.0 |
| HepG2 TGF 48 hrs | 5.0 |
| NC Heps | 6.0 |
| Hep-1 control | 155.0 |
| Hep-2 PMA/Iono 6 hrs | 129.0 |
| Hep 3 TGF 24 hrs | 87.0 |
| Hep 4 TGF 48 hrs | 165.0 |
| Liver NDR 200 | 4.0 |
| Liver CHT 339 | 18.0 |
| Liver Pit 250 | 4.0 |
| LF/NDR 079 | 115.0 |
| LF/NDR 141 | 62.0 |
| LF/NDR 156 | 31.0 |
| LF/NDR 190 | 26.0 |
| LF/NDR 191 | 92.0 |
| LF/NDR 192 | 87.0 |
| LF/NDR 194 | 120.0 |
| LF/NDR 225 | 29.0 |

In order to assess the fibrotic regulation of 2465 in vivo, three animal models for liver fibrosis were used. In one model, the bile duct of rats was surgically ligated, thus causing a fibrosis-like state in the liver by ceasing the flow of bile. RT-PCR was used to assess the expression of the rat ortholog of 2465 at several time points after bile-duct ligation. The results of this analysis are shown in Table 5 below.

TABLE 5

| | Relative |
|---|---|
| | Expression |
| Tissue | LevelEL |
| 3 week BDL (exp | 0.620 |
| A828) | |
| Fibrotic 15 | 0.640 |
| Fibrotic 16 | 1.380 |
| Fibrotic 17 | 0.980 |
| Fibrotic 18 | 0.650 |
| Fibrotic 19 | 1.740 |
| Fibrotic 20 | 2.040 |
| Fibrotic 21 | 3.620 |
| Fibrotic 22 | 1.590 |
| Fibrotic 23 | 1.380 |
| Fibrotic 24 | 0.820 |
| Control 25 | 0.140 |
| Control 26 | 0.050 |
| Control 27 | 0.130 |
| Control 28 | 0.010 |
| Control 29 | 0.040 |
| Control 30 | 0.200 |

In another whole animal model, porcine serum was injected into rats, thus, inducing a fibrotic liver condition. RT-PCR was used to assess the expression of the rat ortholog of 2465 in the fibrotic liver. The results of this analysis are shown in Table 6 below.

TABLE 6

| | Relative |
|---|---|
| | Expression |
| Tissue | LevelEL |
| 5.5 Serum (Exp | 0.020 |
| A2018) control 1 | |
| 5.5 Serum control 3 | 0.220 |
| 5.5 Serum control 4 | 0.150 |
| 5.5 Serum control 5 | 0.090 |
| 5.5 Serum fibrotic 7 | 2.230 |
| 5.5 Serum fibrotic 8 | 2.560 |
| 5.5 Serum fibrotic 9 | 1.620 |
| 5.5 Serum fibrotic | 4.990 |
| 10 | |
| 5.5 Serum fibrotic | 0.380 |
| 16 | |
| 5.5 Serum fibrotic | 0.030 |
| 17 | |
| 5.5 Serum fibrotic | 0.940 |
| 18 | |
| 5.5 Serum fibrotic | 0.150 |
| 19 | |
| 5.5 Serum fibrotic | 0.910 |
| 20 | |

In a third whole animal model, a fibrotic liver condition was induced by injection of a toxin (carbon tetrachloride) into rats. RT-PCR was again used to assess the expression of the rat ortholog of 2465 in the fibrotic liver.

### Example 2

### Regulation of 2465 Expression in Cells Involved in Osteogenesis

The expression of 2465 was assessed in several cell types including cells of adipocyte lineage and those of osteoblast lineage using RT-PCR. In summary, the expression of 2465 was greater in osteoblast lineage cells than in adipocyte or progenitor lineage cell types.

These results were then confirmed by measuring the relative expression levels of 2465 using TaqMan PCR on osteogenic cells and adipogenic cells.

TaqMan PCR was also used to assess the expression of 2465 in several cellular models of osteoporosis. The results of this analysis demonstrate that these osteoporosis models do express 2465 at varying levels.

Expression of 2465 was assessed in several tissues. A relatively high expression of the transcript was found in differentiated osteoblasts, and primary cultured osteoblasts, as well as in skin and testis. Moderate levels of 2465 expression were also demonstrated in thyroid, adipose, vein, kidney, heart and brain samples.

### Example 3

### Expression of Recombinant 2465 Protein in Bacterial Cells

In this example, 2465 is expressed as a recombinant glutathione-S-transferase (GST) fusion polypeptide in E. coli and the fusion polypeptide is isolated and characterized. Specifically, 2465 is fused to GST and this fusion polypeptide is expressed in E. coli, e.g., strain PEB199. Expression of the GST-2465 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant 2465 Protein in COS Cells

To express the 2465 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 2465 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 2465 DNA sequence is amplified by PCR using two primers. The 5′ primer contains the restriction site of interest followed by approximately twenty nucleotides of the 2465 coding sequence starting from the initiation codon; the 3′ end sequence contains complementary sequences to the other restriction site of interest, a translation stop codon, the HA tag or FLAG tag and the last 20 nucleotides of the 2465 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 2465 gene is inserted in the correct orientation. The ligation mixture is transformed into E. coli cells (strains HB101, DH5□, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 2465-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the VR-3 or VR-5 polypeptide is detected by radiolabelling (35S-methionine or 35S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with 35S-methionine (or 35S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 2465 coding sequence is cloned directly into the polylinker of the pCDNA/Amp vector using the appropriate restriction sites. The resulting plasmid is transfected into COS cells in the manner described above, and the expression of the 2465 polypeptide is detected by radiolabelling and immunoprecipitation using a 2465 specific monoclonal antibody.

### Example 5

### Expression of 2465 in Isolated Human Vessels

Reverse Transcriptase PCR (RT-PCR) was used to detect the presence of RNA transcripts corresponding to human 2465 in mRNA prepared from isolated human vessels. The highest expression of the gene was noted in endothelial cells and smooth muscle cells, consistent with a role of this molecule in vascular functions, while expression in adipose tissue (which may contaminate vessel preparations) was low (see Table 7 below).

TABLE 7

| | 2465 | | | |
|---|---|---|---|---|
| Tissues | Mean | dCt | RE | SD |
| Aortic SMC | 24.265 | 2.695 | 154.43 | 6.81 |
| HMVEC | 23.35 | 4.445 | 45.91 | 2.03 |
| H/Adipose | 28.43 | 8.77 | 2.29 | 1.07 |
| H/Artery/Normal/Carotid | 27.645 | 8.345 | 3.08 | 0.05 |
| H/Artery/Normal/Carotid | 28.475 | 7.65 | 4.98 | 1.33 |
| H/Artery/Normal/Muscular | 26.165 | 4.855 | 34.55 | 0.17 |
| H/Artery/Diseased/iliac | 24.45 | 5.08 | 29.56 | 0.87 |
| H/Artery/Diseased/Tibial | 28.81 | 8.44 | 2.88 | 1.14 |
| H/Aorta/Diseased | 27.29 | 5.47 | 22.56 | 1.11 |
| H/Vein/Normal/Saphenous | 28.665 | 7.24 | 6.62 | 4.15 |
| H/Vein/Normal/Saphenous | 24.5 | 5.935 | 16.35 | 2.49 |
| H/Vein/Normal/Saphenous | 22.99 | 5.375 | 24.10 | 2.01 |
| H/Vein/Normal/Saphenous | 28.94 | 8.665 | 2.46 | 0.11 |
| H/Vein/Diseased/Saphenous | 25.28 | 5.865 | 17.16 | 0.08 |
| H/Vein/Normal/ | 29.085 | 7.875 | 4.26 | 0.73 |
| H/Vein/Normal/Saphenous | 27.5 | 7.18 | 6.90 | 1.22 |
| H/Vein/Normal/ | 30.015 | 7.78 | 4.55 | 0.18 |

### Example 6

### Regulation of 2465 Expression in Isolated Human Vessels

Human umbilical vein endothelial cells (HUVEC's) were cultured in vitro under standard conditions, described in, for example, U.S. Pat. No. 5,882,925. Experimental cultures were then exposed to laminar shear stress (LSS) conditions by culturing the cells in a specialized apparatus containing liquid culture medium. Static cultures grown in the same medium served as controls. The in vitro LSS treatment at 10 dyns/cm2 was performed for 24 hours and was designed to simulate the shear stress generated by blood flow in a straight, healthy artery.

The effect of LSS on 2465 expression in endothelial cells was assessed by Taqman analysis. 2465 gene expression was significantly induced in HUVECs exposed to LSS.

In another study, HUVEC or microvascular endothelial cells cultured from human heart (HMVEC-C) or lung (HMVEC-L) were harvested while rapidly proliferating (“prolif”), or after they had reached confluence in their regular growth medium (“conf”) or in growth factor depleted medium (“-GF”). Upregulation of 2465 was observed under proliferating conditions.

In addition, HUVEC cultures were treated with human IL-1β, a factor known to be involved in the inflammatory response, in order to mimic the physiologic conditions involved in the atherosclerotic state. Stimulation of endothelial cells with IL-1β induces the expression of several inflammatory markers. 2465 expression was upregulated by treatment with IL-1β.

Collectively, these data indicate that 2465 may be involved in the regulation of endothelial cell processes such as proliferation, which are relevant to angiogenesis and the development of atherosclerosis. The data also indicate that 2465 may play a role in vascular functions such as in the control of vascular tone.

Equivalents

Those skilled in the art will recognize, or be able to ascertain using no more than routine experimentation, many equivalents to the specific embodiments of the invention described herein. Such equivalents are intended to be encompassed by the following claims.

### III. METHODS FOR USING 14266, A HUMAN G PROTEIN-COUPLED RECEPTOR

### Background of the Invention

G-protein coupled receptors (GPCRs) constitute a major class of proteins responsible for transducing a signal within a cell. GPCRs share three structural features: an amino terminal extracellular domain, a transmembrane region containing seven transmembrane domains, three extracellular loops, and three intracellular loops, and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

GPCR genes and gene-products are potential causative agents of disease (Spiegel et al., J. Clin. Invest. 92:1119-1125 (1993); McKusick et al., J. Med. Genet. 30:1-26 (1993)). Specific defects in the rhodopsin gene and the V2 vasopressin receptor gene have been shown to cause various forms of retinitis pigmentosum (Nathans et al., Annu. Rev. Genet. 26:403-424(1992)), and nephrogenic diabetes insipidus (Holtzman et al., Hum. Mol. Genet. 2:1201-1204 (1993)). These receptors are of critical importance to both the central nervous system and peripheral physiological processes. Evolutionary analyses suggest that the ancestor of these proteins originally developed in concert with complex body plans and nervous systems.

The GPCR protein superfamily can be divided into five families: Family I, which contains receptors typified by rhodopsin and the β2-adrenergic receptor and currently represented by over 200 unique members (Dohlman et al., Annu. Rev. Biochem. 60:653-688 (1991)); Family II, which contains the parathyroid hormone/calcitonin/secretin receptor family (Juppner et al., Science 254:1024-1026 (1991); Lin et al., Science 254:1022-1024 (1991)); Family III, which contains the metabotropic glutamate receptor family (Nakanishi, Science 258 597:603 (1992)); Family IV, which contains the cAMP receptor family, important in the chemotaxis and development of D. discoideum (Klein et al., Science 241:1467-1472 (1988)); and Family V, the fungal mating pheromone receptors such as STE2 (Kurjan, Annu. Rev. Biochem. 61:1097-1129 (1992)).

G-proteins represent a family of heterotrimeric proteins composed of α, β, and γ subunits. These proteins are usually linked to cell surface receptors, e.g., receptors containing seven transmembrane segments. Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cAMP (e.g., by activation of adenyl cyclase), diacylglycerol or inositol phosphates. Greater than 20 different types of α-subunits are known in humans. These subunits associate with a smaller pool of β and γ subunits. Examples of mammalian G proteins include Gi (inhibitory G protein), Go, Gq, Gs (stimulatory G protein) and Gt (transducin). G proteins are described extensively in Lodish et al., Molecular Cell Biology, (Scientific American Books Inc., New York, N.Y., 1995), the contents of which are incorporated herein by reference. GPCRs, G proteins and G protein-linked effector and second messenger systems have been reviewed in The G-Protein Linked Receptor Fact Book, Watson et al., eds., Academic Press (1994).

GPCRs are a major target for drug development. Accordingly, it is valuable to the field of pharmaceutical development to identify methods using GPCRs and tissues and disorders in which GPCRs are differentially expressed.

### Summary of the Invention

It is an object of the invention to identify tissues and disorders in which expression of 14266 is relevant, and to provide methods wherein 14266 is useful as a reagent or target in the diagnosis and treatment of 14266-related disorders.

A specific object of the invention is to identify compounds that act as agonists and antagonists and modulate the expression of 14266 in specific tissues and disorders. A further specific object of the invention is to provide compounds that modulate expression of 14266 for diagnosis and treatment of 14266-mediated or related disorders.

The invention provides methods of screening for compounds that modulate expression or activity of 14266 polypeptides or nucleic acid molecules (RNA or DNA) in the specific tissues or disorders. The invention also provides a process for modulating 14266 polypeptide or nucleic acid molecule expression or activity, especially using the screened compounds. Modulation may be used to treat conditions related to aberrant activity or expression of 14266 polypeptides or nucleic acids.

The invention also provides assays for determining the activity of, or the presence or absence of, 14266 polypeptides or nucleic acid molecules in specific biological samples, including for disease diagnosis.

The invention also provides assays for determining the presence of a mutation in the polypeptides or nucleic acid molecules, including for disease diagnosis.

The invention utilizes isolated 14266 polypeptides, including a polypeptide having the amino acid sequence shown in SEQ ID NO:11, and variant polypeptides having an amino acid sequence that is substantially homologous to the amino acid sequence shown in SEQ ID NO:11.

The invention also utilizes an isolated 14266 nucleic acid molecule having the sequence shown in SEQ ID NO:12, and variant nucleic acid sequences that are substantially homologous to the nucleotide sequence shown in SEQ ID NO:12.

The invention also utilizes fragments of the polypeptide shown in SEQ ID NO:11 and nucleotide sequence shown in SEQ ID NO:12, as well as substantially homologous fragments of the polypeptide or nucleic acid.

The invention further utilizes nucleic acid constructs comprising the nucleic acid molecules described herein. In a preferred embodiment, the nucleic acid molecules of the invention are operatively linked to a regulatory sequence.

The invention also utilizes vectors and host cells that express 14266 and provides methods for expressing 14266 nucleic acid molecules and polypeptides in specific cell types and disorders.

The invention also utilizes methods of making the vectors and host cells and provides methods for using them to assay expression and cellular effects of expression of the 14266 nucleic acid molecules and polypeptides in specific cell types and disorders.

The invention also utilizes antibodies or antigen-binding fragments thereof that selectively bind the 14266 polypeptides and fragments.

### Detailed Description of the Invention

The present invention is based on methods of using molecules referred to herein as 14266, 14266 GPCRS, 14266 receptors, or 14266 nucleic acid or polypeptide molecules. The 14266 receptor shares sequence similarity with the norepipniephrine β3 receptor and the serotonin 5HT-2C receptor, and there are 14266 receptor orthologs in rat and zebrafish, suggesting that the 14266 gene has been highly conserved in vertebrate evolution (Matsumoto et al. (2000) Biochem. Biophys. Res. Comm. 272: 576-582)

The uses, reagents and methods disclosed in detail herein below apply especially to tissues and cell types where 14266 expression is relevant. Analysis using the TaqMan® brand Polymerase Chain Reaction Kit (Applied Biosystems, Foster City, Calif.) demonstrated that 14266 expression is highest in spinal cord and brain (particularly the cortex and hypothalamus). 14266 expression is also detectable in aorta, heart, fetal heart, vein, astrocytes (normal cells and glioblastoma tissue), breast (normal tissue and interductal carcinoma tissue), ovary (normal tissue and ovary tumor tissue), pancreas, prostate (normal tissue and prostate tumor cells), and colon (normal tissue, tumor tissue, and inflammatory bowel disease tissue).

Further analysis using the TaqMan® brand Polymerase Chain Reaction Kit demonstrated high 14266 expression in bone marrow mononuclear cells, granulocyte colony-stimulating factor (G-CSF)-mobilized peripheral blood and adult bone marrow CD34+ haematopoietic progenitor cells, neutrophils isolated from bone marrow from normal and G-CSF treated individuals, and in mature neutrophils generated from CD34+ haematopoietic progenitor cells in vitro. CD34+ haematopoietic progenitor cells from bone marrow from volunteers treated with G-CSF showed significant levels of 14266 expression, as did both early stage and more mature neutrophil lineage cells isolated from bone marrow from both normal and G-CSF treated volunteers. Expression of 14266 was regulated during both in vivo and in vitro generation of blood cells. It was down-regulated in both megakaryocytes and erythroid cells during differentiation, and up-regulated during neutrophil differentiation.

Neutrophils are a special class of granulocytes that are derived from the granulocyte/macrophage progenitor cells (colony-forming cells) which arise from the division and differentiation of myeloid stem cells. Neutrophils play a key role in the non-specific immune response, and they are recruited rapidly to sites of inflammation. Neutrophils are required for host defense against invading micro-organisms, and they respond to injurious agents by the release of granular enzymes and proteins, the production of reactive oxygen intermediates, and by phagocytosis. Patients with neutrophil deficiency disorders, including neutropenia, chronic granulomatous disease, and leukocyte adhesion deficiency, have a tendency to develop recurrent and overwhelming infections. Inadequate or ineffective granulopoiesis can result from suppression of myeloid stem cells (as occurs in aplastic anemia and a variety of infiltrative marrow disorders), suppression of the committed granulocytic precursors (which often occurs after exposure to certain drugs, including alkylating agents and antimetabolites used in cancer treatment), disease states characterized by ineffective granulopoiesis (such as megaloblastic anemias caused by vitamin B12 or folate deficiency and myelodysplastic syndromes) and rare inherited conditions (such as Kostmann syndrome). Excessive neutrophil activation is implicated in several inflammatory disorders, including acute respiratory distress syndrome (ARDS), rheumatoid arthritis, and ischaemia-reperfusion injury (reviewed in Condliffe et al. (1998) Clin. Sci. 94: 461-471. Thus, the regulation of neutrophil differentiation and activation plays a key role in determining the balance between defense and injury, making 14266 a target for the diagnosis and treatment of neutrophil deficiency disorders and disorders associated with excessive neutrophil activation.

G protein-coupled receptors, including 14266 receptors, use one of several signaling pathways to relay their intracellular signal As used herein, a “signaling pathway” refers to one or more signaling steps that lead to the modulation (e.g., stimulation or inhibition) of a cellular function/activity upon the binding of a ligand to the 14266 receptors.

One signaling pathway that may be used by the 14266 receptor is the phosphatidylinositol second messenger pathway, which involves phosphatidylinositol turnover and metabolism. As used herein, “phosphatidylinositol turnover and metabolism” refers to the molecules involved in the turnover and metabolism of phosphatidylinositol 4,5-bisphosphate (PIP2) as well as to the activities of these molecules. PIP2 is a phospholipid found in the cytosolic leaflet of the plasma membrane. Binding of ligand to the 14266 receptor may activate, in some cells, the plasma-membrane enzyme phospholipase C that, in turn, can hydrolyze PIP2 to produce 1,2-diacylglycerol (DAG) and inositol 1,4,5-triphosphate (IP3). Once formed IP3 can diffuse to the endoplasmic reticulum surface where it can bind an IP3 receptor, e.g., a calcium channel protein containing an IP3 binding site. IP3 binding can induce opening of the channel, allowing calcium ions to be released into the cytoplasm. IP3 can also be phosphorylated by a specific kinase to form inositol 1,3,4,5-tetraphosphate (IP4), a molecule which can cause calcium entry into the cytoplasm from the extracellular medium. IP3 and IP4 can subsequently be hydrolyzed very rapidly to the inactive products inositol 1,4-biphosphate (IP2) and inositol 1,3,4-triphosphate, respectively. These inactive products can be recycled by the cell to synthesize PIP2. The other second messenger produced by the hydrolysis of PIP2, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP2 or one of its metabolites.

Another signaling pathway in which the 14266 receptor may participate is the cAMP turnover pathway. As used herein, cAMP turnover and metabolism” refers to the molecules involved in the turnover and metabolism of cAMP as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

The disclosed invention relates to methods and compositions for the modulation, diagnosis, and treatment of diseases related to 14266 receptor malfunction. In addition to variability among individuals in their responses to drugs, several definable diseases arise from disorders in receptors or receptor-effector systems. The loss of a receptor in a highly specialized signaling system may cause a relatively limited phenotypic disorder, such as the genetic deficiency of the androgen receptor in the testicular feminization syndrome (Griffin et al. (1995) The Metabolic and Molecular Bases of Inherited Diseases 7:2967-2998). Deficiencies of more widely used signaling systems have a broader spectrum of effects, as are seen in myasthenia gravis or some forms of insulin-resistant diabetes mellitus, which result from autoimmune depletion of nicotinic cholinergic receptors or insulin receptors, respectively. A lesion in a component of a signaling pathway that is used by many receptors can cause a generalized endocrinopathy. Heterozygous deficiency for G5, the G protein that activates adenyl cyclase in all cells, causes multiple endocrine disorders; the disease is termed pseudohpoparathyroidism type 1a (Spiegel et al. (1995) The Metabolic and Molecular Bases of Inherited Diseases 7:3073-3089). Homozygous deficiency in G5 would presumably be lethal.

The expression of aberrant or ectopic receptors, effectors, or coupling proteins potentially can lead to supersensitivity, subsensitivity, or other untoward responses. Among the most interesting and significant events is the appearance of aberrant receptors as products of oncogenes, which transform otherwise normal cells into malignant cells. Virtually any type of signaling system may have oncogenic potential. The erbA oncogene product is an altered form of a receptor for thyroid hormone, constitutively active because of the loss of its ligand-binding domain (Evans (1988) Science 240:889-895). The ros and erbB oncogene products are activated, uncontrolled forms of the receptors for insulin and epidermal growth factor, both known to enhance cellular proliferation (Yarden et al. (1988) Annu. Rev. Biochem. 57:443-478). The mas oncogene product (Young et al. (1986) Cell. 4:711-719) is a G protein-coupled receptor, probably the receptor for a peptide hormone. Constitutive activation of G protein-coupled receptors due to subtle mutations in receptor structure has been shown to give rise to retinitis pigmentosa, precocious puberty, and malignant hyperthyroidism (Clapham (1993) Cell. 75:1237-1239). G proteins can themselves be oncogenic when either overexpressed or constitutively activated by mutation (Lyons et al (1990) Science 249:655-659).

Acetylcholine is implicated in higher functions of the brain, notably memory and cognition. Consistent with this there is a cholinergic deficiency in Alzheimer's Disease, an illness associated with a severe impairment of cognitive function. Agonists of the acetylcholine receptor have been used clinically in the treatment of glaucoma. Minor uses include suppression of atrial tachycardias, stimulation of intestinal motility and bladder emptying. Antagonists have been used as a premedication in general anesthesia to reduce bronchial and salivary secretions and in the prevention of motion sickness. They have also been used to a limited extent in the treatment of peptic ulcer, to induce pupillary vasodilitation, to aid examination of the eye and in the treatment of certain inflammatory conditions.

Adrenoceptors are affected by clinically important drugs used for asthma, as an anesthetic, for nasal decongestion, for hypertension, for other cardiovascular disorders, for example, angina, certain cardiac dysrhythmias and cardiac infarction, and for the treatment of anxiety and glaucoma.

The angiotensin receptor is the target for compounds effective in the treatment of hypertension.

The bradykinin receptor is a target for treatment of inflammation, asthma, mild pain, and endotoxic shock.

The calcitonin receptor is a target for treatment of Paget's disease of the bone.

The cannabinoid receptor is a potential therapeutic target as an analgesic or antiemetic agent.

The cholecystokinin and gastrin receptors are implicated in the pathogenesis of schizophrenia, Parkinson's disease, drug addiction, and feeding disorders.

Dopamine receptors have been implicated in Parkinson's disease, Huntington's disease and schizophrenia.

The endothelin receptor is a target for several pathophysiological conditions associated with stress including hypertension, myocardial infarction, subarachnoid hemorrhage and renal failure.

The galanin receptor is involved in insulin release induced by glucose and may be the sympathetic mediator of this effect during stress. It is synergistic with opiates in inducing analgesia. It stimulates feeding behavior and release of growth hormone. It may be of use in the treatment of Alzheimer's disease. Galanin agonists may be novel analgesics.

The glucagon receptor is involved in the pathogenesis of diabetes. It is also been implicated in increasing the rate and force of contraction in acute cardiac failure.

The receptors for glucagon-like peptides 1 and 2. These receptors could serve as a target for non-insulin dependent diabetes mellitus and intestinal disorders, respectively.

Glutamate receptors may be important in neuronal plasticity, cognition, memory, learning and some neurological disorders such as epilepsy, stroke and neurodengeneration.

Glycoprotein hormone receptors (FSH, LH/hCG, TSH) can be important in treating infertility in females and for some types of failure of spermatogenesis in males (FSH), and Graves disease (TSH).

Gonadotropin-releasing hormone receptor is a potential target in therapeutic use in the suppression of prostrate cancer, precocious puberty, and endometriosis.

Histamine receptors may be target for a variety of CNS functions including sexual behavior and analgesia. It may also be useful clinically in the treatment of allergic and anaphylactic reactions and various inflammatory conditions for example, hay fever and itching. It may also be useful in treatment of motion sickness. The H2 receptors are found in high levels in stomach and heart. H2 antagonists are used clinically in the treatment of peptic ulceration.

5-hydroxytryptamine receptor may be involved in a vast array of physiological and pathophysiological pathways. It is a mediator of peristalsis and may be involved in platelet aggregation and haemostasis. It may also have a role as an inflammatory mediator and involvement in microvascular control. It could be useful in a wide range of functions including control of appetite, mood, anxiety, hallucination, sleep, vomiting and pain perception and may have clinical use in the treatment of depression, migraine and post-operative vomiting. The 5-Ht1b/5-Ht1d receptor may be the therapeutic substrate of the anti-migraine drug sumitriptan. These sites are also implicated in feeding, behavior, anxiety, depression, cardiac function and movement. Clinically, 5-Ht1a receptors represent potential anxiolytic and anti-hypertensive targets.

Leukotrienes have important physiological roles in the cardiovascular respiratory and immune systems. Some of these are found in high levels in inflammatory conditions, for example, septic shock, inflammatory bowel disease and allergic asthma. They can be found in high levels in bronchial tissue and lung where they may have a pathological role in allergic asthma and respiratory distress syndrome. Accordingly, leukotriene receptors may be useful as targets in these areas. The receptors have been involved in inducing chemostasis and adhesion of neutrophils to vascular endothelium, inducing contraction of gastrointestinal, pulmonary, reproductive, and vascular smooth muscles, and stimulating mucus secretion in bronchial tissue.

Melanocortins include ACTH, α-, β- and λ-melanocyte-stimulating hormones (MSH), and β-endorphin. ACTH and β-endorphin are synthesized and released at times of stress, i.e. cold, infections, etc. and their release leads to metabolism and analgesia. ACTH is used clinically to diagnose adrenocorticol insufficiency and to stimulate adrenocortex function or as an alternative to glucocorticoids to treat inflammatory disorders.

Melatonin regulates a variety of neuroendocrine functions and is believed to have an essential role in circadian rhythms. Drugs that modify the action of melatonin are of potential clinical importance in the modification of in circadian cycles, for example, for the treatment of jet lag.

Neuropeptide Y is one of the most abundant peptides in the mammalian brain, inducing a variety of behavior effects, stimulation of food intake, anxiety, facilitation of learning and memory, and regulation of the cardiovascular and neuroendocrine systems. It has been implicated in the pathophysiology of hypertension, congestive heart failure, affective disorders, and appetite regulation.

Neurotensin induces a variety of effects including antinoception, hypothermia and increased locomotor activity.

Opioid peptides have important roles in the regulation of sensory function (including pain), neuroendocrine activity, the central control of respiration and mood, and the regulation of gut motility. Non-peptide agonists at opioid receptors include codeine, morphine and related substances. Many of these are used clinically in the treatment of pain and constipation. Some opioid receptors are believe to mediate analgesia sedation, mitosis and diuresis.

Parathyroid hormone is involved in calcium homeostasis. Antagonists at the parathyroid hormone receptor are of potential clinical use in the treatment of hyperparathyroidism and short-term hypercalcemic states.

Platelet activating factor is an important mediator in allergic and inflammatory conditions. Platelet activating factor antagonists are potential anti-inflammatory and anti-asthmatic agents.

Prostanoids (prostiglandins and thromboxanes) mediate a wide variety of actions and have important physiological roles in the cardiovascular and immune systems and in pain sensation. At least five classes of prostanoid receptors are known to exist. They mediate relaxation in vascular, gastrointestinal, and uterine smooth muscle in human, inhibit platelet activation, and modify release of hypothalamic and pituitary hormones. Some also inhibit neurotransmitter release in central and autonomic nerves and inhibit secretion in glandular tissues, i.e. acid secretion from gastric mucosa and sodium and water reabsorption in kidney.

Somatostatin is a neurotransmitter/hormone with a wide spectrum of biological actions. It has been used clinically in the treatment of certain tumors, carcinoid syndrome and glucagonoma. A reduction in cortical somatostatin levels has been reported in Alzheimer's disease and Parkinson's disease.

Tachykinins are a family of peptide neurotransmitters. They can stimulate smooth muscle contraction, glandular secretion, induce activation of cells of the immune system, and activate peripheral nerves. They can also regulate dopaminergic neurons and are involved in the transmission of sensory information, including noxious stimuli.

Thrombin has a role in blood clotting. It cleaves a number of substrates involved in coagulation and activates cell surface receptors through proteolytic action. It stimulates aggregation and secretion in blood platelets and has inflammatory and reparative actions. It activates a number of substrates that are involved in the coagulation process. Accordingly, the thrombin receptor is a target for the treatment of clotting disorders, and inflammatory disorders.

Thyrotrophin releasing hormone releases thyroid stimulating hormone and stimulates the synthesis and release of prolactin.

Vasoactive intestinal polypeptide family is grouped with the number of structurally related peptides that share an overlapping profile of biological activity. It induces relaxation in smooth muscle, for example, intestine, blood vessels and trachea. It inhibits secretion in certain tissues for example, stomach. It stimulates secretion in others for example, intestinal epithelium, pancreas, and gall bladder. It modulates activity of cells in the immune system. In the central nervous system it has a wide range of excitatory and inhibitory actions. Some members of the family are involved in secretion of enzymes and ions in pancreas and intestine (secretin) and regulating synthesis and release of growth hormone (growth hormone releasing factor).

Vasopressin and oxytocin are members of a family of peptides found in all mammalian species. Vasopressin controls the water content of the body and acts in the kidney to increase water and sodium absorption. It can stimulate the contraction of vascular smooth muscle, stimulate glycogen breakdown in liver, induce platelet activation, or evoke release or corticotrophin. Vasopressin is used clinically to treat diabetes insipidus. Oxytocin stimulates contraction of uterine smooth muscle, and stimulates milk secretion. It is used clinically to induce labor and to promote lactation.

The presence of genes encoding seven transmembrane proteins in the viral genome may be relevant for virally induced cell transformation and proliferation. Ligands targeted to the polypeptides may represent a novel class of antiviral drugs.

The disclosed invention further relates to the modulation, diagnosis, and treatment of various other disorders. Disorders involving the spleen include, but are not limited to, splenomegaly, including nonspecific acute splenitis, congestive spenomegaly, and spenic infarcts; neoplasms, congenital anomalies, and rupture. Disorders associated with splenomegaly include infections, such as nonspecific splenitis, infectious mononucleosis, tuberculosis, typhoid fever, brucellosis, cytomegalovirus, syphilis, malaria, histoplasmosis, toxoplasmosis, kala-azar, trypanosomiasis, schistosomiasis, leishmaniasis, and echinococcosis; congestive states related to partial hypertension, such as cirrhosis of the liver, portal or splenic vein thrombosis, and cardiac failure; lymphohematogenous disorders, such as Hodgkin disease, non-Hodgkin lymphomas/leukemia, multiple myeloma, myeloproliferative disorders, hemolytic anemias, and thrombocytopenic purpura; immunologic-inflammatory conditions, such as rheumatoid arthritis and systemic lupus erythematosus; storage diseases such as Gaucher disease, Niemann-Pick disease, and mucopolysaccharidoses; and other conditions, such as amyloidosis, primary neoplasms and cysts, and secondary neoplasms.

Disorders involving the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, a1-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Disorders involving the uterus and endometrium include, but are not limited to, endometrial histology in the menstrual cycle; functional endometrial disorders, such as anovulatory cycle, inadequate luteal phase, oral contraceptives and induced endometrial changes, and menopausal and postmenopausal changes; inflammations, such as chronic endometritis; adenomyosis; endometriosis; endometrial polyps; endometrial hyperplasia; malignant tumors, such as carcinoma of the endometrium; mixed Müllerian and mesenchymal tumors, such as malignant mixed Müllerian tumors; tumors of the myometrium, including leiomyomas, leiomyosarcomas, and endometrial stromal tumors.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischernia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B1) deficiency and vitamin B12 deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type I neurofibromatosis (NF1) and TYPE 2 neurofibromatosis (NF2), tuberous sclerosis, and Von Hippel-Lindau disease.

Disorders involving T-cells include, but are not limited to, cell-mediated hypersensitivity, such as delayed type hypersensitivity and T-cell-mediated cytotoxicity, and transplant rejection; autoimmune diseases, such as systemic lupus erythematosus, Sjögren syndrome, systemic sclerosis, inflammatory myopathies, mixed connective tissue disease, and polyarteritis nodosa and other vasculitides; immunologic deficiency syndromes, including but not limited to, primary immunodeficiencies, such as thymic hypoplasia, severe combined immunodeficiency diseases, and AIDS; leukopenia; reactive (inflammatory) proliferations of white cells, including but not limited to, leukocytosis, acute nonspecific lymphadenitis, and chronic nonspecific lymphadenitis; neoplastic proliferations of white cells, including but not limited to lymphoid neoplasms, such as precursor T-cell neoplasms, such as acute lymphoblastic leukemia/lymphoma, peripheral T-cell and natural killer cell neoplasms that include peripheral T-cell lymphoma, unspecified, adult T-cell leukemia/lymphoma, mycosis fungoides and Sezary syndrome, and Hodgkin disease.

Diseases of the skin, include but are not limited to, disorders of pigmentation and melanocytes, including but not limited to, vitiligo, freckle, melasma, lentigo, nevocellular nevus, dysplastic nevi, and malignant melanoma; benign epithelial tumors, including but not limited to, seborrheic keratoses, acanthosis nigricans, fibroepithelial polyp, epithelial cyst, keratoacanthoma, and adnexal (appendage) tumors; premalignant and malignant epidermal tumors, including but not limited to, actinic keratosis, squamous cell carcinoma, basal cell carcinoma, and merkel cell carcinoma; tumors of the dermis, including but not limited to, benign fibrous histiocytoma, dermatofibrosarcoma protuberans, xanthomas, and dermal vascular tumors; tumors of cellular immigrants to the skin, including but not limited to, histiocytosis X, mycosis fungoides (cutaneous T-cell lymphoma), and mastocytosis; disorders of epidermal maturation, including but not limited to, ichthyosis; acute inflammatory dermatoses, including but not limited to, urticaria, acute eczematous dermatitis, and erythema multiforme; chronic inflammatory dermatoses, including but not limited to, psoriasis, lichen planus, and lupus erythematosus; blistering (bullous) diseases, including but not limited to, pemphigus, bullous pemphigoid, dermatitis herpetiformis, and noninflammatory blistering diseases: epidermolysis bullosa and porphyria; disorders of epidermal appendages, including but not limited to, acne vulgaris; panniculitis, including but not limited to, erythema nodosum and erythema induratum; and infection and infestation, such as verrucae, molluscum contagiosum, impetigo, superficial fungal infections, and arthropod bites, stings, and infestations.

In normal bone marrow, the myelocytic series (polymorphoneuclear cells) make up approximately 60% of the cellular elements, and the erythrocytic series, 20-30%. Lymphocytes, monocytes, reticular cells, plasma cells and megakaryocytes together constitute 10-20%. Lymphocytes make up 5-15% of normal adult marrow. In the bone marrow, cell types are add mixed so that precursors of red blood cells (erythroblasts), macrophages (monoblasts), platelets (megakaryocytes), polymorphonuclear leucocytes (myeloblasts), and lymphocytes (lymphoblasts) can be visible in one microscopic field. The various types of cells and stages of each would be known to the person of ordinary skill in the art and are found, for example, on page 42 of Immunology, Immunopathology and Immunity, Fifth Edition, Sell et al. Simon and Schuster (1996), incorporated by reference for its teaching of cell types found in the bone marrow. According, the invention is directed to disorders arising from these cells. These disorders include but are not limited to the following: diseases involving hematopoeitic stem cells; committed lymphoid progenitor cells; lymphoid cells including B and T-cells; committed myeloid progenitors, including monocytes, granulocytes, and megakaryocytes; and committed erythroid progenitors. These include but are not limited to the leukemias, including B-lymphoid leukemias, T-lymphoid leukemias, undifferentiated leukemias; erythroleukemia, megakaryoblastic leukemia, monocytic; [leukemias are encompassed with and without differentiation]; chronic and acute lymphoblastic leukemia, chronic and acute lymphocytic leukemia, chronic and acute myelogenous leukemia, lymphoma, myelo dysplastic syndrome, chronic and acute myeloid leukemia, myelomonocytic leukemia; chronic and acute myeloblastic leukemia, chronic and acute myelogenous leukemia, chronic and acute promyelocytic leukemia, chronic and acute myelocytic leukemia, hematologic malignancies of monocyte-macrophage lineage, such as juvenile chronic myelogenous leukemia; secondary AML, antecedent hematological disorder; refractory anemia; aplastic anemia; reactive cutaneous angioendotheliomatosis; fibrosing disorders involving altered expression in dendritic cells, disorders including systemic sclerosis, E-M syndrome, epidemic toxic oil syndrome, eosinophilic fasciitis localized forms of scleroderma, keloid, and fibrosing colonopathy; angiomatoid malignant fibrous histiocytoma; carcinoma, including primary head and neck squamous cell carcinoma; sarcoma, including kaposi's sarcoma; fibroadanoma and phyllodes tumors, including mammary fibroadenoma; stromal tumors; phyllodes tumors, including histiocytoma; erythroblastosis; neurofibromatosis; diseases of the vascular endothelium; demyelinating, particularly in old lesions; gliosis, vasogenic edema, vascular disease, Alzheimer's and Parkinson's disease; T-cell lymphomas; B-cell lymphomas.

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Disorders involving red cells include, but are not limited to, anemias, such as hemolytic anemias, including hereditary spherocytosis, hemolytic disease due to erythrocyte enzyme defects: glucose-6-phosphate dehydrogenase deficiency, sickle cell disease, thalassemia syndromes, paroxysmal nocturnal hemoglobinuria, immunohemolytic anemia, and hemolytic anemia resulting from trauma to red cells; and anemias of diminished erythropoiesis, including megaloblastic anemias, such as anemias of vitamin B12 deficiency: pernicious anemia, and anemia of folate deficiency, iron deficiency anemia, anemia of chronic disease, aplastic anemia, pure red cell aplasia, and other forms of marrow failure.

Disorders involving the thymus include developmental disorders, such as DiGeorge syndrome with thymic hypoplasia or aplasia; thymic cysts; thymic hypoplasia, which involves the appearance of lymphoid follicles within the thymus, creating thymic follicular hyperplasia; and thymomas, including germ cell tumors, lynphomas, Hodgkin disease, and carcinoids. Thymomas can include benign or encapsulated thymoma, and malignant thymoma Type I (invasive thymoma) or Type II, designated thymic carcinoma.

Disorders involving B-cells include, but are not limited to precursor B-cell neoplasms, such as lymphoblastic leukemia/lymphoma. Peripheral B-cell neoplasms include, but are not limited to, chronic lymphocytic leukemia/small lymphocytic lymphoma, follicular lymphoma, diffuse large B-cell lymphoma, Burkitt lymphoma, plasma cell neoplasms, multiple myeloma, and related entities, lymphoplasmacytic lymphoma (Waldenström macroglobulinemia), mantle cell lymphoma, marginal zone lymphoma (MALToma), and hairy cell leukemia.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TIP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypernephroma, adenocarcinoma of kidney), which includes urothelial carcinomas of renal pelvis.

Disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms.

Disorders in the male breast include, but are not limited to, gynecomastia and carcinoma.

Disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, Leydig (interstitial) cell tumors and sertoli cell tumors (androblastoma), and testicular lymphoma, and miscellaneous lesions of tunica vaginalis.

Disorders involving the prostate include, but are not limited to, inflammations, benign enlargement, for example, nodular hyperplasia (benign prostatic hypertrophy or hyperplasia), and tumors such as carcinoma.

Disorders involving the thyroid include, but are not limited to, hyperthyroidism; hypothyroidism including, but not limited to, cretinism and myxedema; thyroiditis including, but not limited to, hashimoto thyroiditis, subacute (granulomatous) thyroiditis, and subacute lymphocytic (painless) thyroiditis; Graves disease; diffuse and multinodular goiter including, but not limited to, diffuse nontoxic (simple) goiter and multinodular goiter; neoplasms of the thyroid including, but not limited to, adenomas, other benign tumors, and carcinomas, which include, but are not limited to, papillary carcinoma, follicular carcinoma, medullary carcinoma, and anaplastic carcinoma; and cogenital anomalies.

Disorders involving the skeletal muscle include tumors such as rhabdomyosarcoma.

Disorders involving the pancreas include those of the exocrine pancreas such as congenital anomalies, including but not limited to, ectopic pancreas; pancreatitis, including but not limited to, acute pancreatitis; cysts, including but not limited to, pseudocysts; tumors, including but not limited to, cystic tumors and carcinoma of the pancreas; and disorders of the endocrine pancreas such as, diabetes mellitus; islet cell tumors, including but not limited to, insulinomas, gastrinomas, and other rare islet cell tumors.

Disorders involving the small intestine include the malabsorption syndromes such as, celiac sprue, tropical sprue (postinfectious sprue), whipple disease, disaccharidase (lactase) deficiency, abetalipoproteinemia, and tumors of the small intestine including adenomas and adenocarcinoma.

Disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

Disorders involving precursor T-cell neoplasms include precursor T lymphoblastic leukemia/lymphoma. Disorders involving peripheral T-cell and natural killer cell neoplasms include T-cell chronic lymphocytic leukemia, large granular lymphocytic leukemia, mycosis fungoides and Sezary syndrome, peripheral T-cell lymphoma, unspecified, angioimmunoblastic T-cell lymphoma, angiocentric lymphoma (NK/T-cell lymphoma4a), intestinal T-cell lymphoma, adult T-cell leukemia/lymphoma, and anaplastic large cell lymphoma.

Disorders involving the ovary include, for example, polycystic ovarian disease, Stein-leventhal syndrome, Pseudomyxoma peritonei and stromal hyperthecosis; ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Bone-forming cells include the osteoprogenitor cells, osteoblasts, and osteocytes. The disorders of the bone are complex because they may have an impact on the skeleton during any of its stages of development. Hence, the disorders may have variable manifestations and may involve one, multiple or all bones of the body. Such disorders include, congenital malformations, achondroplasia and thanatophoric dwarfism, diseases associated with abnormal matix such as type 1 collagen disease, osteoporosis, Paget disease, rickets, osteomalacia, high-turnover osteodystrophy, low-turnover of aplastic disease, osteonecrosis, pyogenic osteomyelitis, tuberculous osteomyelitism, osteoma, osteoid osteoma, osteoblastoma, osteosarcoma, osteochondroma, chondromas, chondroblastoma, chondromyxoid fibroma, chondrosarcoma, fibrous cortical defects, fibrous dysplasia, fibrosarcoma, malignant fibrous histiocytoma, Ewing sarcoma, primitive neuroectodermal tumor, giant cell tumor, and metastatic tumors.

The 14266 sequences of the invention are members of a family of molecules (the “G-protein coupled receptors” or “GPCRs”) having conserved functional features. The term “family” when referring to the proteins and nucleic acid molecules of the invention is intended to mean two or more proteins or nucleic acid molecules having sufficient amino acid or nucleotide sequence identity as defined herein. Such family members can be naturally occurring and can be from either the same or different species. For example, a family can contain a first protein of murine origin and a homologue of that protein of human origin, as well as a second, distinct protein of human origin and a murine homologue of that protein. Members of a family may also have common functional characteristics.

Methods of Using 14266

The invention provides methods using the 14266 variants, or fragments, including but not limited to use in the cells, tissues, and disorders as disclosed herein.

The protein sequences of the present invention can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-410. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used.

The 14266 polypeptides are useful for producing antibodies specific for the 14266, regions, or fragments.

A. Screening Assays

The invention provides a method (also referred to herein as a “screening assay”) for identifying modulators, i.e., candidate or test compounds or agents (e.g., peptides, peptidomimetics, small molecules, or other drugs) that bind to 14266 receptors or have a stimulatory or inhibitory effect on, for example, 14266 receptor expression or 14266 receptor activity.

The invention provides screening assays, in cell-based or cell-free systems. Cell-based systems can be native, i.e., cells that normally express the 14266 receptor, as a biopsy, or expanded in cell culture. In one embodiment, cell-based assays involve recombinant host cells expressing the 14266 receptor. Accordingly, cells that are useful in this regard include, but are not limited to, those disclosed herein as expressing 1466. Cells containing one or more copies of exogenously-introduced 14266 sequences or cells genetically modified to modulate expression of the endogenous 14266 sequence may also be used.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Determining the ability of the test compound to bind to the 14266 receptor can be accomplished, for example, by coupling the test compound with a radioisotope or enzymatic label such that binding of the test compound to the 14266 receptor or biologically active portion thereof can be determined by detecting the labeled compound in a complex. For example, test compounds can be labeled with 125I, 35S, 14C, or 3H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In a similar manner, one may determine the ability of the 14266 receptor to bind to or interact with a 14266 target molecule. By “target molecule” is intended a molecule with which a 14266 receptor binds or interacts in nature. In a preferred embodiment, the ability of the 14266 receptor to bind to or interact with a 14266 target molecule can be determined by monitoring the activity of the target molecule. For example, the activity of the target molecule can be monitored by detecting induction of a cellular second messenger of the target (e.g., intracellular Ca2+, diacylglycerol, IP3, etc.), detecting catalytic/enzymatic activity of the target on an appropriate substrate, detecting the induction of a reporter gene (e.g., a 14266-responsive regulatory element operably linked to a nucleic acid encoding a detectable marker, e.g. luciferase), or detecting a cellular response, for example, cellular differentiation or cell proliferation.

In yet another embodiment, an assay of the present invention is a cell-free assay comprising contacting a 14266 receptor or biologically active portion thereof with a test compound and determining the ability of the test compound to bind to the 14266 receptor or biologically active portion thereof. Binding of the test compound to the 14266 receptor can be determined either directly or indirectly as described above. In a preferred embodiment, the assay includes contacting the 14266 receptor or biologically active portion thereof with a known compound that binds the 14266 receptor to form an assay mixture, contacting the assay mixture with a test compound, and determining the ability of the test compound to preferentially bind to the 14266 receptor or biologically active portion thereof as compared to the known compound.

In another embodiment, an assay is a cell-free assay comprising contacting the 14266 receptor or biologically active portion thereof with a test compound and determining the ability of the test compound to modulate (e.g., stimulate or inhibit) the activity of the 14266 receptor or biologically active portion thereof. Determining the ability of the test compound to modulate the activity of a 14266 receptor can be accomplished, for example, by determining the ability of the 14266 receptor to bind to a 14266 target molecule as described above for determining direct binding. In an alternative embodiment, determining the ability of the test compound to modulate the activity of a 14266 receptor can be accomplished by determining the ability of the 14266 receptor to further modulate a 14266 target molecule. For example, the catalytic/enzymatic activity of the target molecule on an appropriate substrate can be determined as previously described.

In yet another embodiment, the cell-free assay comprises contacting the 14266 receptor or biologically active portion thereof with a known compound that binds a 14266 receptor to form an assay mixture, contacting the assay mixture with a test compound, and determining the ability of the test compound to preferentially bind to or modulate the activity of a 14266 target molecule.

In the above-mentioned assays, it may be desirable to immobilize either a 14266 receptor or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, glutathione-S-transferase/14266 fusion proteins or glutathione-S-transferase/target fusion proteins can be adsorbed onto glutathione sepharose beads (Sigma Chemical, St. Louis, Mo.) or glutathione-derivatized microtitre plates, which are then combined with the test compound or the test compound and either the nonadsorbed target protein or 14266 receptor, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of 14266 binding or activity determined using standard techniques.

Other techniques for immobilizing proteins on matrices can also be used in the screening assays of the invention. For example, either the 14266 receptor or its target molecule can be immobilized utilizing conjugation of biotin and streptavidin. Biotinylated 14266 molecules or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with a 14266 receptor or target molecules but which do not interfere with binding of the 14266 receptor to its target molecule can be derivatized to the wells of the plate, and unbound target or 14266 receptor trapped in the wells by antibody conjugation. Methods for detecting such complexes, in addition to those described above for the GST-immobilized complexes, include immunodetection of complexes using antibodies reactive with the 14266 receptor or target molecule, as well as enzyme-linked assays that rely on detecting an enzymatic activity associated with the 14266 receptor or target molecule.

In another embodiment, modulators of 14266 expression are identified in a method in which a cell is contacted with a candidate compound and the expression of 14266 mRNA or protein in the cell is determined relative to expression of 14266 mRNA or protein in a cell in the absence of the candidate compound. When expression is greater (statistically significantly greater) in the presence of the candidate compound than in its absence, the candidate compound is identified as a stimulator of 14266 mRNA or protein expression. Alternatively, when expression is less (statistically significantly less) in the presence of the candidate compound than in its absence, the candidate compound is identified as an inhibitor of 14266 mRNA or protein expression. The level of 14266 mRNA or protein expression in the cells can be determined by methods described herein for detecting. 14266 mRNA or protein.

In yet another aspect of the invention, the 14266 receptors can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with 14266 receptor (“14266-binding proteins” or “14266-bp”) and modulate 14266 activity. Such 14266-binding proteins are also likely to be involved in the propagation of signals by the 14266 receptors as, for example, upstream or downstream elements of the signal transduction pathway.

This invention further pertains to novel agents identified by the above-described screening assays and uses thereof for treatments as described herein.

B. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. These applications are described in the subsections below.

1. Diagnostic Assays

One aspect of the present invention relates to diagnostic assays for detecting 14266 receptor and/or nucleic acid expression as well as 14266 activity, in the context of a biological sample. An exemplary method for detecting the presence or absence of 14266 receptors in a biological sample involves obtaining a biological sample from a test subject and contacting the biological sample with a compound or an agent capable of detecting 14266 receptor or nucleic acid (e.g., mRNA, genomic DNA) that encodes 14266 receptor such that the presence of 14266 receptor is detected in the biological sample. Results obtained with a biological sample from the test subject may be compared to results obtained with a biological sample from a control subject.

A preferred agent for detecting 14266 mRNA or genomic DNA is a labeled nucleic acid probe capable of hybridizing to 14266 mRNA or genomic DNA. The nucleic acid probe can be, for example, a full-length nucleic acid of SEQ ID NO:12, or a portion thereof, such as a nucleic acid molecule of at least 15, 30, 50, 100, 250, or 500 nucleotides in length and sufficient to specifically hybridize under stringent conditions to 14266 mRNA or genomic DNA. Other suitable probes for use in the diagnostic assays of the invention are described herein.

A preferred agent for detecting 14266 receptor is an antibody capable of binding to 14266 receptor, preferably an antibody with a detectable label. Antibodies can be polyclonal, or more preferably, monoclonal. An intact antibody, or a fragment thereof (e.g., Fab or F(abN)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 14266 mRNA, protein, or genomic DNA in a biological sample in vitro as well as in vivo. For example, in vitro techniques for detection of 14266 mRNA include Northern hybridizations and in situ hybridizations. In vitro techniques for detection of the 14266 receptor include enzyme linked immunosorbent assays (ELISAs), Western blots, immunoprecipitations, and immunofluorescence. In vitro techniques for detection of 14266 genomic DNA include Southern hybridizations. Furthermore, in vivo techniques for detection of 14266 receptor include introducing into a subject a labeled anti-14266 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject.

The invention also encompasses kits for detecting the presence of 14266 receptors in a biological sample (a test sample). Such kits can be used to determine if a subject is suffering from or is at increased risk of developing a disorder associated with aberrant expression of 14266 receptor. For example, the kit can comprise a labeled compound or agent capable of detecting 14266 receptor or mRNA in a biological sample and means for determining the amount of a 14266 receptor in the sample (e.g., an anti-14266 antibody or an oligonucleotide probe that binds to DNA encoding a 14266 receptor, e.g., encoded by the nucleic acid sequences of SEQ ID NO:12). Kits can also include instructions for observing that the tested subject is suffering from or is at risk of developing a disorder associated with aberrant expression of 14266 sequences if the amount of 14266 receptor or mRNA is above or below a normal level.

For antibody-based kits, the kit can comprise, for example: (1) a first antibody (e.g., attached to a solid support) that binds to the 14266 receptor; and, optionally, (2) a second, different antibody that binds to the 14266 receptor or the first antibody and is conjugated to a detectable agent. For oligonucleotide-based kits, the kit can comprise, for example: (1) an oligonucleotide, e.g., a detectably labeled oligonucleotide, that hybridizes to a 14266 nucleic acid sequence or (2) a pair of primers useful for amplifying a 14266 nucleic acid molecule.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for observing whether the tested subject is suffering from or is at risk of developing a disorder associated with aberrant expression of 14266 receptors.

2. Other Diagnostic Assays

In another aspect, the invention features a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a 14266 nucleic acid, preferably purified, polypeptide, preferably purified, or antibody, and thereby evaluating the plurality of capture probes. Binding, e.g., in the case of a nucleic acid, hybridization, with a capture probe at an address of the plurality, is detected, e.g., by signal generated from a label attached to the 14266 nucleic acid, polypeptide, or antibody. The capture probes can be a set of nucleic acids from a selected sample, e.g., a sample of nucleic acids derived from a control or non-stimulated tissue or cell.

The method can include contacting the 14266 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of a 14266 sequence of the invention. Such methods can be used to diagnose a subject, e.g., to evaluate risk for a disease or disorder, to evaluate suitability of a selected treatment for a subject, to evaluate whether a subject has a disease or disorder.

The method can be used to detect single nucleotide polymorphisms (SNPs), as described below.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express a 14266 polypeptide of the invention or from a cell or subject in which a 14266-mediated response has been elicited, e.g., by contact of the cell with a 14266 nucleic acid or protein of the invention, or administration to the cell or subject a 14266 nucleic acid or protein of the invention; contacting the array with one or more inquiry probes, wherein an inquiry probe can be a nucleic acid, polypeptide, or antibody (which is preferably other than a 14266 nucleic acid, polypeptide, or antibody of the invention); providing a two dimensional array having a plurality of addresses, each address of the plurality being positionally distinguishable from each other address of the plurality, and each address of the plurality having a unique capture probe, e.g., wherein the capture probes are from a cell or subject which does not express a 14266 sequence of the invention (or does not express as highly as in the case of the 14266 positive plurality of capture probes) or from a cell or subject in which a 14266-mediated response has not been elicited (or has been elicited to a lesser extent than in the first sample); contacting the array with one or more inquiry probes (which is preferably other than a 14266 nucleic acid, polypeptide, or antibody of the invention), and thereby evaluating the plurality of capture probes. Binding, e.g., in the case of a nucleic acid, hybridization, with a capture probe at an address of the plurality, is detected, e.g., by signal generated from a label attached to the nucleic acid, polypeptide, or antibody.

In another aspect, the invention features a method of analyzing a 14266 sequence of the invention, e.g., analyzing structure, function, or relatedness to other nucleic acid or amino acid sequences. The method includes: providing a 14266 nucleic acid or amino acid sequence, e.g., the sequence set forth in SEQ ID NO:12 (nucleic acid) or SEQ ID NO:11 (amino acid) or a portion thereof; comparing the 14266 sequence with one or more, preferably a plurality of sequences from a collection of sequences, e.g., a nucleic acid or protein sequence database; to thereby analyze the 14266 sequence of the invention.

The method can include evaluating the sequence identity between a 14266 sequence of the invention and a database sequence. The method can be performed by accessing the database at a second site, e.g., over the internet.

In another aspect, the invention features, a set of oligonucleotides, useful, e.g., for identifying SNP's, or identifying specific alleles of a 14266 sequence of the invention. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotides which hybridizes to one allele provides a signal that is distinguishable from an oligonucleotides which hybridizes to a second allele.

3. Prognostic Assays

The methods described herein can furthermore be utilized as diagnostic or prognostic assays to identify subjects having or at risk of developing a disease or disorder associated with 14266 receptor, 14266 nucleic acid expression, or 14266 activity. Prognostic assays can be used for prognostic or predictive purposes to thereby prophylactically treat an individual prior to the onset of a disorder characterized by or associated with 14266 receptor, 14266 nucleic acid expression, or 14266 activity.

Thus, the present invention provides a method in which a test sample is obtained from a subject, and 14266 receptor or nucleic acid (e.g., mRNA, genomic DNA) is detected, wherein the presence of 14266 receptor or nucleic acid is diagnostic for a subject having or at risk of developing a disease or disorder associated with aberrant 14266 expression or activity. As used herein, a “test sample” refers to a biological sample obtained from a subject of interest. For example, a test sample can be a biological fluid (e.g., serum), cell sample, or tissue.

Furthermore, using the prognostic assays described herein, the present invention provides methods for determining whether a subject can be administered a specific agent (e.g., an agonist, antagonist, peptidomimetic, protein, peptide, antibody, nucleic acid (including an antisense nucleic acid or a ribozyme), small molecule, or other drug candidate) or class of agents (e.g., agents of a type that decrease 14266 activity) to effectively treat a disease or disorder associated with aberrant 14266 expression or activity. In this manner, a test sample is obtained and 14266 receptor or nucleic acid is detected. The presence of 14266 receptor or nucleic acid is diagnostic for a subject that can be administered the agent to treat a disorder associated with aberrant 14266 expression or activity.

The methods of the invention can also be used to detect genetic lesions or mutations in a 14266 gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a 14266 protein, or the misexpression of the 14266 gene. For example, such genetic lesions or mutations can be detected by ascertaining the existence of at least one of: (1) a deletion of one or more nucleotides from a 14266 gene; (2) an addition of one or more nucleotides to a 14266 gene; (3) a substitution of one or more nucleotides of a 14266 gene; (4) a chromosomal rearrangement of a 14266 gene; (5) an alteration in the level of a messenger RNA transcript of a 14266 gene; (6) an aberrant modification of a 14266 gene, such as of the methylation pattern of the genomic DNA; (7) the presence of a non-wild-type splicing pattern of a messenger RNA transcript of a 14266 gene; (8) a non-wild-type level of a 14266-protein; (9) an allelic loss of a 14266 gene; and (10) an inappropriate post-translational modification of a 14266-protein. As described herein, there are a large number of assay techniques known in the art that can be used for detecting lesions in a 14266 gene. Any cell type or tissue, in which 14266 receptors are expressed may be utilized in the prognostic assays described herein.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the 14266-gene (see, e.g., Abravaya et al. (1995) Nucleic Acids Res. 23:675-682). It is anticipated that the PCR and/or LCR may be desirable to use as a preliminary amplification step in conjunction with any of the techniques used for detecting mutations described herein.

Alternative amplification methods include self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 14266 gene from a sample cell can be identified by alterations in restriction enzyme cleavage patterns of isolated test sample and control DNA digested with one or more restriction endonucleases. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in a 14266 molecule can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). In yet another embodiment, any of a variety of sequencing reactions known in the art can be used to directly sequence the 14266 gene and detect mutations by comparing the sequence of the sample 14266 gene with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 14266 gene include methods in which protection from cleavage agents is used to detect mismatched bases in RNA/RNA or RNA/DNA heteroduplexes (Myers et al. (1985) Science 230:1242). See, also Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more “DNA mismatch repair” enzymes that recognize mismatched base pairs in double-stranded DNA in defined systems for detecting and mapping point mutations in 14266 cDNAs obtained from samples of cells. See, e.g., Hsu et al. (1994) Carcinogenesis 15:1657-1662. According to an exemplary embodiment, a probe based on a 14266 sequence, e.g., a wild-type 14266 sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in 14266 genes. For example, single-strand conformation polymorphism (SSCP) may be used to detect differences in electrophoretic mobility between mutant and wild-type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc. Natl. Acad. Sci. USA 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology, which depends on selective PCR amplification, may be used in conjunction with the instant invention. Oligonucleotides used as primers for specific amplification may carry the mutation of interest in the center of the molecule so that amplification depends on differential hybridization (Gibbs et al. (1989) Nucleic Acids Res. 17:2437-2448) or at the extreme 30 end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3□ end of the 5□ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnosed patients exhibiting symptoms or family history of a disease or illness involving a 14266 gene.

4. Pharmacogenomics

Agents, or modulators that have a stimulatory or inhibitory effect on 14266 activity (e.g., 14266 gene expression) as identified by a screening assay described herein, can be administered to individuals to treat (prophylactically or therapeutically) disorders associated with aberrant 14266 activity as well as to modulate the phenotype of a differentiative or cell proliferation disorder. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of 14266 receptor, expression of 14266 nucleic acid, or mutation content of 14266 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (antimalarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, an “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a 14266 receptor of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a 14266 molecule or 14266 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 14266 molecule or 14266 modulator of the invention, such as a modulator identified by one of the exemplary screening assays described herein.

The present invention further provides methods for identifying new agents, or combinations, that are based on identifying agents that modulate the activity of one or more of the gene products encoded by one or more of the 14266 genes of the present invention, wherein these products may be associated with resistance of the cells to a therapeutic agent. Specifically, the activity of the proteins encoded by the 14266 genes of the present invention can be used as a basis for identifying agents for overcoming agent resistance. By blocking the activity of one or more of the resistance proteins, target cells, will become sensitive to treatment with an agent that the unmodified target cells were resistant to. Agents of the present invention include small molecule modulators, antibodies, ribozymes, peptides, and antisense nucleic acid molecules.

Monitoring the influence of agents (e.g., drugs) on the expression or activity of a 14266 receptor can be applied in clinical trials. For example, the effectiveness of an agent determined by a screening assay as described herein to increase 14266 gene expression, protein levels, or upregulate 14266 activity, can be monitored in clinical trials of subjects exhibiting decreased 14266 gene expression, protein levels, or downregulated 14266 activity. Alternatively, the effectiveness of an agent determined by a screening assay to decrease 14266 gene expression, protein levels, or downregulate 14266 activity, can be monitored in clinical trials of subjects exhibiting increased 14266 gene expression, protein levels, or upregulated 14266 activity. In such clinical trials, the expression or activity of a 14266 gene, and preferably, other genes that have been implicated in, for example, a 14266-associated disorder can be used as a “read out” or markers of the phenotype of a particular cell.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of 14266 receptor, expression of 14266 nucleic acid, or mutation content of 14266 genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a 14266 modulator, such as a modulator identified by one of the exemplary screening assays described herein.

5. Monitoring of Effects During Clinical Trials

Monitoring the influence of agents (e.g., drugs, compounds) on the expression or activity of 14266 genes (e.g., the ability to modulate aberrant cell proliferation and/or differentiation) can be applied not only in basic drug screening but also in clinical trials. For example, the effectiveness of an agent, as determined by a screening assay as described herein, to increase or decrease 14266 gene expression, protein levels, or protein activity, can be monitored in clinical trials of subjects exhibiting decreased or increased 14266 gene expression, protein levels, or protein activity. In such clinical trials, 14266 expression or activity and preferably that of other genes that have been implicated in for example, a cellular proliferation disorder, can be used as a marker of the immune responsiveness of a particular cell.

For example, and not by way of limitation, genes that are modulated in cells by treatment with an agent (e.g., compound, drug, or small molecule) that modulates 14266 activity (e.g., as identified in a screening assay described herein) can be identified. Thus, to study the effect of agents on cellular proliferation disorders, for example, in a clinical trial, cells can be isolated and RNA prepared and analyzed for the levels of expression of 14266 genes and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of 14266 genes or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, antibody, nucleic acid (including an antisense oligonucleotide or a ribozyme), small molecule, or other drug candidate identified by the screening assays described herein) comprising the steps of (1) obtaining a preadministration sample from a subject prior to administration of the agent; (2) detecting the level of expression of a 14266 receptor, mRNA, or genomic DNA in the preadministration sample; (3) obtaining one or more postadministration samples from the subject; (4) detecting the level of expression or activity of the 14266 receptor, mRNA, or genomic DNA in the postadministration samples; (5) comparing the level of expression or activity of the 14266 receptor, mRNA, or genomic DNA in the preadministration sample with the 14266 receptor, mRNA, or genomic DNA in the postadministration sample or samples; and (vi) altering the administration of the agent to the subject accordingly to bring about the desired effect, i.e., for example, an increase or a decrease in the expression or activity of a 14266 receptor.

C. Methods of Treatment

The present invention provides for both prophylactic and therapeutic methods of treating a subject at risk of (or susceptible to) a disorder or having a disorder associated with aberrant 14266 expression or activity. Additionally, the compositions of the invention find use in the treatment of disorders described herein. Thus, therapies for disorders associated with CCC are encompassed herein.

1. Prophylactic Methods

In one aspect, the invention provides a method for preventing in a subject a disease or condition associated with an aberrant 14266 expression or activity by administering to the subject an agent that modulates 14266 expression or at least one 14266 gene activity. Subjects at risk for a disease that is caused, or contributed to, by aberrant 14266 expression or activity can be identified by, for example, any or a combination of diagnostic or prognostic assays as described herein. Administration of a prophylactic agent can occur prior to the manifestation of symptoms characteristic of the 14266 aberrancy, such that a disease or disorder is prevented or, alternatively, delayed in its progression. Depending on the type of 14266 aberrancy, for example, a 14266 agonist or 14266 antagonist agent can be used for treating the subject. The appropriate agent can be determined based on screening assays described herein.

2. Therapeutic Methods

Another aspect of the invention pertains to methods of modulating 14266 expression or activity for therapeutic purposes. The modulatory method of the invention involves contacting a cell with an agent that modulates one or more of the activities of 14266 receptor activity associated with the cell. An agent that modulates 14266 receptor activity can be an agent as described herein, such as a nucleic acid or a protein, a naturally-occurring cognate ligand of a 14266 receptor, a peptide, a 14266 peptidomimetic, or other small molecule. In one embodiment, the agent stimulates one or more of the biological activities of 14266 receptor. Examples of such stimulatory agents include active 14266 receptor and a nucleic acid molecule encoding a 14266 receptor that has been introduced into the cell. In another embodiment, the agent inhibits one or more of the biological activities of 14266 receptor. Examples of such inhibitory agents include antisense 14266 nucleic acid molecules and anti-14266 antibodies.

These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a 14266 receptor or nucleic acid molecule. In one embodiment, the method involves administering an agent (e.g., an agent identified by a screening assay described herein), or a combination of agents, that modulates (e.g., upregulates or downregulates) 14266 expression or activity. In another embodiment, the method involves administering a 14266 receptor or nucleic acid molecule as therapy to compensate for reduced or aberrant 14266 expression or activity.

Stimulation of 14266 activity is desirable in situations in which a 14266 receptor is abnormally downregulated and/or in which increased 14266 activity is likely to have a beneficial effect. Conversely, inhibition of 14266 activity is desirable in situations in which 14266 activity is abnormally upregulated and/or in which decreased 14266 activity is likely to have a beneficial effect.

Polypeptides

The invention thus relates to a human 14266 and to the expression of a 14266 having the deduced amino acid sequence shown in (SEQ ID NO:11)

“14266 polypeptide” or “14266 protein” refers to the polypeptide in SEQ ID NO:12. The term “14266 protein” or “14266 polypeptide,” however, further includes the numerous variants described herein, as well as fragments derived from the full-length 14266 and variants.

Preferred 14266 polypeptides of the present invention have an amino acid sequence sufficiently identical to the amino acid sequence encoded by the nucleic acid sequences of SEQ ID NO:12. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 90%, 91%, 92%, 93%, 94%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to 14266 nucleic acid molecules of the invention. BLAST protein searches can be performed with the XBLAST program, score=50, wordlength=3, to obtain amino acid sequences homologous to 14266 protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Accordingly, another embodiment of the invention features isolated 14266 proteins and polypeptides having a 14266 protein activity. As used interchangeably herein, a “14266 protein activity”, “biological activity of a 14266 protein”, or “functional activity of a 14266 protein” refers to an activity exerted by a 14266 protein, polypeptide, or nucleic acid molecule on a 14266 responsive cell as determined in vivo, or in vitro, according to standard assay techniques. A 14266 activity can be a direct activity, such as an association with or an enzymatic activity on a second protein, or an indirect activity, such as a cellular signaling activity mediated by interaction of the 14266 protein with a second protein. In a preferred embodiment, a 14266 activity includes at least one or more of the following activities: (1) modulating (stimulating and/or enhancing or inhibiting) cellular proliferation, differentiation, and/or function; (2) mobilization of intracellular molecules that participate in a signal transduction pathway, e.g., phosphatidylinositol 4,5-bisphosphate (PIP2), inositol 1,4,5-triphosphate (IP3) and adenylate cyclase; (3) polarization of the plasma membrane; (4) production or secretion of molecules; (5) alteration in the structure of a cellular component; (6) cell proliferation, e.g., synthesis of DNA; (7) cell migration; (8) cell differentiation (including neutrophil differentiation); (9) cell survival and (10) ligand binding.

An “isolated” or “purified” 14266 nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated 14266 nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A 14266 protein that is substantially free of cellular material includes preparations of 14266 protein having less than about 30%, 20%, 10%, or 5% (by dry weight) of non-14266 protein (also referred to herein as a “contaminating protein”). When the 14266 protein or biologically active portion thereof is recombinantly produced, preferably, culture medium represents less than about 30%, 20%, 10%, or 5% of the volume of the protein preparation. When 14266 protein is produced by chemical synthesis, preferably the protein preparations have less than about 30%, 20%, 10%, or 5% (by dry weight) of chemical precursors or non-14266 chemicals.

Fragments or biologically active portions of the 14266 receptor are also encompassed within the present invention. By “14266 receptor” is intended a protein having the amino acid sequence encoded by the amino acid sequence set forth in SEQ ID NO:11 as well as fragments, biologically active portions, and variants thereof.

“Fragments” or “biologically active portions” include polypeptide fragments suitable for use as immunogens to raise anti-14266 antibodies. Fragments include peptides comprising amino acid sequences sufficiently identical to or derived from the amino acid sequence of a 14266 protein, or a fragment thereof, of the invention and exhibiting at least one activity of a 14266 protein, but which include fewer amino acids than the 14266 protein encoded by the nucleic acid sequences disclosed herein. Typically, biologically active portions comprise a domain or motif with at least one activity of the 14266 protein. A biologically active portion of a 14266 protein can be a polypeptide which is, for example, 10, 25, 50, 100 or more amino acids in length. Such biologically active portions can be prepared by recombinant techniques and evaluated for one or more of the functional activities of a native 14266 protein. As used here, a fragment comprises at least 5 contiguous amino acids of an amino acid sequence set forth in SEQ ID NO:11. The invention encompasses other fragments, however, such as any fragment in the protein greater than 6, 7, 8, or 9 amino acids.

By “variants” is intended proteins or polypeptides having an amino acid sequence that is at least about 45%, 55%, 65%, preferably about 75%, 85%, 90%, 91%, 92%, 93%, 94%, 95%, 96%, 97%, 98%, or 99% identical to an amino acid sequence set forth in SEQ ID NO:11. Variants also include polypeptides encoded by a nucleic acid molecule that hybridizes to the nucleic acid molecule of SEQ ID NO:12, or a complement thereof, under stringent conditions. Such variants generally retain the functional activity of the 14266 proteins of the invention. Variants include polypeptides that differ in amino acid sequence due to natural allelic variation or mutagenesis.

The invention also provides 14266 chimeric or fusion proteins. As used herein, a 14266 “chimeric protein” or “fusion protein” comprises a 14266 polypeptide operably linked to a non-14266 polypeptide. A “14266 polypeptide” refers to a polypeptide having an amino acid sequence corresponding to a 14266 protein, whereas a “non-14266 polypeptide” refers to a polypeptide having an amino acid sequence corresponding to a protein that is not substantially identical to the 14266 protein, e.g., a protein that is different from the 14266 protein and which is derived from the same or a different organism. Within a 14266 fusion protein, the 14266 polypeptide can correspond to all or a portion of a 14266 protein, preferably at least one biologically active portion of a 14266 protein. Within the fusion protein, the term “operably linked” is intended to indicate that the 14266 polypeptide and the non-14266 polypeptide are fused in-frame to each other. The non-14266 polypeptide can be fused to the N-terminus or C-terminus of the 14266 polypeptide.

One useful fusion protein is a GST-14266 fusion protein in which the 14266 sequences are fused to the C-terminus of the GST sequences. Such fusion proteins can facilitate the purification of recombinant 14266 proteins.

In yet another embodiment, the fusion protein is a 14266-immunoglobulin fusion protein in which all or part of a 14266 protein is fused to sequences derived from a member of the immunoglobulin protein family. The 14266-immunoglobulin fusion proteins of the invention can be incorporated into pharmaceutical compositions and administered to a subject to inhibit an interaction between a 14266 ligand and a 14266 protein on the surface of a cell, thereby suppressing 14266-mediated signal transduction in vivo. The 14266-immunoglobulin fusion proteins can be used to affect the bioavailability of a 14266 cognate ligand. Inhibition of the 14266 ligand/14266 interaction may be useful therapeutically, both for treating proliferative, differentiative, developmental and hemopoietic disorders and for modulating (e.g., promoting or inhibiting) cell survival. Moreover, the 14266-immunoglobulin fusion proteins of the invention can be used as immunogens to produce anti-14266 antibodies in a subject, to purify 14266 ligands, and in screening assays to identify molecules that inhibit the interaction of a 14266 protein with a 14266 ligand.

Preferably, a 14266 chimeric or fusion protein of the invention is produced by standard recombinant DNA techniques. For example, DNA fragments coding for the different polypeptide sequences may be ligated together in-frame, or the fusion gene can be synthesized, such as with automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, e.g., Ausubel et al., eds. (1995) Current Protocols in Molecular Biology) (Greene Publishing and Wiley-Interscience, NY). Moreover, a 14266-encoding nucleic acid can be cloned into a commercially available expression vector such that it is linked in-frame to an existing fusion moiety.

Variants of the 14266 proteins can function as either 14266 agonists (mimetics) or as 14266 antagonists. Variants of the 14266 protein can be generated by mutagenesis, e.g., discrete point mutation or truncation of the 14266 protein. An agonist of the 14266 protein can retain substantially the same, or a subset, of the biological activities of the naturally occurring form of the 14266 protein. An antagonist of the 14266 protein can inhibit one or more of the activities of the naturally occurring form of the 14266 protein by, for example, competitively binding to a downstream or upstream member of a cellular signaling cascade that includes the 14266 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the 14266 proteins.

Variants of a 14266 protein that function as either 14266 agonists or as 14266 antagonists can be identified by screening combinatorial libraries of mutants, e.g., truncation mutants, of a 14266 protein for 14266 protein agonist or antagonist activity. In one embodiment, a variegated library of 14266 variants is generated by combinatorial mutagenesis at the nucleic acid level and is encoded by a variegated gene library. A variegated library of 14266 variants can be produced by, for example, enzymatically ligating a mixture of synthetic oligonucleotides into gene sequences such that a degenerate set of potential 14266 sequences is expressible as individual polypeptides, or alternatively, as a set of larger fusion proteins (e.g., for phage display) containing the set of 14266 sequences therein. There are a variety of methods that can be used to produce libraries of potential 14266 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 14266 sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 14266 protein coding sequence can be used to generate a variegated population of 14266 fragments for screening and subsequent selection of variants of a 14266 protein. In one embodiment, a library of coding sequence fragments can be generated by treating a double-stranded PCR fragment of a 14266 coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the 14266 protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of 14266 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify 14266 variants (Arkin and Yourvan (1992) Proc. Natl. Acad. Sci. USA 89:7811-7815; Delgrave et al. (1993) Protein Engineering 6(3):327-331).

An isolated 14266 polypeptide of the invention can be used as an immunogen to generate antibodies that bind 14266 proteins using standard techniques for polyclonal and monoclonal antibody preparation. The full-length 14266 protein can be used or, alternatively, the invention provides antigenic peptide fragments of 14266 proteins for use as immunogens. The antigenic peptide of a 14266 protein comprises at least 8, preferably 10, 15, 20, or 30 amino acid residues of the amino acid sequence set forth in SEQ ID NO:11 and encompasses an epitope of a 14266 protein such that an antibody raised against the peptide forms a specific immune complex with the 14266 protein. Preferred epitopes encompassed by the antigenic peptide are regions of a 14266 protein that are located on the surface of the protein, e.g., hydrophilic regions.

Accordingly, another aspect of the invention pertains to anti-14266 polyclonal and monoclonal antibodies that bind a 14266 protein. Polyclonal anti-14266 antibodies can be prepared by immunizing a suitable subject (e.g., rabbit, goat, mouse, or other mammal) with a 14266 immunogen. The anti-14266 antibody titer in the immunized subject can be monitored over time by standard techniques, such as with an enzyme linked immunosorbent assay (ELISA) using immobilized 14266 protein. At an appropriate time after immunization, e.g., when the anti-14266 antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal anti-14266 antibody can be identified and isolated by screening a recombinant combinatorial immunoglobulin library (e.g., an antibody phage display library) with a 14266 protein to thereby isolate immunoglobulin library members that bind the 14266 protein. Kits for generating and screening phage display libraries are commercially available (e.g., the Pharmacia Recombinant Phage Antibody System, Catalog No. 27-9400-01; and the Stratagene SurfZAP□ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-14266 antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in PCT Publication Nos. WO 86/101533 and WO 87/02671; European Patent Application Nos. 184,187, 171, 496, 125,023, and 173,494; U.S. Pat. Nos. 4,816,567 and 5,225,539; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) Bio/Techniques 4:214; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

An anti-14266 antibody (e.g., monoclonal antibody) can be used to isolate 14266 proteins by standard techniques, such as affinity chromatography or immunoprecipitation. An anti-14266 antibody can facilitate the purification of natural 14266 protein from cells and of recombinantly produced 14266 protein expressed in host cells. Moreover, an anti-14266 antibody can be used to detect 14266 protein (e.g., in a cellular lysate or cell supernatant) in order to evaluate the abundance and pattern of expression of the 14266 protein. Anti-14266 antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 35S, or 3H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84:Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

Methods for Using the Polynucleotide

The methods and uses described herein below for the 14266 polynucleotide are particularly applicable to the cells and tissues that contain detectable levels of 14266 expression as described above.

These methods pertain to isolated nucleic acid molecules comprising nucleotide sequences encoding 14266 proteins and polypeptides or biologically active portions thereof, as well as nucleic acid molecules sufficient for use as hybridization probes to identify 14266-encoding nucleic acids (e.g., 14266 mRNA) and fragments for use as PCR primers for the amplification or mutation of 14266 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences encoding the 14266 proteins of the present invention include sequence set forth in SEQ ID NO:12 and complements thereof. By “complement” is intended a nucleotide sequence that is sufficiently complementary to a given nucleotide sequence such that it can hybridize to the given nucleotide sequence to thereby form a stable duplex. The corresponding amino acid sequence for the 14266 protein encoded by these nucleotide sequences are also encompassed by the present invention. The invention also encompasses nucleic acid molecules comprising nucleotide sequences encoding partial-length 14266 proteins, including the sequence set forth in SEQ ID NO:12, and complements thereof.

Nucleic acid molecules that are fragments of these 14266 nucleotide sequences are also encompassed by the present invention. By “fragment” is intended a portion of the nucleotide sequence encoding a 14266 protein. A fragment of a 14266 nucleotide sequence may encode a biologically active portion of a 14266 protein, or it may be a fragment that can be used as a hybridization probe or PCR primer using methods disclosed below. A biologically active portion of a 14266 protein can be prepared by isolating a portion of one of the nucleotide sequences of the invention, expressing the encoded portion of the 14266 protein (e.g., by recombinant expression in vitro), and assessing the activity of the encoded portion of the 14266 protein. Nucleic acid molecules that are fragments of a 14266 nucleotide sequence comprise at least about 15, 20, 50, 75, 100, 200, 300, 350, 400, 450, 500, 550, 600, 650, 700, 750, 800, 850, 900, 950, 1000, 1050, 1100, 1150, 1200, nucleotides, or up to the number of nucleotides present in the 14266 nucleotide sequence disclosed herein depending upon the intended use.

It is understood that isolated fragments include any contiguous sequence not disclosed prior to the invention as well as sequences that are substantially the same and which are not disclosed. Accordingly, if an isolated fragment is disclosed prior to the present invention, that fragment is not intended to be encompassed by the invention. When a sequence is not disclosed prior to the present invention, an isolated nucleic acid fragment is at least about 12, 15, 20, 25, or 30 contiguous nucleotides. Other regions of the nucleotide sequence may comprise fragments of various sizes, depending upon potential homology with previously disclosed sequences.

A fragment of a 14266 nucleotide sequence that encodes a biologically active portion of a 14266 protein of the invention will encode at least about 15, 25, 30, 50, 75, 100, 125, 150, 175, 200, 250, or 300 contiguous amino acids, or up to the total number of amino acids present in a full-length 14266 protein of the invention. Fragments of a 14266 nucleotide sequence that are useful as hybridization probes for PCR primers generally need not encode a biologically active portion of a 14266 protein.

Nucleic acid molecules that are variants of the 14266 nucleotide sequences disclosed herein are also encompassed by the present invention. “Variants” of the 14266 nucleotide sequences include those sequences that encode the 14266 proteins disclosed herein but that differ conservatively because of the degeneracy of the genetic code. These naturally occurring allelic variants can be identified with the use of well-known molecular biology techniques, such as the polymerase chain reaction (PCR) and hybridization techniques as outlined below. Variant nucleotide sequences also include synthetically derived nucleotide sequences that have been generated, for example, by using site-directed mutagenesis but which still encode the 14266 proteins disclosed in the present invention as discussed below. Generally, nucleotide sequence variants of the invention will have at least about 45%, 55%, 65%, 75%, 85%, 90%, 91%, 92%, 93%, 94%, 95%, 96%, 97%, 98%, or 99% identity to a particular nucleotide sequence disclosed herein. A variant 14266 nucleotide sequence will encode a 14266 protein that has an amino acid sequence having at least about 45%, 55%, 65%, 75%, 85%, 90%, 91%, 92%, 93%, 94%, 95%, 96%, 97%, 98%, or 99% identity to the amino acid sequence of a 14266 protein disclosed herein.

In addition to the 14266 nucleotide sequence shown in SEQ ID NO:12, it will be appreciated by those skilled in the art that DNA sequence polymorphisms that lead to changes in the amino acid sequences of 14266 proteins may exist within a population (e.g., the human population). Such genetic polymorphism in a 14266 gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a 14266 protein, preferably a mammalia 14266 protein. As used herein, the phrase “allelic variant” refers to a nucleotide sequence that occurs at a 14266 locus or to a polypeptide encoded by the nucleotide sequence. Such natural allelic variations can typically result in 1-5% variance in the nucleotide sequence of the 14266 gene. Any and all such nucleotide variations and resulting amino acid polymorphisms or variations in a 14266 sequence that are the result of natural allelic variation and that do not alter the functional activity of 14266 proteins are intended to be within the scope of the invention.

Moreover, nucleic acid molecules encoding 14266 proteins from other species (14266 homologues), which have a nucleotide sequence differing from that of the 14266 sequences disclosed herein, are intended to be within the scope of the invention. For example, nucleic acid molecules corresponding to natural allelic variants and homologues of the human 14266 cDNA of the invention can be isolated based on their identity to the human 14266 nucleic acid disclosed herein using the human cDNA, or a portion thereof, as a hybridization probe according to standard hybridization techniques under stringent hybridization conditions as disclosed below.

In addition to naturally-occurring allelic variants of the 14266 sequences that may exist in the population, the skilled artisan will further appreciate that changes can be introduced by mutation into the nucleotide sequences of the invention thereby leading to changes in the amino acid sequence of the encoded 14266 proteins, without altering the biological activity of the 14266 proteins. Thus, an isolated nucleic acid molecule encoding a 14266 protein having a sequence that differs from the amino acid sequence encoded by the nucleotide sequence of SEQ ID NO:12 can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of a 14266 protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif.

Alternatively, variant 14266 nucleotide sequences can be made by introducing mutations randomly along all or part of a 14266 coding sequence, such as by saturation mutagenesis, and the resultant mutants can be screened for 14266 biological activity to identify mutants that retain activity. Following mutagenesis, the encoded protein can be expressed recombinantly, and the activity of the protein can be determined using standard assay techniques.

Thus the nucleotide sequences of the invention include the sequences disclosed herein as well as fragments and variants thereof. The 14266 nucleotide sequences of the invention, and fragments and variants thereof, can be used as probes and/or primers to identify and/or clone 14266 homologues in other cell types, e.g., from other tissues, as well as 14266 homologues from other mammals. Such probes can be used to detect transcripts or genomic sequences encoding the same or identical proteins. These probes can be used as part of a diagnostic test kit for identifying cells or tissues that misexpress a 14266 protein, such as by measuring levels of a 14266-encoding nucleic acid in a sample of cells from a subject, e.g., detecting 14266 mRNA levels or determining whether a genomic 14266 gene has been mutated or deleted.

In this manner, methods such as PCR, hybridization, and the like can be used to identify such sequences having substantial identity to the sequences of the invention. See, for example, Sambrook et al. (1989) Molecular Cloning: Laboratory Manual (2d ed., Cold Spring Harbor Laboratory Press, Plainview, N.Y.) and Innis, et al. (1990) PCR Protocols: A Guide to Methods and Applications (Academic Press, NY). 14266 nucleotide sequences isolated based on their sequence identity to the 14266 nucleotide sequences set forth herein or to fragments and variants thereof are encompassed by the present invention.

In a hybridization method, all or part of a known 14266 nucleotide sequence can be used to screen cDNA or genomic libraries. Methods for construction of such cDNA and genomic libraries are generally known in the art and are disclosed in Sambrook et al. (1989) Molecular Cloning: A Laboratory Manual (2d ed., Cold Spring Harbor Laboratory Press, Plainview, N.Y.). The so-called hybridization probes may be genomic DNA fragments, cDNA fragments, RNA fragments, or other oligonucleotides, and may be labeled with a detectable group such as 32P, or any other detectable marker, such as other radioisotopes, a fluorescent compound, an enzyme, or an enzyme co-factor. Probes for hybridization can be made by labeling synthetic oligonucleotides based on the known 14266 nucleotide sequence disclosed herein. Degenerate primers designed on the basis of conserved nucleotides or amino acid residues in a known 14266 nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, or 400 consecutive nucleotides of a 14266 nucleotide sequence of the invention or a fragment or variant thereof. Preparation of probes for hybridization is generally known in the art and is disclosed in Sambrook et al. (1989) Molecular Cloning: A Laboratory Manual (2d ed., Cold Spring Harbor Laboratory Press, Plainview, N.Y.), herein incorporated by reference.

For example, in one embodiment, a previously unidentified 14266 nucleic acid molecule hybridizes under stringent conditions to a probe that is a nucleic acid molecule comprising one of the 14266 nucleotide sequences of the invention or a fragment thereof. In another embodiment, the previously unknown 14266 nucleic acid molecule is at least about 300, 325, 350, 375, 400, 425, 450, 500, 550, 600, 650, 700, 800, 900, 1000, 2,000, 3,000, 4,000 or 5,000 nucleotides in length and hybridizes under stringent conditions to a probe that is a nucleic acid molecule comprising one of the 14266 nucleotide sequences disclosed herein or a fragment thereof.

Accordingly, in another embodiment, an isolated previously unknown 14266 nucleic acid molecule of the invention is at least about 300, 325, 350, 375, 400, 425, 450, 500, 550, 600, 650, 700, 800, 900, 1000, 1,100, 1,200, 1,300, or 1,400 nucleotides in length and hybridizes under stringent conditions to a probe that is a nucleic acid molecule comprising one of the nucleotide sequences of the invention, preferably the coding sequence of the nucleotides sequences set forth in SEQ ID NO:12 or a complement, fragment, or variant thereof.

As used herein, the term “hybridizes under stringent conditions” is intended to describe conditions for hybridization and washing under which nucleotide sequences having at least about 60%, 65%, 70%, preferably 75% identity to each other typically remain hybridized to each other. Such stringent conditions are known to those skilled in the art and can be found in Current Protocols in Molecular Biology (John Wiley & Sons, New York (1989)), 6.3.1-6.3.6. A preferred, non-limiting example of stringent hybridization conditions is hybridization in 6× sodium chloride/sodium citrate (SSC) at about 45□C, followed by one or more washes in 0.2×SSC, 0.1% SDS at 50-65□C. In another preferred embodiment, stringent conditions comprise hybridization in 6×SSC at 42□C, followed by washing with 1×SSC at 55□C. Preferably, an isolated nucleic acid molecule that hybridizes under stringent conditions to a 14266 sequence of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Thus, in addition to the 14266 nucleotide sequences disclosed herein and fragments and variants thereof, the isolated nucleic acid molecules of the invention also encompass homologous DNA sequences identified and isolated from other cells and/or organisms by hybridization with entire or partial sequences obtained from the 14266 nucleotide sequences disclosed herein or variants and fragments thereof.

The present invention also encompasses antisense nucleic acid molecules, i.e., molecules that are complementary to a sense nucleic acid encoding a protein, e.g., complementary to the coding strand of a double-stranded cDNA molecule, or complementary to an mRNA sequence. Accordingly, an antisense nucleic acid can hydrogen bond to a sense nucleic acid. The antisense nucleic acid can be complementary to an entire 14266 coding strand, or to only a portion thereof, e.g., all or part of the protein coding region (or open reading frame). An antisense nucleic acid molecule can be antisense to a noncoding region of the coding strand of a nucleotide sequence encoding a 14266 protein. The noncoding regions are the 5□ and 3□ sequences that flank the coding region and are not translated into amino acids.

Antisense nucleic acids of the invention can be designed according to the rules of Watson and Crick base pairing. The antisense nucleic acid molecule can be complementary to the entire coding region of 14266 mRNA, but more preferably is an oligonucleotide that is antisense to only a portion of the coding or noncoding region of 14266 mRNA. For example, the antisense oligonucleotide can be complementary to the region surrounding the translation start site of 14266 mRNA. An antisense oligonucleotide can be, for example, about 5, 10,15, 20, 25, 30, 35, 40, 45, or 50 nucleotides in length. An antisense nucleic acid of the invention can be constructed using chemical synthesis and enzymatic ligation procedures known in the art.

For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, including, but not limited to, for example e.g., phosphorothioate derivatives and acridine substituted nucleotides. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When used therapeutically, the antisense nucleic acid molecules of the invention are typically administered to a subject or generated in situ such that they hybridize with or bind to cellular mRNA and/or genomic DNA encoding a 14266 protein to thereby inhibit expression of the protein, e.g., by inhibiting transcription and/or translation. An example of a route of administration of antisense nucleic acid molecules of the invention includes direct injection at a tissue site. Alternatively, antisense nucleic acid molecules can be modified to target selected cells and then administered systemically. For example, antisense molecules can be linked to peptides or antibodies to form a complex that specifically binds to receptors or antigens expressed on a selected cell surface. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes, which are catalytic RNA molecules with ribonuclease activity that are capable of cleaving a single-stranded nucleic acid, such as an mRNA, to which they have a complementary region. Ribozymes (e.g., hammerhead ribozymes (described in Haselhoff and Gerlach (1988) Nature 334:585-591) can be used to catalytically cleave 14266 mRNA transcripts to thereby inhibit translation of 14266 mRNA. A ribozyme having specificity for a 14266-encoding nucleic acid can be designed based upon the nucleotide sequence of a 14266 cDNA disclosed herein. See, e.g., Cech et al., U.S. Pat. No. 4,987,071; and Cech et al., U.S. Pat. No. 5,116,742. Alternatively, 14266 mRNA can be used to select a catalytic RNA having a specific ribonuclease activity from a pool of RNA molecules. See, e.g., Bartel and Szostak (1993) Science 261:1411-1418.

The invention also encompasses nucleic acid molecules that form triple helical structures. For example, 14266 gene expression can be inhibited by targeting nucleotide sequences complementary to the regulatory region of the 14266 protein (e.g., the 14266 promoter and/or enhancers) to form triple helical structures that prevent transcription of the 14266 gene in target cells. See generally Helene (1991) Anticancer Drug Des. 6(6):569; Helene (1992) Ann. N.Y. Acad. Sci. 660:27; and Maher (1992) Bioassays 14(12):807.

In preferred embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid-phase peptide synthesis protocols as described, for example, in Hyrup et al. (1996), supra; Perry-O'Keefe et al. (1996) Proc. Natl. Acad. Sci. USA 93:14670.

PNAs of a 14266 molecule can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA-directed PCR clamping; as artificial restriction enzymes when used in combination with other enzymes, e.g., S1 nucleases (Hyrup (1996), supra); or as probes or primers for DNA sequence and hybridization (Hyrup (1996), supra; Perry-O'Keefe et al. (1996), supra).

In another embodiment, PNAs of a 14266 molecule can be modified, e.g., to enhance their stability, specificity, or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra; Finn et al. (1996) Nucleic Acids Res. 24(17):3357-63; Mag et al. (1989) Nucleic Acids Res. 17:5973; and Peterson et al. (1975) Bioorganic Med. Chem. Lett. 5:1119.

Methods Using Vectors and Host Cells

The methods using vectors and host cells are particularly relevant where vectors are expressed in the cells and tissues with detectable levels of 14266 expression as described herein, or where the host cells are those that naturally express the gene or which may be the native or a recombinant cell expressing the gene.

It is understood that “host cells” and “recombinant host cells” refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

The host cells expressing the polypeptides described herein, and particularly recombinant host cells, have a variety of uses. First, the cells are useful for producing 14266 proteins or polypeptides that can be further purified to produce desired amounts of 14266 protein or fragments. Thus, host cells containing expression vectors are useful for polypeptide production, as well as cells producing significant amounts of the polypeptide. Such cells and tissues have been described herein above.

Host cells are also useful for conducting cell-based assays involving the 14266 or 14266 fragments. Thus, a recombinant host cell expressing a native 14266 is useful to assay for compounds that stimulate or inhibit 14266 function. This includes substrate, coenzyme, or 14266 subunit binding, and gene expression at the level of transcription or translation.

Host cells are also useful for identifying 14266 mutants in which these functions are affected. If the mutants naturally occur and give rise to a pathology, host cells containing the mutations are useful to assay compounds that have a desired effect on the mutant 14266 (for example, stimulating or inhibiting function) which may not be indicated by their effect on the native 14266.

Recombinant host cells are also useful for expressing the chimeric polypeptides described herein to assess compounds that activate or suppress activation by means of a heterologous domain, segment, site, and the like, as disclosed herein.

Further, mutant 14266s can be designed in which one or more of the various functions is engineered to be increased or decreased (e.g., substrate or coenzyme binding) and used to augment or replace 14266 proteins in an individual. Thus, host cells can provide a therapeutic benefit by replacing an aberrant 14266 or providing an aberrant 14266 that provides a therapeutic result. In one embodiment, the cells provide 14266s that are abnormally active.

In another embodiment, the cells provide a 14266 that is abnormally inactive. This 14266 can compete with endogenous 14266 in the individual.

In another embodiment, cells expressing 14266s that cannot be activated are introduced into an individual in order to compete with endogenous 14266 for cAMP. For example, in the case in which excessive substrates such as β-hydroxysteroid is part of a treatment modality, it may be necessary to inactivate this molecule at a specific point in treatment. Providing cells that compete for the molecule, but which cannot be affected by 14266 activation would be beneficial.

Homologously recombinant host cells can also be produced that allow the in situ alteration of endogenous 14266 polynucleotide sequences in a host cell genome. The host cell includes, but is not limited to, a stable cell line, cell in vivo, or cloned microorganism. This technology is more fully described in WO 93/09222, WO 91/12650, WO 91/06667, U.S. Pat. No. 5,272,071, and U.S. Pat. No. 5,641,670. Briefly, specific polynucleotide sequences corresponding to the 14266 polynucleotides or sequences proximal or distal to a 14266 gene are allowed to integrate into a host cell genome by homologous recombination where expression of the gene can be affected. In one embodiment, regulatory sequences are introduced that either increase or decrease expression of an endogenous sequence. Accordingly, a 14266 protein can be produced in a cell not normally producing it. Alternatively, increased expression of 14266 protein can be effected in a cell normally producing the protein at a specific level. Further, expression can be decreased or eliminated by introducing a specific regulatory sequence. The regulatory sequence can be heterologous to the 14266 protein sequence or can be a homologous sequence with a desired mutation that affects expression. Alternatively, the entire gene can be deleted. The regulatory sequence can be specific to the host cell or capable of functioning in more than one cell type. Still further, specific mutations can be introduced into any desired region of the gene to produce mutant 14266 proteins. Such mutations could be introduced, for example, into the specific functional regions such as the cyclic nucleotide-binding site.

In one embodiment, the host cell can be a fertilized oocyte or embryonic stem cell that can be used to produce a transgenic animal containing the altered 14266 gene. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous 14266 gene is selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a 14266 protein and identifying and evaluating modulators of 14266 protein activity.

Other examples of transgenic animals include non-human primates, sheep, dogs, cows, goats, chickens, and amphibians.

In one embodiment, a host cell is a fertilized oocyte or an embryonic stem cell into which 14266 polynucleotide sequences have been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the 14266 nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the 14266 protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems, which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of S. cerevisiae (O'Gorman et al. (1991) Science 251:1351-1355). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could affect substrate binding or coenzyme bind may not be evident from in vitro cell-free or cell-based assays. Accordingly, it is useful to provide non-human transgenic animals to assay in vivo 14266 function, including substrate interaction, the effect of specific mutant 14266s on 14266 function and interaction, and the effect of chimeric 14266s. It is also possible to assess the effect of null mutations, that is mutations that substantially or completely eliminate one or more 14266 functions.

In general, methods for producing transgenic animals include introducing a nucleic acid sequence according to the present invention, the nucleic acid sequence capable of expressing the protein in a transgenic animal, into a cell in culture or in vivo. When introduced in vivo, the nucleic acid is introduced into an intact organism such that one or more cell types and, accordingly, one or more tissue types, express the nucleic acid encoding the protein. Alternatively, the nucleic acid can be introduced into virtually all cells in an organism by transfecting a cell in culture, such as an embryonic stem cell, as described herein for the production of transgenic animals, and this cell can be used to produce an entire transgenic organism. As described, in a further embodiment, the host cell can be a fertilized oocyte. Such cells are then allowed to develop in a female foster animal to produce the transgenic organism.

Vectors/Host Cells

The methods using the vectors and host cells discussed above are based on the vectors and host cells including, but not limited to, those described below.

The invention also provides methods using vectors containing the 14266 polynucleotides. The term “vector” refers to a vehicle, preferably a nucleic acid molecule that can transport the 14266 polynucleotides. When the vector is a nucleic acid molecule, the 14266 polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the 14266 polynucleotides. Alternatively, the vector may integrate into the host cell genome and produce additional copies of the 14266 polynucleotides when the host cell replicates.

The invention provides vectors for the maintenance (cloning vectors) or vectors for expression (expression vectors) of the 14266 polynucleotides. The vectors can function in procaryotic or eukaryotic cells or in both (shuttle vectors).

Expression vectors contain cis-acting regulatory regions that are operably linked in the vector to the 14266 polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the 14266 polynucleotides from the vector. Alternatively, a trans-acting factor may be supplied by the host cell. Finally, a trans-acting factor can be produced from the vector itself.

It is understood, however, that in some embodiments, transcription and/or translation of the 14266 polynucleotides can occur in a cell-free system.

The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al. (1989) Molecular Cloning: A Laboratory Manual 2nd. ed., Cold Spring Harbor Laboratory Press, Cold Spring Harbor, N.Y.

A variety of expression vectors can be used to express a 14266 polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al. (1989) Molecular Cloning: A Laboratory Manual 2nd. ed., Cold Spring Harbor Laboratory Press, Cold Spring Harbor, N.Y.

The regulatory sequence may provide constitutive expression in one or more host cells (i.e., tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The 14266 polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the 14266 polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al. (1988) Gene 69:301-315) and pET 11d (Studier et al. (1990) Gene Expression Technology: Methods in Enzymology 185:60-89).

Recombinant protein expression can be maximized in a host bacteria by providing a genetic background wherein the host cell has an impaired capacity to proteolytically cleave the recombinant protein. (Gottesman, S. (1990) Gene Expression Technology: Methods in Enzymology 185, Academic Press, San Diego, Calif. 119-128). Alternatively, the sequence of the polynucleotide of interest can be altered to provide preferential codon usage for a specific host cell, for example E. coli. (Wada et al. (1992) Nucleic Acids Res. 20:2111-2118).

The 14266 polynucleotides can also be expressed by expression vectors that are operative in yeast. Examples of vectors for expression in yeast e.g., S. cerevisiae include pYepSec1 (Baldari et al. (1987) EMBO J. 6:229-234), pMFa (Kurjan et al. (1982) Cell 30:933-943), pJRY88 (Schultz et al. (1987) Gene 54:113-123), and pYES2 (Invitrogen Corporation, San Diego, Calif.).

The 14266 polynucleotides can also be expressed in insect cells using, for example, baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow et al. (1989) Virology 170:31-39).

In certain embodiments of the invention, the polynucleotides described herein are expressed in mammalian cells using mammalian expression vectors. Examples of mammalian expression vectors include pCDM8 (Seed, B. (1987) Nature 329:840) and pMT2PC (Kaufman et al. (1987) EMBO J. 6:187-195).

The expression vectors listed herein are provided by way of example only of the well-known vectors available to those of ordinary skill in the art that would be useful to express the 14266 polynucleotides. The person of ordinary skill in the art would be aware of other vectors suitable for maintenance propagation or expression of the polynucleotides described herein. These are found for example in Sambrook et al. (1989) Molecular Cloning: A Laboratory Manual 2nd, ed., Cold Spring Harbor Laboratory, Cold Spring Harbor Laboratory Press, Cold Spring Harbor, N.Y.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook et al. (Molecular Cloning: A Laboratory Manual, 2d ed., Cold Spring Harbor Laboratory, Cold Spring Harbor Laboratory Press, Cold Spring Harbor, N.Y.).

Host cells can contain more than one vector. Thus, different nucleotide sequences can be introduced on different vectors of the same cell. Similarly, the 14266 polynucleotides can be introduced either alone or with other polynucleotides that are not related to the 14266 polynucleotides such as those providing trans-acting factors for expression vectors. When more than one vector is introduced into a cell, the vectors can be introduced independently, co-introduced or joined to the 14266 polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the 14266 polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

Pharmaceutical Compositions

The receptor-like nucleic acid molecules, receptor-like proteins, and anti-receptor-like antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions of the invention are useful to treat any of the disorders discussed herein. The compositions are provided in therapeutically effective amounts. By “therapeutically effective amounts” is intended an amount sufficient to modulate the desired response. As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL□ (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a receptor-like protein or anti-receptor-like antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. Depending on the type and severity of the disease, about 1 μg/kg to about 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### IV. LIGAND RECEPTORS AND USES THEREFOR

### Background of the Invention

G-protein coupled receptors (GPCRs) are one of the major class of proteins that are responsible for transducing a signal within a cell. GPCRs are proteins that have seven transmembrane domains. Upon binding of a ligand to the extracellular domain of a GPCR, a signal is transduced within the cell which results in a change in a biological or physiological property of the cell.

GPCRs, along with G-proteins and effectors (intracellular enzymes and channels which are modulated by G-proteins), are the components of a modular signaling system that connects the state of intracellular second messengers to extracellular inputs. These genes and gene-products are potential causative agents of disease (Spiegel et al. (1993) J. Clin. Invest. 92:1119-1125; McKusick and Amberger (1993) J. Med. Genet. 30:1-26). Specific defects in the rhodopsin gene and the V2 vasopressin receptor gene have been shown to cause various forms of autosomal dominant and autosomal recessive retinitis pigmentosa (see Nathans et al. (1992) Annu. Rev. Genet. 26:403-424), nephrogenic diabetes insipidus (Holtzman et al. (1993) Hum. Mol. Genet. 2:1201-1204 and references therein). These receptors are of critical importance to both the central nervous system and peripheral physiological processes. Evolutionary analyses suggest that the ancestor of these proteins originally developed in concert with complex body plans and nervous systems.

The GPCR protein superfamily now contains over 250 types of paralogues, receptors that represent variants generated by gene duplications (or other processes), as opposed to orthologues, the same receptor from different species. The superfamily can be broken down into five families: Family I, receptors typified by rhodopsin and the beta2-adrenergic receptor and currently represented by over 200 unique members (reviewed by Dohlman et al. (1991) Annu. Rev. Biochem. 60:653-688 and references therein); Family II, the recently characterized parathyroid hormone/calcitonin/secretin receptor family (Juppner et al. (1991) Science 254:1024-1026; Lin et al. (1991) Science 254:1022-1024); Family III, the metabotropic glutamate receptor family in mammals (Nakanishi (1992) Science 258:597-603); Family IV, the cAMP receptor family, important in the chemotaxis and development of D. discoideum (Klein et al. (1988) Science 241:1467-1472); and Family V, the fungal mating pheromone receptors such as STE2 (reviewed by Kurjan (1992) Annu. Rev. Biochem. 61:1097-1129).

In addition to these groups of GPCRs, there are a small number of other proteins which present seven putative hydrophobic segments and appear to be unrelated to GPCRs; however, they have not been shown to couple to G-proteins. Drosophila express a photoreceptor-specific protein bride of sevenless (boss), a seven-transmembrane-segment protein which has been extensively studied and does not show evidence of being a GPCR (Hart et al. (1993) Proc. Natl. Acad. Sci. USA 90:5047-5051 (1993)). The gene frizzled (fz) in Drosophila is also thought to be a protein with seven transmembrane segments. Like boss, fz has not been shown to couple to G-proteins (Vinson et al. (1989) Nature 338:263-264).

G proteins represent a family of heterotrimeric proteins composed of α, β and γ subunits, which bind guanine nucleotides. These proteins are usually linked to cell surface receptors, e.g., receptors containing seven transmembrane domains, such as the ligand receptors. Following ligand binding to the receptor, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt. G proteins are described extensively in Lodish H. et al. Molecular Cell Biology, (Scientific American Books Inc., New York, N.Y., 1995), the contents of which are incorporated herein by reference

GPCRs are a major target for drug action and development. Accordingly, it is valuable to the field of pharmaceutical development to identify and characterize previously unknown GPCRs. The present invention advances the state of the art by providing a previously unidentified GPCR which is expressed predominantly in the brain.

### Summary of the Invention

The present invention is based on the identification of a novel G-protein coupled receptor (GPCR) that is expressed predominantly in the brain and nucleic acid molecules that encoded the GPCR, referred to herein as the flh2882 protein and flh2882 gene respectively. Based on this identification, the present invention provides: 1) isolated flh2882 protein; 2) isolated nucleic acid molecules that encode an flh2882 protein; 3) antibodies that selectively bind to the flh2882 protein; 4) methods of isolating allelic variants of the flh2882 protein and gene; 5) methods of identifying cells and tissues that express the flh2882 protein/gene; 6) methods of identifying agents and cellular compounds that bind to the flh2882 protein; 7) methods of identifying agents that modulate the expression of the flh2882 gene; and 8) methods of modulating the activity of the flh2882 protein in a cell or organism.

### Detailed Description of the Invention

The present invention is based on the discovery of a novel G-protein coupled receptor (GPCR) molecule that is expressed predominantly in the brain, the flh2882 protein, and nucleic acid molecules that encode the flh2882 protein, the flh2882 gene or flh2882 nucleic acid molecule. Specifically, an EST was first identified in a public database that had low homology to G-protein coupled receptors. PCR primers were then designed based on this sequence and a cDNA was identified by screening a human fetal cDNA library (See Example 1). Positive clones were sequenced and contigs were assembled. Analysis of the assembled sequence revealed that the cloned cDNA molecule encoded a GPCR, denoted herein as the flh2882 protein. The flh2882 protein is a GPCR and plays a role in and function in signaling pathways within cells that express the flh2882 protein, particularly brain cells.

Various aspects of the invention are described in further detail in the following subsections:

I. Isolated flh2882 Protein

The present invention provides isolated flh2882 protein as well as peptide fragments of an flh2882 protein.

As used herein, a protein is said to be “isolated” or “purified” when it is substantially free of cellular when it is isolated from recombinant and non-recombinant cells, or free of chemical precursors or other chemicals when it is chemically synthesized. The language “substantially free of cellular material” includes preparations of flh2882 protein in which the protein is separated from cellular components of the cells in which it is naturally or recombinantly produced. In one embodiment, the language “substantially free of cellular material” includes preparations of an flh2882 protein having less than about 30% (by dry weight) of non-flh2882 protein (also referred to herein as a “contaminating protein”), more preferably less than about 20% of non-flh2882 protein, still more preferably less than about 10% of non-flh2882 protein, and most preferably less than about 5% non-flh2882 protein. When the flh2882 protein or biologically active fragment thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of flh2882 protein in which the protein is separated from chemical precursors or other chemicals that are involved in the synthesis of the protein. In one embodiment, the language “substantially free of chemical precursors or other chemicals” includes preparations of flh2882 protein having less than about 30% (by dry weight) of chemical precursors or non-flh2882 chemicals, more preferably less than about 20% chemical precursors or non-flh2882 chemicals, still more preferably less than about 10% chemical precursors or non-flh2882 chemicals, and most preferably less than about 5% chemical precursors or non-flh2882 chemicals. In preferred embodiments, isolated proteins or biologically active fragments thereof lack contaminating proteins from the same animal from which the flh2882 protein is derived. Typically, such proteins are produced by recombinant expression of, for example, a human flh2882 protein in a non-human cell.

As used herein, an flh2882 protein is defined as a protein that comprises: 1) the amino acid sequence shown in SEQ ID NO:13; 2) functional and non-functional naturally occurring allelic variants of human flh2882 protein; 3) recombinantly produced variants of human flh2882 protein; and 4) flh2882 proteins isolated from organisms other than humans (orthologues of human flh2882 protein.)

As used herein, an allelic variant of human flh2882 protein is defined as: 1) a protein isolated from human cells or tissues; 2) a protein encoded by the same genetic locus as that encoding the human flh2882 protein; and 3) a protein that contains substantially homology to human flh2882.

As used herein, two proteins are substantially homologous when the amino acid sequence of the two protein (or a region of the proteins) are at least about 60-65%, typically at least about 70-75%, more typically at least about 80-85%, and most typically at least about 90-95% or more homologous to each other. To determine the percent homology of two amino acid sequences (e.g., SEQ ID NO:13 and an allelic variant thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., SEQ ID NO:13) is occupied by the same amino acid residue or nucleotide as the corresponding position in the other sequence (e.g., an allelic variant of the human flh2882 protein), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

Allelic variants of human flh2882 include both functional and non-functional flh2882 proteins. Functional allelic variants are naturally occurring amino acid sequence variants of the human flh2882 protein that maintain the ability to bind an flh2882 ligand and transduce a signal within a cell. Functional allelic variants will typically contain only conservative substitution of one or more amino acids of SEQ ID NO:13 or substitution, deletion or insertion of non-critical residues in non-critical regions of the protein.

Non-functional allelic variants are naturally occurring amino acid sequence variants of human flh2882 protein that do not have the ability to either bind ligand and/or transduce a signal within a cell. Non-functional allelic variants will typically contain a non-conservative substitution, a deletion, or insertion or premature truncation of the amino acid sequence of SEQ. ID. NO:13 or a substitution, insertion or deletion in critical residues or critical regions.

The present invention further provides non-human orthologues of human flh2882 protein. Orthologues of human flh2882 protein are proteins that are isolated from non-human organisms and possess the same ligand binding and signaling capabilities of the human flh2882 protein. Orthologues of the human flh2882 protein can readily be identified as comprising an amino acid sequence that is substantially homologous to SEQ ID NO:13.

The flh2882 protein is a GPCR that participates in signaling pathways within cells. As used herein, a signaling pathway refers to the modulation (e.g., stimulated or inhibited) of a cellular function/activity upon the binding of a ligand to the GPCR (flh2882 protein). Examples of such functions include mobilization of intracellular molecules that participate in a signal transduction pathway, e.g., phosphatidylinositol 4,5-bisphosphate (PIP2), inositol 1,4,5-triphosphate (IP3) or adenylate cyclase; polarization of the plasma membrane; production or secretion of molecules; alteration in the structure of a cellular component; cell proliferation, e.g., synthesis of DNA; cell migration; cell differentiation; and cell survival. Since the flh2882 protein is expressed substantially in the brain, examples of cells participating in an flh2882 signaling pathway include neural cells, e.g., peripheral nervous system and central nervous system cells such as brain cells, e.g., limbic system cells, hypothalamus cells, hippocampus cells, substantia nigra cells, cortex cells, brain stem cells, neocortex cells, basal ganglion cells, caudate putamen cells, olfactory tubercle cells, and superior colliculi cells.

Depending on the type of cell, the response mediated by the flh2882 protein/ligand binding may be different. For example, in some cells, binding of a ligand to an flh2882 protein may stimulate an activity such as adhesion, migration, differentiation, etc. through phosphatidylinositol or cyclic AMP metabolism and turnover while in other cells, the binding of the ligand to the flh2882 protein will produce a different result. Regardless of the cellular activity modulated by flh2882, it is universal that the flh2882 protein is a GPCR and interacts with a “G protein” to produce one or more secondary signals in a variety of intracellular signal transduction pathways, e.g., through phosphatidylinositol or cyclic AMP metabolism and turnover, in a cell. G proteins represent a family of heterotrimeric proteins composed of α, β and γ subunits, which bind guanine nucleotides. These proteins are usually linked to cell surface receptors, e.g., receptors containing seven transmembrane domains, such as the ligand receptors. Following ligand binding to the receptor, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt. G proteins are described extensively in Lodish H. et al. Molecular Cell Biology, (Scientific American Books Inc., New York, N.Y., 1995), the contents of which are incorporated herein by reference.

As used herein, “phosphatidylinositol turnover and metabolism” refers to the molecules involved in the turnover and metabolism of phosphatidylinositol 4,5-bisphosphate (PIP2) as well as to the activities of these molecules. PIP2 is a phospholipid found in the cytosolic leaflet of the plasma membrane. Binding of a ligand to the flh2882 activates, in some cells, the plasma-membrane enzyme phospholipase C that in turn can hydrolyze PIP2 to produce 1,2-diacylglycerol (DAG) and inositol 1,4,5-triphosphate (IP3). Once formed IP3 can diffuse to the endoplasmic reticulum surface where it can bind an IP3 receptor, e.g., a calcium channel protein containing an IP3 binding site. IP3 binding can induce opening of the channel, allowing calcium ions to be released into the cytoplasm. IP3 can also be phosphorylated by a specific kinase to form inositol 1,3,4,5-tetraphosphate (IP4), a molecule which can cause calcium entry into the cytoplasm from the extracellular medium. IP3 and IP4 can subsequently be hydrolyzed very rapidly to the inactive products inositol 1,4-biphosphate (IP2) and inositol 1,3,4-triphosphate, respectively. These inactive products can be recycled by the cell to synthesize PIP2. The other second messenger produced by the hydrolysis of PIP2, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP2 or one of its metabolites.

Another signaling pathway in which the flh2882 protein may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand induced stimulation of certain G protein coupled receptors. In the ligand signaling pathway, binding of ligand to a ligand receptor can lead to the activation of the enzyme adenylate cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

The present invention further provides fragments of flh2882 proteins. As used herein, a fragment comprises at least 8 contiguous amino acids from an flh2882 protein. Preferred fragments are fragments that possess one or more of the biological activities of the flh2882 protein, for example the ability to bind to a G-protein, as well as fragments that can be used as an immunogen to generate anti-flh2882 antibodies.

Biologically active fragments of the flh2882 protein include peptides comprising amino acid sequences derived from the amino acid sequence of an flh2882 protein, e.g., the amino acid sequence shown in SEQ ID NO:13 or the amino acid sequence of a protein homologous to the flh2882 protein, which include less amino acids than the full length flh2882 protein or the full length protein which is homologous to the flh2882 protein, and exhibit at least one activity of the flh2882 protein. Typically, biologically active fragments (peptides, e.g., peptides which are, for example, 5, 10, 15, 20, 30, 35, 36, 37, 38, 39, 40, 50, 100 or more amino acids in length) comprise a domain or motif, e.g., a transmembrane domain or G-protein binding domain.

The isolated flh2882 proteins can be purified from cells that naturally express the protein, purified from cells that have been altered to express the flh2882 protein, or synthesized using known protein synthesis methods. Preferably, as described below, the isolated flh2882 protein is produced by recombinant DNA techniques. For example, a nucleic acid molecule encoding the protein is cloned into an expression vector (as described above), the expression vector is introduced into a host cell (as described above) and the flh2882 protein is expressed in the host cell. The flh2882 protein can then be isolated from the cells by an appropriate purification scheme using standard protein purification techniques. Alternative to recombinant expression, the flh2882 protein or fragment can be synthesized chemically using standard peptide synthesis techniques. Lastly, native flh2882 protein can be isolated from cells that naturally express the flh2882 protein (e.g., hippocampal cells, or substantia nigra cells).

The present invention further provides flh2882 chimeric or fusion proteins. As used herein, an flh2882 “chimeric protein” or “fusion protein” comprises an flh2882 protein operatively linked to a non-flh2882 protein. An “flh2882 protein” refers to a protein having an amino acid sequence corresponding to an flh2882 protein, whereas a “non-flh2882 protein” refers to a heterologous protein having an amino acid sequence corresponding to a protein which is not substantially homologous to the flh2882 protein, e.g., a protein which is different from the flh2882 protein. Within the context of fusion proteins, the term “operatively linked” is intended to indicate that the flh2882 protein and the non-flh2882 protein are fused in-frame to each other. The non-flh2882 protein can be fused to the N-terminus or C-terminus of the flh2882 protein. For example, in one embodiment the fusion protein is a GST-flh2882 fusion protein in which the flh2882 sequences are fused to the C-terminus of the GST sequences. Other types of fusion proteins include, but are not limited to, enzymatic fusion proteins, for example beta-galactosidase fusions, yeast two-hybrid GAL fusions, poly-His fusions and Ig fusions. Such fusion proteins, particularly poly-His fusions, can facilitate the purification of recombinant flh2882 protein. In another embodiment, the fusion protein is an flh2882 protein containing a heterologous signal sequence at its N-terminus. In certain host cells (e.g., mammalian host cells), expression and/or secretion of an flh2882 protein can be increased by using a heterologous signal sequence.

Preferably, an flh2882 chimeric or fusion protein is produced by standard recombinant DNA techniques. For example, DNA fragments coding for the different protein sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST protein). An flh2882-encoding nucleic acid can be cloned into such an expression vector such that the fusion moiety is linked in-frame to the flh2882 protein.

The present invention also provides altered forms of flh2882 proteins that have been generated using recombinant DNA or mutagenic methods/agents. Altered forms of an flh2882 protein can be generated by mutagenesis, e.g., discrete point mutation or truncation of the flh2882 protein and recombinant DNA method that are well known in the art.

II. Antibodies that Bind to an flh2882 Protein

The present invention further provides antibodies that selectively bind to an flh2882 protein. As used herein, an antibody is said to selectively bind to an flh2882 protein when the antibody binds to flh2882 proteins and does not substantially bind to unrelated proteins. A skilled artisan will readily recognize that an antibody may be considered to substantially bind an flh2882 protein even if it binds to proteins that share homology with a fragment or domain of the flh2882 protein.

The term “antibody” as used herein refers to immunoglobulin molecules and immunologically active fragments of immunoglobulin molecules, i.e., molecules that contain an antigen binding site which specifically binds (immunoreacts with) an antigen, such as flh2882. Examples of immunologically active fragments of immunoglobulin molecules include F(ab) and F(ab′)2 fragments which can be generated by treating the antibody with an enzyme such as pepsin. The invention provides polyclonal and monoclonal antibodies that bind flh2882. The term “monoclonal antibody” or “monoclonal antibody composition”, as used herein, refers to a population of antibody molecules that contain only one species of an antigen binding site capable of immunoreacting with a particular epitope of flh2882. A monoclonal antibody composition thus typically displays a single binding affinity for a particular flh2882 protein with which it immunoreacts.

To generate anti-flh2882 antibodies, an isolated flh2882 protein, or a fragment thereof, is used as an immunogen to generate antibodies that bind flh2882 using standard techniques for polyclonal and monoclonal antibody preparation. The full-length flh2882 protein can be used or, alternatively, an antigenic peptide fragment of flh2882 can be used as an immunogen. An antigenic fragment of the flh2882 protein will typically comprises at least 8 contiguous amino acid residues of an flh2882 protein, e.g. 8 contiguous amino acids from SEQ ID NO:13. Preferably, the antigenic peptide comprises at least 10 amino acid residues, more preferably at least 15 amino acid residues, even more preferably at least 20 amino acid residues, and most preferably at least 30 amino acid residues of an flh2882 protein. Preferred fragments for generating anti-flh2882 antibodies are regions of flh2882 that are located on the surface of the protein, e.g., hydrophilic regions.

An flh2882 immunogen typically is used to prepare antibodies by immunizing a suitable subject, (e.g., rabbit, goat, mouse or other mammal) with the immunogen. An appropriate immunogenic preparation can contain, for example, recombinantly expressed flh2882 protein or a chemically synthesized flh2882 peptide. The preparation can further include an adjuvant, such as Freund's complete or incomplete adjuvant, or similar immunostimulatory agent. Immunization of a suitable subject with an immunogenic flh2882 preparation induces a polyclonal anti-flh2882 antibody response.

Polyclonal anti-flh2882 antibodies can be prepared as described above by immunizing a suitable subject with an flh2882 immunogen. The anti-flh2882 antibody titer in the immunized subject can be monitored over time by standard techniques, such as with an enzyme linked immunosorbent assay (ELISA) using immobilized flh2882. If desired, the antibody molecules directed against flh2882 can be isolated from the mammal (e.g., from the blood) and further purified by well known techniques, such as protein A chromatography to obtain the IgG fraction. At an appropriate time after immunization, e.g., when the anti-flh2882 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an flh2882 immunogen as described above, and the culture supernatants of the resulting hybridoma cells are screened to identify a hybridoma producing a monoclonal antibody that binds flh2882.

Any of the many well known protocols used for fusing lymphocytes and immortalized cell lines can be applied for the purpose of generating an anti-flh2882 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind flh2882, e.g., using a standard ELISA assay.

Alternative to preparing monoclonal antibody-secreting hybridomas, a monoclonal anti-flh2882 antibody can be identified and isolated by screening a recombinant combinatorial immunoglobulin library (e.g., an antibody phage display library) with flh2882 to thereby isolate immunoglobulin library members that bind flh2882. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-flh2882 antibodies, such as chimeric and humanized monoclonal antibodies, comprising both human and non-human fragments, which can be made using standard recombinant DNA techniques, are within the scope of the invention. Such chimeric and humanized monoclonal antibodies can be produced by recombinant DNA techniques known in the art, for example using methods described in Robinson et al. PCT International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-flh2882 antibody (e.g., monoclonal antibody) can be used to isolate flh2882 proteins by standard techniques, such as affinity chromatography or immunoprecipitation. An anti-flh2882 antibody can facilitate the purification of natural flh2882 protein from cells and recombinantly produced flh2882 protein expressed in host cells. Moreover, an anti-flh2882 antibody can be used to detect flh2882 protein (e.g., in a cellular lysate or cell supernatant) in order to evaluate the abundance and pattern of expression of the flh2882 protein. The detection of circulating fragments of an flh2882 protein can be used to identify flh2882 protein turnover in a subject. Anti-flh2882 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

III. Isolated flh2882 Nucleic Acid Molecules

The present invention further provides isolated nucleic acid molecules that encode an flh2882 protein, hereinafter the flh2882 gene or flh2882 nucleic acid molecule, as well as fragments of an flh2882 gene.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

As used herein, an “isolated” nucleic acid molecule is one that is separated from other nucleic acid molecules that are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated flh2882 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., a substantia nigra cell). Moreover, an “isolated” nucleic acid molecule, such as a cDNA molecule, can be substantially free of other cellular material, or culture medium when produced by recombinant techniques, or chemical precursors or other chemicals when chemically synthesized. However, the flh2882 nucleic acid molecule can be fused to other protein encoding or regulatory sequences and still be considered isolated.

The isolated nucleic acid molecules of the present invention encode an flh2882 protein. As described above, an flh2882 protein is defined as a protein comprising the amino acid sequence depicted in SEQ ID NO:13 (human flh2882 protein), allelic variants of human flh2882 protein, and orthologues of the human flh2882 protein. A preferred flh2882 nucleic acid molecule comprises the nucleotide sequence shown in SEQ ID NO:14. The sequence of SEQ ID NO:14 corresponds to the human flh2882 cDNA. This cDNA comprises sequences encoding the human flh2882 protein (i.e., “the coding region”, from nucleotides 184 to 1194 of SEQ ID NO:14), as well as 5′ untranslated sequences (nucleotides 1 to 183 of SEQ ID NO:14) and 3′ untranslated sequences (nucleotides 1195 to 2581 of SEQ ID NO:14). Alternatively, the nucleic acid molecule can comprise only the coding region of SEQ ID NO:14 (e.g., nucleotides 184 to 1194 shown separately as SEQ ID NO:15).

The human flh2882 gene is approximately 2581 nucleotides in length and encodes a full length protein having a molecular weight of approximately 38.7 KDa and which is approximately 337 amino acid residues in length. The human flh2882 protein is expressed primarily in the brain, particularly the substantia nigra. Based on structural analysis, amino acid residues 11-28 (SEQ ID NO:16), 43-62 (SEQ ID NO:17), 80-102 (SEQ ID NO:18), 121-146 (SEQ ID NO:19), 169-190 (SEQ ID NO:20), 247-265 (SEQ ID NO:21), and 280-300 (SEQ ID NO:22) comprise transmembrane domains. As used herein, the term “transmembrane domain” refers to a structural amino acid motif which includes a hydrophobic helix that spans the plasma membrane.

The invention further encompasses nucleic acid molecules that differ from the nucleotide sequence shown in SEQ ID NO:14 (and fragments thereof) due to degeneracy of the genetic code and thus encode the same flh2882 protein as that encoded by the nucleotide sequence shown in SEQ ID NO:14.

In another preferred embodiment, an isolated nucleic acid molecule of the invention comprises a nucleic acid molecule which is a complement of the nucleotide sequence shown in SEQ ID NO:14, or a fragment of either of these nucleotide sequences. A nucleic acid molecule which is complementary to the nucleotide sequence shown in SEQ ID NO:14 is one which is sufficiently complementary to the nucleotide sequence shown in SEQ ID NO:14 such that it can hybridize to the nucleotide sequence shown in SEQ ID NO:14, thereby forming a stable duplex.

Orthologues and allelic variants of the human flh2882 gene can readily be identified using methods well known in the art. Allelic variants and orthologues of the human flh2882 gene will comprise a nucleotide sequence that is at least about 60-65%, typically at least about 70-75%, more typically at least about 80-85%, and most typically at least about 90-95% or more homologous to the nucleotide sequence shown in SEQ ID NO:14, or a fragment of these nucleotide sequences. Such nucleic acid molecules can readily be identified as being able to hybridize, preferably under stringent conditions, to the nucleotide sequence shown in SEQ ID NO:14, or a fragment of either of these nucleotide sequences.

Moreover, the nucleic acid molecule of the invention can comprise only a fragment of the coding region of an flh2882 gene, such as a fragment of SEQ ID NO:14. The nucleotide sequence determined from the cloning of the human flh2882 gene allows for the generation of probes and primers designed for use in identifying and/or cloning flh2882 gene homologues from other cell types, e.g., from other tissues, as well as flh2882 gene orthologues from other mammals. A probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of SEQ ID NO:14 sense, an anti-sense sequence of SEQ ID NO:14, or naturally occurring mutants thereof. Primers based on the nucleotide sequence in SEQ ID NO:14 can be used in PCR reactions to clone flh2882 gene homologues. Probes based on the flh2882 nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an flh2882 protein, such as by measuring a level of an flh2882-encoding nucleic acid in a sample of cells from a subject e.g., detecting flh2882 mRNA levels or determining whether a genomic flh2882 gene has been mutated or deleted.

In addition to the flh2882 nucleotide sequence shown in SEQ ID NO:14, it will be appreciated by those skilled in the art that DNA sequence polymorphisms that lead to changes in the amino acid sequences of an flh2882 protein may exist within a population (e.g., the human population). Such genetic polymorphism in the flh2882 gene may exist among individuals within a population due to natural allelic variation. As used herein, the terms “gene” and “recombinant gene” refer to nucleic acid molecules comprising an open reading frame encoding an flh2882 protein, preferably a mammalian flh2882 protein. Such natural allelic variations can typically result in 1-5% variance in the nucleotide sequence of the flh2882 gene. Any and all such nucleotide variations and resulting amino acid polymorphisms in an flh2882 gene that are the result of natural allelic variation are intended to be within the scope of the invention. Such allelic variation includes both active allelic variants as well as non-active or reduced activity allelic variants, the later two types typically giving rise to a pathological disorder. Moreover, nucleic acid molecules encoding flh2882 proteins from other species, and thus which have a nucleotide sequence which differs from the human sequence of SEQ ID NO:14, are intended to be within the scope of the invention. Nucleic acid molecules corresponding to natural allelic variants and non-human orthologues of the human flh2882 cDNA of the invention can be isolated based on their homology to the human flh2882 nucleic acid disclosed herein using the human cDNA, or a fragment thereof, as a hybridization probe according to standard hybridization techniques under stringent hybridization conditions.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:14. In other embodiments, the nucleic acid is at least 30, 50, 100, 250 or 500 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:14 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural human flh2882.

In addition to naturally-occurring allelic variants of the flh2882 nucleic acid sequence that may exist in the population, the skilled artisan will further appreciate that changes can be introduced by mutation into the nucleotide sequence of SEQ ID NO:14, thereby leading to changes in the amino acid sequence of the encoded flh2882 protein, without altering the functional ability of the flh2882 protein. For example, nucleotide substitutions leading to amino acid substitutions at “non-essential” amino acid residues can be made in the sequence of SEQ ID NO:14. A “non-essential” amino acid residue is a residue that can be altered from the wild-type sequence of an flh2882 protein (e.g., the sequence of SEQ ID NO:13) without altering the activity of flh2882, whereas an “essential” amino acid residue is required for flh2882 protein activity. For example, conserved amino acid residues, e.g., aspartates, prolines, threonines, and tyrosines, in the transmembrane domains of the flh2882 protein are most likely important for binding to ligand and are thus essential residues of the flh2882 protein. Other amino acid residues, however, (e.g., those that are not conserved or only semi-conserved in the transmembrane domain) may not be essential for activity and thus are likely to be amenable to alteration without altering flh2882 protein activity.

Accordingly, another aspect of the invention pertains to nucleic acid molecules encoding flh2882 proteins that contain changes in amino acid residues that are not essential for flh2882 activity. Such flh2882 proteins differ in amino acid sequence from SEQ ID NO:13 yet retain at least one of the flh2882 activities described herein. In one embodiment, the isolated nucleic acid molecule comprises a nucleotide sequence encoding a protein, wherein the protein comprises an amino acid sequence at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the amino acid sequence of SEQ ID NO:13.

An isolated nucleic acid molecule encoding an flh2882 protein homologous to the protein of SEQ ID NO:13 can be created by introducing one or more nucleotide substitutions, additions or deletions into the nucleotide sequence of SEQ ID NO:14, such that one or more amino acid substitutions, additions or deletions are introduced into the encoded protein. Mutations can be introduced into SEQ ID NO:14 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in flh2882 is preferably replaced with another amino acid residue from the same side chain family. Alternatively, in another embodiment, mutations can be introduced randomly along all or part of an flh2882 coding sequence, such as by saturation mutagenesis, and the resultant mutants can be screened for an flh2882 activity described herein to identify mutants that retain flh2882 activity. Following mutagenesis of SEQ ID NO:14, the encoded protein can be expressed recombinantly (e.g., as described in Examples 3 and 4) and the activity of the protein can be determined using, for example, assays described herein.

In addition to the nucleic acid molecules encoding flh2882 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire flh2882 coding strand, or to only a fragment thereof. In one embodiment, an antisense nucleic acid molecule is antisense to a “coding region” of the coding strand of a nucleotide sequence encoding an flh2882 protein.

The term “coding region” refers to the region of the nucleotide sequence comprising codons which are translated into amino acid residues, e.g., the entire coding region of SEQ ID NO:14 comprises nucleotides 184 to 1194 (shown separately as SEQ ID NO:15). In another embodiment, the antisense nucleic acid molecule is antisense to a “noncoding region” of the coding strand of a nucleotide sequence encoding an flh2882 protein. The term “noncoding region” refers to 5′ and 3′ sequences which flank the coding region that are not translated into amino acids (i.e., also referred to as 5′ and 3′ untranslated regions).

Given the coding strand sequence encoding the flh2882 protein disclosed herein (e.g., SEQ ID NO:14), antisense nucleic acids of the invention can be designed according to the rules of Watson and Crick base pairing. The antisense nucleic acid molecule can be complementary to the entire coding region of flh2882 mRNA, but more preferably is an oligonucleotide which is antisense to only a fragment of the coding or noncoding region of flh2882 mRNA. For example, the antisense oligonucleotide can be complementary to the region surrounding the translation start site of flh2882 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an flh2882 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave flh2882 mRNA transcripts to thereby inhibit translation of flh2882 mRNA. A ribozyme having specificity for an flh2882-encoding nucleic acid can be designed based upon the nucleotide sequence of an flh2882 cDNA disclosed herein (i.e., SEQ ID NO:14). For example, a derivative of a Tetrahymena L-19 IVS RNA can be constructed in which the nucleotide sequence of the active site is complementary to the nucleotide sequence to be cleaved in an flh2882-encoding mRNA. See, e.g., Cech et al. U.S. Pat. No. 4,987,071 and Cech et al. U.S. Pat. No. 5,116,742. Alternatively, flh2882 mRNA can be used to select a catalytic RNA having a specific ribonuclease activity from a pool of RNA molecules. See, e.g., Bartel, D. and Szostak, J. W. (1993) Science 261:1411-1418.

Alternatively, flh2882 gene expression can be inhibited by targeting nucleotide sequences complementary to the regulatory region of the flh2882 gene (e.g., the flh2882 gene promoter and/or enhancers) to form triple helical structures that prevent transcription of the flh2882 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

IV. Recombinant Expression Vectors and Host Cells

Another aspect of the invention pertains to vectors, preferably expression vectors, containing a nucleic acid encoding an flh2882 protein (or a fragment thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., flh2882 proteins, altered forms of flh2882 proteins, fusion proteins, and the like).

The recombinant expression vectors of the invention can be designed for expression of an flh2882 protein in prokaryotic or eukaryotic cells. For example, an flh2882 protein can be expressed in bacterial cells such as E. coli, insect cells (e.g., using baculovirus expression vectors) yeast cells or mammalian cells. Suitable host cells are discussed further in Goeddel, Gene Expression Technology Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the flh2882 gene is cloned into a pGEX expression vector to create a vector encoding a fusion protein comprising, from the N-terminus to the C-terminus, GST-thrombin cleavage site-flh2882 protein. The fusion protein can be purified by affinity chromatography using glutathione-agarose resin. Recombinant flh2882 protein unfused to GST can be recovered by cleavage of the fusion protein with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the flh2882 gene expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, an flh2882 gene can be expressed in insect cells using, for example, baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the O-fetoprotein promoter (Campes and Tilghman (1989) Genes Dev. 3:537-546).

The invention further provides a recombinant expression vector comprising a DNA molecule encoding an flh2882 protein cloned into the expression vector in an antisense orientation. That is, the DNA molecule is operatively linked to a regulatory sequence in a manner which allows for expression (by transcription of the DNA molecule) of an RNA molecule which is antisense to flh2882 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, flh2882 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the flh2882 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) flh2882 protein. Accordingly, the invention further provides methods for producing flh2882 protein using the host cells of the invention. In one embodiment, the method comprises culturing the host cell of invention (into which a recombinant expression vector encoding an flh2882 protein has been introduced) in a suitable medium until the flh2882 protein is produced. In another embodiment, the method further comprises isolating the flh2882 protein from the medium or the host cell.

The host cells of the invention can also be used to produce non-human transgenic animals. The non-human transgenic animals can be used in screening assays designed to identify agents or compounds, e.g., drugs, pharmaceuticals, etc., which are capable of ameliorating detrimental symptoms of selected disorders such as nervous system disorders, e.g., psychiatric disorders or disorders affecting circadian rhythms and the sleep-wake cycle. For example, in one embodiment, a host cell of the invention is a fertilized oocyte or an embryonic stem cell into which flh2882 protein-coding sequences have been introduced. Such host cells can then be used to create non-human transgenic animals in which exogenous flh2882 gene sequences have been introduced into their genome or homologous recombinant animals in which endogenous flh2882 gene sequences have been altered. Such animals are useful for studying the function and/or activity of an flh2882 protein and for identifying and/or evaluating modulators of flh2882 protein activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous flh2882 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing flh2882 protein encoding nucleic acid into the male pronuclei of a fertilized oocyte, e.g., by microinjection, retroviral infection, and allowing the oocyte to develop in a pseudopregnant female foster animal. The human flh2882 cDNA sequence of SEQ ID NO:14 can be introduced as a transgene into the genome of a non-human animal. Moreover, a non-human homologue of the human flh2882 gene, such as a mouse flh2882 gene, can be isolated based on hybridization to the human flh2882 cDNA (described further above) and used as a transgene. Intronic sequences and polyadenylation signals can also be included in the transgene to increase the efficiency of expression of the transgene. A tissue-specific regulatory sequence(s) can be operably linked to the flh2882 transgene to direct expression of an flh2882 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the flh2882 transgene in its genome and/or expression of flh2882 mRNA in tissues or cells of the animals. A transgenic founder animal can then be used to breed additional animals carrying the transgene. Moreover, transgenic animals carrying a transgene encoding an flh2882 protein can further be bred to other transgenic animals carrying other transgenes.

To create a homologous recombinant animal, a vector is prepared which contains at least a fragment of an flh2882 gene into which a deletion, addition or substitution has been introduced to thereby alter, e.g., functionally disrupt, the flh2882 gene. The flh2882 gene can be a human gene (e.g., from a human genomic clone isolated from a human genomic library screened with the cDNA of SEQ ID NO:14), but more preferably is a non-human homologue of a human flh2882 gene. For example, a mouse flh2882 gene can be isolated from a mouse genomic DNA library using the flh2882 cDNA of SEQ ID NO:14 as a probe. The mouse flh2882 gene then can be used to construct a homologous recombination vector suitable for altering an endogenous flh2882 gene in the mouse genome. In a preferred embodiment, the vector is designed such that, upon homologous recombination, the endogenous flh2882 gene is functionally disrupted (i.e., no longer encodes a functional protein; also referred to as a “knock out” vector). Alternatively, the vector can be designed such that, upon homologous recombination, the endogenous flh2882 gene is mutated or otherwise altered but still encodes functional protein (e.g., the upstream regulatory region can be altered to thereby alter the expression of the endogenous flh2882 protein). In the homologous recombination vector, the altered fragment of the flh2882 gene is flanked at its 5′ and 3′ ends by additional nucleic acid of the flh2882 gene to allow for homologous recombination to occur between the exogenous flh2882 gene carried by the vector and an endogenous flh2882 gene in an embryonic stem cell. The additional flanking flh2882 nucleic acid is of sufficient length for successful homologous recombination with the endogenous gene. Typically, several kilobases of flanking DNA (both at the 5′ and 3′ ends) are included in the vector (see for example, Thomas, K. R. and Capecchi, M. R. (1987) Cell 51:503 for a description of homologous recombination vectors). The vector is introduced into an embryonic stem cell line (e.g., by electroporation) and cells in which the introduced flh2882 gene has homologously recombined with the endogenous flh2882 gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; WO 92/0968; and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

V. Uses and Methods of the Invention

The nucleic acid molecules, proteins, protein homologues, modulators, and antibodies described herein can be used in one or more of the following methods: a) drug screening assays; b) diagnostic assays particularly in disease identification, allelic screening and pharmocogenetic testing; c) methods of treatment; d) pharmacogenomics; and e) monitoring of effects during clinical trials. An flh2882 protein of the invention can be used as a drug target for developing agents to modulate the activity of the flh2882 protein (a GPCR). The isolated nucleic acid molecules of the invention can be used to express flh2882 protein (e.g., via a recombinant expression vector in a host cell or in gene therapy applications), to detect flh2882 mRNA (e.g., in a biological sample) or a naturally occurring or recombinantly generated genetic mutation in an flh2882 gene, and to modulate flh2882 protein activity, as described further below. In addition, the flh2882 proteins can be used to screen drugs or compounds which modulate flh2882 protein activity. Moreover, the anti-flh2882 antibodies of the invention can be used to detect and isolate an flh2882 protein, particularly fragments of an flh2882 protein present in a biological sample, and to modulate flh2882 protein activity.

a. Drug Screening Assays:

The invention provides methods for identifying compounds or agents that can be used to treat disorders characterized by (or associated with) aberrant or abnormal flh2882 nucleic acid expression and/or flh2882 protein activity. These methods are also referred to herein as drug screening assays and typically include the step of screening a candidate/test compound or agent to identify compounds that are an agonist or antagonist of an flh2882 protein, and specifically for the ability to interact with (e.g., bind to) an flh2882 protein, to modulate the interaction of an flh2882 protein and a target molecule, and/or to modulate flh2882 nucleic acid expression and/or flh2882 protein activity. Candidate/test compounds or agents which have one or more of these abilities can be used as drugs to treat disorders characterized by aberrant or abnormal flh2882 nucleic acid expression and/or flh2882 protein activity. Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); and 4) small organic and inorganic molecules (e.g., molecules obtained from combinatorial and natural product libraries).

In one embodiment, the invention provides assays for screening candidate/test compounds which interact with (e.g., bind to) an flh2882 protein. Typically, the assays are recombinant cell based or cell-free assays which include the steps of combining a cell expressing an flh2882 protein or a bioactive fragment thereof, or an isolated flh2882 protein, and a candidate/test compound, e.g., under conditions which allow for interaction of (e.g., binding of) the candidate/test compound to the flh2882 protein or fragment thereof to form a complex, and detecting the formation of a complex, in which the ability of the candidate compound to interact with (e.g., bind to) the flh2882 protein or fragment thereof is indicated by the presence of the candidate compound in the complex. Formation of complexes between the flh2882 protein and the candidate compound can be detected using competition binding assays, and can be quantitated, for example, using standard immunoassays.

In another embodiment, the invention provides screening assays to identify candidate/test compounds which modulate (e.g., stimulate or inhibit) the interaction (and most likely flh2882 protein activity as well) between an flh2882 protein and a molecule (target molecule) with which the flh2882 protein normally interacts. Examples of such target molecules include proteins in the same signaling path as the flh2882 protein, e.g., proteins which may function upstream (including both stimulators and inhibitors of activity) or downstream of the flh2882 protein in, for example, a cognitive function signaling pathway or in a pathway involving flh2882 protein activity, e.g., a G protein or other interactor involved in cAMP or phosphatidylinositol turnover, and/or adenylate cyclase or phospholipase C activation. Typically, the assays are recombinant cell based assays which include the steps of combining a cell expressing an flh2882 protein, or a bioactive fragment thereof, an flh2882 protein target molecule (e.g., an flh2882 ligand) and a candidate/test compound, e.g., under conditions wherein but for the presence of the candidate compound, the flh2882 protein or biologically active fragment thereof interacts with (e.g., binds to) the target molecule, and detecting the formation of a complex which includes the flh2882 protein and the target molecule or detecting the interaction/reaction of the flh2882 protein and the target molecule. Detection of complex formation can include direct quantitation of the complex by, for example, measuring inductive effects of the flh2882 protein. A statistically significant change, such as a decrease, in the interaction of the flh2882 protein and target molecule (e.g., in the formation of a complex between the flh2882 protein and the target molecule) in the presence of a candidate compound (relative to what is detected in the absence of the candidate compound) is indicative of a modulation (e.g., stimulation or inhibition) of the interaction between the flh2882 protein and the target molecule. Modulation of the formation of complexes between the flh2882 protein and the target molecule can be quantitated using, for example, an immunoassay.

To perform cell free drug screening assays, it is desirable to immobilize either the flh2882 protein or its target molecule to facilitate separation of complexes from uncomplexed forms of one or both of the proteins, as well as to accommodate automation of the assay. Interaction (e.g., binding of) of the flh2882 protein to a target molecule, in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows the protein to be bound to a matrix. For example, glutathione-S-transferase/flh2882 fusion proteins can be adsorbed onto glutathione sepharose beads (Sigma Chemical, St. Louis, Mo.) or glutathione derivatized microtitre plates, which are then combined with the cell lysates (e.g., 35S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of flh2882-binding protein found in the bead fraction quantitated from the gel using standard electrophoretic techniques.

Other techniques for immobilizing proteins on matrices can also be used in the drug screening assays of the invention. For example, either the flh2882 protein or its target molecule can be immobilized utilizing conjugation of biotin and streptavidin. Biotinylated flh2882 protein molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with an flh2882 protein but which do not interfere with binding of the protein to its target molecule can be derivatized to the wells of the plate, and flh2882 protein trapped in the wells by antibody conjugation. As described above, preparations of an flh2882-binding protein and a candidate compound are incubated in the flh2882 protein-presenting wells of the plate, and the amount of complex trapped in the well can be quantitated. Methods for detecting such complexes, in addition to those described above for the GST-immobilized complexes, include immunodetection of complexes using antibodies reactive with the flh2882 protein target molecule, or which are reactive with flh2882 protein and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant or abnormal flh2882 nucleic acid expression or flh2882 protein activity. This method typically includes the step of assaying the ability of the compound or agent to modulate the expression of the flh2882 nucleic acid or the activity of the flh2882 protein thereby identifying a compound for treating a disorder characterized by aberrant or abnormal flh2882 nucleic acid expression or flh2882 protein activity. Methods for assaying the ability of the compound or agent to modulate the expression of the flh2882 nucleic acid or activity of the flh2882 protein are typically cell-based assays. For example, cells which are sensitive to ligands which transduce signals via a pathway involving an flh2882 protein can be induced to overexpress an flh2882 protein in the presence and absence of a candidate compound. Candidate compounds which produce a statistically significant change in flh2882 protein-dependent responses (either stimulation or inhibition) can be identified. In one embodiment, expression of the flh2882 nucleic acid or activity of an flh2882 protein is modulated in cells and the effects of candidate compounds on the readout of interest (such as cAMP or phosphatidylinositol turnover) are measured. For example, the expression of genes which are up- or down-regulated in response to an flh2882 protein-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of an flh2882 protein or flh2882 protein target molecules can also be measured, for example, by immunoblotting.

Alternatively, modulators of flh2882 gene expression (e.g., compounds which can be used to treat a disorder characterized by aberrant or abnormal flh2882 nucleic acid expression or flh2882 protein activity) can be identified in a method wherein a cell is contacted with a candidate compound and the expression of flh2882 mRNA or protein in the cell is determined. The level of expression of flh2882 mRNA or protein in the presence of the candidate compound is compared to the level of expression of flh2882 mRNA or protein in the absence of the candidate compound. The candidate compound can then be identified as a modulator of flh2882 nucleic acid expression based on this comparison and be used to treat a disorder characterized by aberrant flh2882 nucleic acid expression. For example, when expression of flh2882 mRNA or protein is greater (statistically significantly greater) in the presence of the candidate compound than in its absence, the candidate compound is identified as a stimulator of flh2882 nucleic acid expression. Alternatively, when flh2882 nucleic acid expression is less (statistically significantly less) in the presence of the candidate compound than in its absence, the candidate compound is identified as an inhibitor of flh2882 nucleic acid expression. The level of flh2882 nucleic acid expression in the cells can be determined by methods described herein for detecting flh2882 mRNA or protein.

In yet another aspect of the invention, the flh2882 proteins, or fragments thereof, can be used as “bait proteins” in a two-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with the flh2882 protein (“flh2882-binding proteins” or “flh2882-bp”) and modulate flh2882 protein activity. Such flh2882-binding proteins are also likely to be involved in the propagation of signals by the flh2882 proteins as, for example, upstream or downstream elements of the flh2882 protein pathway.

The two-hybrid system is based on the modular nature of most transcription factors, which consist of separable DNA-binding and activation domains. Bartel, P. et al. “Using the Two-Hybrid System to Detect Protein-Protein Interactions” in Cellular Interactions in Development: A Practical Approach, Hartley, D. A. ed. (Oxford University Press, Oxford, 1993) pp. 153-179. Briefly, the assay utilizes two different DNA constructs. In one construct, the gene that encode an flh2882 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an flh2882-protein dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the flh2882 protein.

Modulators of flh2882 protein activity and/or flh2882 nucleic acid expression identified according to these drug screening assays can be used to treat, for example, nervous system disorders. These methods of treatment include the steps of administering the modulators of flh2882 protein activity and/or nucleic acid expression, e.g., in a pharmaceutical composition as described in subsection IV above, to a subject in need of such treatment, e.g., a subject with a disorder described herein.

b. Diagnostic Assays:

The invention further provides a method for detecting the presence of an flh2882 protein or flh2882 nucleic acid molecule, or fragment thereof, in a biological sample. The method involves contacting the biological sample with a compound or an agent capable of detecting flh2882 protein or mRNA such that the presence of flh2882 protein/encoding nucleic acid molecule is detected in the biological sample. A preferred agent for detecting flh2882 mRNA is a labeled or labelable nucleic acid probe capable of hybridizing to flh2882 mRNA. The nucleic acid probe can be, for example, the full-length flh2882 cDNA of SEQ ID NO:14, or a fragment thereof, such as an oligonucleotide of at least 15, 30, 50, 100, 250 or 500 nucleotides in length and sufficient to specifically hybridize under stringent conditions to flh2882 mRNA. A preferred agent for detecting flh2882 protein is a labeled or labelable antibody capable of binding to flh2882 protein. Antibodies can be polyclonal, or more preferably, monoclonal. An intact antibody, or a fragment thereof (e.g., Fab or F(ab′)2) can be used. The term “labeled or labelable”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect flh2882 mRNA or protein in a biological sample in vitro as well as in vivo. For example, in vitro techniques for detection of flh2882 mRNA include Northern hybridizations and in situ hybridizations. In vitro techniques for detection of flh2882 protein include enzyme linked immunosorbent assays (ELISAs), Western blots, immunoprecipitations and immunofluorescence. Alternatively, flh2882 protein can be detected in vivo in a subject by introducing into the subject a labeled anti-flh2882 antibody. For example, the antibody can be labeled with a radioactive marker whose presence and location in a subject can be detected by standard imaging techniques. Particularly useful are methods which detect the allelic variant of an flh2882 protein expressed in a subject and methods which detect fragments of an flh2882 protein in a sample.

The invention also encompasses kits for detecting the presence of an flh2882 protein in a biological sample. For example, the kit can comprise reagents such as a labeled or labelable compound or agent capable of detecting flh2882 protein or mRNA in a biological sample; means for determining the amount of flh2882 protein in the sample; and means for comparing the amount of flh2882 protein in the sample with a standard. The compound or agent can be packaged in a suitable container. The kit can further comprise instructions for using the kit to detect flh2882 mRNA or protein.

The methods of the invention can also be used to detect naturally occurring genetic mutations in an flh2882 gene, thereby determining if a subject with the mutated gene is at risk for a disorder characterized by aberrant or abnormal flh2882 nucleic acid expression or flh2882 protein activity as described herein. In preferred embodiments, the methods include detecting, in a sample of cells from the subject, the presence or absence of a genetic mutation characterized by at least one of an alteration affecting the integrity of a gene encoding an flh2882 protein, or the misexpression of the flh2882 gene. For example, such genetic mutations can be detected by ascertaining the existence of at least one of 1) a deletion of one or more nucleotides from an flh2882 gene; 2) an addition of one or more nucleotides to an flh2882 gene; 3) a substitution of one or more nucleotides of an flh2882 gene, 4) a chromosomal rearrangement of an flh2882 gene; 5) an alteration in the level of a messenger RNA transcript of an flh2882 gene, 6) aberrant modification of an flh2882 gene, such as of the methylation pattern of the genomic DNA, 7) the presence of a non-wild type splicing pattern of a messenger RNA transcript of an flh2882 gene, 8) a non-wild type level of an flh2882-protein, 9) allelic loss of an flh2882 gene, and 10) inappropriate post-translational modification of an flh2882-protein. As described herein, there are a large number of assay techniques known in the art that can be used for detecting mutations in an flh2882 gene.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the flh2882-gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to an flh2882 gene under conditions such that hybridization and amplification of the flh2882-gene (if present) occurs, and detecting the presence or absence of an amplification product, or detecting the size of the amplification product and comparing the length to a control sample.

In an alternative embodiment, mutations in an flh2882 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In yet another embodiment, any of a variety of sequencing reactions known in the art can be used to directly sequence the flh2882 gene and detect mutations by comparing the sequence of the sample flh2882 gene with the corresponding wild-type (control) sequence. Examples of sequencing reactions include those based on techniques developed by Maxim and Gilbert ((1977) PNAS 74:560) or Sanger ((1977) PNAS 74:5463). A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the flh2882 gene include methods in which protection from cleavage agents is used to detect mismatched bases in RNA/RNA or RNA/DNA duplexes (Myers et al. (1985) Science 230:1242); Cotton et al. (1988) PNAS 85:4397; Saleeba et al. (1992) Meth. Enzymol. 217:286-295), electrophoretic mobility of mutant and wild type nucleic acid is compared (Orita et al. (1989) PNAS 86:2766; Cotton (1993) Mutat. Res. 285:125-144; and Hayashi (1992) Genet. Anal. Tech. Appl. 9:73-79), and movement of mutant or wild-type fragments in polyacrylamide gels containing a gradient of denaturant is assayed using denaturing gradient gel electrophoresis (Myers et al (1985) Nature 313:495). Examples of other techniques for detecting point mutations include, selective oligonucleotide hybridization, selective amplification, and selective primer extension.

c. Methods of Treatment

Another aspect of the invention pertains to methods for treating a subject, e.g., a human, having a disease or disorder characterized by (or associated with) aberrant or abnormal flh2882 nucleic acid expression and/or flh2882 protein activity. These methods include the step of administering an flh2882 protein/gene modulator (agonist or antagonist) to the subject such that treatment occurs. The language “aberrant or abnormal flh2882 protein expression” refers to expression of a non-wild-type flh2882 protein or a non-wild-type level of expression of an flh2882 protein. Aberrant or abnormal flh2882 protein activity refers to a non-wild-type flh2882 protein activity or a non-wild-type level of flh2882 protein activity. As the flh2882 protein is involved in a pathway involving signaling within cells, aberrant or abnormal flh2882 protein activity or expression interferes with the normal regulation of functions mediated by flh2882 protein signaling, and in particular brain cells.

The terms “treating” or “treatment”, as used herein, refer to reduction or alleviation of at least one adverse effect or symptom of a disorder or disease, e.g., a disorder or disease characterized by or associated with abnormal or aberrant flh2882 protein activity or flh2882 nucleic acid expression.

As used herein, an flh2882 protein/gene modulator is a molecule which can modulate flh2882 nucleic acid expression and/or flh2882 protein activity. For example, an flh2882 gene or protein modulator can modulate, e.g., upregulate (activate/agonize) or down-regulate (suppress/antagonize), flh2882 nucleic acid expression. In another example, an flh2882 protein/gene modulator can modulate (e.g., stimulate/agonize or inhibit/antagonize) flh2882 protein activity. If it is desirable to treat a disorder or disease characterized by (or associated with) aberrant or abnormal (non-wild-type) flh2882 nucleic acid expression and/or flh2882 protein activity by inhibiting flh2882 nucleic acid expression, an flh2882 modulator can be an antisense molecule, e.g., a ribozyme, as described herein. Examples of antisense molecules which can be used to inhibit flh2882 nucleic acid expression include antisense molecules which are complementary to a fragment of the 5′ untranslated region of SEQ ID NO:14 which also includes the start codon and antisense molecules which are complementary to a fragment of the 3′ untranslated region of SEQ ID NO:14. An example of an antisense molecule which is complementary to a fragment of the 5′ untranslated region of SEQ ID NO:14 and which also includes the start codon is a nucleic acid molecule which includes nucleotides which are complementary to nucleotides 171 to 186 of SEQ ID NO:14. This antisense molecule has the following nucleotide sequence: 5′CGGGGCGCGCACCATG 3′ (SEQ ID NO:23). An additional example of an antisense molecule which is complementary to a fragment of the 5′ untranslated region of SEQ ID NO:14 and which also includes the start codon is a nucleic acid molecule which includes nucleotides which are complementary to nucleotides 180 to 196 of SEQ ID NO:14. This antisense molecule has the following nucleotide sequence: 5′CACCATGAACTCGTGGG 3′ (SEQ ID NO:24). An example of an antisense molecule which is complementary to a fragment of the 3′ untranslated region of SEQ ID NO:14 is a nucleic acid molecule which includes nucleotides which are complementary to nucleotides 1195 to 1210 of SEQ ID NO:14. This antisense molecule has the following sequence: 5′ TGAAGGACCGCGCTCC 3′ (SEQ ID NO:25). An additional example of an antisense molecule which is complementary to a fragment of the 3′ untranslated region of SEQ ID NO:14 is a nucleic acid molecule which includes nucleotides which are complementary to nucleotides 1189 to 1204 of SEQ ID NO:14. This antisense molecule has the following sequence: 5′ TCTGAGTGAAGGACCG 3′ (SEQ ID NO:26).

An flh2882 modulator that inhibits flh2882 nucleic acid expression can also be a small molecule or other drug, e.g., a small molecule or drug identified using the screening assays described herein, which inhibits flh2882 nucleic acid expression. If it is desirable to treat a disease or disorder characterized by (or associated with) aberrant or abnormal (non-wild-type) flh2882 nucleic acid expression and/or flh2882 protein activity by stimulating flh2882 nucleic acid expression, an flh2882 modulator can be, for example, a nucleic acid molecule encoding an flh2882 protein (e.g., a nucleic acid molecule comprising a nucleotide sequence homologous to the nucleotide sequence of SEQ ID NO:14) or a small molecule or other drug, e.g., a small molecule (peptide) or drug identified using the screening assays described herein, which stimulates flh2882 nucleic acid expression.

Alternatively, if it is desirable to treat a disease or disorder characterized by (or associated with) aberrant or abnormal (non-wild-type) flh2882 nucleic acid expression and/or flh2882 protein activity by inhibiting flh2882 protein activity, an flh2882 modulator can be an anti-flh2882 antibody or a small molecule or other drug, e.g., a small molecule or drug identified using the screening assays described herein, which inhibits flh2882 protein activity. If it is desirable to treat a disease or disorder characterized by (or associated with) aberrant or abnormal (non-wild-type) flh2882 nucleic acid expression and/or flh2882 protein activity by stimulating flh2882 protein activity, an flh2882 modulator can be an active flh2882 protein or fragment thereof (e.g., an flh2882 protein or fragment thereof having an amino acid sequence which is homologous to the amino acid sequence of SEQ ID NO:13 or a fragment thereof) or a small molecule or other drug, e.g., a small molecule or drug identified using the screening assays described herein, which stimulates flh2882 protein activity.

Other aspects of the invention pertain to methods for modulating an flh2882 protein mediated cell activity. These methods include contacting the cell with an agent (or a composition which includes an effective amount of an agent) which modulates flh2882 protein activity or flh2882 nucleic acid expression such that an flh2882 protein mediated cell activity is altered relative to normal levels (for example, cAMP or phosphatidylinositol metabolism). As used herein, “an flh2882 protein mediated cell activity” refers to a normal or abnormal activity or function of a cell. Examples of flh2882 protein mediated cell activities include phosphatidylinositol turnover, production or secretion of molecules, such as proteins, contraction, proliferation, migration, differentiation, and cell survival. In a preferred embodiment, the cell is neural cell of the brain, e.g., a hippocampal cell. The term “altered” as used herein refers to a change, e.g., an increase or decrease, of a cell associated activity particularly cAMP or phosphatidylinositol turnover, and adenylate cyclase or phospholipase C activation. In one embodiment, the agent stimulates flh2882 protein activity or flh2882 nucleic acid expression. In another embodiment, the agent inhibits flh2882 protein activity or flh2882 nucleic acid expression. These modulatory methods can be performed in vitro (e.g., by culturing the cell with the agent) or, alternatively, in vivo (e.g., by administering the agent to a subject). In a preferred embodiment, the modulatory methods are performed in vivo, i.e., the cell is present within a subject, e.g., a mammal, e.g., a human, and the subject has a disorder or disease characterized by or associated with abnormal or aberrant flh2882 protein activity or flh2882 nucleic acid expression.

A nucleic acid molecule, a protein, an flh2882 modulator, a compound etc. used in the methods of treatment can be incorporated into an appropriate pharmaceutical composition described below and administered to the subject through a route which allows the molecule, protein, modulator, or compound etc. to perform its intended function.

d. Pharmacogenomics

Test/candidate compounds, or modulators which have a stimulatory or inhibitory effect on flh2882 protein activity (e.g., flh2882 gene expression) as identified by a screening assay described herein can be administered to individuals to treat (prophylactically or therapeutically) disorders (e.g., CNS disorders) associated with aberrant flh2882 protein activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permit the selection of effective compounds (e.g., drugs) for prophylactic or therapeutic treatments based on a consideration of the individual's genotype. Such pharmacogenomics can further be used to determine appropriate dosages and therapeutic regimens. Accordingly, the activity of flh2882 protein, expression of flh2882 nucleic acid, or mutation content of flh2882 genes in an individual can be determined to thereby select appropriate compound(s) for therapeutic or prophylactic treatment of the individual.

Pharmacogenomics deal with clinically significant hereditary variations in the response to drugs due to altered drug disposition and abnormal action in affected persons. See, e.g., Eichelbaum, M. (1996) Clin. Exp. Pharmacol. Physiol. 23(10-11):983-985 and Linder, M. W. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of flh2882 protein, expression of flh2882 nucleic acid, or mutation content of flh2882 genes in an individual can be determined to thereby select appropriate agent(s) for therapeutic or prophylactic treatment of a subject. In addition, pharmacogenetic studies can be used to apply genotyping of polymorphic alleles encoding drug-metabolizing enzymes to the identification of a subject's drug responsiveness phenotype. This knowledge, when applied to dosing or drug selection, can avoid adverse reactions or therapeutic failure and thus enhance therapeutic or prophylactic efficiency when treating a subject with an flh2882 modulator, such as a modulator identified by one of the exemplary screening assays described herein.

e. Monitoring of Effects During Clinical Trials

Monitoring the influence of compounds (e.g., drugs) on the expression or activity of flh2882 protein/gene can be applied not only in basic drug screening, but also in clinical trials. For example, the effectiveness of an agent determined by a screening assay, as described herein, to increase flh2882 gene expression, protein levels, or up-regulate flh2882 activity, can be monitored in clinical trials of subjects exhibiting decreased flh2882 gene expression, protein levels, or down-regulated flh2882 protein activity. Alternatively, the effectiveness of an agent, determined by a screening assay, to decrease flh2882 gene expression, protein levels, or down-regulate flh2882 protein activity, can be monitored in clinical trials of subjects exhibiting increased flh2882 gene expression, protein levels, or up-regulated flh2882 protein activity. In such clinical trials, the expression or activity of an flh2882 protein and, preferably, other genes which have been implicated in, for example, a nervous system related disorder can be used as a “read out” or markers of the ligand responsiveness of a particular cell.

For example, and not by way of limitation, genes, including an flh2882 gene, which are modulated in cells by treatment with a compound (e.g., drug or small molecule) which modulates flh2882 protein/gene activity (e.g., identified in a screening assay as described herein) can be identified. Thus, to study the effect of compounds on CNS disorders, for example, in a clinical trial, cells can be isolated and RNA prepared and analyzed for the levels of expression of an flh2882 gene and other genes implicated in the disorder. The levels of gene expression (i.e., a gene expression pattern) can be quantified by Northern blot analysis or RT-PCR, as described herein, or alternatively by measuring the amount of protein produced, by one of the methods described herein, or by measuring the levels of activity of an flh2882 protein or other genes. In this way, the gene expression pattern can serve as an marker, indicative of the physiological response of the cells to the compound. Accordingly, this response state may be determined before, and at various points during, treatment of the individual with the compound.

In a preferred embodiment, the present invention provides a method for monitoring the effectiveness of treatment of a subject with a compound (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the compound; (ii) detecting the level of expression of an flh2882 protein, mRNA, or genomic DNA in the preadministration sample; (iii) obtaining one or more post-administration samples from the subject; (iv) detecting the level of expression or activity of the flh2882 protein, mRNA, or genomic DNA in the post-administration samples; (v) comparing the level of expression or activity of the flh2882 protein, mRNA, or genomic DNA in the pre-administration sample with the flh2882 protein, mRNA, or genomic DNA in the post administration sample or samples; and (vi) altering the administration of the compound to the subject accordingly. For example, increased administration of the compound may be desirable to increase the expression or activity of an flh2882 protein/gene to higher levels than detected, i.e., to increase the effectiveness of the agent. Alternatively, decreased administration of the agent may be desirable to decrease expression or activity of flh2882 to lower levels than detected, i.e. to decrease the effectiveness of the compound.

VI. Pharmaceutical Compositions

The flh2882 nucleic acid molecules, flh2882 proteins (particularly fragments of flh2882), modulators of an flh2882 protein, and anti-flh2882 antibodies (also referred to herein as “active compounds”) of the invention can be incorporated into pharmaceutical compositions suitable for administration to a subject, e.g., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an flh2882 protein or anti-flh2882 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

VII. Uses of Partial flh2882 Sequences

Fragments or fragments of the cDNA sequences identified herein (and the corresponding complete gene sequences) can be used in numerous ways as polynucleotide reagents. For example, these sequences can be used to: (a) map their respective genes on a chromosome; and, thus, locate gene regions associated with genetic disease; (b) identify an individual from a minute biological sample (tissue typing); and (c) aid in forensic identification of a biological sample. These applications are described in the subsections below.

a. Chromosome Mapping

Once the sequence (or a fragment of the sequence) of a gene has been isolated, this sequence can be used to map the location of the gene on a chromosome. This process is called chromosome mapping. Accordingly, fragments of an flh2882 nucleic acid sequences can be used to map the location of the flh2882 gene, respectively, on a chromosome. The mapping of the flh2882 sequence to chromosomes is an important first step in correlating these sequence with genes associated with disease.

Briefly, the flh2882 gene can be mapped to a chromosome by preparing PCR primers (preferably 15-25 bp in length) from the flh2882 gene sequence. Computer analysis of the flh2882 gene sequence can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the flh2882 gene sequence will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the flh2882 gene sequence to design oligonucleotide primers, sublocalization can be achieved with panels of fragments from specific chromosomes. Other mapping strategies which can similarly be used to map an flh2882 gene sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York, 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data (such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the flh2882 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

b. Tissue Typing

The flh2882 gene sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected fragments of an individual's genome. Thus, the flh2882 sequences described herein can be used to prepare two PCR primers from the 5′ and 3′ ends of the sequences. These primers can then be used to amplify an individual's DNA and subsequently sequence it.

Panels of corresponding DNA sequences from individuals prepared in this manner can provide unique individual identifications, as each individual will have a unique set of such DNA sequences due to allelic differences. The sequences of the present invention can be used to obtain such identification sequences from individuals and from tissue. The flh2882 gene sequences of the invention uniquely represent fragments of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequence of SEQ ID NO:14, can comfortably provide positive individual identification with a panel of perhaps 10 to 1,000 primers which each yield a noncoding amplified sequence of 100 bases. If a predicted coding sequence, such as that in SEQ ID NO:15, is used, a more appropriate number of primers for positive individual identification would be 500-2,000.

If a panel of reagents from the flh2882 gene sequences described herein is used to generate a unique identification database for an individual, those same reagents can later be used to identify tissue from that individual. Using the unique identification database, positive identification of the individual, living or dead, can be made from extremely small tissue samples.

c. Use of Partial flh2882 Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to the noncoding region of SEQ ID NO:14 are particularly appropriate for this use as greater numbers of polymorphisms occur in the noncoding regions, making it easier to differentiate individuals using this technique. Examples of polynucleotide reagents include the flh2882 sequences or fragments thereof, e.g., fragments derived from the noncoding region of SEQ ID NO:14, having a length of at least 20 bases, preferably at least 30 bases.

The flh2882 sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such flh2882 probes can be used to identify tissue by species and/or by organ type.

In a similar fashion, these reagents, e.g., flh2882 primers or probes can be used to screen tissue culture for contamination (i.e., screen for the presence of a mixture of different types of cells in a culture).

This invention is further illustrated by the following examples which should not be construed as limiting. The contents of all references, patent applications, patents, and published patent applications cited throughout this application are hereby incorporated by reference.

### EXAMPLES

### Example 1

### Identification of Human flh2882 cDNA

In this example, the human flh2882 nucleic acid molecule was identified by screening appropriate cDNA libraries. A non-annotated EST was first identified and used to screen a human fetal cDNA library. Several positive clones were identified, sequenced, and the sequences were assembled. BLAST analysis of nucleic acid databases in the public domain showed homologies only to the 3′ untranslated region of the flh2882 nucleic acid molecule and the original EST (GenBank™ Accession number T09060).

### Example 2

### Tissue Expression of the flh2882 Gene

Northern Analysis Using RNA from Human Tissue

Human brain multiple tissue northern (MTN) blots, human MTN I, II, and III blots (Clontech, Palo Alto, Calif.), containing 2 ###g of poly A+ RNA per lane were probed with human flh2882-specific primers (probes). The filters were prehybridized in 10 ml of Express Hyb hybridization solution (Clontech, Palo Alto, Calif.) at 68° C. for 1 hour, after which 100 ng of 32P labeled probe was added. The probe was generated using the Stratagene Prime-It kit, Catalog Number 300392 (Clontech, Palo Alto, Calif.). Hybridization was allowed to proceed at 68° C. for approximately 2 hours. The filters were washed in a 0.05% SDS/2×SSC solution for 15 minutes at room temperature and then twice with a 0.1% SDS/0.1×SSC solution for 20 minutes at 50° C. and then exposed to autoradiography film overnight at −80° C. with one screen. The human tissues tested included: heart, brain (regions of the brain tested included cerebellum, cerebral cortex, medulla, spinal cord, occipital pole, frontal lobe, temporal lobe, putamen, amygdala, caudate nucleus, hippocampus, corpus callosum, substantia nigra, subthalamic nucleus and thalamus), placenta, lung, liver, skeletal muscle, kidney, pancreas, spleen, thymus, prostate, testis, uterus, small intestine, colon (mucosal lining), and peripheral blood leukocyte.

There was a strong hybridization to human whole brain, and the substantia nigra indicating that the approximately 2.6 kb flh2882 gene transcript is expressed in these tissues.

### Example 3

### Expression of Recombinant flh2882 Protein in Bacterial Cells

In this example, flh2882 is expressed as a recombinant glutathione-S-transferase (GST) fusion protein in E. coli and the fusion protein is isolated and characterized. Specifically, flh2882 is fused to GST and this fusion protein is expressed in E. coli, e.g., strain PEB199. As the human protein is predicted to be approximately 38.7 kDa, and GST is predicted to be 26 kDa, the fusion protein is predicted to be approximately 64.7 kDa, in molecular weight. Expression of the GST-flh2882 fusion protein in PEB199 is induced with IPTG. The recombinant fusion protein is purified from crude bacterial lysates of the induced PEB199 strain by affinity chromatography on glutathione beads. Using polyacrylamide gel electrophoretic analysis of the protein purified from the bacterial lysates, the molecular weight of the resultant fusion protein is determined.

### Example 4

### Expression of Recombinant flh2882 Protein in COS Cells

To express the flh2882 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire flh2882 protein and a HA tag (Wilson et al. (1984) Cell 37:767) fused in-frame to its 3′ end of the fragment is cloned into the polylinker region of the vector, thereby placing the expression of the recombinant protein under the control of the CMV promoter.

To construct the plasmid, the flh2882 DNA sequence is amplified by PCR using two primers. The 5′ primer contains the restriction site of interest followed by approximately twenty nucleotides of the flh2882 coding sequence starting from the initiation codon; the 3′ end sequence contains complementary sequences to the other restriction site of interest, a translation stop codon, the HA tag and the last 20 nucleotides of the flh2882 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the flh2882 gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the flh2882-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the flh2882 protein is detected by radiolabelling (35S-methionine or 35S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with 35S-methionine (or 35S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the flh2882 coding sequence is cloned directly into the polylinker of the pCDNA/Amp vector using the appropriate restriction sites. The resulting plasmid is transfected into COS cells in the manner described above, and the expression of the flh2882 protein is detected by radiolabelling and immunoprecipitation using an flh2882 specific monoclonal antibody

### Example 5

### Characterization of the Human flh2882 Protein

In this example, the amino acid sequence of the human flh2882 protein was compared to amino acid sequences of known proteins and various motifs were identified.

The human flh2882 protein, the amino acid sequence of which is shown in (SEQ ID NO:13), is a novel protein which includes 337 amino acid residues. Hydrophobicity analysis indicated that the human flh2882 protein contains seven transmembrane domains between amino acid residues 11-28 (SEQ ID NO:16), 43-62 (SEQ ID NO:17), 80-102 (SEQ ID NO:18), 121-146 (SEQ ID NO:19), 169-190 (SEQ ID NO:20), 247-265 (SEQ ID NO:21), and 280-300 (SEQ ID NO:22). The nucleotide sequence of the human flh2882 was used as a database query using the BLASTN program (BLASTN1.3 MP, Altschul et al. (1990) J. Mol. Biol. 215:403). The closest hit was to the mouse 5HT5B receptor (GenBank™ Accession Number P31387). The highest similarity is 24n7 amino acid identities.

Equivalents

Those skilled in the art will recognize, or be able to ascertain using no more than routine experimentation, many equivalents to the specific embodiments of the invention described herein. Such equivalents are intended to be encompassed by the following claims.

### V. 52871, A NOVEL HUMAN G PROTEIN COUPLED RECEPTOR AND USES THEREOF

### Background of the Invention

Molecular cloning studies have shown that G protein-coupled receptors (“GPCRs”) form one of the largest protein superfamilies found in nature, and it is estimated that greater than 1000 different such receptors exist in mammals. Upon binding of extracellular ligands, GPCRs interact with a specific subset of heterotrimeric G-proteins that can then, in their activated forms, inhibit or activate various effector enzymes and/or ion channels. The ligands for many of these receptors are known although there exists an ever-increasing number of GPCRs which have been identified in the sequencing of the human genome for which no ligands have yet been identified. This latter subfamily of GPCRs is called the orphan family of GPCRs. In addition to both GPCRs with known ligands, as well as orphan GPCRs, there exist a family of GPCR-like molecules which share significant homology as well as many of the structural properties of the GPCR superfamily. For example, a family of GPCR-like proteins which arises from three alternatively-spliced forms of a gene occurring between the CD4 and triosephosphate isomerase genes at human chromosome 12p13, has been recently identified (Ansari-Lari et al. (1996) Genome Res. 6:314-326). Comparative sequence analysis of the syntenic region in mouse chromosome 6 has further revealed a murine homologue of one of these GPCR splice variants (Ansari-Lari et al. (1998) Genome Res. (1):29-40.

The fundamental knowledge that GPCRs play a role in regulating activities in virtually every cell in the human body has fostered an extensive search for modulators of such receptors for use as human therapeutics. In fact, the superfamily of GPCRs has proven to be among the most successful drug targets. Consequently, it has been recognized that the newly isolated orphan GPCRs, as well as the GPCR-like proteins, have great potential for drug discovery. With the identification of each new GPCR, orphan GPCR, and GPCR-like proteins, there exists a need for identifying the surrogate ligands for such molecules as well as for modulators of such molecules for use in regulating a variety of cellular responses.

### Summary of the Invention

The present invention is based, at least in part, on the discovery of novel G Protein Coupled Receptor family members, referred to herein as “52871” nucleic acid and protein molecules. The 52871 nucleic acid and protein molecules of the present invention (as well as modulators of said 52871 nucleic acid and protein molecules) are useful as agents in regulating a variety of cellular processes, e.g., cellular proliferation, growth, differentiation, nociception, and signaling (e.g., pain signaling). The 52871 nucleic acid and protein molecules (and modulators thereof) are also useful in regulating physiologic processes, for example, pain and/or pain disorders. Accordingly, in one aspect, this invention provides isolated nucleic acid molecules encoding 52871 proteins or biologically active portions thereof, as well as nucleic acid fragments suitable as primers or hybridization probes for the detection of 52871-encoding nucleic acids.

In one embodiment, the invention features an isolated nucleic acid molecule that includes the nucleotide sequence set forth in SEQ ID NO:27 or SEQ ID NO:29. In another embodiment, the invention features an isolated nucleic acid molecule that encodes a polypeptide including the amino acid sequence set forth in SEQ ID NO:28.

In still other embodiments, the invention features isolated nucleic acid molecules including nucleotide sequences that are substantially identical (e.g., 60% identical) to the nucleotide sequence set forth as SEQ ID NO:27 or SEQ ID NO:29. The invention further features isolated nucleic acid molecules including at least 30 contiguous nucleotides of the nucleotide sequence set forth as SEQ ID NO:27 or SEQ ID NO:29. In another embodiment, the invention features isolated nucleic acid molecules which encode a polypeptide including an amino acid sequence that is substantially identical (e.g., 60% identical) to the amino acid sequence set forth as SEQ ID NO:28. Also features are nucleic acid molecules which encode allelic variants of the polypeptide having the amino acid sequence set forth as SEQ ID NO:28. In addition to isolated nucleic acid molecules encoding full-length polypeptides, the present invention also features nucleic acid molecules which encode fragments, for example biologically active or antigenic fragments, of the full-length polypeptides of the present invention (e.g., fragments including at least 10 contiguous amino acid residues of the amino acid sequence of SEQ ID NO:28). In still other embodiments, the invention features nucleic acid molecules that are complementary to, are antisense to, or hybridize under stringent conditions to the isolated nucleic acid molecules described herein.

In a related aspect, the invention provides vectors including the isolated nucleic acid molecules described herein (e.g., 52871-encoding nucleic acid molecules). Such vectors can optionally include nucleotide sequences encoding heterologous polypeptides. Also featured are host cells including such vectors (e.g., host cells including vectors suitable for producing 52871 nucleic acid molecules and polypeptides).

In another aspect, the invention features isolated 52871 polypeptides and/or biologically active or antigenic fragments thereof. Exemplary embodiments feature a polypeptide including the amino acid sequence set forth as SEQ ID NO:28, a polypeptide including an amino acid sequence at least 60% identical to the amino acid sequence set forth as SEQ ID NO:28, a polypeptide encoded by a nucleic acid molecule including a nucleotide sequence at least 60% identical to the nucleotide sequence set forth as SEQ ID NO:27 or SEQ ID NO:29. Also featured are fragments of the full-length polypeptides described herein (e.g., fragments including at least 10 contiguous amino acid residues of the sequence set forth as SEQ ID NO:28) as well as fragments of allelic variants of the polypeptide having the amino acid sequence set forth as SEQ ID NO:28.

The 52871 polypeptides and/or biologically active or antigenic fragments thereof, are useful, for example, as reagents or targets in assays applicable to treatment and/or diagnosis of 52871 mediated or related disorders (e.g., pain disorders). In one embodiment, a 52871 polypeptide or fragment thereof has a 52871 activity. In another embodiment, a 52871 polypeptide or fragments thereof has a transmembrane domain, and/or a “7 transmembrane receptor profile” and optionally, has a 52871 activity. In a related aspect, the invention features antibodies (e.g., antibodies which specifically bind to any one of the polypeptides, as described herein) as well as fusion polypeptides including all or a fragment of a polypeptide described herein.

The present invention further features methods for detecting 52871 polypeptides and/or 52871 nucleic acid molecules, such methods featuring, for example, a probe, primer or antibody described herein. Also featured are kits for the detection of 52871 polypeptides and/or 52871 nucleic acid molecules. In a related aspect, the invention features methods for identifying compounds which bind to and/or modulate the activity of a 52871 polypeptide or 52871 nucleic acid molecule described herein. Also featured are methods for modulating a 52871 activity.

The present invention also provides diagnostic assays for identifying the presence or absence of a genetic alteration characterized by at least one of (i) aberrant modification or mutation of a gene encoding a 52871 protein; (ii) mis-regulation of the gene; and (iii) aberrant post-translational modification of a 52871 protein, wherein a wild-type form of the gene encodes a protein with a 52871 activity.

The invention further provides methods for identifying 52871 modulators. In one embodiment, the invention provides a method for identifying a compound which binds to a 52871 polypeptide by contacting the polypeptide, or a cell expressing the polypeptide with a test compound, and determining whether the polypeptide binds to the test compound. In another embodiment, the invention provides a method for identifying a compound which modulates the activity of a 52871 polypeptide comprising contacting a 52871 polypeptide or a cell which expresses a 52871 polypeptide with a test compound and determining the effect of the test compound on the activity of the polypeptide. In another embodiment, the 52871 activity is modulation of nociception. In yet another embodiment, the 52871 activity is modulation of pain signaling.

Accordingly, in a further aspect, the invention provides a method for identifying a compound which modulates pain comprising contacting a 52871 polypeptide or a cell which expresses a 52871 polypeptide with a test compound with a test compound and identifying the compound as a modulator of pain by determining the effect of the test compound on the activity of the polypeptide. In yet another aspect, the invention provides a method for identifying a compound capable of modulating nociception comprising contacting a 52871 polypeptide or a cell which expresses a 52871 polypeptide with a test compound and identifying the compound as a modulator of nociception by determining the effect of the test compound on the activity of the polypeptide.

The present invention further features a method for treating a subject having pain or a pain disorder comprising administering to the subject a 52871 modulator. In one embodiment, the 52871 modulator is a small molecule. In another embodiment, the 52871 modulator is administered in a pharmaceutically acceptable formulation. In yet another embodiment the 52871 modulator is administered using a gene therapy vector.

Other features and advantages of the invention will be apparent from the following detailed description and claims.

### Detailed Description of the Invention

The present invention is based, at least in part, on the discovery of novel G Protein Coupled Receptor (GPCR) molecules, referred to herein as “52871” nucleic acid and protein molecules, which are novel members of a family of receptors which possess the ability to associate with G protein molecules in order to function in their biological capacity (e.g., to modulate target enzymes or ion channels). These novel molecules are capable of participating in signaling pathways (e.g., as a hormone receptor, as a neurotransmitter receptor, as an modulator of intracellular signaling) and, thus, play a role in or function in a variety of cellular processes, e.g., cellular signaling. GPCRs act as the receptors for various different families of neuropeptides (Pheng and Regoli (2000) Life Sci 67:847; Rozengurt, E. (1998) J. Cell Physiology 177:507; Larhammar, et al. (1993) Drug Des. Discovery 9:179; Elshourbagy, et al. (2000) J Biol Chem 275:25965). Neuropeptides are known to be involved in nociception (e.g., chemical, mechanical, or thermal nociception), and thereby function to modulate pain elicitation (Bannon et al. (2000) Brain Res 868:79). 52871 mRNA is predominantly expressed in the brain, spinal cord, dorsal root ganglia (DRG), and skin, as compared to other tissues. Based at least in part on the specific expression pattern of 52871, in addition to the fact that GPCRs are known to be involved in the modulation of nociceptive neurons and pain signaling (through interaction with neuropeptides), the novel 52871 molecules of the present invention can be used as targets for developing novel diagnostic targets and therapeutic agents to control pain and pain disorders.

As used herein, the term “G protein coupled receptor” (referred to herein interchangeably as “GPCR”) includes a protein, peptide, or enzyme which is able to interact with one or more G protein molecules (e.g., neuropeptides) and or one or more signaling molecules, in order to carry out its function(s), e.g., recognition of signaling molecules, modulation of intracellular signaling, modulation of nociception, and/or modulation of pain. GPCR molecules are involved in the transduction of signals that are transmitted to cells from without by important signaling molecules, including peptides, hormones, growth factors, and neurotransmitters. GPCR proteins act as cell-surface receptors for these molecules, with an extracellular domain which interacts with the signaling molecule, and an intracellular domain which can further activate signaling events. The activity of the intracellular domain is typically sensitive to the binding state of the extracellular domain (e.g., ligand bound, ligand unbound). Normally the intracellular domain can interact with guanine nucleotide-binding (G) proteins, thus activating it. Thereupon, the activated G protein can modulate the activity of one or more enzymes. This enzyme can pass the signal on directly by catalyzing the production of a second messenger (e.g., cyclic AMP; cAMP), or it can catalyze the production of a soluble mediator (e.g., inositol triphosphate; IP3) which can in turn release other second messengers (e.g., Ca2+ from the endoplasmic reticulum). Examples of GPCR molecules include prokaryotic, plant, and mammalian GPCR molecules. As transmembrane receptor protein, the 52871 molecules of the present invention provide novel diagnostic targets and therapeutic agents to control GPCR-associated disorders.

Preferably such GPCR proteins comprise a family of GPCR molecules. The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., mouse or monkey proteins. Members of a family also have common functional characteristics.

For example, the family of G protein-coupled receptors (GPCRs), to which the 52871 proteins of the present invention bear significant homology, comprise an N-terminal extracellular domain, seven transmembrane domains (also referred to as membrane-spanning domains), three extracellular domains (also referred to as extracellular loops), three cytoplasmic domains (also referred to as cytoplasmic loops), and a C-terminal cytoplasmic domain (also referred to as a cytoplasmic tail). Members of the GPCR family also share certain conserved amino acid residues, some of which have been determined to be critical to receptor function and/or G protein signaling. For example, GPCRs contain the following features: a conserved asparagine residue in the first transmembrane domain; a cysteine residue in the first extracellular loop which is believed to form a disulfide bond with a conserved cysteine residue in the second extracellular loop; a conserved leucine and aspartate residue in the second transmembrane domain; an aspartate-arginine-tyrosine motif (DRY motif) at the interface of the third transmembrane domain and the second cytoplasmic loop of which the arginine residue is almost invariant (members of the rhodopsin subfamily of GPCRs comprise a histidine-arginine-methionine motif (HRM motif) as compared to a DRY motif); a conserved tryptophan and proline residue in the fourth transmembrane domain; a conserved phenylalanine residue which is commonly found as part of the motif FXXCXXP (SEQ ID NO:30); and a conserved leucine residue in the seventh transmembrane domain which is commonly found as part of the motif DPXXY (SEQ IF NO:31) or NPXXY (SEQ ID NO:32). Table 8 depicts an alignment of the seven transmembrane domains (TM1-TM7) of 5 known GPCRs. The conserved residues described herein are indicated by asterices.

TABLE 8
ALIGNMENT OF:

| | SEQ | | GenBank |
|---|---|---|---|
| Name | ID | species | Acc No. |
| thrombin | (69) | human | P25116 |
| rhodopsin | (70) | human | P08100 |
| m1ACh | (71) | rat | P08482 |
| IL-8A | (72) | human | P25024 |
| octopamine | (73) | Drosophila | P22270 |
| | | melanogaster | |

TM1

| | | | |
|---|---|---|---|
| | | * | |
| 4. | 102 | TLFVPSVYTGVFVVSLPLNIMAIVVFILKMK | 132 |
| 5. | 37 | FSMLAAYMFLLIVLGFPINFLTLYVTVQHKK | 67 |
| 6. | 25 | VAFIGITTGLLSLATVTGNLLVLISFKVNTE | 55 |
| 7. | 39 | KYVVIIAYALVFLLSLLGNSLVMLVILYSRV | 69 |
| 8. | 109 | ALLTALVLSVIIVLTIIGNILVILSVFTYKP | 139 |
| TM2 | | | |
| | | * * | |
| 4. | 138 | VVYMLHLATADVLFVSVLPFKISYYFSG | 165 |
| 5. | 73 | NYILLNLAVADLFMVLGGFTSTLYTSLH | 100 |
| 6. | 61 | NYFLLSLACADLIIGTFSMNLYTTYLLM | 88 |
| 7. | 75 | DVYLLNLALADLLFALTLPIWAASKVNG | 102 |
| 8. | 145 | NFFIVSLAVADLTVALLVLPFNVAYSIL | 172 |
| TM3 | | | |
| | | * | |
| 4. | 176 | RFVTAAFYCNMYASILLMTVISIDR | 200 |
| 5. | 111 | NLEGFFATLGGEIALWSLVVLAIER | 135 |
| 6. | 99 | DLWLALDYVASNASVMNLLLISFDR | 123 |
| 7. | 111 | KVVSLLKEVNFYSGILLLACISVDR | 135 |
| 8. | 183 | KLWLTCDVLCCTSSILNLCAIALDR | 207 |
| TM4 | | | |
| | | * * | |
| 4. | 215 | TLGRASFTCLAIWALAIAGVVPLVLKE | 241 |
| 5. | 149 | GENHAIMGVAFTWVMALACAAPPLAGW | 175 |
| 6. | 138 | TPRRAALMTGLAWLVSFVLWAPAILFW | 164 |
| 7. | 149 | KRHLVKFVCLGCWGLSMNLSLPFFLFR | 175 |
| 8. | 222 | TVGRVLLLISGVWLLSLLISSPPLIGW | 248 |
| TM5 | | | |
| | | * * * | |
| 4. | 268 | AYYFSAFSAVFFFVPLIISTVCYVSIIRC | 296 |
| 5. | 201 | ESFVIYMFVVHFTIPMIIIFFCYGQLVFT | 229 |
| 6. | 186 | PIITFGTAMAAFYLPVTVMCTLYWRIYRE | 214 |
| 7. | 200 | MVLRILPHTFGFIVPLFVMLFCYGFTLRT | 228 |
| 8. | 267 | RGYVIYSSLGSFFIPLAIMTIVYIEIFVA | 295 |
| TM6 | | | |
| | | * * * | |
| 4. | 313 | FLSAAVFCIFIICFGPTNVLLIAHYSFL | 340 |
| 5. | 252 | RMVIIMVIAFLICWVPYASVAFYIFTHQ | 279 |
| 6. | 365 | RTLSAILLAFILTWTPYNIMVLVSTFCK | 397 |
| 7. | 242 | RVIFAVVLIFLLCWLPYNLVLLADTLMR | 269 |
| 8. | 529 | RTLGIIMGVFVICWLPFFLMYVILPFCQ | 556 |
| TM7 | | | |
| | | ** * | |
| 4. | 347 | EAAYFAYLLCVCVSSISSCIDPLIYYYASSECQ | 379 |
| 5. | 282 | NFGPIFMTIPAFFAKSAAIYNPVIYIMMNKQFR | 314 |
| 6. | 394 | CVPETLWELGYWLCYVNSTVNPMCYALCNKAFR | 426 |
| 7. | 281 | NNIGRALDATEILGFLHSCLNPIIYAFIGQNFR | 313 |
| 8. | 559 | CPTNKFKNFITWLGYINSGLNPVIYTIFNLDYR | 591 |

Accordingly, GPCR-like proteins such as the 52871 proteins of the present invention contain a significant number of structural characteristics of the GPCR family. For instance, the 52871 proteins of the present invention contain conserved cysteines found in the first 2 extracellular loops (prior to the third and fifth transmembrane domains) of most GPCRs (cys 121 and cys 197 of SEQ ID NO:28). A highly conserved asparagine residue in the first transmembrane domain is present (asn 67 in SEQ ID NO:28). Transmembrane domain two of the 52871 proteins contains a highly conserved leucine (leu 90 of SEQ ID NO:28). The two cysteine residues are believed to form a disulfide bond that stabilizes the functional protein structure. A highly conserved tryptophan and proline in the fourth transmembrane domain of the 52871 proteins is present (trp 171 and pro 180 of SEQ ID NO:28). The third cytoplasmic loop contains 40 amino acid residues and is thus the longest cytoplasmic loop of the three, characteristic of G protein coupled receptors. Moreover, a highly conserved proline in the sixth transmembrane domain is present (pro 289 of SEQ ID NO:28). The proline residues in the fourth, fifth, sixth, and seventh transmembrane domains are thought to introduce kinks in the alpha-helices and may be important in the formation of the ligand binding pocket. Moreover, an almost invariant proline is present in the seventh transmembrane domain of 52871 (pro327 of SEQ ID NO:28).

In one embodiment, the 52871 proteins of the present invention are proteins having an amino acid sequence of about 200-475, preferably about 250-425, more preferably about 275-400, more preferably about 300-375, or about 330-350 amino acids in length. In another embodiment, the 52871 proteins of the present invention contain at least one transmembrane domain. As used herein, the term “transmembrane domain” includes an amino acid sequence having at least about 10, preferably about 13, preferably about 16, more preferably about 19, 21, 23, 25, 30, 35 or 40 amino acid residues, of which at least about 50-60%, 60-70%, preferably about 70-80% more preferably about 80-90%, or about 90-95% of the amino acid residues contain non-polar side chains, for example, alanine, valine, leucine, isoleucine, proline, phenylalanine, tryptophan, and methionine. A transmembrane domain is lipophilic in nature. Transmembrane domains are described in, for example, Zagotta W. N. et al., (1996) Annual Rev. Neurosci. 19:235-63, the contents of which are incorporated herein by reference. For example, a transmembrane domain can be found at about amino acids 53-75 of SEQ ID NO:28. In a preferred embodiment, a 52871 protein of the present invention has more than one transmembrane domain, preferably 2, 3, 4, 5, 6, or 7 transmembrane domains. For example, transmembrane domains can be found at about amino acids 53-75, 90-108, 126-144, 165-186, 210-234, 275-293, and 309-333 of SEQ ID NO:28. In a particularly preferred embodiment, a 52871 protein of the present invention has 7 transmembrane domains.

In another embodiment, a 52871 family member is identified based on the presence of at least one cytoplasmic loop, also referred to herein as a cytoplasmic domain. In another embodiment, a 52871 family member is identified based on the presence of at least one extracellular loop. As defined herein, the term “loop” includes an amino acid sequence having a length of at least about 4, preferably about 5-10, preferably about 10-20, and more preferably about 20-30, 30-40, 40-50, 50-60, 60-70, 70-80, 80-90, 90-100, or 100-150 amino acid residues, and has an amino acid sequence that connects two transmembrane domains within a protein or polypeptide. Accordingly, the N-terminal amino acid of a loop is adjacent to a C-terminal amino acid of a transmembrane domain in a naturally-occurring GPCR or GPCR-like molecule, and the C-terminal amino acid of a loop is adjacent to an N-terminal amino acid of a transmembrane domain in a naturally-occurring GPCR or GPCR-like molecule.

As used herein, a “cytoplasmic loop” includes an amino acid sequence located within a cell or within the cytoplasm of a cell. For example, a cytoplasmic loop is found at about amino acids 76-89, 145-164, and 235-274 of SEQ ID NO:28. Also as used herein, an “extracellular loop” includes an amino acid sequence located outside of a cell, or extracellularly. For example, an extracellular loop can be found at about amino acid residues 109-125, 187-209, and 294-308 of SEQ ID NO:28.

In another embodiment of the invention, a 52871 family member is identified based on the presence of a “C-terminal cytoplasmic domain”, also referred to herein as a C-terminal cytoplasmic tail, in the sequence of the protein. As used herein, a “C-terminal cytoplasmic domain” includes an amino acid sequence having a length of at least 5 amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring GPCR or GPCR-like protein.

In another embodiment, a 52871 family member is identified based on the presence of an “N-terminal extracellular domain”, also referred to herein as an N-terminal extracellular loop in the amino acid sequence of the protein. As used herein, an “N-terminal extracellular domain” includes an amino acid sequence having about 1-500, preferably about 1-400, more preferably about 1-300, more preferably about 1-200, even more preferably about 1-100, and even more preferably about 1-60 amino acid residues in length and is located outside of a cell or extracellularly. The C-terminal amino acid residue of a “N-terminal extracellular domain” is adjacent to an N-terminal amino acid residue of a transmembrane domain in a naturally-occurring GPCR or GPCR-like protein. For example, an N-terminal cytoplasmic domain is found at about amino acid residues 1-52 of SEQ ID NO:28.

Accordingly in one embodiment of the invention, a 52871 family member includes at least one transmembrane domain and/or at least one cytoplasmic loop, and/or at least one extracellular loop. In another embodiment, the 52871 family member further includes an N-terminal extracellular domain and/or a C-terminal cytoplasmic domain. In another embodiment, the 52871 family member can include up to six transmembrane domains, three cytoplasmic loops, and two extracellular loops, or can include up to six transmembrane domains, three extracellular loops, and two cytoplasmic loops. The former embodiment can further include an N-terminal extracellular domain. The latter embodiment can further include a C-terminal cytoplasmic domain. In another embodiment, the 52871 family member can include seven transmembrane domains, three cytoplasmic loops, and three extracellular loops and can further include an N-terminal extracellular domain or a C-terminal cytoplasmic domain.

In another embodiment, a 52871 family member is identified based on the presence of at least one “7 transmembrane receptor profile”, also referred to as a “7-TMR profile”, in the protein or corresponding nucleic acid molecule. As used herein, the term “7-TMR profile” includes an amino acid sequence having at least about 100-400, preferably about 150-350, more preferably about 200-300 amino acid residues, or at least about 250-260 amino acids in length and having a bit score for the alignment of the sequence to the “7tm_{—}1” family Hidden Markov Model (HMM) of at least 100, preferably 100-110, more preferably 110-120, more preferably 120-130, 130-140, 140-150, 150-160 or greater. The 7tm_{—}1 family HMM has been assigned the PFAM Accession PF00001.

To identify the presence of a 7-TMR profile in a 52871 family member, the amino acid sequence of the protein family member is searched against a database of HMMs (e.g., the Pfam database, release 2.1) using the default parameters. For example, the search can be performed using the hmmsf program (family specific) using the default parameters (e.g., a threshold score of 15) for determining a hit. hmmsf is available as part of the HMMER package of search programs (HMMER 2.1.1, December 1998) which is freely distributed by the Washington University School of Medicine. Alternatively, the threshold score for determining a hit can be lowered (e.g., to 8 bits). For example, a search using the amino acid sequence of SEQ ID NO:28 was performed against the HMM database resulting in the identification of a 7tm_{—}1 receptor profile in the amino acid sequence of human 52871 (SEQ ID NO:28) at about residues 66-330 having a score of 164.0.

A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

In a preferred embodiment, a 52871 molecule, as described herein is characterized by the presence of a “G-protein coupled receptor signature.” As used herein, the term “G-protein coupled receptor signature” includes a motif having the consensus sequence [GSTALIVMFYWC]-[GSTANCPDE]-{EDPKRH}-X(2)-[LIVMNQGA]-X(2)-[LIVMFT]-[GSTANC]-[LIVMFYWSTAC]-[DENH]-R-[FYWCSH]-X(2)-[LIVM] (SEQ ID NO:74) and is described under Prosite entry number PDOC00210. A G-protein coupled receptor signature can be found, for example, within the 7-TMR profile of the 52871 protein of SEQ ID NO:28 at about residues 134-150. The consensus sequences described herein are described according to standard Prosite Signature designation (e.g., all amino acids are indicated according to their universal single letter designation; X designates any amino acid; X(n) designates any n amino acids, e.g., X(2) designates any 2 amino acids; [LIVM] indicates any one of the amino acids appearing within the brackets, e.g., any one of L, I, V, or M, in the alternative, any one of Leu, Ile, Val, or Met.); and {LIVM} indicates any amino acid except the amino acids appearing within the brackets, e.g., not L, not I, not V, and not M.

Isolated proteins of the present invention, for example 52871 proteins, preferably have an amino acid sequence sufficiently identical to the amino acid sequence of SEQ ID NO:28, or are encoded by a nucleotide sequence sufficiently identical to SEQ ID NO:27 or 29. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 85%, 90%, 91%, 92%, 93%, 94%, 95%, 96%, 97%, 98%, 99% or more homology or identity across the amino acid sequences of the domains and contain at least one and preferably two structural domains or motifs, are defined herein as sufficiently identical. Furthermore, amino acid or nucleotide sequences which share at least 50%, 55%, 60%, 65%, 70%, 75%, 80%, 85%, 85%, 90%, 91%, 92%, 93%, 94%, 95%, 96%, 97%, 98%, 99% or more homology or identity and share a common functional activity are defined herein as sufficiently identical.

In a preferred embodiment, a 52871 protein includes at least one or more of the following domains: a transmembrane domain, and/or a 7-TMR profile, and has an amino acid sequence at least about 50%, 55%, 60%, 65%, 70%, 75%, 80%, 85%, 85%, 90%, 91%, 92%, 93%, 94%, 95%, 96%, 97%, 98%, 99% or more homologous or identical to the amino acid sequence of SEQ ID NO:28. In yet another preferred embodiment, a 52871 protein includes at least one or more of the following domains: a transmembrane domain, a 7-TMR profile, and is encoded by a nucleic acid molecule having a nucleotide sequence which hybridizes under stringent hybridization conditions to a complement of a nucleic acid molecule comprising the nucleotide sequence of SEQ ID NO:27 or 29. In another preferred embodiment, a 52871 protein includes at least one or more of the following domains: a transmembrane domain, a 7-TMR profile, and has a 52871 activity.

As used interchangeably herein, a “52871 activity”, “biological activity of 52871” or “functional activity of 52871”, refers to an activity exerted by a 52871 protein, polypeptide or nucleic acid molecule on a 52871 responsive cell as determined in vivo, or in vitro, according to standard techniques. In one embodiment, a 52871 activity is a direct activity, such as an association with a 52871-traget molecule. As used herein, a “target molecule” or “binding partner” or “ligand” or “substrate” is a molecule with which a 52871 protein binds or interacts in nature, such that 52871-mediated function is achieved. A 52871 target molecule can be a non-52871 molecule or a 52871 protein or polypeptide of the present invention. In an exemplary embodiment, a 52871 target molecule is a 52871 ligand such as a hormone, a neurotransmitter, a growth factor, an opioid, a pheremone, a peptide (e.g., a cytokine, a chemokine, a neuropeptide), a biogenic amine, an eicosanoid, a lipid (e.g., a leukotriene, a cannabinoids), an excitatory amino acid (e.g., GABA, glutamate), an ion (e.g., calcium), or a retinoid). Examples of 52871 substrates also include molecules that are essential for 52871 intracellular function, e.g., G protein, adenyl cyclase, enzymes involved in the inositol triphosphate signaling pathways. Alternatively, a 52871 activity is an indirect activity, such as a cellular signaling activity (e.g., ligand recognition, modulation of intracellular signaling mechanisms, modulation of nociception, modulation of pain) mediated by interaction of the 52871 protein with a 52871 ligand.

In a preferred embodiment, a 52871 activity is at least one or more of the following activities: (i) interaction of a 52871 protein with soluble 52871 ligand; (ii) interaction of a 52871 protein with a membrane-bound non-52871 protein; (iii) interaction of a 52871 protein with an intracellular protein (e.g., an intracellular enzyme or signal transduction molecule); (iv) indirect interaction of a 52871 protein with an intracellular protein (e.g., a downstream signal transduction molecule; (v) modulation of intra- or intercellular signaling or feedback mechanisms; (vi) modulation of the intracellular levels and/or homeostatic balance of signaling molecule pools (e.g., Ca2+, diacylglycerol, IP3, cAMP); (vii) regulation of cellular proliferation; (viii) regulation of cellular differentiation; (ix) regulation of development; (x) regulation of gene expression in a cell which expresses a 52871 protein; (xi) modulation of nociception; (xii) modulation of pain; and (xiii) regulation of cell death.

Accordingly, another embodiment of the invention features isolated 52871 proteins and polypeptides having a 52871 activity. Other preferred proteins are 52871 proteins having one or more of the following domains: a transmembrane domain, a 7-TMR profile, and, preferably, a 52871 activity.

Additional preferred proteins have at least one transmembrane domain, one 7-TMR profile, and are, preferably, encoded by a nucleic acid molecule having a nucleotide sequence which hybridizes under stringent hybridization conditions to a complement of a nucleic acid molecule comprising the nucleotide sequence of SEQ ID NO:27 or 29.

The human 52871 gene, which is approximately 1731 nucleotides in length and encodes a protein which is approximately 348 amino acid residues in length.

Various aspects of the invention are described in further detail in the following subsections:

I. Isolated Nucleic Acid Molecules

One aspect of the invention pertains to isolated nucleic acid molecules that encode 52871 proteins or biologically active portions thereof, as well as nucleic acid fragments sufficient for use as hybridization probes to identify 52871-encoding nucleic acid molecules (e.g., 52871 mRNA) and fragments for use as PCR primers for the amplification or mutation of 52871 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated 52871 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:27 or 29, or a portion thereof, can be isolated using standard molecular biology techniques and the sequence information provided herein. Using all or portion of the nucleic acid sequence of SEQ ID NO:27 or 29, 52871 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y. 1989).

Moreover, a nucleic acid molecule encompassing all or a portion of SEQ ID NO:27 or 29, can be isolated by the polymerase chain reaction (PCR) using synthetic oligonucleotide primers designed based upon the sequence of SEQ ID NO:27 or 29.

A nucleic acid of the invention can be amplified using cDNA, mRNA or, alternatively, genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to 52871 nucleotide sequences can be prepared by standard synthetic techniques, e.g., using an automated DNA synthesizer.

In one embodiment, an isolated nucleic acid molecule of the invention comprises the nucleotide sequence shown in SEQ ID NO:27 or 29. This cDNA may comprise sequences encoding the human 52871 protein (i.e., “the coding region”, from nucleotides 201-1247), as well as 5′ untranslated sequences (nucleotides 1-200) and 3′ untranslated sequences (nucleotides 1248-1731) of SEQ ID NO:27. Alternatively, the nucleic acid molecule can comprise only the coding region of SEQ ID NO:27 (e.g., nucleotides 201-1247, corresponding to SEQ ID NO:29). Accordingly, in another embodiment, an isolated nucleic acid molecule of the invention comprises SEQ ID NO:29 and nucleotides 1-200 of SEQ ID NO:27. In yet another embodiment, the isolated nucleic acid molecule comprises SEQ ID NO:29 and nucleotides 1248-1731 of SEQ ID NO:27. In yet another embodiment, the nucleic acid molecule consists of the nucleotide sequence set forth as SEQ ID NO:27 or SEQ ID NO:29.

In still another embodiment, an isolated nucleic acid molecule of the invention comprises a nucleic acid molecule which is a complement of the nucleotide sequence shown in SEQ ID NO:27 or 29, or a portion of any of these nucleotide sequences. A nucleic acid molecule which is complementary to the nucleotide sequence shown in SEQ ID NO:27 or 29, is one which is sufficiently complementary to the nucleotide sequence shown in SEQ ID NO:27 or 29, such that it can hybridize to a complement of the nucleotide sequence shown in SEQ ID NO:27 or 29, respectively, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 91%, 92%, 93%, 94%, 95%, 96%, 97%, 98%, 99% or more identical to the nucleotide sequence shown in SEQ ID NO:27 or 29 (e.g., to the entire length of the nucleotide sequence), or to a portion or complement of any of these nucleotide sequences. In one embodiment, a nucleic acid molecule of the present invention comprises a nucleotide sequence which is at least (or no greater than) 50-100, 100-250, 250-500, 500-750, 750-1000, 1000-1250, 1250-1500, 1500-1700 or more nucleotides in length and hybridizes under stringent hybridization conditions to a complement of a nucleic acid molecule of SEQ ID NO:27 or 29.

Moreover, the nucleic acid molecule of the invention can comprise only a portion of the nucleic acid sequence of SEQ ID NO:27 or 29, for example, a fragment which can be used as a probe or primer or a fragment encoding a portion of a 52871 protein, e.g., a biologically active portion of a 52871 protein. The nucleotide sequence determined from the cloning of the 52871 gene allows for the generation of probes and primers designed for use in identifying and/or cloning other 52871 family members, as well as 52871 homologues from other species. The probe/primer typically comprises substantially purified oligonucleotide. The probe/primer (e.g., oligonucleotide) typically comprises a region of nucleotide sequence that hybridizes under stringent conditions to a complement of at least about 12 or 15, preferably about 20 or 25, more preferably about 30, 35, 40, 45, 50, 55, 60, 65, or 75 consecutive nucleotides of a sense sequence of SEQ ID NO:27 or 29, or the anti-sense sequence of SEQ ID NO:27 or 29, of a naturally occurring allelic variant or mutant of SEQ ID NO:27 or 29.

Exemplary probes or primers are at least (or no greater than) 12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the 52871 nucleotide sequences can be used to detect (e.g., specifically detect) transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of a 52871 sequence, e.g., a domain, region, site or other sequence described herein. The primers should be at least 5, 10, or 50 base pairs in length and less than 100, or less than 200, base pairs in length. The primers should be identical, or differs by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which misexpress a 52871 protein, such as by measuring a level of a 52871-encoding nucleic acid in a sample of cells from a subject e.g., detecting 52871 mRNA levels or determining whether a genomic 52871 gene has been mutated or deleted.

A nucleic acid fragment encoding a “biologically active portion of a 52871 protein” can be prepared by isolating a portion of the nucleotide sequence of SEQ ID NO:27 or 3, which encodes a polypeptide having a 52871 biological activity (the biological activities of the 52871 proteins are described herein), expressing the encoded portion of the 52871 protein (e.g., by recombinant expression in vitro) and assessing the activity of the encoded portion of the 52871 protein. In an exemplary embodiment, the nucleic acid molecule is at least 50-100, 100-250, 250-500, 500-700, 750-1000, 1000-1250, 1250-1500, 1500-1700 or more nucleotides in length and encodes a protein having a 52871 activity (as described herein).

The invention further encompasses nucleic acid molecules that differ from the nucleotide sequence shown in SEQ ID NO:27 or 29. Such differences can be due to degeneracy of the genetic code, thus resulting in a nucleic acid which encodes the same 52871 proteins as those encoded by the nucleotide sequence shown in SEQ ID NO:27 or 29. In another embodiment, an isolated nucleic acid molecule of the invention has a nucleotide sequence encoding a protein having an amino acid sequence which differs by at least 1, but no greater than 5, 10, 20, 50 or 100 amino acid residues from the amino acid sequence shown in SEQ ID NO:28. In yet another embodiment, the nucleic acid molecule encodes the amino acid sequence of human 52871. If an alignment is needed for this comparison, the sequences should be aligned for maximum homology.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

Allelic variants result, for example, from DNA sequence polymorphisms within a population (e.g., the human population) that lead to changes in the amino acid sequences of the 52871 proteins. Such genetic polymorphism in the 52871 genes may exist among individuals within a population due to natural allelic variation. As used herein, the terms “gene” and “recombinant gene” refer to nucleic acid molecules which include an open reading frame encoding a 52871 protein, preferably a mammalian 52871 protein, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:28, wherein the nucleic acid molecule hybridizes to a complement of a nucleic acid molecule comprising SEQ ID NO:27 or SEQ ID NO:29, for example, under stringent hybridization conditions.

Allelic variants of human 52871 include both functional and non-functional 52871 proteins. Functional allelic variants are naturally occurring amino acid sequence variants of the human 52871 protein that maintain the ability to process a 52871 substrate (e.g., carboxylation, decarboxylation). Functional allelic variants will typically contain only conservative substitution of one or more amino acids of SEQ ID NO:28, or substitution, deletion or insertion of non-critical residues in non-critical regions of the protein.

Non-functional allelic variants are naturally occurring amino acid sequence variants of the human 52871 protein that do not have the ability to bind or process a 52871 substrate (e.g., signal molecule recognition, signal transduction), and/or carry out any of the 52871 activities described herein. Non-functional allelic variants will typically contain a non-conservative substitution, a deletion, or insertion or premature truncation of the amino acid sequence of SEQ ID NO:28, or a substitution, insertion or deletion in critical residues or critical regions of the protein.

The present invention further provides non-human orthologues (e.g., non-human orthologues of the human 52871 protein). Orthologues of the human 52871 protein are proteins that are isolated from non-human organisms and possess the same 52871 substrate binding and/or modulation of membrane excitability activities of the human 52871 protein. Orthologues of the human 52871 protein can readily be identified as comprising an amino acid sequence that is substantially identical to SEQ ID NO:28.

Moreover, nucleic acid molecules encoding other 52871 family members and, thus, which have a nucleotide sequence which differs from the 52871 sequences of SEQ ID NO:27 or 29. For example, another 52871 cDNA can be identified based on the nucleotide sequence of human 52871. Moreover, nucleic acid molecules encoding 52871 proteins from different species, and which, thus, have a nucleotide sequence which differs from the 52871 sequences of SEQ ID NO:27 or 29. For example, a mouse or monkey 52871 cDNA can be identified based on the nucleotide sequence of a human 52871.

Nucleic acid molecules corresponding to natural allelic variants and homologues of the 52871 cDNAs of the invention can be isolated based on their homology to the 52871 nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the 52871 cDNAs of the invention can further be isolated by mapping to the same chromosome or locus as the 52871 gene.

Orthologues, homologues and allelic variants can be identified using methods known in the art (e.g., by hybridization to an isolated nucleic acid molecule of the present invention, for example, under stringent hybridization conditions). In one embodiment, an isolated nucleic acid molecule of the invention is at least 15, 20, 25, 30 or more nucleotides in length and hybridizes under stringent conditions to a complement of the nucleic acid molecule comprising the nucleotide sequence of SEQ ID NO:27 or 29. In another embodiment, the nucleic acid is at least 50-100, 100-250, 250-500, 500-750, 750-1000, 1000-1250, 1250-1500, 1500-1700 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions are hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions are hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions are hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH2PO4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH2PO4, 7% SDS at about 65° C., followed by one or more washes at 0.02M NaH2PO4, 1% SDS at 65° C. (see e.g., Church and Gilbert (1984) Proc. Natl. Acad. Sci. USA 81:1991-1995).

Preferably, an isolated nucleic acid molecule of the invention that hybridizes under stringent conditions to a complement of the sequence of SEQ ID NO:27 or 29, and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the 52871 sequences that may exist in the population, the skilled artisan will further appreciate that changes can be introduced by mutation into the nucleotide sequences of SEQ ID NO:27 or 29, thereby leading to changes in the amino acid sequence of the encoded 52871 proteins, without altering the functional ability of the 52871 proteins. For example, nucleotide substitutions leading to amino acid substitutions at “non-essential” amino acid residues can be made in the sequence of SEQ ID NO:27 or 29. A “non-essential” amino acid residue is a residue that can be altered from the wild-type sequence of 52871 (e.g., the sequence of SEQ ID NO:28) without altering the biological activity, whereas an “essential” amino acid residue is required for biological activity. For example, amino acid residues that are conserved among the 52871 proteins of the present invention (for example, within transmembrane domains, the 7-TMR profile, or the G-protein coupled receptor signature), are predicted to be particularly unamenable to alteration. Furthermore, additional amino acid residues that are conserved between the 52871 proteins of the present invention and other members of the 52871 family are not likely to be amenable to alteration.

Accordingly, another aspect of the invention pertains to nucleic acid molecules encoding 52871 proteins that contain changes in amino acid residues that are not essential for activity. Such 52871 proteins differ in amino acid sequence from SEQ ID NO:28, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to SEQ ID NO:28, e.g., to the entire length of SEQ ID NO:28).

An isolated nucleic acid molecule encoding a 52871 protein identical to the protein of SEQ ID NO:28 can be created by introducing one or more nucleotide substitutions, additions or deletions into the nucleotide sequence of SEQ ID NO:27 or 29, additions or deletions are introduced into the encoded protein. Mutations can be introduced into SEQ ID NO:27 or 29, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 52871 protein is preferably replaced with another amino acid residue from the same side chain family. Alternatively, in another embodiment, mutations can be introduced randomly along all or part of a 52871 coding sequence, such as by saturation mutagenesis, and the resultant mutants can be screened for 52871 biological activity to identify mutants that retain activity. Following mutagenesis of SEQ ID NO:27 or 29, the encoded protein can be expressed recombinantly and the activity of the protein can be determined.

In a preferred embodiment, a mutant 52871 protein can be assayed for the ability to metabolize or catabolize biochemical molecules necessary for energy production or storage, permit intra- or intercellular signaling, metabolize or catabolize metabolically important biomolecules (e.g. amino acids, nucleic acids), and to detoxify potentially harmful compounds, or to facilitate the neutralization of these molecules.

In addition to the nucleic acid molecules encoding 52871 proteins described above, another aspect of the invention pertains to isolated nucleic acid molecules which are antisense thereto. In an exemplary embodiment, the invention provides an isolated nucleic acid molecule which is antisense to a 52871 nucleic acid molecule (e.g., is antisense to the coding strand of a 52871 nucleic acid molecule). An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 52871 coding strand, or to only a portion thereof. In one embodiment, an antisense nucleic acid molecule is antisense to a “coding region” of the coding strand of a nucleotide sequence encoding a 52871. The term “coding region” refers to the region of the nucleotide sequence comprising codons which are translated into amino acid residues (e.g., the coding region of human 52871 corresponds to SEQ ID NO:29). In another embodiment, the antisense nucleic acid molecule is antisense to a “noncoding region” of the coding strand of a nucleotide sequence encoding 52871. The term “noncoding region” refers to 5′ and 3′ sequences which flank the coding region that are not translated into amino acids (i.e., also referred to as 5′ and 3′ untranslated regions).

Given the coding strand sequences encoding 52871 disclosed herein (e.g., SEQ ID NO:29), antisense nucleic acids of the invention can be designed according to the rules of Watson and Crick base pairing. The antisense nucleic acid molecule can be complementary to the entire coding region of 52871 mRNA, but more preferably is an oligonucleotide which is antisense to only a portion of the coding or noncoding region of 52871 mRNA. For example, the antisense oligonucleotide can be complementary to the region surrounding the translation start site of 52871 mRNA (e.g., between the −10 and +10 regions of the start site of a gene nucleotide sequence). An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a 52871 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) F.E.B.S. Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 52871 mRNA transcripts to thereby inhibit translation of 52871 mRNA. A ribozyme having specificity for a 52871-encoding nucleic acid can be designed based upon the nucleotide sequence of a 52871 cDNA disclosed herein (i.e., SEQ ID NO:27 or 29). For example, a derivative of a Tetrahymena L-19 IVS RNA can be constructed in which the nucleotide sequence of the active site is complementary to the nucleotide sequence to be cleaved in a 52871-encoding mRNA. See, e.g., Cech et al. U.S. Pat. No. 4,987,071; and Cech et al. U.S. Pat. No. 5,116,742. Alternatively, 52871 mRNA can be used to select a catalytic RNA having a specific ribonuclease activity from a pool of RNA molecules. See, e.g., Bartel, D. and Szostak, J. W. (1993) Science 261:1411-1418.

Alternatively, 52871 gene expression can be inhibited by targeting nucleotide sequences complementary to the regulatory region of the 52871 (e.g., the 52871 promoter and/or enhancers; e.g., nucleotides 1-107 of SEQ ID NO:27) to form triple helical structures that prevent transcription of the 52871 gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioassays 14(12):807-15.

In yet another embodiment, the 52871 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of 52871 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 52871 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of 52871 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 52871 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous 52871 gene within a cell line or microorganism may be modified by inserting a heterologous DNA regulatory element into the genome of a stable cell line or cloned microorganism such that the inserted regulatory element is operatively linked with the endogenous 52871 gene. For example, an endogenous 52871 gene which is normally “transcriptionally silent”, i.e., a 52871 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous 52871 gene may be activated by insertion of a promiscuous regulatory element that works across cell types.

A heterologous regulatory element may be inserted into a stable cell line or cloned microorganism, such that it is operatively linked with an endogenous 52871 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated 52871 Proteins and Anti-52871 Antibodies

One aspect of the invention pertains to isolated or recombinant 52871 proteins and polypeptides, and biologically active portions thereof, as well as polypeptide fragments suitable for use as immunogens to raise anti-52871 antibodies. In one embodiment, native 52871 proteins can be isolated from cells or tissue sources by an appropriate purification scheme using standard protein purification techniques. In another embodiment, 52871 proteins are produced by recombinant DNA techniques. Alternative to recombinant expression, a 52871 protein or polypeptide can be synthesized chemically using standard peptide synthesis techniques.

An “isolated” or “purified” protein or biologically active portion thereof is substantially free of cellular material or other contaminating proteins from the cell or tissue source from which the 52871 protein is derived, or substantially free from chemical precursors or other chemicals when chemically synthesized. The language “substantially free of cellular material” includes preparations of 52871 protein in which the protein is separated from cellular components of the cells from which it is isolated or recombinantly produced. In one embodiment, the language “substantially free of cellular material” includes preparations of 52871 protein having less than about 30% (by dry weight) of non-52871 protein (also referred to herein as a “contaminating protein”), more preferably less than about 20% of non-52871 protein, still more preferably less than about 10% of non-52871 protein, and most preferably less than about 5% non-52871 protein. When the 52871 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of 52871 protein in which the protein is separated from chemical precursors or other chemicals which are involved in the synthesis of the protein. In one embodiment, the language “substantially free of chemical precursors or other chemicals” includes preparations of 52871 protein having less than about 30% (by dry weight) of chemical precursors or non-52871 chemicals, more preferably less than about 20% chemical precursors or non-52871 chemicals, still more preferably less than about 10% chemical precursors or non-52871 chemicals, and most preferably less than about 5% chemical precursors or non-52871 chemicals.

As used herein, a “biologically active portion” of a 52871 protein includes a fragment of a 52871 protein which participates in an interaction between a 52871 molecule and a non-52871 molecule. Biologically active portions of a 52871 protein include peptides comprising amino acid sequences sufficiently identical to or derived from the amino acid sequence of the 52871 protein, e.g., the amino acid sequence shown in SEQ ID NO:28, which include less amino acids than the full length 52871 protein, and exhibit at least one activity of a 52871 protein. Typically, biologically active portions comprise a domain or motif with at least one activity of the 52871 protein, e.g., signal molecule recognition, signal transduction, modulation of intracellular signaling, modulation of nociception, and/or modulation of pain. A biologically active portion of a 52871 protein can be a polypeptide which is, for example, 25, 50, 75, 100, 125, 150, 175, 200, 250, 300 or more amino acids in length. Biologically active portions of a 52871 protein can be used as targets for developing agents which modulate a 52871 mediated activity, e.g., intercellular signaling, modulation of nociception, and/or modulation of pain.

It is to be understood that a preferred biologically active portion of a 52871 protein of the present invention may contain one or more of the following domains: a transmembrane domain, a 7-TMR profile, a G-protein coupled receptor signature. Moreover, other biologically active portions, in which other regions of the protein are deleted, can be prepared by recombinant techniques and evaluated for one or more of the functional activities of a native 52871 protein.

Another aspect of the invention features fragments of the protein having the amino acid sequence of SEQ ID NO:28, for example, for use as immunogens. In one embodiment, a fragment comprises at least 5 amino acids (e.g., contiguous or consecutive amino acids) of the amino acid sequence of SEQ ID NO:28. In another embodiment, a fragment comprises at least 10, 15, 20, 25, 30, 35, 40, 45, 50 or more amino acids (e.g., contiguous or consecutive amino acids) of the amino acid sequence of SEQ ID NO:28.

In a preferred embodiment, a 52871 protein has an amino acid sequence shown in SEQ ID NO:28. In other embodiments, the 52871 protein is substantially identical to SEQ ID NO:28, and retains the functional activity of the protein of SEQ ID NO:28, yet differs in amino acid sequence due to natural allelic variation or mutagenesis, as described in detail in subsection I above. In another embodiment, the 52871 protein is a protein which comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to SEQ ID NO:28.

In another embodiment, the invention features a 52871 protein which is encoded by a nucleic acid molecule consisting of a nucleotide sequence at least about 50%, 55%, 60%, 65%, 70%, 75%, 80%, 85%, 90%, 91%, 92%, 93%, 94%, 95%, 96%, 97%, 98%, 99% or more identical to a nucleotide sequence of SEQ ID NO:27 or SEQ ID NO:29, or a complement thereof. This invention further features a 52871 protein which is encoded by a nucleic acid molecule consisting of a nucleotide sequence which hybridizes under stringent hybridization conditions to a complement of a nucleic acid molecule comprising the nucleotide sequence of SEQ ID NO:27 or SEQ ID NO:29, or a complement thereof.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 52871 amino acid sequence of SEQ ID NO:28 having 400 amino acid residues, at least 50, preferably at least 100, more preferably at least 150, even more preferably at least 200, and even more preferably at least 300 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of Meyers and Miller (Comput. Appl. Biosci., 4:11-17 (1988)) which has been incorporated into the ALIGN program (version 2.0 or version 2.U), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 52871 nucleic acid molecules of the invention. BLAST protein searches can be performed with the XBLAST program, score=100, wordlength=3 to obtain amino acid sequences homologous to 52871 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The invention also provides 52871 chimeric or fusion proteins. As used herein, a 52871 “chimeric protein” or “fusion protein” comprises a 52871 polypeptide operatively linked to a non-52871 polypeptide. An “52871 polypeptide” refers to a polypeptide having an amino acid sequence corresponding to a 52871 molecule, whereas a “non-52871 polypeptide” refers to a polypeptide having an amino acid sequence corresponding to a protein which is not substantially homologous to the 52871 protein, e.g., a protein which is different from the 52871 protein and which is derived from the same or a different organism. Within a 52871 fusion protein the 52871 polypeptide can correspond to all or a portion of a 52871 protein. In a preferred embodiment, a 52871 fusion protein comprises at least one biologically active portion of a 52871 protein. In another preferred embodiment, a 52871 fusion protein comprises at least two biologically active portions of a 52871 protein. Within the fusion protein, the term “operatively linked” is intended to indicate that the 52871 polypeptide and the non-52871 polypeptide are fused in-frame to each other. The non-52871 polypeptide can be fused to the N-terminus or C-terminus of the 52871 polypeptide.

For example, in one embodiment, the fusion protein is a GST-52871 fusion protein in which the 52871 sequences are fused to the C-terminus of the GST sequences. Such fusion proteins can facilitate the purification of recombinant 52871.

In another embodiment, the fusion protein is a 52871 protein containing a heterologous signal sequence at its N-terminus. In certain host cells (e.g., mammalian host cells), expression and/or secretion of 52871 can be increased through use of a heterologous signal sequence.

The 52871 fusion proteins of the invention can be incorporated into pharmaceutical compositions and administered to a subject in vivo. The 52871 fusion proteins can be used to affect the bioavailability of a 52871 substrate. Use of 52871 fusion proteins may be useful therapeutically for the treatment of disorders caused by, for example, (i) aberrant modification or mutation of a gene encoding a 52871 protein; (ii) mis-regulation of the 52871 gene; and (iii) aberrant post-translational modification of a 52871 protein.

Moreover, the 52871-fusion proteins of the invention can be used as immunogens to produce anti-52871 antibodies in a subject for use in screening assays to identify molecules which inhibit the interaction of 52871 with a 52871 substrate.

Preferably, a 52871 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons, 1992). Moreover, many expression vectors are commercially available that already encode a fusion moiety (e.g., a GST polypeptide). A 52871-encoding nucleic acid can be cloned into such an expression vector such that the fusion moiety is linked in-frame to the 52871 protein.

The present invention also pertains to variants of the 52871 proteins which function as either 52871 agonists (mimetics) or as 52871 antagonists. Variants of the 52871 proteins can be generated by mutagenesis, e.g., discrete point mutation or truncation of a 52871 protein. An agonist of the 52871 proteins can retain substantially the same, or a subset, of the biological activities of the naturally occurring form of a 52871 protein. An antagonist of a 52871 protein can inhibit one or more of the activities of the naturally occurring form of the 52871 protein by, for example, competitively modulating a 52871-mediated activity of a 52871 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 52871 protein.

In one embodiment, variants of a 52871 protein which function as either 52871 agonists (mimetics) or as 52871 antagonists can be identified by screening combinatorial libraries of mutants, e.g., truncation mutants, of a 52871 protein for 52871 protein agonist or antagonist activity. In one embodiment, a variegated library of 52871 variants is generated by combinatorial mutagenesis at the nucleic acid level and is encoded by a variegated gene library. A variegated library of 52871 variants can be produced by, for example, enzymatically ligating a mixture of synthetic oligonucleotides into gene sequences such that a degenerate set of potential 52871 sequences is expressible as individual polypeptides, or alternatively, as a set of larger fusion proteins (e.g., for phage display) containing the set of 52871 sequences therein. There are a variety of methods which can be used to produce libraries of potential 52871 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 52871 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a 52871 protein coding sequence can be used to generate a variegated population of 52871 fragments for screening and subsequent selection of variants of a 52871 protein. In one embodiment, a library of coding sequence fragments can be generated by treating a double stranded PCR fragment of a 52871 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 52871 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 52871 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 52871 variants (Arkin and Yourvan (1992) Proc. Natl. Acad. Sci. USA 89:7811-7815; Delgrave et al. (1993) Protein Engineering 6(3): 327-331).

In one embodiment, cell based assays can be exploited to analyze a variegated 52871 library. For example, a library of expression vectors can be transfected into a cell line, e.g., a neuronal cell line, which ordinarily responds to a 52871 ligand in a particular 52871 ligand-dependent manner. The transfected cells are then contacted with a 52871 ligand and the effect of expression of the mutant on, e.g., membrane excitability of 52871 can be detected. Plasmid DNA can then be recovered from the cells which score for inhibition, or alternatively, potentiation of signaling by the 52871 ligand, and the individual clones further characterized.

An isolated 52871 protein, or a portion or fragment thereof, can be used as an immunogen to generate antibodies that bind 52871 using standard techniques for polyclonal and monoclonal antibody preparation. A full-length 52871 protein can be used or, alternatively, the invention provides antigenic peptide fragments of 52871 for use as immunogens. The antigenic peptide of 52871 comprises at least 8 amino acid residues of the amino acid sequence shown in SEQ ID NO:28 and encompasses an epitope of 52871 such that an antibody raised against the peptide forms a specific immune complex with the 52871 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 52871 that are located on the surface of the protein, e.g., hydrophilic regions, as well as regions with high antigenicity.

A 52871 immunogen typically is used to prepare antibodies by immunizing a suitable subject, (e.g., rabbit, goat, mouse or other mammal) with the immunogen. An appropriate immunogenic preparation can contain, for example, recombinantly expressed 52871 protein or a chemically synthesized 52871 polypeptide. The preparation can further include an adjuvant, such as Freund's complete or incomplete adjuvant, or similar immunostimulatory agent. Immunization of a suitable subject with an immunogenic 52871 preparation induces a polyclonal anti-52871 antibody response.

Accordingly, another aspect of the invention pertains to anti-52871 antibodies. The term “antibody” as used herein refers to immunoglobulin molecules and immunologically active portions of immunoglobulin molecules, i.e., molecules that contain an antigen binding site which specifically binds (immunoreacts with) an antigen, such as a 52871. Examples of immunologically active portions of immunoglobulin molecules include F(ab) and F(ab′)2 fragments which can be generated by treating the antibody with an enzyme such as pepsin. The invention provides polyclonal and monoclonal antibodies that bind 52871 molecules. The term “monoclonal antibody” or “monoclonal antibody composition”, as used herein, refers to a population of antibody molecules that contain only one species of an antigen binding site capable of immunoreacting with a particular epitope of 52871. A monoclonal antibody composition thus typically displays a single binding affinity for a particular 52871 protein with which it immunoreacts.

Polyclonal anti-52871 antibodies can be prepared as described above by immunizing a suitable subject with a 52871 immunogen. The anti-52871 antibody titer in the immunized subject can be monitored over time by standard techniques, such as with an enzyme linked immunosorbent assay (ELISA) using immobilized 52871. If desired, the antibody molecules directed against 52871 can be isolated from the mammal (e.g., from the blood) and further purified by well known techniques, such as protein A chromatography to obtain the IgG fraction. At an appropriate time after immunization, e.g., when the anti-52871 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a 52871 immunogen as described above, and the culture supernatants of the resulting hybridoma cells are screened to identify a hybridoma producing a monoclonal antibody that binds 52871.

Any of the many well known protocols used for fusing lymphocytes and immortalized cell lines can be applied for the purpose of generating an anti-52871 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from ATCC Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 52871, e.g., using a standard ELISA assay.

Alternative to preparing monoclonal antibody-secreting hybridomas, a monoclonal anti-52871 antibody can be identified and isolated by screening a recombinant combinatorial immunoglobulin library (e.g., an antibody phage display library) with 52871 to thereby isolate immunoglobulin library members that bind 52871. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-52871 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-52871 antibody (e.g., monoclonal antibody) can be used to isolate 52871 by standard techniques, such as affinity chromatography or immunoprecipitation. An anti-52871 antibody can facilitate the purification of natural 52871 from cells and of recombinantly produced 52871 expressed in host cells. Moreover, an anti-52871 antibody can be used to detect 52871 protein (e.g., in a cellular lysate or cell supernatant) in order to evaluate the abundance and pattern of expression of the 52871 protein. Anti-52871 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

III. Recombinant Expression Vectors and Host Cells

Another aspect of the invention pertains to vectors, for example recombinant expression vectors, containing a 52871 nucleic acid molecule or vectors containing a nucleic acid molecule which encodes a 52871 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 52871 proteins, mutant forms of 52871 proteins, fusion proteins, and the like).

Accordingly, an exemplary embodiment provides a method for producing a protein, preferably a 52871 protein, by culturing in a suitable medium a host cell of the invention (e.g., a mammalian host cell such as a non-human mammalian cell) containing a recombinant expression vector, such that the protein is produced.

The recombinant expression vectors of the invention can be designed for expression of 52871 proteins in prokaryotic or eukaryotic cells. For example, 52871 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 52871 activity assays, (e.g., direct assays or competitive assays described in detail below), or to generate antibodies specific for 52871 proteins, for example. In a preferred embodiment, a 52871 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the 52871 expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, 52871 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the □-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 52871 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a 52871 nucleic acid molecule of the invention is introduced, e.g., a 52871 nucleic acid molecule within a vector (e.g., a recombinant expression vector) or a 52871 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 52871 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a 52871 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a 52871 protein. Accordingly, the invention further provides methods for producing a 52871 protein using the host cells of the invention. In one embodiment, the method comprises culturing the host cell of the invention (into which a recombinant expression vector encoding a 52871 protein has been introduced) in a suitable medium such that a 52871 protein is produced. In another embodiment, the method further comprises isolating a 52871 protein from the medium or the host cell.

The host cells of the invention can also be used to produce non-human transgenic animals. For example, in one embodiment, a host cell of the invention is a fertilized oocyte or an embryonic stem cell into which 52871-coding sequences have been introduced. Such host cells can then be used to create non-human transgenic animals in which exogenous 52871 sequences have been introduced into their genome or homologous recombinant animals in which endogenous 52871 sequences have been altered. Such animals are useful for studying the function and/or activity of a 52871 and for identifying and/or evaluating modulators of 52871 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 52871 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a 52871-encoding nucleic acid into the male pronuclei of a fertilized oocyte, e.g., by microinjection, retroviral infection, and allowing the oocyte to develop in a pseudopregnant female foster animal. The 52871 cDNA sequence of SEQ ID NO:27 can be introduced as a transgene into the genome of a non-human animal. Alternatively, a nonhuman homologue of a human 52871 gene, such as a mouse or rat 52871 gene, can be used as a transgene. Alternatively, a 52871 gene homologue, such as another 52871 family member, can be isolated based on hybridization to the 52871 cDNA sequences of SEQ ID NO:27 or 29. Intronic sequences and polyadenylation signals can also be included in the transgene to increase the efficiency of expression of the transgene. A tissue-specific regulatory sequence(s) can be operably linked to a 52871 transgene to direct expression of a 52871 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 52871 transgene in its genome and/or expression of 52871 mRNA in tissues or cells of the animals. A transgenic founder animal can then be used to breed additional animals carrying the transgene. Moreover, transgenic animals carrying a transgene encoding a 52871 protein can further be bred to other transgenic animals carrying other transgenes.

To create a homologous recombinant animal, a vector is prepared which contains at least a portion of a 52871 gene into which a deletion, addition or substitution has been introduced to thereby alter, e.g., functionally disrupt, the 52871 gene. The 52871 gene can be a human gene (e.g., the cDNA of SEQ ID NO:29), but more preferably, is a non-human homologue of a human 52871 gene (e.g., a cDNA isolated by stringent hybridization with the nucleotide sequence of SEQ ID NO:27). For example, a mouse 52871 gene can be used to construct a homologous recombination nucleic acid molecule, e.g., a vector, suitable for altering an endogenous 52871 gene in the mouse genome. In a preferred embodiment, the homologous recombination nucleic acid molecule is designed such that, upon homologous recombination, the endogenous 52871 gene is functionally disrupted (i.e., no longer encodes a functional protein; also referred to as a “knock out” vector). Alternatively, the homologous recombination nucleic acid molecule can be designed such that, upon homologous recombination, the endogenous 52871 gene is mutated or otherwise altered but still encodes functional protein (e.g., the upstream regulatory region can be altered to thereby alter the expression of the endogenous 52871 protein). In the homologous recombination nucleic acid molecule, the altered portion of the 52871 gene is flanked at its 5′ and 3′ ends by additional nucleic acid sequence of the 52871 gene to allow for homologous recombination to occur between the exogenous 52871 gene carried by the homologous recombination nucleic acid molecule and an endogenous 52871 gene in a cell, e.g., an embryonic stem cell. The additional flanking 52871 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 52871 gene has homologously recombined with the endogenous 52871 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/oxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/oxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G0 phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The 52871 nucleic acid molecules, 52871 proteins, fragments thereof, anti-52871 antibodies, and 52871 modulators (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a 52871 protein or an anti-52871 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, modulators, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a 52871 protein of the invention has one or more of the following activities: (i) interaction of a 52871 protein with soluble 52871 ligand; (ii) interaction of a 52871 protein with a membrane-bound non-52871 protein; (iii) interaction of a 52871 protein with an intracellular protein (e.g., an intracellular enzyme or signal transduction molecule); (iv) indirect interaction of a 52871 protein with an intracellular protein (e.g., a downstream signal transduction molecule); v) modulation of intra- or intercellular signaling or feedback mechanisms; yl) modulation of the intracellular levels and/or homeostatic balance of signaling molecule pools (e.g., Ca2+, diacylglycerol, IP3, cAMP); vii) regulation of cellular proliferation; viii) regulation of cellular differentiation; ix) regulation of development; x) regulation of gene expression in a cell which expresses a 52871 protein; xi) regulation of nociception; xii) regulation of pain signaling; and xiii) regulation of cell death. As such, a 52871 molecule of the invention (or modulator thereof) can be used, for example, in at least one of the following activities: 1) modulation of intra- or intercellular signaling or feedback mechanisms; 2) modulation of the intracellular levels and/or homeostatic balance of signaling molecule pools (e.g., Ca2+, diacylglycerol, IP3, cAMP); 3) modulation of cellular proliferation; 4) modulation of cellular differentiation or development; 5) modulation of gene expression in a cell which expresses a 52871 protein; 6) modulation of nociception; 7) modulation of pain; and 8) modulation of cell death.

Modulation of 52871 activity has particular applicability in treating, for example, disorders characterized by insufficient or excessive production of 52871 protein or production of 52871 protein forms which have decreased, or aberrant or unwanted activity compared to 52871 wild type protein, preferably GPCR-associated disorders. Moreover, modulation of 52871 activity has particular application in treating pain and or pain disorders. Modulation of 52871 activity includes, but is not limited to, increasing or enhancing the activity of 52871 (e.g., increasing or enhancing 52871 signaling), decreasing or inhibiting the activity of 52871 (e.g., decreasing or inhibiting 52871 signaling), regulating 52871 cellular localization, trafficking and/or desensitization of 52871.

As used herein, a “GPCR-associated disorder” (or a “52871-associated disorder”) includes a disorder, disease or condition which is caused or characterized by a misregulation (e.g., downregulation or upregulation) of a G protein coupled receptor activity (e.g., GPCR-mediated activity), for example, signal molecule recognition activity or a signal transduction activity. GPCR-associated disorders can detrimentally affect cellular functions such as cellular proliferation, growth, differentiation, or migration, cellular regulation of homeostasis, inter- or intra-cellular communication; nociception; pain; tissue function, such as cardiac function or musculoskeletal function; systemic responses in an organism, such as nervous system responses, hormonal responses (e.g., insulin response), or immune responses; and protection of cells from toxic compounds (e.g., carcinogens, toxins, mutagens, and toxic byproducts of metabolic activity (e.g., reactive oxygen species). As used herein, the term “pain disorder” includes disorders characterized by aberrant (e.g., excessive or amplified) pain signaling in addition to symptoms and/or phenotypes which result from wild-type, or normal, pain signaling mechanisms. Examples of pain disorders include posttherapeutic neuralgia, diabetic neuropathy, postmastectomy pain syndrome, stump pain, reflex sympathetic dystrophy, trigeminal neuralgia, neuropathic pain, orofacial neuropathic pain, osteoarthritis, rheumatoid arthritis, fibromyalgia syndrome, tension myalgia, Guillian-Barre syndrome, Meralgia paraesthetica, burning mouth syndrome, fibrocitis, myofascial pain syndrome, idiopathic pain disorder, temporomandibular joint syndrome, atypical odontalgia, loin pain, haematuria syndrome, non-cardiac chest pain, low back pain, chronic nonspecific pain, psychogenic pain, musculoskeletal pain disorder, chronic pelvic pain, nonorganic chronic headache, tension-type headache, cluster headache, migraine, complex regional pain syndrome, vaginismus, nerve trunk pain, somatoform pain disorder, cyclical mastalgia, chronic fatigue syndrome, multiple somatization syndrome, chronic pain disorder, somatization disorder, Syndrome X, facial pain, idiopathic pain disorder, posttraumatic rheumatic pain modulation disorder (fibrositis syndrome), hyperalgesia, and Tangier disease. As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes. The 52781 molecules of the present invention are expressed in brain, spinal cord, skin and dorsal root ganglia, and, thus, may be involved in the detection of these noxious chemical, mechanical, or thermal stimuli and/or in the transduction of this information into membrane depolarization events. Thus, the 52781 molecules, by participating in pain signaling mechanisms, may modulate pain elicitation and act as targets for developing novel diagnostic targets and therapeutic agents to control pain and pain disorders.

Further examples of GPCR- or 52871-associated disorders include CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

Further examples of GPCR- or 52871-associated disorders include cardiac-related disorders. Cardiovascular system disorders in which the 52871 molecules of the invention may be directly or indirectly involved include arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrillation, Jervell syndrome, Lange-Nielsen syndrome, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, and arrhythmia. 52871-mediated or related disorders also include disorders of the musculoskeletal system such as paralysis and muscle weakness, e.g., ataxia, myotonia, and myokymia.

GPCR- or 52871-associated disorders also include cellular proliferation, growth, differentiation, or migration disorders. Cellular proliferation, growth, differentiation, or migration disorders include those disorders that affect cell proliferation, growth, differentiation, or migration processes. As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus. The 52871 molecules of the present invention are involved in signal transduction mechanisms, which are known to be involved in cellular growth, differentiation, and migration processes. Thus, the 52871 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; hepatic disorders; and hematopoietic and/or myeloproliferative disorders.

GPCR- or 52871-associated or related disorders also include hormonal disorders, such as conditions or diseases in which the production and/or regulation of hormones in an organism is aberrant. Examples of such disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

52871-associated or related disorders also include immune disorders, such as autoimmune disorders or immune deficiency disorders, e.g., congenital X-linked infantile hypogammaglobulinemia, transient hypogammaglobulinemia, common variable immunodeficiency, selective IgA deficiency, chronic mucocutaneous candidiasis, or severe combined immunodeficiency. 52871-associated or related disorders also include disorders affecting tissues in which 52871 protein is expressed (e.g., brain, spinal cord, dorsal root ganglia, or skin tissue).

In addition, 52871-associated or related disorders also include disorders such as retinitis pigmentosa, stationary night blindness, color blindness, isolated glucocorticoid deficiency, hyperfunctioning thyroid adenoma, familial precocious puberty, familial hypocalciuric hypercalcemia, neonatal severe hyperparathroidism, and nephrogenic diabetes insipidus.

The isolated nucleic acid molecules of the invention can be used, for example, to express 52871 protein (e.g., via a recombinant expression vector in a host cell in gene therapy applications), to detect 52871 mRNA (e.g., in a biological sample) or a genetic alteration in a 52871 gene, and to modulate 52871 activity, as described further below. The 52871 proteins can be used to treat disorders characterized by insufficient or excessive production of a 52871 protein and/or 52871 ligand. In addition, the 52871 proteins can be used to screen drugs or compounds which modulate the 52871 activity as well as to treat disorders characterized by insufficient or excessive production of 52871 protein or production of 52871 protein forms which have decreased or aberrant activity compared to 52871 wild type protein. Moreover, the anti-52871 antibodies of the invention can be used to detect and isolate 52871 proteins, regulate the bioavailability of 52871 proteins, and modulate 52871 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to 52871 proteins, or have a stimulatory or inhibitory effect on, for example, 52871 expression or 52871 activity.

In one embodiment, the invention provides assays for screening candidate or test compounds which bind to or modulate the activity of a 52871 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. 1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 52871 protein on the cell surface is contacted with a test compound and the ability of the test compound to bind to the 52871 protein determined. The cell, for example, can be of mammalian origin or a yeast cell. Determining the ability of the test compound to bind to a 52871 protein can be accomplished, for example, by coupling the test compound with a radioisotope or enzymatic label such that binding of the test compound to the 52871 protein can be determined by detecting the labeled compound in a complex. For example, test compounds can be labeled with 125I, 35S, 14C, or 3H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with a 52871 protein without the labeling of any of the interactants. For example, a microphysiometer can be used to detect the interaction of a test compound with a 52871 protein without the labeling of either the test compound or the receptor. McConnell et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and receptor.

In a preferred embodiment, the assay comprises contacting a cell which expresses a 52871 protein or biologically active portion thereof, on the cell surface with a 52871 ligand (e.g., a peptide, a neurotransmitter, or a hormone), to form an assay mixture, contacting the assay mixture with a test compound, and determining the ability of the test compound to interact with the 52871 protein or biologically active portion thereof, wherein determining the ability of the test compound to interact with the 52871 protein or biologically active portion thereof, comprises determining the ability of the test compound to preferentially bind to the 52871 protein or biologically active portion thereof, as compared to the ability of the 52871 ligand to bind to the 52871 protein or biologically active portion thereof. Determining the ability of the 52871 ligand or 52871 modulator to bind to or interact with a 52871 protein or biologically active portion thereof, can be accomplished by one of the methods described above for determining direct binding. In a preferred embodiment, determining the ability of the 52871 ligand or modulator to bind to or interact with a 52871 protein or biologically active portion thereof, can be accomplished by determining the activity of a 52871 protein or of a downstream 52871 target molecule. For example, the target molecule can be a cellular second messenger, and the activity of the target molecule can be determined by detecting induction of the target (i.e. intracellular Ca2+, diacylglycerol, IP3, etc.), detecting catalytic/enzymatic activity of the target on an appropriate substrate, detecting the induction of a reporter gene (comprising a 52871-responsive regulatory element operatively linked to a nucleic acid encoding a detectable marker, e.g., luciferase), or detecting a cellular response, for example, a proliferative response, a differentiation response, or a signaling response. Accordingly, in one embodiment, the present invention involves a method of identifying a compound which modulates the activity of a 52871 protein, comprising contacting a cell which expresses a 52871 protein with a test compound, determining the ability of the test compound to modulate the activity the 52871 protein, and identifying the compound as a modulator of 52871 activity. In another embodiment, the present invention involves a method of identifying a compound which modulates the activity of a 52871 protein, comprising contacting a cell which expresses a 52871 protein with a test compound, determining the ability of the test compound to modulate the activity of a downstream 52871 target molecule, and identifying the compound as a modulator of 52871 activity.

In yet another embodiment, an assay of the present invention is a cell-free assay in which a 52871 protein or biologically active portion thereof is contacted with a test compound and the ability of the test compound to bind to the 52871 protein or biologically active portion thereof is determined. Binding of the test compound to the 52871 protein can be determined either directly or indirectly as described above.

Binding of the test compound to the 52871 protein can also be accomplished using a technology such as real-time Biomolecular Interaction Analysis (BIA). Sjolander, S. and Urbaniczky, C. (1991) Anal. Chem. 3:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In a preferred embodiment, the assay includes contacting the 52871 protein or biologically active portion thereof with a known ligand (e.g., a peptide, a neurotransmitter, or a hormone) which binds 52871 to form an assay mixture, contacting the assay mixture with a test compound, and determining the ability of the test compound to interact with a 52871 protein, wherein determining the ability of the test compound to interact with a 52871 protein comprises determining the ability of the test compound to preferentially bind to 52871 or biologically active portion thereof as compared to the known ligand.

In another embodiment, the assay is a cell-free assay in which a 52871 protein or biologically active portion thereof is contacted with a test compound and the ability of the test compound to modulate (e.g., stimulate or inhibit) the activity of the 52871 protein or biologically active portion thereof is determined. Determining the ability of the test compound to modulate the activity of a 52871 protein can be accomplished, for example, by determining the ability of the 52871 protein to modulate the activity of a downstream 52871 target molecule by one of the methods described above for cell-based assays. For example, the catalytic/enzymatic activity of the target molecule on an appropriate substrate can be determined as previously described. In yet another embodiment, the cell-free assay involves contacting a 52871 protein or biologically active portion thereof with a known ligand (e.g., a peptide, a neurotransmitter, or a hormone) which binds the 52871 protein to form an assay mixture, contacting the assay mixture with a test compound, and determining the ability of the test compound to interact with the 52871 protein, wherein determining the ability of the test compound to interact with the 52871 protein comprises determining the ability of the test compound to preferentially bind to or modulate the activity of a 52871 target molecule, as compared to the known ligand.

The cell-free assays of the present invention are amenable to use of both soluble and/or membrane-bound forms of isolated proteins (e.g. 52871 proteins or biologically active portions thereof or 52871 proteins). In the case of cell-free assays in which a membrane-bound form an isolated protein is used (e.g., a 52871 protein) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either 52871 or its target molecule to facilitate separation of complexed from uncomplexed forms of one or both of the proteins, as well as to accommodate automation of the assay. Binding of a test compound to a 52871 protein, or interaction of a 52871 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/52871 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 52871 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 52871 binding or activity determined using standard techniques.

Other techniques for immobilizing proteins on matrices can also be used in the screening assays of the invention. For example, either a 52871 protein or a 52871 target molecule can be immobilized utilizing conjugation of biotin and streptavidin. Biotinylated 52871 protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with 52871 protein or target molecules but which do not interfere with binding of the 52871 protein to its target molecule can be derivatized to the wells of the plate, and unbound target or 52871 protein trapped in the wells by antibody conjugation. Methods for detecting such complexes, in addition to those described above for the GST-immobilized complexes, include immunodetection of complexes using antibodies reactive with the 52871 protein or target molecule, as well as enzyme-linked assays which rely on detecting an enzymatic activity associated with the 52871 protein or target molecule.

In another embodiment, modulators of 52871 expression are identified in a method wherein a cell is contacted with a candidate compound and the expression of 52871 mRNA or protein in the cell is determined. The level of expression of 52871 mRNA or protein in the presence of the candidate compound is compared to the level of expression of 52871 mRNA or protein in the absence of the candidate compound. The candidate compound can then be identified as a modulator of 52871 expression based on this comparison. For example, when expression of 52871 mRNA or protein is greater (statistically significantly greater) in the presence of the candidate compound than in its absence, the candidate compound is identified as a stimulator of 52871 mRNA or protein expression. Alternatively, when expression of 52871 mRNA or protein is less (statistically significantly less) in the presence of the candidate compound than in its absence, the candidate compound is identified as an inhibitor of 52871 mRNA or protein expression. The level of 52871 mRNA or protein expression in the cells can be determined by methods described herein for detecting 52871 mRNA or protein.

In yet another aspect of the invention, the 52871 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins, which bind to or interact with 52871 (“52871-binding proteins” or “52871-bp”) and are involved in 52871 activity. Such 52871-binding proteins are also likely to be involved in the propagation of signals by the 52871 proteins as, for example, downstream elements of a 52871-mediated signaling pathway. Alternatively, such 52871-binding proteins are likely to be cell-surface molecules associated with non-52871 expressing cells, wherein such 52871-binding proteins are involved in chemoattraction.

The two-hybrid system is based on the modular nature of most transcription factors, which consist of separable DNA-binding and activation domains. Briefly, the assay utilizes two different DNA constructs. In one construct, the gene that codes for a 52871 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 52871-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 52871 protein.

The present invention further features assays (e.g., secondary screening assays or validation assays) designed to confirm the activity of a test compound, for example, as a 52871 modulator. In one embodiment, the invention features screening assays (e.g., secondary screening assays or validation assays) which include administering a test compound, for example, a test compound that demonstrates binding to a 52871 protein or modulation of a 52871 activity in at least one of the above-described cell-based or cell-free assays, to an animal and determining the ability of the test compound to modulate 52871 activity in vivo. Determining the ability of a compound to modulate activity in vivo can include, for example, determining the ability of the compound to modulate signaling activity. Exemplary animals for determining 52871 modulatory activity include normal animals as well as animal models which have one or more signaling dysfunctions. It is also within the scope of this invention to use an agent or compound as described herein (e.g., a 52871 modulating agent, an antisense 52871 nucleic acid molecule, a 52871-specific antibody, or a 52871-binding partner) in an animal model to determine the efficacy, toxicity, or side effects of treatment with such an agent. Alternatively, an agent identified as described herein can be used in an animal model to determine the mechanism of action of such an agent.

Models for studying pain in vivo include rat models of neuropathic pain caused by methods such as intraperitoneal administration of Taxol (Authier et al. (2000) Brain Res. 887:239-249), chronic constriction injury (CCl), partial sciatic nerve transection (Linenlaub and Sommer (2000) Pain 89:97-106), transection of the tibial and sural nerves (Lee et al. (2000) Neurosci. Lett. 291:29-32), the spared nerve injury model (Decosterd and Woolf (2000) Pain 87:149-158), cuffing the sciatic nerve (Pitcher and Henry (2000) Eur. J. Neurosci. 12:2006-2020), unilateral tight ligation (Esser and Sawynok (2000) Eur. J. Pharmacol. 399:131-139), L5 spinal nerve ligation (Honroe et al. (2000) Neurosci. 98:585-598), and photochemically induced ischemic nerve injury (Hao et al. (2000) Exp. Neurol. 163:231-238); rat models of nociceptive pain caused by methods such as the Chung Method, the Bennett Method, and intraperitoneal administration of complete Freund's adjuvant (CFA) (Abdi et al. (2000) Anesth. Analg. 91:955-959); rat models of post-incisional pain caused by incising the skin and fascia of a hind paw (Olivera and Prado (2000) Braz. J. Med. Biol. Res. 33:957-960); rat models of cancer pain caused by methods such as injecting osteolytic sarcoma cells into the femur (Honroe et al. (2000) Neurosci. 98:585-598); and rat models of visceral pain caused by methods such as intraperitoneal administration of cyclophosphamide.

Various methods of determining an animal's response to pain are known in the art. Examples of such methods include, but are not limited to brief intense exposure to a focused heat source, administration of a noxious chemical subcutaneously, the tail flick test, the hot plate test, the formalin test, Von Frey threshold, and testing for stress-induced analgesia (et al., by restraint, foot shock, and/or cold water swim) (Crawley (2000) What's Wrong With My Mouse? Wiley-Liss pp. 72-75).

This invention further pertains to novel agents identified by the above-described screening assays. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the 52871 nucleotide sequences, described herein, can be used to map the location of the 52871 genes on a chromosome. The mapping of the 52871 sequences to chromosomes is an important first step in correlating these sequences with genes associated with disease.

Briefly, 52871 genes can be mapped to chromosomes by preparing PCR primers (preferably 15-25 bp in length) from the 52871 nucleotide sequences. Computer analysis of the 52871 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the 52871 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the 52871 nucleotide sequences to design oligonucleotide primers, sublocalization can be achieved with panels of fragments from specific chromosomes. Other mapping strategies which can similarly be used to map a 52871 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature 325:783-787.

Moreover, differences in the DNA sequences between individuals affected and unaffected with a disease associated with the 52871 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The 52871 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the 52871 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The 52871 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of 27NO:1 can comfortably provide positive individual identification with a panel of perhaps 10 to 1,000 primers which each yield a noncoding amplified sequence of 100 bases. If predicted coding sequences, such as those in SEQ ID NO:29 are used, a more appropriate number of primers for positive individual identification would be 500-2,000.

If a panel of reagents from 52871 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of 52871 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:27 are particularly appropriate for this use as greater numbers of polymorphisms occur in the noncoding regions, making it easier to differentiate individuals using this technique. Examples of polynucleotide reagents include the 52871 nucleotide sequences or portions thereof, e.g., fragments derived from the noncoding regions of SEQ ID NO:27 having a length of at least 20 bases, preferably at least 30 bases.

The 52871 nucleotide sequences described herein can further be used to provide polynucleotide reagents, e.g., labeled or labelable probes which can be used in, for example, an in situ hybridization technique, to identify a specific tissue, e.g., thymus or brain tissue. This can be very useful in cases where a forensic pathologist is presented with a tissue of unknown origin. Panels of such 52871 probes can be used to identify tissue by species and/or by organ type.

In a similar fashion, these reagents, e.g., 52871 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 52871 protein and/or nucleic acid expression as well as 52871 activity, in the context of a biological sample (e.g., blood, serum, cells, tissue) to thereby determine whether an individual is afflicted with a disease or disorder, or is at risk of developing a disorder, associated with aberrant or unwanted 52871 expression or activity. The invention also provides for prognostic (or predictive) assays for determining whether an individual is at risk of developing a disorder associated with 52871 protein, nucleic acid expression or activity. For example, mutations in a 52871 gene can be assayed in a biological sample. Such assays can be used for prognostic or predictive purpose to thereby prophylactically treat an individual prior to the onset of a disorder characterized by or associated with 52871 protein, nucleic acid expression or activity.

Another aspect of the invention pertains to monitoring the influence of agents (e.g., drugs, compounds) on the expression or activity of 52871 in clinical trials.

These and other agents are described in further detail in the following sections.

1. Diagnostic Assays.

An exemplary method for detecting the presence or absence of 52871 protein, polypeptide or nucleic acid in a biological sample involves obtaining a biological sample from a test subject and contacting the biological sample with a compound or an agent capable of detecting 52871 protein, polypeptide or nucleic acid (e.g., mRNA, or genomic DNA) that encodes 52871 protein such that the presence of 52871 protein, polypeptide or nucleic acid is detected in the biological sample. In another aspect, the present invention provides a method for detecting the presence of 52871 activity in a biological sample by contacting the biological sample with an agent capable of detecting an indicator of 52871 activity such that the presence of 52871 activity is detected in the biological sample. A preferred agent for detecting 52871 mRNA or genomic DNA is a labeled nucleic acid probe capable of hybridizing to 52871 mRNA or genomic DNA. The nucleic acid probe can be, for example, the 52871 nucleic acid set forth in SEQ ID NO:27 or 29, or a portion thereof, such as an oligonucleotide of at least 15, 30, 50, 100, 250 or 500 nucleotides in length and sufficient to specifically hybridize under stringent conditions to 52871 mRNA or genomic DNA or complements thereof. Other suitable probes for use in the diagnostic assays of the invention are described herein.

A preferred agent for detecting 52871 protein is an antibody capable of binding to 52871 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect 52871 mRNA, protein, or genomic DNA in a biological sample in vitro as well as in vivo. For example, in vitro techniques for detection of 52871 mRNA include Northern hybridizations and in situ hybridizations. In vitro techniques for detection of 52871 protein include enzyme linked immunosorbent assays (ELISAs), Western blots, immunoprecipitations and immunofluorescence. In vitro techniques for detection of 52871 genomic DNA include Southern hybridizations. Furthermore, in vivo techniques for detection of 52871 protein include introducing into a subject a labeled anti-52871 antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding a 52871 protein; (ii) aberrant expression of a gene encoding a 52871 protein; (iii) mis-regulation of the gene; and (iii) aberrant post-translational modification of a 52871 protein, wherein a wild-type form of the gene encodes a protein with a 52871 activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting 52871 protein, mRNA, or genomic DNA, such that the presence of 52871 protein, mRNA or genomic DNA is detected in the biological sample, and comparing the presence of 52871 protein, mRNA or genomic DNA in the control sample with the presence of 52871 protein, mRNA or genomic DNA in the test sample.

The invention also encompasses kits for detecting the presence of 52871 in a biological sample. For example, the kit can comprise a labeled compound or agent capable of detecting 52871 protein or mRNA in a biological sample; means for determining the amount of 52871 in the sample; and means for comparing the amount of 52871 in the sample with a standard.

The compound or agent can be packaged in a suitable container. The kit can further comprise instructions for using the kit to detect 52871 protein or nucleic acid.

2. Prognostic Assays

The diagnostic methods described herein can furthermore be utilized to identify subjects having or at risk of developing a disease or disorder associated with aberrant or unwanted 52871 expression or activity. As used herein, the term “aberrant” includes a 52871 expression or activity which deviates from the wild type 52871 expression or activity. Aberrant expression or activity includes increased or decreased expression or activity, as well as expression or activity which does not follow the wild type developmental pattern of expression or the subcellular pattern of expression. For example, aberrant 52871 expression or activity is intended to include the cases in which a mutation in the 52871 gene causes the 52871 gene to be under-expressed or over-expressed and situations in which such mutations result in a non-functional 52871 protein or a protein which does not function in a wild-type fashion, e.g., a protein which does not interact with a 52871 substrate, or one which interacts with a non-52871 substrate. As used herein, the term “unwanted” includes an unwanted phenomenon involved in a biological response such as cellular proliferation. For example, the term unwanted includes a 52871 expression or activity which is undesirable in a subject.

The assays described herein, such as the preceding diagnostic assays or the following assays, can be utilized to identify a subject having or at risk of developing a disorder associated with a misregulation in 52871 protein activity or nucleic acid expression, such as a CNS disorder (e.g., a cognitive or neurodegenerative disorder), a pain disorder, a cellular proliferation, growth, differentiation, or migration disorder, a cardiovascular disorder, musculoskeletal disorder, an immune disorder, or a hormonal disorder. Alternatively, the prognostic assays can be utilized to identify a subject having or at risk for developing a disorder associated with a misregulation in 52871 protein activity or nucleic acid expression, such as a CNS disorder, a pain disorder, a cellular proliferation, growth, differentiation, or migration disorder, a musculoskeletal disorder, a cardiovascular disorder, an immune disorder, or a hormonal disorder. Thus, the present invention provides a method for identifying a disease or disorder associated with aberrant or unwanted 52871 expression or activity in which a test sample is obtained from a subject and 52871 protein or nucleic acid (e.g., mRNA or genomic DNA) is detected, wherein the presence of 52871 protein or nucleic acid is diagnostic for a subject having or at risk of developing a disease or disorder associated with aberrant or unwanted 52871 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 52871 expression or activity. For example, such methods can be used to determine whether a subject can be effectively treated with an agent for a CNS disorder, a pain disorder, a muscular disorder, a cellular proliferation, growth, differentiation, or migration disorder, an immune disorder, or a hormonal disorder. Thus, the present invention provides methods for determining whether a subject can be effectively treated with an agent for a disorder associated with aberrant or unwanted 52871 expression or activity in which a test sample is obtained and 52871 protein or nucleic acid expression or activity is detected (e.g., wherein the abundance of 52871 protein or nucleic acid expression or activity is diagnostic for a subject that can be administered the agent to treat a disorder associated with aberrant or unwanted 52871 expression or activity).

The methods of the invention can also be used to detect genetic alterations in a 52871 gene, thereby determining if a subject with the altered gene is at risk for a disorder characterized by misregulation in 52871 protein activity or nucleic acid expression, such as a CNS disorder, a pain disorder, a musculoskeletal disorder, a cellular proliferation, growth, differentiation, or migration disorder, a cardiovascular disorder, an immune disorder, or a hormonal disorder. In preferred embodiments, the methods include detecting, in a sample of cells from the subject, the presence or absence of a genetic alteration characterized by at least one of an alteration affecting the integrity of a gene encoding a 52871-protein, or the mis-expression of the 52871 gene. For example, such genetic alterations can be detected by ascertaining the existence of at least one of 1) a deletion of one or more nucleotides from a 52871 gene; 2) an addition of one or more nucleotides to a 52871 gene; 3) a substitution of one or more nucleotides of a 52871 gene, 4) a chromosomal rearrangement of a 52871 gene; 5) an alteration in the level of a messenger RNA transcript of a 52871 gene, 6) aberrant modification of a 52871 gene, such as of the methylation pattern of the genomic DNA, 7) the presence of a non-wild type splicing pattern of a messenger RNA transcript of a 52871 gene, 8) a non-wild type level of a 52871-protein, 9) allelic loss of a 52871 gene, and 10) inappropriate post-translational modification of a 52871-protein. As described herein, there are a large number of assays known in the art which can be used for detecting alterations in a 52871 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 52871 gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a 52871 gene under conditions such that hybridization and amplification of the 52871 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990). Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 52871 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 52871 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 52871 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 52871 gene and detect mutations by comparing the sequence of the sample 52871 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 52871 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type 52871 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 52871 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 52871 sequence, e.g., a wild-type 52871 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 52871 genes. For example, single strand conformation polymorphism (SSCP) may be used to detect differences in electrophoretic mobility between mutant and wild type nucleic acids (Orita et al. (1989) Proc Natl Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 52871 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a 52871 gene.

Furthermore, any cell type or tissue in which 52871 is expressed may be utilized in the prognostic assays described herein.

3. Monitoring of Effects During Clinical Trials

Monitoring the influence of agents (e.g., drugs) on the expression or activity of a 52871 protein (e.g., the maintenance of cellular homeostasis) can be applied not only in basic drug screening, but also in clinical trials. For example, the effectiveness of an agent determined by a screening assay as described herein to increase 52871 gene expression, protein levels, or upregulate 52871 activity, can be monitored in clinical trials of subjects exhibiting decreased 52871 gene expression, protein levels, or downregulated 52871 activity. Alternatively, the effectiveness of an agent determined by a screening assay to decrease 52871 gene expression, protein levels, or downregulate 52871 activity, can be monitored in clinical trials of subjects exhibiting increased 52871 gene expression, protein levels, or upregulated 52871 activity. In such clinical trials, the expression or activity of a 52871 gene, and preferably, other genes that have been implicated in, for example, a 52871-associated disorder can be used as a “read out” or markers of the phenotype of a particular cell.

For example, and not by way of limitation, genes, including 52871, that are modulated in cells by treatment with an agent (e.g., compound, drug or small molecule) which modulates 52871 activity (e.g., identified in a screening assay as described herein) can be identified. Thus, to study the effect of agents on 52871-associated disorders (e.g., disorders characterized by deregulated cell proliferation and/or migration or pain disorders), for example, in a clinical trial, cells can be isolated and RNA prepared and analyzed for the levels of expression of 52871 and other genes implicated in the 52871-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of 52871 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a 52871 protein, mRNA, or genomic DNA in the preadministration sample; (iii) obtaining one or more post-administration samples from the subject; (iv) detecting the level of expression or activity of the 52871 protein, mRNA, or genomic DNA in the post-administration samples; (v) comparing the level of expression or activity of the 52871 protein, mRNA, or genomic DNA in the pre-administration sample with the 52871 protein, mRNA, or genomic DNA in the post administration sample or samples; and (vi) altering the administration of the agent to the subject accordingly. For example, increased administration of the agent may be desirable to increase the expression or activity of 52871 to higher levels than detected, i.e., to increase the effectiveness of the agent. Alternatively, decreased administration of the agent may be desirable to decrease expression or activity of 52871 to lower levels than detected, i.e. to decrease the effectiveness of the agent. According to such an embodiment, 52871 expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted 52871 expression or activity, e.g., a GPCR-associated disorder, a cell-signaling disorder, pain, or a pain disorder. “Treatment”, or “treating” as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. A therapeutic agent includes, but is not limited to, small molecules, peptides, antibodies, ribozymes and antisense oligonucleotides. With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the 52871 molecules of the present invention or 52871 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted 52871 expression or activity, by administering to the subject a 52871 or an agent which modulates 52871 expression or at least one 52871 activity. Subjects at risk for a disease which is caused or contributed to by aberrant or unwanted 52871 expression or activity can be identified by, for example, any or a combination of diagnostic or prognostic assays as described herein. Administration of a prophylactic agent can occur prior to the manifestation of symptoms characteristic of the 52871 aberrancy, such that a disease or disorder is prevented or, alternatively, delayed in its progression. Depending on the type of 52871 aberrancy, for example, a 52871, 52871 agonist or 52871 antagonist agent can be used for treating the subject. The appropriate agent can be determined based on screening assays described herein.

2. Therapeutic Methods

Another aspect of the invention pertains to methods of modulating 52871 expression or activity for therapeutic purposes. Accordingly, in an exemplary embodiment, the modulatory method of the invention involves contacting a cell capable of expressing 52871 with an agent that modulates one or more of the activities of 52871 protein activity associated with the cell, such that 52871 activity in the cell is modulated. An agent that modulates 52871 protein activity can be an agent as described herein, such as a nucleic acid or a protein, a naturally-occurring substrate molecule of a 52871 protein (e.g., energy transduction metabolites, urea cycle metabolites, lipid metabolism metabolites, amino acid precursors, nucleic acid precursors), a 52871 antibody, a 52871 agonist or antagonist, a peptidomimetic of a 52871 agonist or antagonist, or other small molecule. In one embodiment, the agent stimulates one or more 52871 activities. Examples of such stimulatory agents include active 52871 protein and a nucleic acid molecule encoding 52871 that has been introduced into the cell. In another embodiment, the agent inhibits one or more 52871 activities. Examples of such inhibitory agents include antisense 52871 nucleic acid molecules, anti-52871 antibodies, and 52871 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 52871 protein or nucleic acid molecule. In one embodiment, the method involves administering an agent (e.g., an agent identified by a screening assay described herein), or combination of agents that modulates (e.g., upregulates or downregulates) 52871 expression or activity. In another embodiment, the method involves administering a 52871 protein or nucleic acid molecule as therapy to compensate for reduced, aberrant, or unwanted 52871 expression or activity.

Stimulation of 52871 activity is desirable in situations in which 52871 is abnormally downregulated and/or in which increased 52871 activity is likely to have a beneficial effect. Likewise, inhibition of 52871 activity is desirable in situations in which 52871 is abnormally upregulated and/or in which decreased 52871 activity is likely to have a beneficial effect.

3. Pharmacogenomics

The 52871 molecules of the present invention, as well as agents, or modulators which have a stimulatory or inhibitory effect on 52871 activity (e.g., 52871 gene expression) as identified by a screening assay described herein can be administered to individuals to treat (prophylactically or therapeutically) 52871-associated disorders (e.g., proliferative disorders, CNS disorders, pain or pain disorders, cardiac disorders, metabolic disorders, or muscular disorders) associated with aberrant or unwanted 52871 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 52871 molecule or 52871 modulator as well as tailoring the dosage and/or therapeutic regimen of treatment with a 52871 molecule or 52871 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug target is known (e.g., a 52871 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a 52871 molecule or 52871 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 52871 molecule or 52871 modulator, such as a modulator identified by one of the exemplary screening assays described herein.

4. Use of 52871 Molecules as Surrogate Markers

The 52871 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 52871 molecules of the invention may be detected, and may be correlated with one or more biological states in vivo. For example, the 52871 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 52871 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 52871 marker) transcription or expression, the amplified marker may be in a quantity which is more readily detectable than the drug itself. Also, the marker may be more easily detected due to the nature of the marker itself; for example, using the methods described herein, anti-52871 antibodies may be employed in an immune-based detection system for a 52871 protein marker, or 52871-specific radiolabeled probes may be used to detect a 52871 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The 52871 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 52871 protein or RNA) for specific tumor markers in a subject, a drug or course of treatment may be selected that is optimized for the treatment of the specific tumor likely to be present in the subject. Similarly, the presence or absence of a specific sequence mutation in 52871 DNA may correlate 52871 drug response. The use of pharmacogenomic markers therefore permits the application of the most appropriate treatment for each subject without having to administer the therapy.

VI. Electronic Apparatus Readable Media and Arrays

Electronic apparatus readable media comprising 52871 sequence information is also provided. As used herein, “52871 sequence information” refers to any nucleotide and/or amino acid sequence information particular to the 52871 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said 52871 sequence information includes detection of the presence or absence of a sequence (e.g., detection of expression of a sequence, fragment, polymorphism, etc.), determination of the level of a sequence (e.g., detection of a level of expression, for example, a quantative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon 52871 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 52871 sequence information.

A variety of software programs and formats can be used to store the sequence information on the electronic apparatus readable medium. For example, the sequence information can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the 52871 sequence information.

By providing 52871 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a 52871-associated disease or disorder or a pre-disposition to a 52871-associated disease or disorder, wherein the method comprises the steps of determining 52871 sequence information associated with the subject and based on the 52871 sequence information, determining whether the subject has a 52871-associated disease or disorder or a pre-disposition to a 52871-associated disease or disorder and/or recommending a particular treatment for the disease, disorder or pre-disease condition.

The present invention further provides in an electronic system and/or in a network, a method for determining whether a subject has a 52871-associated disease or disorder or a pre-disposition to a disease associated with a 52871 wherein the method comprises the steps of determining 52871 sequence information associated with the subject, and based on the 52871 sequence information, determining whether the subject has a 52871-associated disease or disorder or a pre-disposition to a 52871-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a 52871-associated disease or disorder or a pre-disposition to a 52871 associated disease or disorder associated with 52871, said method comprising the steps of receiving 52871 sequence information from the subject and/or information related thereto, receiving phenotypic information associated with the subject, acquiring information from the network corresponding to 52871 and/or a 52871-associated disease or disorder, and based on one or more of the phenotypic information, the 52871 information (e.g., sequence information and/or information related thereto), and the acquired information, determining whether the subject has a 52871-associated disease or disorder or a pre-disposition to a 52871-associated disease or disorder (e.g., a pain disorder). The method may further comprise the step of recommending a particular treatment for the disease, disorder or pre-disease condition.

The present invention also provides a business method for determining whether a subject has a 52871-associated disease or disorder or a pre-disposition to a 52871-associated disease or disorder, said method comprising the steps of receiving information related to 52871 (e.g., sequence information and/or information related thereto), receiving phenotypic information associated with the subject, acquiring information from the network related to 52871 and/or related to a 52871-associated disease or disorder, and based on one or more of the phenotypic information, the 52871 information, and the acquired information, determining whether the subject has a 52871-associated disease or disorder or a pre-disposition to a 52871-associated disease or disorder. The method may further comprise the step of recommending a particular treatment for the disease, disorder or pre-disease condition.

The invention also includes an array comprising a 52871 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be 52871. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a 52871-associated disease or disorder, progression of 52871-associated disease or disorder, and processes, such a cellular transformation associated with the 52871-associated disease or disorder.

The array is also useful for ascertaining the effect of the expression of a gene on the expression of other genes in the same cell or in different cells (e.g., ascertaining the effect of 52871 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including 52871) that could serve as a molecular target for diagnosis or therapeutic intervention.

This invention is further illustrated by the following examples which should not be construed as limiting. The contents of all references, patents and published patent applications cited throughout this application are incorporated herein by reference.

### EXAMPLES

### Example 1

### Identification and Characterization of Human

52871 cDNA

In this example, the identification and characterization of the gene encoding human 52871 (clone Fbh52871) is described.

Isolation of the 52871 cDNA

The invention is based, at least in part, on the discovery of a human gene encoding a novel GPCR protein, referred to herein as 52871. The entire sequence of human clone Fbh52871, was determined and found to contain an open reading frame termed human “52871.” The 52871 protein sequence set forth in SEQ ID NO:28 comprises about 348 amino acids. The coding region (open reading frame) of SEQ ID NO:27, is set forth as SEQ ID NO:29.

Analysis of the Human 52871 Molecule

An analysis of the possible cellular localization of the 52871 protein based on its amino acid sequence was performed using the methods and algorithms described in Nakai and Kanehisa (1992) Genomics 14:897-911. The results of the analysis predict that human 52871 (SEQ ID NO:28) is localized intracellularly (probabilities are shown for localization to, e.g., 44.4% in the endoplasmic reticulum, 22.2% in the vacuoles, 11.1% in the golgi apparatus, 11.1% in the vesicles of the secretory system, and 11.1% in the mitochondria).

A search of the amino acid sequence of 52871 was performed against the Memsat database. This search resulted in the identification of seven transmembrane domains in the amino acid sequence of human 52871 (SEQ ID NO:28) at about residues 53-75, 90-108, 126-144, 165-186, 210-234, 275-293, and 309-333.

Examination of the amino acid sequence of 52871 (SEQ ID NO:28) revealed that the present invention contains conserved cysteines found in the first 2 extracellular loops (prior to the third and fifth transmembrane domains) of most GPCRs (cys 121 and cys 197 of SEQ ID NO:28). A highly conserved asparagine residue in the first transmembrane domain is present (asn 67 in SEQ ID NO:28). Transmembrane domain two of the 52871 protein contains a highly conserved leucine (leu90 of SEQ ID NO:28). The two cysteine residues are believed to form a disulfide bond that stabilizes the functional protein structure. A highly conserved tryptophan and proline in the fourth transmembrane domain of the 52871 proteins is present (trp171 and pro 180 of SEQ ID NO:28). The third cytoplasmic loop contains 40 amino acid residues and is thus the longest cytoplasmic loop of the three, characteristic of G protein coupled receptors. Moreover, a highly conserved proline in the sixth transmembrane domain is present (pro 289 of SEQ ID NO:28). The proline residues in the fourth, fifth, sixth, and seventh transmembrane domains are thought to introduce kinks in the alpha-helices and may be important in the formation of the ligand binding pocket. Moreover, an almost invariant proline is present in the seventh transmembrane domain of 52871 (pro 327 of SEQ ID NO:28).

A search of the amino acid sequence of 52871 was also performed against the HMM database). This search resulted in the identification of a “7-TMR profile” (“7tm_{—}1” domain) in the amino acid sequence of 52871 (SEQ ID NO:28) at about residues 66-330 (score: 164.0).

Further domain motifs were identified by using the amino acid sequence of 52871 (SEQ ID NO:28) to search the Propom database. Numerous matches against protein domains described as G-protein transmembrane domains and the like were identified.

A search was also performed against the Prosite database, and resulted in the identification of potential N-glycosylation sites at about residues 4-7 and 250-253, (Prosite accession number PS0001). This search also identified the presence of a potential glycosaminoglycan attachment site (Prosite accession number PS0002) at about residues 13-16, two potential cAMP- and cGMP-dependant protein kinase phosphorylation sites at about residues 78-81 and 240-243 (Prosite accession number PS0004), two potential protein kinase C phosphorylation sites at about residues 47-49 and 74-76 (Prosite accession number PS0005), three potential casein kinase II phosphorylation sites at about residues 31-34, 115-118, and 191-194 (Prosite accession number PS0006), and two potential N-myristoylation sites at about residues 8-13, and 14-19 (Prosite accession number PS0008). Furthermore, this search also identified the presence of a potential amidation site at residues 185-188 (Prosite accession number PS0009). This search also identified the presence of a G-protein coupled receptor signature motif at residues 134-150 (Prosite accession number PS00237). A structural, hydrophobicity, and antigenicity analysis of the human Fbh52871 protein was undertaken.

Tissue Distribution of Human 52871 mRNA by Northern Analysis

This example describes the tissue distribution of 52871 mRNA, as determined by Northern analysis.

Northern blot hybridizations with the various RNA samples are performed under standard conditions and washed under stringent conditions, i.e., 0.2×SSC at 65° C. The DNA probe is radioactively labeled with 32P-dCTP using the Prime-It kit (Stratagene, La Jolla, Calif.) according to the instructions of the supplier. Filters containing human mRNA (MultiTissue Northern I and MultiTissue Northern II from Clontech, Palo Alto, Calif.) are probed in ExpressHyb hybridization solution (Clontech) and washed at high stringency according to manufacturer's recommendations.

Tissue Distribution of Human 52871 mRNA by In Situ Analysis

For in situ analysis, various tissues, e.g. tissues obtained from brain, spinal cord and skin from human, monkey, and rat, were first frozen on dry ice. Ten-micrometer-thick sections of the tissues were post-fixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections were rinsed in DEPC 2×SSC (1×SSC is 0.15M NaCl plus 0.015M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with 35S-radiolabeled (5×107 cpm/ml) cRNA probes. Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10% g of RNase A per ml for 30 minutes, and finally in TNE for 10 minutes. Slides were then rinsed with 2×SSC at room temperature, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

Results showed 52871 mRNA expression in rat brain (cortex and hippocampus), spinal cord, and dorsal root ganglia neurons, and testis. Results also showed 52871 mRNA expression in monkey cortex, dorsal root ganglia neurons, spinal cord, and testis. 52871 mRNA expression was also shown in human brain, spinal cord, DRG, and skin. In situ hybridization in monkey and rat tissues was performed with human 52871 probes. This cross-reactivity indicates that 52871 orthologues are likely highly conserved.

In situ hybridization in an animal model of pain, including models of pain caused by axotomized DRG, chronic constriction injury (CCl), and intraperitoneal administration of complete Freund's adjuvant (CFA), showed no regulation of 52871 mRNA expression, indicating that modulation of nociception by 52871 likely correlates with changes in 52871 activity as compared to changes in 52871 nucleic acid expression.

Tissue Expression Analysis of Human 52871 mRNA Using Taqman Analysis

This example describes the tissue distribution of human 52871 mRNA in a variety of cells and tissues, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., brain, testis, spinal cord, skin, dorsal root ganglia, placenta, etc., and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

A human normal tissue panel indicated that human 52871 is expressed at very low levels. The highest expression was in human brain, followed by spinal cord and dorsal root ganglia (DRG) (see Table 9, below).

TABLE 9
Tissue Expression Analysis of Human 52871 mRNA Using Taqman
Analysis

| | | Beta 2 | |
|---|---|---|---|
| Tissue | 52871 | Mean | Expression |
| Adrenal Gland | 38.77 | 20.17 | 0.00 |
| Brain | 33.02 | 20.42 | 0.28 |
| Heart | 39.46 | 20.42 | 0.00 |
| Kidney | 40.00 | 19.46 | 0.00 |
| Liver | 40.00 | 20.14 | 0.00 |
| Lung | 40.00 | 18.37 | 0.00 |
| Mammary Gland | 40.00 | 19.23 | 0.00 |
| Placenta | 36.91 | 20.11 | 0.02 |
| Prostate | 39.46 | 19.41 | 0.00 |
| Salivary Gland | 40.00 | 20.53 | 0.00 |
| Muscle | 40.00 | 22.24 | 0.01 |
| Small Intestine | 40.00 | 19.52 | 0.00 |
| Spleen | 39.12 | 17.99 | 0.00 |
| Stomach | 40.00 | 19.83 | 0.00 |
| Testes | 32.84 | 21.36 | 0.61 |
| Thymus | 38.63 | 19.57 | 0.00 |
| Trachea | 40.00 | 20.77 | 0.00 |
| Uterus | 40.00 | 20.49 | 0.00 |
| Spinal Cord | 34.24 | 20.79 | 0.16 |
| Skin | 37.03 | 18.68 | 0.01 |
| DRG | 35.11 | 20.51 | 0.07 |

### Example 2

### Expression of Recombinant 52871 Protein Inbacterial Cells

In this example, 52871 is expressed as a recombinant glutathione-S-transferase (GST) fusion polypeptide in E. coli and the fusion polypeptide is isolated and characterized. Specifically, 52871 is fused to GST and this fusion polypeptide is expressed in E. coli, e.g., strain PEB199. Expression of the GST-52871 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant 52871 Protein in COS Cells

To express the 52871 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 52871 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 52871 DNA sequence is amplified by PCR using two primers. The 5′ primer contains the restriction site of interest followed by approximately twenty nucleotides of the 52871 coding sequence starting from the initiation codon; the 3′ end sequence contains complementary sequences to the other restriction site of interest, a translation stop codon, the HA tag or FLAG tag and the last 20 nucleotides of the 52871 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 52871 gene is inserted in the correct orientation. The ligation mixture is transformed into E. coli cells (strains HB101, DH5□, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 52871-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 52871 polypeptide is detected by radiolabelling (35S-methionine or 35S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with 35S-methionine (or 35S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA-specific monoclonal antibody. Precipitated polypeptides are then analyzed by SDS-PAGE.

Alternatively, DNA containing the 52871 coding sequence is cloned directly into the polylinker of the pCDNA/Amp vector using the appropriate restriction sites. The resulting plasmid is transfected into COS cells in the manner described above, and the expression of the 52871 polypeptide is detected by radiolabelling and immunoprecipitation using a 52871 specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

### VI. MUSCARINIC RECEPTORS AND USES THEREFOR

### Background of the Invention

Muscarinic receptors, so named because the actions of acetylcholine on such receptors are similar to those produced by the mushroom alkaloid muscarine, mediate most of the inhibitory and excitatory effects of the neurotransmitter acetylcholine in the heart, smooth muscle, glands and in neurons (both presynaptic and postsynaptic) in the autonomic and the central nervous system (Eglen, R. and Watson, N. (1996) Pharmacology & Toxicology 78:59-68). The muscarinic receptors belong to the G protein-coupled receptor superfamily (Wess, J. et al. (1990) Comprehensive Medicinal Chemistry 3:423-491). Like all other G protein-coupled receptors, the muscarinic receptors are predicted to conform to a generic protein fold consisting of seven hydrophobic transmembrane helices joined by alternative intracellular and extracellular loops, an extracellular amino-terminal domain, and a cytoplasmic carboxyl-terminal domain. The mammalian muscarinic receptors display a high degree of sequence identity, particularly in the transmembrane domains, sharing approximately 145 invariant amino acids (Wess, J. (1993) TIPS 14:308-313). Moreover, all of the mammalian muscarinic receptors contain a very large third cytoplasmic loop which, except for the membrane-proximal portions, displays virtually no sequence identity among the different family members (Bonner, T. I. (1989) Trends Neurosci. 12:148-151). Ligand binding to the receptor is believed to trigger conformational changes within the helical bundle, which are then transmitted to the cytoplasmic domain, where the interaction with specific G proteins occurs.

Molecular cloning studies have revealed the existence of five molecularly distinct mammalian muscarinic receptor proteins, termed the M1-M5 receptors (Bonner, T. I. (1989) Trends Neurosci. 12:148-151; and Hulme, E. C. et al. (1990) Annu. Rev. Pharmacol. Toxicol. 30:633-673). The M1 receptor is expressed primarily in the brain (cerebral cortex, olfactory bulb, olfactory tubercle, basal forebrain/septum, amygdala, and hippocampus) and in exocrine glands (Buckley, N. J. et al. (1988) J. Neurosci. 8:4646-4652). The M2 receptor is expressed in the brain (olfactory bulb, basal forebrain/septum, thalamus and amygdala), and in the ileum and the heart. The M3 receptor is expressed in the brain (cerebral cortex, olfactory tubercle, thalamus and hippocampus) the lung, the ileum, and in exocrine glands. The M4 receptor is expressed in the brain (olfactory bulb, olfactory tubercle, hippocampus and striatum) and in the lung. Finally, the M5 receptor is expressed primarily in the brain (substantia nigra) (Hulme, E. C. et al. (1990) A. Rev. Pharmac. Toxic. 30:633-673).

The two enzymes with which muscarinic receptors interact most directly are adenylate cyclase and phospholipase C. Studies with cloned receptors have shown that the M1, M3, and M5 muscarinic receptors are coupled to the types of G proteins known as Go (a stimulatory protein linked to phospholipase C) or Gq and that their activation results in the activation of phospholipase C. The M2 and M4 muscarinic receptors are coupled to a Gi protein (an inhibitory protein linked to adenylate cyclase), and their activation results in the inhibition of adenylate cyclase. Through these signal transduction pathways, the muscarinic receptors are responsible for a variety of physiological functions including the regulation of neurotransmitter release (including acetylcholine release) from the brain, the regulation of digestive enzyme and insulin secretion in the pancreas, the regulation of amylase secretion by the parotid gland, and the regulation of contraction in cardiac and smooth muscle (Caulfield, M. P. (1993) Pharmac. Ther. 58:319-379).

### Summary of the Invention

This invention provides a novel nucleic acid molecule which encodes a polypeptide, referred to herein as muscarinic acetylcholine receptor 6 (“mACHR-6”) polypeptide or protein, which is capable of, for example, modulating the effects of acetylcholine on acetylcholine responsive cells e.g., by modulating phospholipase C signaling/activity. Nucleic acid molecules encoding an mACHR-6 polypeptide are referred to herein as mACHR-6 nucleic acid molecules. In a preferred embodiment, the mACHR-6 polypeptide interacts with (e.g., binds to) a protein which is a member of the G family of proteins. Examples of such proteins include Go, Gi, Gs, Gq and Gt. These proteins are described in Lodish H. et al. Molecular Cell Biology, (Scientific American Books Inc., New York, N.Y., 1995); Dolphin A. C. et al. (1987) Trends Neurosci. 10:53; and Birnbaumer L. et al. (1992) Cell 71:1069, the contents of which are expressly incorporated herein by reference.

In a preferred embodiment, the mACHR-6 polypeptide interacts with (e.g., binds to) acetylcholine. Acetylcholine is the predominant neurotransmitter in the sympathetic and parasympathetic preganglionic synapses, as well as in the parasympathetic postganglionic synapses and in some sympathetic postganglionic synapses. Synapses in which acetylcholine is the neurotransmitter are called cholinergic synapses. Acetylcholine acts to regulate smooth muscle contraction, heart rate, glandular function such as gastric acid secretion, and neural function such as release of neurotransminers from the brain. The mACHR-6 polypeptide of the present invention binds to acetylcholine and serves to mediate the acetylcholine induced signal to the cell. Thus, mACHR-6 molecules can be used as targets to modulate acetylcholine induced functions and thus to treat disorders associated with, for example, abnormal acetylcholine levels, or abnormal or aberrant mACHR-6 polypeptide activity or nucleic acid expression.

Accordingly, one aspect of the invention pertains to isolated nucleic acid molecules (e.g., cDNAs) comprising a nucleotide sequence encoding an mACHR-6 polypeptide or biologically active portions thereof, as well as nucleic acid fragments suitable as primers or hybridization probes for the detection of mACHR-6-encoding nucleic acid (e.g., mRNA). In particularly preferred embodiments, the isolated nucleic acid molecule comprises the nucleotide sequence of SEQ ID NO:33, 34, or 35, or the coding region or a complement of either of these nucleotide sequences. In other particularly preferred embodiments, the isolated nucleic acid molecule of the invention comprises a nucleotide sequence which encodes naturally occurring allelic variants, genetically altered variants and non-human and non-rat homologues of the mACHR-6 polypeptides described herein. Such nucleic acid molecules are identifiable as being able to hybridize to or which are at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the nucleotide sequence shown in SEQ ID NO:33, 34, or 35, or a portion of either of these nucleotide sequences. In other preferred embodiments, the isolated nucleic acid molecule encodes the amino acid sequence of SEQ ID NO:36, 37, or 38. The preferred mACHR-6 polypeptides of the present invention also preferably possess at least one of the mACHR-6 activities described herein.

In another embodiment, the isolated nucleic acid molecule encodes a polypeptide or portion thereof wherein the polypeptide or portion thereof includes an amino acid sequence which is sufficiently homologous to an amino acid sequence of SEQ ID NO:36, 37, or 38, e.g., sufficiently homologous to an amino acid sequence of SEQ ID NO:36, 37, or 38 such that the polypeptide or portion thereof maintains an mACHR-6 activity. Preferably, the polypeptide or portion thereof encoded by the nucleic acid molecule maintains the ability to modulate an acetylcholine response in an acetylcholine responsive cell. In one embodiment, the polypeptide encoded by the nucleic acid molecule is at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the amino acid sequence of SEQ ID NO:36, 37, or 38 (e.g., the entire amino acid sequence of SEQ ID NO:36, 37, or 38). In another preferred embodiment the nucleic acid molecule encodes a polypeptide fragment comprising at least 15 contiguous amino acids of SEQ ID NO:36, 37, or 38. In yet another preferred embodiment, the polypeptide is a full length human polypeptide which is substantially homologous to the entire amino acid sequence of SEQ ID NO:36, 37, or 38 (encoded by the open reading frame shown in SEQ ID NO:39, 40, or 41, respectively). In still another preferred embodiment, the nucleic acid molecule encodes a naturally occurring allelic variant of the polypeptide of SEQ ID NO:36, 37, or 38 and hybridizes under stringent conditions to a nucleic acid molecule comprising the nucleotide sequence of SEQ ID NO:33, 34, or 35, respectively.

In yet another embodiment, the isolated nucleic acid molecule is derived from a human and encodes a portion of a polypeptide which includes a transmembrane domain. Preferably, the transmembrane domain encoded by the human nucleic acid molecule is at least about 50-55%, preferably at least about 60-65%, more preferably at least about 70-75%, even more preferably at least about 80-85%, and most preferably at least about 90-95% or more homologous to any of the human transmembrane domains (i.e., amino acid residues 34-59, 109-130, 152-174, 197-219, or 396-416) of SEQ ID NO:36 which are shown as separate sequences designated SEQ ID NOs:42, 43, 44, 45, and 46, respectively, or to any of the rat transmembrane domains (i.e., amino acid residues 34-59, 73-91, 109-130, 152-174, 197-219, 360-380, or 396-416 of SEQ ID NO:37 which are shown as separate sequences designated SEQ ID NOs:47, 48, 49, 50, 51, 52, and 53, respectively or amino acid residues 1-8, 26-47, 69-91, 114-136, 277-297, or 313-333 of SEQ ID NO:38 which are shown as separate sequences designated SEQ ID NOs:54, 55, 56, 57, 58, or 59, respectively). More preferably, the transmembrane domain encoded by the human nucleic acid molecule is at least about 75-80%, preferably at least about 80-85%, more preferably at least about 85-90%, and most preferably at least about 90-95% or more homologous to the transmembrane domain (i.e., amino acid residues 360-380) of SEQ ID NO:36 which is shown as a separate sequence designated SEQ ID NO:60, or at least about 80-85%, more preferably at least about 85-90%, and most preferably at least about 90-95% or more homologous to the transmembrane domain (i.e., amino acid residues 73-91) of SEQ ID NO:36 which is shown as a separate sequence designated SEQ ID NO:61.

In another preferred embodiment, the isolated nucleic acid molecule is derived from a human and encodes a polypeptide (e.g., an mACHR-6 fusion polypeptide such as an mACHR-6 polypeptide fused with a heterologous polypeptide) which includes a transmembrane domain which is at least about 75% or more homologous to SEQ ID NO:42-46, or to the corresponding rat sequences shown as SEQ ID NOs:47-53 and has one or more of the following mACHR-6 activities: 1) it can interact with (e.g., bind to) acetylcholine; 2) it can interact with (e.g., bind to) a G protein or another protein which naturally binds to mACHR-6; 3) it can modulate the activity of an ion channel (e.g., a potassium channel or a calcium channel); 4) it can modulate cytosolic ion, e.g., calcium, concentration; 5) it can modulate the release of a neurotransmitter, e.g., acetylcholine, from a neuron, e.g., a presynaptic neuron; 6) it can modulate an acetylcholine response in an acetylcholine responsive cell (e.g., a smooth muscle cell or a gland cell) to, for example, beneficially affect the acetylcholine responsive cell, e.g., a neuron; 7) it can signal ligand binding via phosphatidylinositol turnover; and 8) it can modulate, e.g., activate or inhibit, phospholipase C activity.

In another embodiment, the isolated nucleic acid molecule is at least 15 nucleotides, e.g., at least 15 contiguous nucleotides, in length and hybridizes under stringent conditions to a nucleic acid molecule comprising the nucleotide sequence of SEQ ID NO:33, 34, or 35. Preferably, the isolated nucleic acid molecule corresponds to a naturally-occurring nucleic acid molecule. More preferably, the isolated nucleic acid encodes naturally-occurring human mACHR-6 or a biologically active portion thereof. Moreover, given the disclosure herein of an mACHR-6-encoding cDNA sequence (e.g., SEQ ID NO:33, 34, or 35), antisense nucleic acid molecules (e.g., molecules which are complementary to the coding strand of the mACHR-6 cDNA sequence) are also provided by the invention.

Another aspect of the invention pertains to vectors, e.g., recombinant expression vectors, containing the nucleic acid molecules of the invention and host cells into which such vectors have been introduced. In one embodiment, such a host cell is used to produce an mACHR-6 polypeptide by culturing the host cell in a suitable medium. If desired, the mACHR-6 polypeptide can then be isolated from the medium or the host cell.

Yet another aspect of the invention pertains to transgenic non-human animals in which an mACHR-6 gene has been introduced or altered. In one embodiment, the genome of the non-human animal has been altered by introduction of a nucleic acid molecule of the invention encoding mACHR-6 as a transgene. In another embodiment, an endogenous mACHR-6 gene within the genome of the non-human animal has been altered, e.g., functionally disrupted, by homologous recombination.

Still another aspect of the invention pertains to an isolated mACHR-6 polypeptide or a portion, e.g., a biologically active portion, thereof. In a preferred embodiment, the isolated mACHR-6 polypeptide or portion thereof can modulate an acetylcholine response in an acetylcholine responsive cell. In another preferred embodiment, the isolated mACHR-6 polypeptide or portion thereof is sufficiently homologous to an amino acid sequence of SEQ ID NO:36, 37, or 38 such that the polypeptide or portion thereof maintains the ability to modulate an acetylcholine response in an acetylcholine responsive cell.

In one embodiment, the biologically active portion of the mACHR-6 polypeptide includes a domain or motif, preferably a domain or motif which has an mACHR-6 activity. The domain can be transmembrane domain. If the active portion of the polypeptide which comprises the transmembrane domain is isolated or derived from a human, it is preferred that the transmembrane domain be at least about 75-80%, preferably at least about 80-85%, more preferably at least about 85-90%, and most preferably at least about 90-95% or more homologous to SEQ ID NO:42, 61, 43, 44, 45, 60, 46, 47, 48, 49, 50, 51, 52, 53, 54, 55, 56, 57, 58, or 59. Preferably, the biologically active portion of the mACHR-6 polypeptide which includes a transmembrane domain also has one of the following mACHR-6 activities: 1) it can interact with (e.g., bind to) acetylcholine; 2) it can interact with (e.g., bind to) a G protein or another protein which naturally binds to mACHR-6; 3) it can modulate the activity of an ion channel (e.g., a potassium channel or a calcium channel); 4) it can modulate cytosolic ion, e.g., calcium, concentration; 5) it can modulate the release of a neurotransmitter, e.g., acetylcholine, from a neuron, e.g., a presynaptic neuron; 6) it can modulate an acetylcholine response in an acetylcholine responsive cell (e.g., a smooth muscle cell or a gland cell) to, for example, beneficially affect the acetylcholine responsive cell, e.g., a neuron; 7) it can signal ligand binding via phosphatidylinositol turnover; and 8) it can modulate, e.g., activate or inhibit, phospholipase C activity.

The invention also provides an isolated preparation of an mACHR-6 polypeptide. In preferred embodiments, the mACHR-6 polypeptide comprises the amino acid sequence of SEQ ID NO:36, 37, or 38. In another preferred embodiment, the invention pertains to an isolated full length polypeptide which is substantially homologous to the entire amino acid sequence of SEQ ID NO:36, 37, or 38 (encoded by the open reading frame shown in SEQ ID NO:39, 40, or 41, respectively) such as a naturally occurring allelic variant of the mACHR-6 polypeptides described herein. In yet another embodiment, the polypeptide is at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the entire amino acid sequence of SEQ ID NO:36, 37, or 38 such as a non-human or non-rat homologue of the mACHR-6 polypeptides described herein. In other embodiments, the isolated mACHR-6 polypeptide comprises an amino acid sequence which is at least about 30-40% or more homologous to the amino acid sequence of SEQ ID NO:36, 37, or 38 and has an one or more of the following mACHR-6 activities: 1) it can interact with (e.g., bind to) acetylcholine; 2) it can interact with (e.g., bind to) a G protein or another protein which naturally binds to mACHR-6; 3) it can modulate the activity of an ion channel (e.g., a potassium channel or a calcium channel); 4) it can modulate cytosolic ion, e.g., calcium, concentration; 5) it can modulate the release of a neurotransmitter, e.g., acetylcholine, from a neuron, e.g., a presynaptic neuron; 6) it can modulate an acetylcholine response in an acetylcholine responsive cell (e.g., a smooth muscle cell or a gland cell) to, for example, beneficially affect the acetylcholine responsive cell, e.g., a neuron; 7) it can signal ligand binding via phosphatidylinositol turnover; and 8) it can modulate, e.g., activate or inhibit, phospholipase C activity.

Alternatively, the isolated mACHR-6 polypeptide can comprise an amino acid sequence which is encoded by a nucleotide sequence which hybridizes, e.g., hybridizes under stringent conditions, or is at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the nucleotide sequence of SEQ ID NO:33, 34, or 35, such as the allelic variants and non-human and non-rat homologues of the mACHR-6 polypeptides described herein as well as genetically altered variants generated by recombinant DNA methodologies. It is also preferred that the preferred forms of mACHR-6 also have one or more of the mACHR-6 activities described herein.

The mACHR-6 polypeptide (or protein) or a biologically active portion thereof can be operatively linked to a non-mACHR-6 polypeptide (e.g., a polypeptide comprising heterologous amino acid sequences) to form a fusion polypeptide. In addition, the mACHR-6 polypeptide or a biologically active portion thereof can be incorporated into a pharmaceutical composition comprising the polypeptide and a pharmaceutically acceptable carrier.

The mACHR-6 polypeptide of the invention, or portions or fragments thereof, can be used to prepare anti-mACHR-6 antibodies. Accordingly, the invention also provides an antigenic peptide of mACHR-6 which comprises at least 8 amino acid residues of the amino acid sequence shown in SEQ ID NO:36, 37, or 38 and encompasses an epitope of mACHR-6 such that an antibody raised against the peptide forms a specific immune complex with mACHR-6. Preferably, the antigenic peptide comprises at least 10 amino acid residues, more preferably at least 15 amino acid residues, even more preferably at least 20 amino acid residues, and most preferably at least 30 amino acid residues. The invention further provides an antibody that specifically binds mACHR-6. In one embodiment, the antibody is monoclonal. In another embodiment, the antibody is coupled to a detectable substance. In yet another embodiment, the antibody is incorporated into a pharmaceutical composition comprising the antibody and a pharmaceutically acceptable carrier.

Another aspect of the invention pertains to methods for modulating a cell activity mediated by mACHR-6, e.g., biological processes mediated by phosphatidylinositol turnover and signaling; secretion of a molecule, e.g., a neurotransmitter from a brain cell, or an enzyme from a gland cell; or contraction of a smooth muscle cell, e.g., an ileum smooth muscle cell or a cardiac cell, e.g., a cardiomyocyte. Such methods include contacting the cell with an agent which modulates mACHR-6 polypeptide activity or mACHR-6 nucleic acid expression such that an mACHR-6-mediated cell activity is altered relative to the same cellular activity which occurs in the absence of the agent. In a preferred embodiment, the cell (e.g., a smooth muscle cell or a neural cell) is capable of responding to acetylcholine through a signaling pathway involving an mACHR-6 polypeptide. The agent which modulates mACHR-6 activity can be an agent which stimulates mACHR-6 polypeptide activity or mACHR-6 nucleic acid expression. Examples of agents which stimulate mACHR-6 polypeptide activity or mACHR-6 nucleic acid expression include small molecules, active mACHR-6 polypeptides, and nucleic acids encoding mACHR-6 that have been introduced into the cell. Examples of agents which inhibit mACHR-6 activity or expression include small molecules, antisense mACHR-6 nucleic acid molecules, and antibodies that specifically bind to mACHR-6. In a preferred embodiment, the cell is present within a subject and the agent is administered to the subject.

The present invention also pertains to methods for treating subjects having various disorders, e.g., disorders mediated by abnormal mACHR-6 polypeptide activity, such as conditions caused by over, under, or inappropriate expression of mACHR-6. For example, the invention pertains to methods for treating a subject having a disorder characterized by aberrant mACHR-6 polypeptide activity or nucleic acid expression such as a nervous system disorder, e.g., a cognitive disorder, a sleep disorder, a movement disorder, a schizo-effective disorder, a disorder affecting pain generation mechanisms, a drinking disorder, or an eating disorder; a smooth muscle related disorder, e.g., irritable bowel syndrome, a cardiac muscle related disorder, e.g., bradycardia, or a gland related disorder, e.g., xerostomia. These methods include administering to the subject an mACHR-6 modulator (e.g., a small molecule) such that treatment of the subject occurs.

In other embodiments, the invention pertains to methods for treating a subject having a disorder mediated by abnormal mACHR-6 polypeptide activity, such as conditions caused by over, under, or inappropriate expression of mACHR-6, e.g., a nervous system disorder, e.g., a cognitive disorder, a sleep disorder, a movement disorder, a schizo-effective disorder, a disorder affecting pain generation mechanisms, a drinking disorder, or an eating disorder; a smooth muscle related disorder, e.g., irritable bowel syndrome; a cardiac muscle related disorder, e.g., bradycardia; or a gland related disorder, e.g., xerostomia. The method includes administering to the subject an mACHR-6 polypeptide or portion thereof such that treatment occurs. A nervous system disorder, smooth muscle related disorder, cardiac muscle related disorder or a gland related disorder can also be treated according to the invention by administering to the subject having the disorder a nucleic acid encoding an mACHR-6 polypeptide or portion thereof such that treatment occurs.

The invention also pertains to methods for detecting naturally occurring and recombinantly created genetic mutations in an mACHR-6 gene, thereby determining if a subject with the mutated gene is at risk for (or is predisposed to have) a disorder characterized by aberrant or abnormal mACHR-6 nucleic acid expression or mACHR-6 polypeptide activity, e.g., a nervous system disorder, a smooth muscle related disorder, a cardiac muscle related disorder or a gland related disorder. In preferred embodiments, the methods include detecting, in a sample of cells from the subject, the presence or absence of a genetic mutation characterized by an alteration affecting the integrity of a gene encoding an mACHR-6 polypeptide, or the misexpression of the mACHR-6 gene, such as that caused by a nucleic acid base substitution, deletion or addition, or gross sequence changes caused by a genetic translation, inversion or insertion.

Another aspect of the invention pertains to methods for detecting the presence of mACHR-6, or allelic variants thereof, in a biological sample. In a preferred embodiment, the methods involve contacting a biological sample (e.g., a brain or smooth muscle cell sample) with a compound or an agent capable of detecting mACHR-6 polypeptide or mACHR-6 mRNA such that the presence of mACHR-6 is detected in the biological sample. The compound or agent can be, for example, a labeled or labelable nucleic acid probe capable of hybridizing to mACHR-6 mRNA or a labeled or labelable antibody capable of binding to mACHR-6 polypeptide. The invention further provides methods for diagnosis of a subject with, for example, a nervous system disorder, a smooth muscle related disorder, a cardiac muscle related disorder or a gland related disorder, based on detection of mACHR-6 polypeptide or mRNA. In one embodiment, the method involves contacting a cell or tissue sample (e.g., a brain or smooth muscle cell sample) from the subject with an agent capable of detecting mACHR-6 polypeptide or mRNA, determining the amount of mACHR-6 polypeptide or mRNA expressed in the cell or tissue sample, comparing the amount of mACHR-6 polypeptide or mRNA expressed in the cell or tissue sample to a control sample and forming a diagnosis based on the amount of mACHR-6 polypeptide or mRNA expressed in the cell or tissue sample as compared to the control sample. Preferably, the cell sample is a brain cell sample. Kits for detecting mACHR-6 in a biological sample which include agents capable of detecting mACHR-6 polypeptide or mRNA are also within the scope of the invention.

Still another aspect of the invention pertains to methods, e.g., screening assays, for identifying a compound, e.g., a test compound, for treating a disorder characterized by aberrant mACHR-6 nucleic acid expression or polypeptide activity, e.g., a nervous system disorder, a smooth muscle related disorder, a cardiac muscle related disorder or a gland related disorder. These methods typically include assaying the ability of the compound or agent to modulate the expression of the mACHR-6 gene or the activity of the mACHR-6 polypeptide thereby identifying a compound for treating a disorder characterized by aberrant mACHR-6 nucleic acid expression or polypeptide activity. In a preferred embodiment, the method involves contacting a biological sample, e.g., a cell or tissue sample, e.g., a brain or smooth muscle cell sample, obtained from a subject having the disorder with the compound or agent, determining the amount of mACHR-6 polypeptide expressed and/or measuring the activity of the mACHR-6 polypeptide in the biological sample, comparing the amount of mACHR-6 polypeptide expressed in the biological sample and/or the measurable mACHR-6 biological activity in the cell to that of a control sample. An alteration in the amount of mACHR-6 polypeptide expression or mACHR-6 activity in the cell exposed to the compound or agent in comparison to the control is indicative of a modulation of mACHR-6 expression and/or mACHR-6 activity.

The invention also pertains to methods for identifying a compound or agent, e.g., a test compound or agent, which interacts with (e.g., binds to) an mACHR-6 polypeptide. These methods can include the steps of contacting the mACHR-6 polypeptide with the compound or agent under conditions which allow binding of the compound to the mACHR-6 polypeptide to form a complex and detecting the formation of a complex of the mACHR-6 polypeptide and the compound in which the ability of the compound to bind to the mACHR-6 polypeptide is indicated by the presence of the compound in the complex.

The invention further pertains to methods for identifying a compound or agent, e.g., a test compound or agent, which modulates, e.g., stimulates or inhibits, the interaction of the mACHR-6 polypeptide with a target molecule, e.g., acetylcholine, or a cellular protein involved in phosphatidylinositol turnover and signaling. In these methods, the mACHR-6 polypeptide is contacted, in the presence of the compound or agent, with the target molecule under conditions which allow binding of the target molecule to the mACHR-6 polypeptide to form a complex. An alteration, e.g., an increase or decrease, in complex formation between the mACHR-6 polypeptide and the target molecule as compared to the amount of complex formed in the absence of the compound or agent is indicative of the ability of the compound or agent to modulate the interaction of the mACHR-6 polypeptide with a target molecule.

### Detailed Description of the Invention

The present invention is based on the discovery of novel molecules, referred to herein as mACHR-6 nucleic acid and polypeptide molecules, which play a role in or function in acetylcholine signaling pathways. In one embodiment, the mACHR-6 molecules modulate the activity of one or more proteins involved in a neurotransmitter signaling pathway, e.g., an acetylcholine signaling pathway. In a preferred embodiment, the mACHR-6 molecules of the present invention are capable of modulating the activity of proteins involved in the acetylcholine signaling pathway to thereby modulate the effects of acetylcholine on acetylcholine responsive cells.

As used herein, the phrase “acetylcholine responsive cells” refers to cells which have a function which can be modulated (e.g., stimulated or inhibited) by the neurotransmitter acetylcholine. Examples of such functions include mobilization of intracellular molecules which participate in a signal transduction pathway, e.g., phosphatidylinositol 4,5-bisphosphate (PIP2) or inositol 1,4,5-triphosphate (IP3), polarization of the plasma membrane, production or secretion of molecules, alteration in the structure of a cellular component, cell proliferation, cell migration, cell differentiation, and cell survival. Acetylcholine responsive cells preferably express an acetylcholine receptor, e.g., a muscarinic receptor. Examples of acetylcholine responsive cells include neural cells, e.g., central nervous system and peripheral nervous system cells (such as sympathetic and parasympathetic neurons); smooth muscle cells, e.g., smooth muscle cells in the digestive tract, the urinary tract, the blood vessels, the airways and the lungs, or the uterus; cardiac muscle cells, e.g., cardiomyocytes; and gland cells such as exocrine gland cells, e.g., pancreatic gland cells, e.g., pancreatic beta cells, tear gland cells, sweat gland cells, or parotid gland cells.

Depending on the type of cell, the response elicited by acetylcholine is different. For example, in neural cells, acetylcholine regulates ion channels, and neural signal to noise ratio. Inhibition or over stimulation of the activity of proteins involved in the acetylcholine signaling pathway or misexpression of acetylcholine can lead to hypo- or hyperpolarization of the neural plasma membrane and to perturbed neural signal to noise ratio, which can in turn lead to nervous system related disorders. Examples of nervous system related disorders include cognitive disorders, e.g., memory and learning disorders, such as amnesia, apraxia, agnosia, amnestic dysnomia, amnestic spatial disorientation, Kluver-Bucy syndrome, Alzheimer's related memory loss (Eglen R. M. (1996) Pharmacol. and Toxicol. 78(2):59-68; Perry E. K. (1995) Brain and Cognition 28(3):240-58) and learning disability; disorders affecting consciousness, e.g., visual hallucinations, perceptual disturbances, or delerium associated with Lewy body dementia; schitzo-effective disorders (Dean B. (1996) Mol. Psychiatry 1(1):54-8), schizophrenia with mood swings (Bymaster F. P. (1997) J. Clin. Psychiatry 58 (suppl. 10):28-36; Yeomans J. S. (1995) Neuropharmacol. 12(1):3-16; Reimann D. (1994) J. Psychiatric Res. 28(3):195-210), depressive illness (primary or secondary); affective disorders (Janowsky D. S. (1994) Am. J. Med. Genetics 54(4):335-44); sleep disorders (Kimura F. (1997) J. Neurophysiol. 77(2):709-16), e.g., REM sleep abnormalities in patients suffering from, for example, depression (Riemann D. (1994) J. Psychosomatic Res. 38 Suppl. 1:15-25; Bourgin P. (1995) Neuroreport 6(3): 532-6), paradoxical sleep abnormalities (Sakai K. (1997) Eur. J. Neuroscience 9(3):415-23), sleep-wakefulness, and body temperature or respiratory depression abnormalities during sleep (Shuman S. L. (1995) Am. J. Physiol. 269(2 Pt 2):R308-17; Mallick B. N. (1997) Brain Res. 750(1-2):311-7). Other examples of nervous system related disorders include disorders affecting pain generation mechanisms, e.g., pain related to irritable bowel syndrome (Mitch C. H. (1997) J. Med. Chem. 40(4):538-46; Shannon H. E. (1997) J. Pharmac. and Exp. Therapeutics 281(2):884-94; Bouaziz H. (1995) Anesthesia and Analgesia 80(6):1140-4; or Guimaraes A. P. (1994) Brain Res. 647(2):220-30) or chest pain; movement disorders (Monassi C. R. (1997) Physiol. and Behav. 62(1):53-9), e.g., Parkinson's disease related movement disorders (Finn M. (1997) Pharmacol. Biochem. & Behavior 57(1-2):243-9; Mayorga A. J. (1997) Pharmacol. Biochem. & Behavior 56(2):273-9); eating disorders, e.g., insulin hypersecretion related obesity (Maccario M. (1997) J. Endocrinol. Invest. 20(1):8-12; Premawardhana L. D. (1994) Clin. Endocrinol. 40(5): 617-21); or drinking disorders, e.g., diabetic polydipsia (Murzi E. (1997) Brain Res. 752(1-2):184-8; Yang X. (1994) Pharmacol. Biochem. & Behavior 49(1):1-6).

In smooth muscle, acetylcholine regulates (e.g., stimulates or inhibits) contraction. Inhibition or overstimulation of the activity of proteins involved in the acetylcholine signaling pathway or misexpression of acetylcholine can lead to smooth muscle related disorders such as irritable bowel syndrome, diverticular disease, urinary incontinence, oesophageal achalasia, or chronic obstructive airways disease.

In cardiac muscle, acetylcholine induces a reduction in the heart rate and in cardiac contractility. Inhibition or overstimulation of the activity of proteins involved in the acetylcholine signaling pathway or misexpression of acetylcholine can lead to heart muscle related disorders such as pathologic bradycardia or tachycardia, arrhythmia, flutter or fibrillation.

In glands such as exocrine glands, acetylcholine regulates the secretion of enzymes or hormones, e.g., in the parotid gland acetylcholine induces the release of amylase, and in the pancreas acetylcholine induces the release of digestive enzymes and insulin. Inhibition or over stimulation of the activity of proteins involved in the acetylcholine signaling pathway or misexpression of acetylcholine can lead to gland related disorders such as xerostomia, or diabetes mellitus.

In a particularly preferred embodiment, the mACHR-6 molecules are capable of modulating the activity of G proteins, as well as phosphatidylinositol metabolism and turnover in acetylcholine responsive cells. As used herein, a “G protein” is a protein which participates, as a secondary signal, in a variety of intracellular signal transduction pathways, e.g., in the acetylcholine signaling pathway primarily through phosphatidylinositol metabolism and turnover. G proteins represent a family of heterotrimeric proteins composed of □, □ and □ subunits, which bind guanine nucleotides. These proteins are usually linked to cell surface receptors, e.g., receptors containing seven transmembrane domains, such as the muscarinic receptors. Following ligand binding to the receptor, a conformational change is transmitted to the G protein, which causes the □-subunit to exchange a bound GDP molecule for a GTP molecule and to dissociate from the □□-subunits. The GTP-bound form of the □-subunit typically functions as an effector-modulating moiety, leading to the production of second messengers, such as cyclic AMP (e.g., by activation of adenylate cyclase), diacylglycerol or inositol phosphates. Greater than 20 different types of □-subunits are known in man, which associate with a smaller pool of □ and □ subunits. Examples of mammalian G proteins include Gi, Go, Gq, Gs and Gt. G proteins are described extensively in Lodish H. et al. Molecular Cell Biology, (Scientific American Books Inc., New York, N.Y., 1995).

As used herein, “phosphatidylinositol turnover and metabolism” refers to the molecules involved in the turnover and metabolism of phosphatidylinositol 4,5-bisphosphate (PIP2) as well as to the activities of these molecules. PIP2 is a phospholipid found in the cytosolic leaflet of the plasma membrane. Binding of acetylcholine to a muscarinic receptor activates the plasma-membrane enzyme phospholipase C which in turn can hydrolyze PIP2 to produce 1,2-diacylglycerol (DAG) and inositol 1,4,5-triphosphate (IP3). Once formed IP3 can diffuse to the endoplasmic reticulum surface where it can bind an IP3 receptor, e.g., a calcium channel protein containing an IP3 binding site. IP3 binding can induce opening of the channel, allowing calcium ions to be released into the cytoplasm. IP3 can also be phosphorylated by a specific kinase to form inositol 1,3,4,5-tetraphosphate (IP4), a molecule which can cause calcium entry into the cytoplasm from the extracellular medium. IP3 and IP4 can subsequently be hydrolyzed very rapidly to the inactive products inositol 1,4-biphosphate (IP2) and inositol 1,3,4-triphosphate, respectively. These inactive products can be recycled by the cell to synthesize PIP2. The other second messenger produced by the hydrolysis of PIP2, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP2 or one of its metabolites.

mACHR-6 nucleic acid molecules were identified by screening appropriate cDNA libraries (described in detail in Example 1). The rat mACHR-6 nucleic acid molecule was identified by screening a rat brain cDNA library. Positive clones were sequenced and the partial sequences were analyzed by comparison with sequences in a nucleic acid sequence data base. This analysis indicated that the sequences were homologous to the muscarinic family of receptors. A longer rat clone was then isolated and sequenced. The human mACHR-6 nucleic acid molecule was identified by screening a human cerebellum cDNA library using probes designed based on the rat sequence.

Because of its ability to interact with (e.g., bind to) acetylcholine, G proteins and other proteins involved in the acetylcholine signaling pathway, the mACHR-6 polypeptide is also a polypeptide which functions in the acetylcholine signaling pathway.

The nucleotide sequence of the isolated human mACHR-6 cDNA and the predicted amino acid sequence of the human mACHR-6 polypeptide are shown in SEQ ID NOs:33 and 36, respectively.

The nucleotide sequence of the isolated rat mACHR-6 cDNA and the predicted amino acid sequence of the rat mACHR-6 polypeptide are shown in SEQ ID NOs:34 and 37, respectively.

The nucleotide sequence of the isolated partial rat mACHR-6 cDNA and the predicted amino acid sequence of the partial rat mACHR-6 polypeptide are shown in SEQ ID NOs:35 and 38, respectively.

The human mACHR-6 gene, which is approximately 2689 nucleotides in length, encodes a full length polypeptide having a molecular weight of approximately 51.2 KDa and which is approximately 445 amino acid residues in length. The human mACHR-6 polypeptide is expressed at least in the brain, in particular, regions of the brain such as the cerebellum, the cerebral cortex, the medulla, the occipital pole, the frontal lobe, the temporal lobe, the putamen, the corpus callosum the amygdala, the caudate nucleus, the hippocampus, the substantia nigra, the subthalamic nucleus and the thalamus; spinal cord, placenta, lungs, spleen, liver, skeletal muscle, kidney, and testis. Based on structural analysis, amino acid residues 34-59 (SEQ ID NO:42), 73-91 (SEQ ID NO:61), 109-130 (SEQ ID NO:43), 152-174 (SEQ ID NO:44), 197-219 (SEQ ID NO:45), 360-380 (SEQ ID NO:60), and 396-416 (SEQ ID NO:46) comprise transmembrane domains. As used herein, the term “transmembrane domain” refers to a structural amino acid motif which includes a hydrophobic helix that spans the plasma membrane. A transmembrane domain also preferably includes a series of conserved serine, threonine, and tyrosine residues. For example, the transmembrane domains between residues 109-130 (SEQ ID NO:43), 197-219 (SEQ ID NO:45), 360-380 (SEQ ID NO:60), and 396-416 (SEQ ID NO:46), contain threonine and tyrosine residues (located about 1-2 helical turns away from the membrane surface), which are important for ligand, e.g., acetylcholine, binding. Other important residues in the transmembrane domains include the conserved aspartate residue in the transmembrane domain between residues 109-130 (SEQ ID NO:43) and the conserved proline residue in the transmembrane domain between residues 152-174 (SEQ ID NO:44), which are also important for ligand, e.g., acetylcholine, binding. A skilled artisan will readily appreciate that the beginning and ending amino acid residue recited for various domains/fragments of mACHR-6 are based on structural analysis and that the actual beginning/ending amino acid for each may vary by a few amino acids from that identified herein.

The rat mACHR-6 gene, which is approximately 3244 nucleotides in length, encodes a full length polypeptide having a molecular weight of approximately 51.2 kDa and which is at least about 445 amino acid residues in length. The rat mACHR-6 polypeptide is expressed in the brain. Amino acid residues 34-59 (SEQ ID NO:47), 73-91 (SEQ ID NO:48), 109-130 (SEQ ID NO:49), 152-174 (SEQ ID NO:50), 197-219 (SEQ ID NO:51), 360-380 (SEQ ID NO:52) and 396-416 (SEQ ID NO:53) comprise transmembrane domains.

The rat mACHR-6 gene, which is at least about 2218 nucleotides in length, encodes a full length polypeptide having a molecular weight of at least about 41.6 kDa and which is at least about 362 amino acid residues in length. The rat mACHR-6 polypeptide is expressed in the brain. Amino acid residues 1-8 (SEQ ID NO:47), 26-47 (SEQ ID NO:48), 69-91 (SEQ ID NO:49), 114-136 (SEQ ID NO:50), 277-297 (SEQ ID NO:51), and 313-333 (SEQ ID NO:52) comprise transmembrane domains.

The partial rat mACHR-6 gene, which is at least about 2218 nucleotides in length, encodes a polypeptide having a molecular weight of at least about 41.6 kDa and which is at least about 362 amino acid residues in length. The rat mACHR-6 polypeptide is expressed in the brain. Amino acid residues 1-8 (SEQ ID NO:91), 26-47 (SEQ ID NO:92), 69-91 (SEQ ID NO:93), 114-136 (SEQ ID NO:94), 277-297 (SEQ ID NO:95), and 313-333 (SEQ ID NO:96) comprise transmembrane domains.

The mACHR-6 polypeptide, a biologically active portion or fragment of the polypeptide, or an allelic variant thereof can have one or more of the following mACHR-6 activities: 1) it can interact with (e.g., bind to) acetylcholine; 2) it can interact with (e.g., bind to) a G protein or another protein which naturally binds to mACHR-6; 3) it can modulate the activity of an ion channel (e.g., a potassium channel or a calcium channel); 4) it can modulate cytosolic ion, e.g., calcium, concentration; 5) it can modulate the release of a neurotransmitter, e.g., acetylcholine, from a neuron, e.g., a presynaptic neuron; 6) it can modulate an acetylcholine response in an acetylcholine responsive cell (e.g., a smooth muscle cell or a gland cell) to, for example, beneficially affect the acetylcholine responsive cell, e.g., a neuron; 7) it can signal ligand binding via phosphatidylinositol turnover; and 8) it can modulate, e.g., activate or inhibit, phospholipase C activity.

Various aspects of the invention are described in further detail in the following subsections:

I. Isolated Nucleic Acid Molecules

One aspect of the invention pertains to isolated nucleic acid molecules that encode mACHR-6 or biologically active portions thereof, as well as nucleic acid fragments sufficient for use as hybridization probes to identify mACHR-6-encoding nucleic acid (e.g., mACHR-6 mRNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated mACHR-6 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., a hippocampal cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having the nucleotide sequence of SEQ ID NO:33, 34, or 35, or a portion thereof, can be isolated using standard molecular biology techniques and the sequence information provided herein. For example, a human mACHR-6 cDNA can be isolated from a human hippocampus library using all or portion of SEQ ID NO:33, 34, or 35 as a hybridization probe and standard hybridization techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of SEQ ID NO:33, 34, or 35 can be isolated by the polymerase chain reaction using oligonucleotide primers designed based upon the sequence of SEQ ID NO:33, 34, or 35. For example, mRNA can be isolated from normal brain cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO:33, 34, or 35. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an mACHR-6 nucleotide sequence can be prepared by standard synthetic techniques, e.g., using an automated DNA synthesizer.

In a preferred embodiment, an isolated nucleic acid molecule of the invention comprises the nucleotide sequence shown in SEQ ID NO:33, 34, or 35. The sequence of SEQ ID NO:33 corresponds to the human mACHR-6 cDNA. This cDNA comprises sequences encoding the human mACHR-6 polypeptide (i.e., “the coding region”, from nucleotides 291 to 1628 of SEQ ID NO:33), as well as 5′ untranslated sequences (nucleotides 1 to 290 of SEQ ID NO:33) and 3′ untranslated sequences (nucleotides 1629 to 2689 of SEQ ID NO:33). Alternatively, the nucleic acid molecule can comprise only the coding region of SEQ ID NO:33 (e.g., nucleotides 291 to 1628 of SEQ ID NO:33, shown separately as SEQ ID NO:39). The sequence of SEQ ID NO:34 corresponds to the rat mACHR-6 cDNA. This cDNA comprises sequences encoding the rat mACHR-6 polypeptide (i.e., “the coding region”, from nucleotides 778 to 2112 of SEQ ID NO:34), as well as 5′ untranslated sequences (nucleotides 1 to 777 of SEQ ID NO:34), and 3′ untranslated sequences (nucleotides 2113 to 3244 of SEQ ID NO:34). Alternatively, the nucleic acid molecule can comprise only the coding region of SEQ ID NO:34 (e.g., nucleotides 778 to 2112 of SEQ ID NO:34, shown separately as SEQ ID NO:40). The sequence of SEQ ID NO:35 corresponds to the partial rat mACHR-6 cDNA. This cDNA comprises sequences encoding part of the rat mACHR-6 polypeptide (i.e., part of “the coding region”, from nucleotides 1 to 1089 of SEQ ID NO:35), and 3′ untranslated sequences (nucleotides 1090 to 2218 of SEQ ID NO:35). Alternatively, the nucleic acid molecule can comprise only the partial coding region of SEQ ID NO:35 (e.g., nucleotides 1 to 1089, shown separately as SEQ ID NO:41).

In another preferred embodiment, an isolated nucleic acid molecule of the invention comprises a nucleic acid molecule which is a complement of the nucleotide sequence shown in SEQ ID NO:33, 34, or 35, or a portion of either of these nucleotide sequences. A nucleic acid molecule which is complementary to the nucleotide sequence shown in SEQ ID NO:33, 34, or 35 is one which is sufficiently complementary to the nucleotide sequence shown in SEQ ID NO: 33, 34, or 35 such that it can hybridize to the nucleotide sequence shown in SEQ ID NO: 33, 34, or 35, respectively, thereby forming a stable duplex.

In still another preferred embodiment, an isolated nucleic acid molecule of the invention comprises a nucleotide sequence which is at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the nucleotide sequence shown in SEQ ID NO: 33, 34, or 35, or a portion of these nucleotide sequences. Preferably, such nucleic acid molecules encode functionally active or inactive allelic variants of mACHR-6. In an additional preferred embodiment, an isolated nucleic acid molecule of the invention comprises a nucleotide sequence which hybridizes, e.g., hybridizes under stringent conditions, to the nucleotide sequence shown in SEQ ID NO:33, 34, or 35, or a portion of either of these nucleotide sequences.

Moreover, the nucleic acid molecule of the invention can comprise only a portion of the coding region of SEQ ID NO:33, 34, or 35, for example a fragment which can be used as a probe or primer or a fragment encoding a biologically active portion of mACHR-6. The nucleotide sequence determined from the cloning of the mACHR-6 gene from a mammal allows for the generation of probes and primers designed for use in identifying and/or cloning mACHR-6 homologues in other cell types, e.g., from other tissues, as well as mACHR-6 homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of SEQ ID NO:33, 34, or 35 sense, an anti-sense sequence of SEQ ID NO:33, 34, or 35, or naturally occurring mutants thereof. Primers based on the nucleotide sequence in SEQ ID NO:33, 34, or 35 can be used in PCR reactions to clone mACHR-6 homologues. Probes based on the mACHR-6 nucleotide sequences can be used to detect transcripts or genomic sequences encoding the same or homologous polypeptides. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an mACHR-6 polypeptide, such as by measuring a level of an mACHR-6-encoding nucleic acid in a sample of cells from a subject e.g., detecting mACHR-6 mRNA levels or determining whether a genomic mACHR-6 gene has been mutated or deleted.

In one embodiment, the nucleic acid molecule of the invention encodes a polypeptide or portion thereof which includes an amino acid sequence which is sufficiently homologous to an amino acid sequence of SEQ ID NO:36, 37, or 38 such that the polypeptide or portion thereof maintains the ability to modulate an acetylcholine response in an acetylcholine responsive cell (e.g., naturally occurring allelic variants of the rat and human mACHR-6 polypeptides described herein). As used herein, the language “sufficiently homologous” refers to polypeptides or portions thereof which have amino acid sequences which include a minimum number of identical or equivalent (e.g., an amino acid residue which has a similar side chain as an amino acid residue in SEQ ID NO: 36, 37, or 38) amino acid residues to an amino acid sequence of SEQ ID NO:36, 37, or 38 or portion thereof is able to modulate an acetylcholine response in an acetylcholine responsive cell or a skilled artisan would clearly recognize it as a non-functional allelic variant of the rat and human mACHR-6 polypeptides described herein. Acetylcholine, as described herein, initiates a variety of responses in many different cell types. Examples of such responses are also described herein. In another embodiment, the polypeptide is at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the amino acid sequence of SEQ ID NO:36, 37, or 38.

Portions of polypeptides encoded by the mACHR-6 nucleic acid molecule of the invention are preferably biologically active portions of the mACHR-6 polypeptide. As used herein, the term “biologically active portion of mACHR-6” is intended to include a portion, e.g., a domain/motif, of mACHR-6 that has one or more of the following mACHR-6 activities: 1) it can interact with (e.g., bind to) acetylcholine; 2) it can interact with (e.g., bind to) a G protein or another protein which naturally binds to mACHR-6; 3) it can modulate the activity of an ion channel (e.g., a potassium channel or a calcium channel); 4) it can modulate cytosolic ion, e.g., calcium, concentration; 5) it can modulate the release of a neurotransmitter, e.g., acetylcholine, from a neuron, e.g., a presynaptic neuron; 6) it can modulate an acetylcholine response in an acetylcholine responsive cell (e.g., a smooth muscle cell or a gland cell) to, for example, beneficially affect the acetylcholine responsive cell, e.g., a neuron; 7) it can signal ligand binding via phosphatidylinositol turnover; and 8) it can modulate, e.g., activate or inhibit, phospholipase C activity.

Standard binding assays, e.g., immunoprecipitations and yeast two-hybrid assays as described herein, can be performed to determine the ability of an mACHR-6 polypeptide or a biologically active portion thereof to interact with (e.g., bind to) a binding partner such as a G protein. To determine whether an mACHR-6 polypeptide or a biologically active portion thereof can modulate an acetylcholine response in an acetylcholine responsive cell, such cells can be transfected with a construct driving the overexpression of an mACHR-6 polypeptide or a biologically active portion thereof. Methods for the preparation of acetylcholine responsive cells, e.g., intact smooth muscle cells or extracts from such cells are known in the art and described in Glukhova et al. (1987) Tissue Cell 19 (5):657-63, Childs et al. (1992) J. Biol. Chem. 267 (32):22853-9, and White et al. (1996) J. Biol. Chem. 271 (25):15008-17. The cells can be subsequently treated with acetylcholine, and a biological effect of acetylcholine on the cells, such as phosphatidylinositol turnover or cytosolic calcium concentration can be measured using methods known in the art (see Hartzell H. C. et al. (1988) Prog. Biophys. Mol. Biol. 52:165-247). Alternatively, transgenic animals, e.g., mice overexpressing an mACHR-6 polypeptide or a biologically active portion thereof, can be used. Tissues from such animals can be obtained and treated with acetylcholine. For example, methods for preparing detergent-skinned muscle fiber bundles are known in the art (Strauss et al. (1992) Am. J. Physiol. 262:1437-45). The contractility of these tissues in response to acetylcholine can be determined using, for example, isometric force measurements as described in Strauss et al., supra. Similarly, to determine whether an mACHR-6 polypeptide or a biologically active portion thereof can modulate an acetylcholine response in an acetylcholine responsive cell such as a gland cell, gland cells, e.g., parotid gland cells grown in tissue culture, can be transfected with a construct driving the overexpression of an mACHR-6 polypeptide or a biologically active portion thereof. The cells can be subsequently treated with acetylcholine, and the effect of the acetylcholine on amylase secretion from such cells can be determined using, for example an enzymatic assay with a labeled substrate. The preferred assays used for mACHR-6 activity will be based on phosphatidylinositol turnover such as those developed for the M1, M3 and M5 classes of receptors (see E. Watson et al. The G Protein Linked Receptor: FactsBook (Academic Press, Boston, Mass., 1994), the contents of which are incorporated herein by reference).

In one embodiment, the biologically active portion of mACHR-6 comprises a transmembrane domain. Preferably, the transmembrane domain is encoded by a nucleic acid molecule derived from a human and is at least about 50-55%, preferably at least about 60-65%, more preferably at least about 70-75%, even more preferably at least about 80-85%, and most preferably at least about 90-95% or more homologous to any of the transmembrane domains (i.e., amino acid residues 34-59, 109-130, 152-174, 197-219, or 396-416) of SEQ ID NO:36 which are shown as separate sequences designated SEQ ID NOs:42, 43, 44, 45, and 46, respectively, or to the rat transmembrane domains (i.e., amino acid residues 34-59, 73-91, 109-130, 152-174, 197-219, 360-380, or 396-416 of SEQ ID NO:37 which are shown as separate sequences designated SEQ ID NOs:47, 48,49,50, 51,52, and 53, respectively or amino acid residues 1-8, 26-47, 69-91, 114-136, 277-297, or 313-333 of SEQ ID NO:38 which are shown as separate sequences designated SEQ ID NOs:91, 92, 93, 94, 95, or 96, respectively). More preferably, the transmembrane domain encoded by the human nucleic acid molecule is at least about 75-80%, preferably at least about 80-85%, more preferably at least about 85-90%, and most preferably at least about 90-95% or more homologous to the transmembrane domain (i.e., amino acid residues 360-380) of SEQ ID NO:36 which is shown as a separate sequence designated SEQ ID NO:60, or at least about 80-85%, more preferably at least about 85-90%, and most preferably at least about 90-95% or more homologous to the transmembrane domain (i.e., amino acid residues 73-91) of SEQ ID NO:36 which is shown as a separate sequence designated SEQ ID NO:61. In a preferred embodiment, the biologically active portion of the polypeptide which includes the transmembrane domain can modulate the activity of a G protein or other binding partner in a cell and/or modulate an acetylcholine response in an acetylcholine responsive cell, e.g., a brain cell, to thereby beneficially affect the cell. In a preferred embodiment, the biologically active portion comprises a transmembrane domain of the human mACHR-6 as represented by amino acid residues 34-59 (SEQ ID NO:42), 73-91 (SEQ ID NO:61), 109-130 (SEQ ID NO:43), 152-174 (SEQ ID NO:44), 197-219 (SEQ ID NO:45), 360-380 (SEQ ID NO:60), and 396-416 (SEQ ID NO:46), a transmembrane domain of the full length rat mACHR-6 as represented by amino acid residues 34-59 (SEQ ID NO:47), 73-91 (SEQ ID NO:48), 109-130 (SEQ ID NO:49), 152-174 (SEQ ID NO:50), 197-219 (SEQ ID NO:51), 360-380 (SEQ ID NO:52), and 396-416 (SEQ ID NO:53), or a transmembrane domain of the partial rat mACHR-6 as represented by amino residues 1-8 (SEQ ID NO:91), 26-47 (SEQ ID NO:92), 69-91 (SEQ ID NO:93), 114-136 (SEQ ID NO:94), 277-297 (SEQ ID NO:95), and 313-333 (SEQ ID NO:96). Additional nucleic acid fragments encoding biologically active portions of mACHR-6 can be prepared by isolating a portion of SEQ ID NO:33, 34, or 35, expressing the encoded portion of mACHR-6 polypeptide or peptide (e.g., by recombinant expression in vitro) and assessing the activity of the encoded portion of mACHR-6 polypeptide or peptide.

The invention further encompasses nucleic acid molecules that differ from the nucleotide sequence shown in SEQ ID NO:33, 34, or 35 (and portions thereof) due to degeneracy of the genetic code and thus encode the same mACHR-6 polypeptide as that encoded by the nucleotide sequence shown in SEQ ID NO:33, 34, or 35. In another embodiment, an isolated nucleic acid molecule of the invention has a nucleotide sequence encoding a polypeptide having an amino acid sequence shown in SEQ ID NO:36, 37, or 38. In a still further embodiment, the nucleic acid molecule of the invention encodes a full length human polypeptide which is substantially homologous to the amino acid sequence of SEQ ID NO:36, 37 or 38 (encoded by the open reading frame shown in SEQ ID NO:39, 40, or 41, respectively).

In addition to the mACHR-6 nucleotide sequence shown in SEQ ID NO:33, 34, or 35, it will be appreciated by those skilled in the art that DNA sequence polymorphisms that lead to changes in the amino acid sequences of mACHR-6 may exist within a population (e.g., the human population). Such genetic polymorphism in the mACHR-6 gene may exist among individuals within a population due to natural allelic variation. As used herein, the terms “gene” and “recombinant gene” refer to nucleic acid molecules comprising an open reading frame encoding an mACHR-6 polypeptide, preferably a mammalian mACHR-6 polypeptide. Such natural allelic variations can typically result in 1-5% variance in the nucleotide sequence of the mACHR-6 gene. Any and all such nucleotide variations and resulting amino acid polymorphisms in mACHR-6 that are the result of natural allelic variation are intended to be within the scope of the invention. Such allelic variation includes both active allelic variants as well as non-active or reduced activity allelic variants, the later two types typically giving rise to a pathological disorder. Moreover, nucleic acid molecules encoding mACHR-6 polypeptides from other species, and thus which have a nucleotide sequence which differs from the human sequence of SEQ ID NO:33, are intended to be within the scope of the invention. Nucleic acid molecules corresponding to natural allelic variants and non-human homologues of the human mACHR-6 cDNA of the invention can be isolated based on their homology to the human mACHR-6 nucleic acid disclosed herein using the human cDNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising the nucleotide sequence of SEQ ID NO:33. In other embodiments, the nucleic acid is at least 30, 50, 100, 250, 300, 400, 500, 600, 700, 800, 900, or 1000 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:33 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural polypeptide). In one embodiment, the nucleic acid encodes a natural human mACHR-6.

In addition to naturally-occurring allelic variants of the mACHR-6 sequence that may exist in the population, the skilled artisan will further appreciate that changes can be introduced by mutation into the nucleotide sequence of SEQ ID NO:33, 34, or 35, thereby leading to changes in the amino acid sequence of the encoded mACHR-6 polypeptide, without altering the functional ability of the mACHR-6 polypeptide. For example, nucleotide substitutions leading to amino acid substitutions at “non-essential” amino acid residues can be made in the sequence of SEQ ID NO:33, 34, or 35. A “non-essential” amino acid residue is a residue that can be altered from the wild-type sequence of mACHR-6 (e.g., the sequence of SEQ ID NO:36, 37, or 38) without altering the activity of mACHR-6, whereas an “essential” amino acid residue is required for mACHR-6 activity. For example, conserved amino acid residues, e.g., aspartates, prolines threonines and tyrosines, in the transmembrane domains of mACHR-6 are most likely important for binding to acetylcholine and are thus essential residues of mACHR-6. Other amino acid residues, however, (e.g., those that are not conserved or only semi-conserved in the transmembrane domain) may not be essential for activity and thus are likely to be amenable to alteration without altering mACHR-6 activity.

Accordingly, another aspect of the invention pertains to nucleic acid molecules encoding mACHR-6 polypeptides that contain changes in amino acid residues that are not essential for mACHR-6 activity. Such mACHR-6 polypeptides differ in amino acid sequence from SEQ ID NO:36, 37, or 38 yet retain at least one of the mACHR-6 activities described herein. In one embodiment, the isolated nucleic acid molecule comprises a nucleotide sequence encoding a polypeptide, wherein the polypeptide comprises an amino acid sequence at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the amino acid sequence of SEQ ID NO:36, 37, or 38.

To determine the percent homology of two amino acid sequences (e.g., SEQ ID NO:36, 37, or 38 and a mutant form thereof) or of two nucleic acids, the sequences are aligned for optimal comparison purposes (e.g., gaps can be introduced in the sequence of one polypeptide or nucleic acid for optimal alignment with the other polypeptide or nucleic acid). The amino acid residues or nucleotides at corresponding amino acid positions or nucleotide positions are then compared. When a position in one sequence (e.g., SEQ ID NO:36, 37, or 38) is occupied by the same amino acid residue or nucleotide as the corresponding position in the other sequence (e.g., a mutant form of mACHR-6), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. BLAST nucleotide searches can be performed performed with the NBLAST program, score=100, wordlength=12 to obtain nucleotide sequences homologous to mACHR-6 nucleic acid molecules of the invention. BLAST protein searches can be performed with the XBLAST program, score=50, wordlength=3 to obtain amino acid sequences homologous to mACHR-6 protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

An isolated nucleic acid molecule encoding an mACHR-6 polypeptide homologous to the polypeptide of SEQ ID NO:36, 37, or 38 can be created by introducing one or more nucleotide substitutions, additions or deletions into the nucleotide sequence of SEQ ID NO:33, 34, or 35, respectively, such that one or more amino acid substitutions, additions or deletions are introduced into the encoded polypeptide. Mutations can be introduced into SEQ ID NO:33, 34, or 35 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in mACHR-6 is preferably replaced with another amino acid residue from the same side chain family. Alternatively, in another embodiment, mutations can be introduced randomly along all or part of an mACHR-6 coding sequence, such as by saturation mutagenesis, and the resultant mutants can be screened for an mACHR-6 activity described herein to identify mutants that retain mACHR-6 activity. Following mutagenesis of SEQ ID NO:33, 34, or 35, the encoded polypeptide can be expressed recombinantly (e.g., as described in Examples 3 and 4) and the activity of the polypeptide can be determined using, for example, assays described herein.

In addition to the nucleic acid molecules encoding mACHR-6 polypeptides described above, another aspect of the invention pertains to isolated nucleic acid molecules which are antisense thereto. An “antisense” nucleic acid comprises a nucleotide sequence which is complementary to a “sense” nucleic acid encoding a polypeptide, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire mACHR-6 coding strand, or to only a portion thereof. In one embodiment, an antisense nucleic acid molecule is antisense to a “coding region” of the coding strand of a nucleotide sequence encoding mACHR-6.

The term “coding region” refers to the region of the nucleotide sequence comprising codons which are translated into amino acid residues, e.g., the entire coding region of SEQ ID NO:33 comprises nucleotides 291 to 1628 (shown separately as SEQ ID NO:39) and the coding region of SEQ ID NO:34 comprises nucleotides 778 to 2112 (shown separately as SEQ ID NO:40). In another embodiment, the antisense nucleic acid molecule is antisense to a “noncoding region” of the coding strand of a nucleotide sequence encoding mACHR-6. The term “noncoding region” refers to 5′ and 3′ sequences which flank the coding region that are not translated into amino acids (i.e., also referred to as 5′ and 3′ untranslated regions).

Given the coding strand sequences encoding mACHR-6 disclosed herein (e.g., SEQ ID NOs:33, 34, or 35), antisense nucleic acids of the invention can be designed according to the rules of Watson and Crick base pairing. The antisense nucleic acid molecule can be complementary to the entire coding region of mACHR-6 mRNA, but more preferably is an oligonucleotide which is antisense to only a portion of the coding or noncoding region of mACHR-6 mRNA. For example, the antisense oligonucleotide can be complementary to the region surrounding the translation start site of mACHR-6 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an mACHR-6 polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mACHR-mRNA transcripts to thereby inhibit translation of mACHR-6 mRNA. A ribozyme having specificity for an mACHR-6-encoding nucleic acid can be designed based upon the nucleotide sequence of an mACHR-6 cDNA disclosed herein (i.e., SEQ ID NO:33, 34, or 35). For example, a derivative of a Tetrahymena L-19 IVS RNA can be constructed in which the nucleotide sequence of the active site is complementary to the nucleotide sequence to be cleaved in an mACHR-6-encoding mRNA. See, e.g., Cech et al. U.S. Pat. No. 4,987,071 and Cech et al. U.S. Pat. No. 5,116,742. Alternatively, mACHR-6 mRNA can be used to select a catalytic RNA having a specific ribonuclease activity from a pool of RNA molecules. See, e.g., Bartel, D. and Szostak, J. W. (1993) Science 261:1411-1418.

Alternatively, mACHR-6 gene expression can be inhibited by targeting nucleotide sequences complementary to the regulatory region of the mACHR-6 (e.g., the mACHR-6 promoter and/or enhancers) to form triple helical structures that prevent transcription of the mACHR-6 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

II. Recombinant Expression Vectors and Host Cells

Another aspect of the invention pertains to vectors, preferably expression vectors, containing a nucleic acid encoding mACHR-6 (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein (e.g., mACHR-6 polypeptides, mutant forms of mACHR-6, fusion polypeptides, and the like).

The recombinant expression vectors of the invention can be designed for expression of mACHR-6 in prokaryotic or eukaryotic cells. For example, mACHR-6 can be expressed in bacterial cells such as E. coli, insect cells (e.g., using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant polypeptide. In one embodiment, the coding sequence of the mACHR-6 is cloned into a pGEX expression vector to create a vector encoding a fusion polypeptide comprising, from the N-terminus to the C-terminus, GST-thrombin cleavage site-mACHR-6. The fusion polypeptide can be purified by affinity chromatography using glutathione-agarose resin. Recombinant mACHR-6 unfused to GST can be recovered by cleavage of the fusion polypeptide with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant polypeptide expression in E. coli is to express the polypeptide in a host bacteria with an impaired capacity to proteolytically cleave the recombinant polypeptide (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the mACHR-6 expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, mACHR-6 can be expressed in insect cells using, for example, baculovirus expression vectors. Baculovirus vectors available for expression of polypeptides in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the □-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to mACHR-6 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, mACHR-6 polypeptide can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding mACHR-6 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) mACHR-6 polypeptide. Accordingly, the invention further provides methods for producing mACHR-6 polypeptide using the host cells of the invention. In one embodiment, the method comprises culturing the host cell of invention (into which a recombinant expression vector encoding mACHR-6 has been introduced) in a suitable medium until mACHR-6 is produced. In another embodiment, the method further comprises isolating mACHR-6 from the medium or the host cell.

The host cells of the invention can also be used to produce non-human transgenic animals. The non-human transgenic animals can be used in screening assays designed to identify agents or compounds, e.g., drugs, pharmaceuticals, etc., which are capable of ameliorating detrimental symptoms of selected disorders such as nervous system disorders, smooth muscle related disorders, cardiac muscle related disorders and gland related disorders. For example, in one embodiment, a host cell of the invention is a fertilized oocyte or an embryonic stem cell into which mACHR-6-coding sequences have been introduced. Such host cells can then be used to create non-human transgenic animals in which exogenous mACHR-6 sequences have been introduced into their genome or homologous recombinant animals in which endogenous mACHR-6 sequences have been altered. Such animals are useful for studying the function and/or activity of mACHR-6 and for identifying and/or evaluating modulators of mACHR-6 activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous mACHR-6 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing mACHR-6-encoding nucleic acid into the male pronuclei of a fertilized oocyte, e.g., by microinjection, retroviral infection, and allowing the oocyte to develop in a pseudopregnant female foster animal. The human mACHR-6 cDNA sequence of SEQ ID NO:33 can be introduced as a transgene into the genome of a non-human animal. Furthermore, the rat mACHR-6 cDNA sequence of SEQ ID NO:34 can be introduced as a transgene into the genome of a non-rat animal. Moreover, a non-human homologue of the human mACHR-6 gene, such as a mouse mACHR-6 gene, can be isolated based on hybridization to the human or rat mACHR-6 cDNA (described further in subsection I above) and used as a transgene. Intronic sequences and polyadenylation signals can also be included in the transgene to increase the efficiency of expression of the transgene. A tissue-specific regulatory sequence(s) can be operably linked to the mACHR-6 transgene to direct expression of mACHR-6 polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the mACHR-6 transgene in its genome and/or expression of mACHR-6 mRNA in tissues or cells of the animals. A transgenic founder animal can then be used to breed additional animals carrying the transgene. Moreover, transgenic animals carrying a transgene encoding mACHR-6 can further be bred to other transgenic animals carrying other transgenes.

To create a homologous recombinant animal, a vector is prepared which contains at least a portion of an mACHR-6 gene into which a deletion, addition or substitution has been introduced to thereby alter, e.g., functionally disrupt, the mACHR-6 gene. The mACHR-6 gene can be a human gene (e.g., from a human genomic clone isolated from a human genomic library screened with the cDNA of SEQ ID NO:33), but more preferably, is a rat mACHR-6 gene of SEQ ID NO:34 or 35, or another non-human homologue of a human mACHR-6 gene. For example, a mouse mACHR-6 gene can be isolated from a mouse genomic DNA library using the mACHR-6 cDNA of SEQ ID NO:33, 4, or 31 as a probe. The mouse mACHR-6 gene then can be used to construct a homologous recombination vector suitable for altering an endogenous mACHR-6 gene in the mouse genome. In a preferred embodiment, the vector is designed such that, upon homologous recombination, the endogenous mACHR-6 gene is functionally disrupted (i.e., no longer encodes a functional polypeptide; also referred to as a “knock out” vector). Alternatively, the vector can be designed such that, upon homologous recombination, the endogenous mACHR-6 gene is mutated or otherwise altered but still encodes functional polypeptide (e.g., the upstream regulatory region can be altered to thereby alter the expression of the endogenous mACHR-6 polypeptide). In the homologous recombination vector, the altered portion of the mACHR-6 gene is flanked at its 5′ and 3′ ends by additional nucleic acid of the mACHR-6 gene to allow for homologous recombination to occur between the exogenous mACHR-6 gene carried by the vector and an endogenous mACHR-6 gene in an embryonic stem cell. The additional flanking mACHR-6 nucleic acid is of sufficient length for successful homologous recombination with the endogenous gene. Typically, several kilobases of flanking DNA (both at the 5′ and 3′ ends) are included in the vector (see for example, Thomas, K. R. and Capecchi, M. R. (1987) Cell 51:503 for a description of homologous recombination vectors). The vector is introduced into an embryonic stem cell line (e.g., by electroporation) and cells in which the introduced mACHR-6 gene has homologously recombined with the endogenous mACHR-6 gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; WO 92/0968; and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected polypeptide are required. Such animals can be provided through the construction of “double” transgenic animals, e.g., by mating two transgenic animals, one containing a transgene encoding a selected polypeptide and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

III. Isolated mACHR-6 Polypeptides and Anti-mACHR-6 Antibodies

Another aspect of the invention pertains to isolated mACHR-6 polypeptides, and biologically active portions thereof, as well as peptide fragments suitable for use as immunogens to raise anti-mACHR-6 antibodies. An “isolated” or “purified” polypeptide or biologically active portion thereof is substantially free of cellular material when produced by recombinant DNA techniques, or chemical precursors or other chemicals when chemically synthesized. The language “substantially free of cellular material” includes preparations of mACHR-6 polypeptide in which the polypeptide is separated from cellular components of the cells in which it is naturally or recombinantly produced. In one embodiment, the language “substantially free of cellular material” includes preparations of mACHR-6 polypeptide having less than about 30% (by dry weight) of non-mACHR-6 polypeptide (also referred to herein as a “contaminating polypeptide”), more preferably less than about 20% of non-mACHR-6 polypeptide, still more preferably less than about 10% of non-mACHR-6 polypeptide, and most preferably less than about 5% non-mACHR-6 polypeptide. When the mACHR-6 polypeptide or biologically active portion thereof is recombinantly produced, it is also preferably substantially free of culture medium, i.e., culture medium represents less than about 20%, more preferably less than about 10%, and most preferably less than about 5% of the volume of the polypeptide preparation. The language “substantially free of chemical precursors or other chemicals” includes preparations of mACHR-6 polypeptide in which the polypeptide is separated from chemical precursors or other chemicals which are involved in the synthesis of the polypeptide. In one embodiment, the language “substantially free of chemical precursors or other chemicals” includes preparations of mACHR-6 polypeptide having less than about 30% (by dry weight) of chemical precursors or non-mACHR-6 chemicals, more preferably less than about 20% chemical precursors or non-mACHR-6 chemicals, still more preferably less than about 10% chemical precursors or non-mACHR-6 chemicals, and most preferably less than about 5% chemical precursors or non-mACHR-6 chemicals. In preferred embodiments, isolated polypeptides or biologically active portions thereof lack contaminating polypeptides from the same animal from which the mACHR-6 polypeptide is derived. Typically, such polypeptides are produced by recombinant expression of, for example, a human mACHR-6 polypeptide in a non-human cell.

An isolated mACHR-6 polypeptide or a portion thereof of the invention can modulate an acetylcholine response in an acetylcholine responsive cell or be a naturally occurring, non-functional allelic variant of an mACHR-6 polypeptide. In preferred embodiments, the polypeptide or portion thereof comprises an amino acid sequence which is sufficiently homologous to an amino acid sequence of SEQ ID NO:36, 37, or 38 such that the polypeptide or portion thereof maintains the ability to modulate an acetylcholine response in an acetylcholine responsive cell. The portion of the polypeptide is preferably a biologically active portion as described herein. In another preferred embodiment, the human mACHR-6 polypeptide (i.e., amino acid residues 1-398 of SEQ ID NO:36) or the rat mACHR-6 polypeptide (i.e., amino acid residues 1-445 of SEQ ID NO:37 or amino acid residues 1-401 of SEQ ID NO:38) has an amino acid sequence shown in SEQ ID NO:36, 37, or 38. In yet another preferred embodiment, the mACHR-6 polypeptide has an amino acid sequence which is encoded by a nucleotide sequence which hybridizes. In still another preferred embodiment, the mACHR-6 polypeptide has an amino acid sequence which is encoded by a nucleotide sequence that is at least about 30-35%, preferably at least about 40-45%, more preferably at least about 50-55%, even more preferably at least about 60-65%, yet more preferably at least about 70-75%, still more preferably at least about 80-85%. The preferred mACHR-6 polypeptides of the present invention also preferably possess at least one of the mACHR-6 activities described herein. For example, a preferred mACHR-6 polypeptide of the present invention includes an amino acid sequence encoded by a nucleotide sequence which hybridizes, e.g., hybridizes under stringent conditions.

In other embodiments, the mACHR-6 polypeptide is substantially homologous to the amino acid sequence of SEQ ID NO:36, 37, or 38 and retains the functional activity of the polypeptide of SEQ ID NO: 36, 37, or 38 yet differs in amino acid sequence due to natural allelic variation or mutagenesis, as described in detail in subsection I above. Accordingly, in another embodiment, the mACHR-6 polypeptide is a polypeptide which comprises an amino acid sequence which is at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the amino acid sequence of SEQ ID NO: 36, 37, or 38 and which has at least one of the mACHR-6 activities described herein. In still other embodiments, the invention pertains to a full length human polypeptide which is substantially homologous to the entire amino acid sequence of SEQ ID NO: 36, 37, or 38. In still another embodiment, the invention pertains to nonfunctional, naturally occurring allelic variants of the mACHR-6 polypeptides described herein. Such allelic variants will typically contain a non-conservative substitution, a deletion, or insertion or premature truncation of the amino acid sequence of SEQ ID NO: 36, 37, or 38.

Biologically active portions of the mACHR-6 polypeptide include peptides comprising amino acid sequences derived from the amino acid sequence of the mACHR-6 polypeptide, e.g., the amino acid sequence shown in SEQ ID NO: 36, 37, or 38 or the amino acid sequence of a polypeptide homologous to the mACHR-6 polypeptide, which include less amino acids than the full length mACHR-6 polypeptide or the full length polypeptide which is homologous to the mACHR-6 polypeptide, and exhibit at least one activity of the mACHR-6 polypeptide. Typically, biologically active portions (peptides, e.g., peptides which are, for example, 5, 10, 15, 20, 30, 35, 36, 37, 38, 39, 40, 50, 100 or more amino acids in length) comprise a domain or motif, e.g., a transmembrane domain, with at least one activity of the mACHR-6 polypeptide. Preferably, the domain is a transmembrane domain derived from a human and is at least about 75-80%, preferably at least about 80-85%, more preferably at least about 85-90%, and most preferably at least about 90-95% or more homologous to SEQ ID NO:42, 61, 43, 44, 45, 60 or 46 or to the corresponding rat sequences. In a preferred embodiment, the biologically active portion of the polypeptide which includes the transmembrane domain can modulate the activity of a G protein in a cell and/or modulate an acetylcholine response in a cell, e.g., an acetylcholine responsive cell, e.g., a brain cell, to thereby beneficially affect the acetylcholine responsive cell. In a preferred embodiment, the biologically active portion comprises a transmembrane domain of mACHR-6 as represented by amino acid residues 34-59 (SEQ ID NO:42), 73-91 (SEQ ID NO:61), 109-130 (SEQ ID NO:43), 152-174 (SEQ ID NO:44), 197-219 (SEQ ID NO:45), 360-380 (SEQ ID NO:60), and 396-416 (SEQ ID NO:46), or the corresponding rat sequences shown in SEQ ID NOs:47-53 and 91-96. Moreover, other biologically active portions, in which other regions of the polypeptide are deleted, can be prepared by recombinant techniques and evaluated for one or more of the activities described herein. Preferably, the biologically active portions of the mACHR-6 polypeptide include one or more selected domains/motifs or portions thereof having biological activity.

mACHR-6 polypeptides are preferably produced by recombinant DNA techniques. For example, a nucleic acid molecule encoding the polypeptide is cloned into an expression vector (as described above), the expression vector is introduced into a host cell (as described above) and the mACHR-6 polypeptide is expressed in the host cell. The mACHR-6 polypeptide can then be isolated from the cells by an appropriate purification scheme using standard polypeptide purification techniques. Alternative to recombinant expression, an mACHR-6 polypeptide, protein, or peptide r can be synthesized chemically using standard peptide synthesis techniques. Moreover, native mACHR-6 polypeptide can be isolated from cells (e.g., hippocampal cells, substantia nigra cells, or parotid gland cells), for example using an anti-mACHR-6 antibody (described further below).

The invention also provides mACHR-6 chimeric or fusion polypeptides. As used herein, an mACHR-6 “chimeric polypeptide” or “fusion polypeptide” comprises an mACHR-6 polypeptide operatively linked to a non-mACHR-6 polypeptide. An “mACHR-6 polypeptide” refers to a polypeptide having an amino acid sequence corresponding to mACHR-6, whereas a “non-mACHR-6 polypeptide” refers to a heterologous polypeptide having an amino acid sequence corresponding to a polypeptide which is not substantially homologous to the mACHR-6 polypeptide, e.g., a polypeptide which is different from the mACHR-6 polypeptide and which is derived from the same or a different organism. Within the fusion polypeptide, the term “operatively linked” is intended to indicate that the mACHR-6 polypeptide and the non-mACHR-6 polypeptide are fused in-frame to each other. The non-mACHR-6 polypeptide can be fused to the N-terminus or C-terminus of the mACHR-6 polypeptide. For example, in one embodiment the fusion polypeptide is a GST-mACHR-6 fusion polypeptide in which the mACHR-6 sequences are fused to the C-terminus of the GST sequences. Other types of fusion polypeptides include, but are not limited to, enzymatic fusion polypeptides, for example beta-galactosidase fusions, yeast two-hybrid GAL fusions, poly His fusions and Ig fusions. Such fusion polypeptides, particularly poly His fusions, can facilitate the purification of recombinant mACHR-6. In another embodiment, the fusion polypeptide is an mACHR-6 polypeptide containing a heterologous signal sequence at its N-terminus. In certain host cells (e.g., mammalian host cells), expression and/or secretion of mACHR-6 can be increased through use of a heterologous signal sequence.

Preferably, an mACHR-6 chimeric or fusion polypeptide of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An mACHR-6-encoding nucleic acid can be cloned into such an expression vector such that the fusion moiety is linked in-frame to the mACHR-6 polypeptide.

The present invention also pertains to homologues of the mACHR-6 polypeptides which function as either an mACHR-6 agonist (mimetic) or an mACHR-6 antagonist. In a preferred embodiment, the mACHR-6 agonists and antagonists stimulate or inhibit, respectively, a subset of the biological activities of the naturally occurring form of the mACHR-6 polypeptide. Thus, specific biological effects can be elicited by treatment with a homologue of limited function. In one embodiment, treatment of a subject with a homologue having a subset of the biological activities of the naturally occurring form of the polypeptide has fewer side effects in a subject relative to treatment with the naturally occurring form of the mACHR-6 polypeptide.

Homologues of the mACHR-6 polypeptide can be generated by mutagenesis, e.g., discrete point mutation or truncation of the mACHR-6 polypeptide. As used herein, the term “homologue” refers to a variant form of the mACHR-6 polypeptide which acts as an agonist or antagonist of the activity of the mACHR-6 polypeptide. An agonist of the mACHR-6 polypeptide can retain substantially the same, or a subset, of the biological activities of the mACHR-6 polypeptide. An antagonist of the mACHR-6 polypeptide can inhibit one or more of the activities of the naturally occurring form of the mACHR-6 polypeptide, by, for example, competitively binding to a downstream or upstream member of the mACHR-6 cascade which includes the mACHR-6 polypeptide. Thus, the mammalian mACHR-6 polypeptide and homologues thereof of the present invention can be either positive or negative regulators of acetylcholine responses in acetylcholine responsive cells.

In an alternative embodiment, homologues of the mACHR-6 polypeptide can be identified by screening combinatorial libraries of mutants, e.g., truncation mutants, of the mACHR-6 polypeptide for mACHR-6 polypeptide agonist or antagonist activity. In one embodiment, a variegated library of mACHR-6 variants is generated by combinatorial mutagenesis at the nucleic acid level and is encoded by a variegated gene library. A variegated library of mACHR-6 variants can be produced by, for example, enzymatically ligating a mixture of synthetic oligonucleotides into gene sequences such that a degenerate set of potential mACHR-6 sequences is expressible as individual polypeptides, or alternatively, as a set of larger fusion polypeptides (e.g., for phage display) containing the set of mACHR-6 sequences therein. There are a variety of methods which can be used to produce libraries of potential mACHR-6 homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential mACHR-6 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the mACHR-6 polypeptide coding can be used to generate a variegated population of mACHR-6 fragments for screening and subsequent selection of homologues of an mACHR-6 polypeptide. In one embodiment, a library of coding sequence fragments can be generated by treating a double stranded PCR fragment of an mACHR-6 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the mACHR-6 polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of mACHR-6 homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify mACHR-6 homologues (Arkin and Yourvan (1992) PNAS 89:7811-7815; Delgrave et al. (1993) Protein Engineering 6(3):327-331).

In one embodiment, cell based assays can be exploited to analyze a variegated mACHR-6 library. For example, a library of expression vectors can be transfected into a cell line ordinarily responsive to acetylcholine. The transfected cells are then contacted with acetylcholine and the effect of the mACHR-6 mutant on signaling by acetylcholine can be detected, e.g., by measuring intracellular calcium concentration. Plasmid DNA can then be recovered from the cells which score for inhibition, or alternatively, potentiation of acetylcholine induction, and the individual clones further characterized.

An isolated mACHR-6 polypeptide, or a portion or fragment thereof, can be used as an immunogen to generate antibodies that bind mACHR-6 using standard techniques for polyclonal and monoclonal antibody preparation. The full-length mACHR-6 polypeptide can be used or, alternatively, the invention provides antigenic peptide fragments of mACHR-6 for use as immunogens. The antigenic peptide of mACHR-6 comprises at least 8 amino acid residues of the amino acid sequence shown in SEQ ID NO:36, 37, or 38 and encompasses an epitope of mACHR-6 such that an antibody raised against the peptide forms a specific immune complex with mACHR-6. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of mACHR-6 that are located on the surface of the polypeptide, e.g., hydrophilic regions.

An mACHR-6 immunogen typically is used to prepare antibodies by immunizing a suitable subject, (e.g., rabbit, goat, mouse or other mammal) with the immunogen. An appropriate immunogenic preparation can contain, for example, recombinantly expressed mACHR-6 polypeptide or a chemically synthesized mACHR-6 peptide. The preparation can further include an adjuvant, such as Freund's complete or incomplete adjuvant, or similar immunostimulatory agent. Immunization of a suitable subject with an immunogenic mACHR-6 preparation induces a polyclonal anti-mACHR-6 antibody response.

Accordingly, another aspect of the invention pertains to anti-mACHR-6 antibodies. The term “antibody” as used herein refers to immunoglobulin molecules and immunologically active portions of immunoglobulin molecules, i.e., molecules that contain an antigen binding site which specifically binds (immunoreacts with) an antigen, such as mACHR-6. Examples of immunologically active portions of immunoglobulin molecules include F(ab) and F(ab′)2 fragments which can be generated by treating the antibody with an enzyme such as pepsin. The invention provides polyclonal and monoclonal antibodies that bind mACHR-6. The term “monoclonal antibody” or “monoclonal antibody composition”, as used herein, refers to a population of antibody molecules that contain only one species of an antigen binding site capable of immunoreacting with a particular epitope of mACHR-6. A monoclonal antibody composition thus typically displays a single binding affinity for a particular mACHR-6 polypeptide with which it immunoreacts.

Polyclonal anti-mACHR-6 antibodies can be prepared as described above by immunizing a suitable subject with an mACHR-6 immunogen. The anti-mACHR-6 antibody titer in the immunized subject can be monitored over time by standard techniques, such as with an enzyme linked immunosorbent assay (ELISA) using immobilized mACHR-6. If desired, the antibody molecules directed against mACHR-6 can be isolated from the mammal (e.g., from the blood) and further purified by well known techniques, such as protein A chromatography to obtain the IgG fraction. At an appropriate time after immunization, e.g., when the anti-mACHR-6 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an mACHR-6 immunogen as described above, and the culture supernatants of the resulting hybridoma cells are screened to identify a hybridoma producing a monoclonal antibody that binds mACHR-6.

Any of the many well known protocols used for fusing lymphocytes and immortalized cell lines can be applied for the purpose of generating an anti-mACHR-6 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from ATCC®. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind mACHR-6, e.g., using a standard ELISA assay.

Alternative to preparing monoclonal antibody-secreting hybridomas, a monoclonal anti-mACHR-6 antibody can be identified and isolated by screening a recombinant combinatorial immunoglobulin library (e.g., an antibody phage display library) with mACHR-6 to thereby isolate immunoglobulin library members that bind mACHR-6. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-mACHR-6 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. PCT International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-mACHR-6 antibody (e.g., monoclonal antibody) can be used to isolate mACHR-6 by standard techniques, such as affinity chromatography or immunoprecipitation. An anti-mACHR-6 antibody can facilitate the purification of natural mACHR-6 from cells and of recombinantly produced mACHR-6 expressed in host cells. Moreover, an anti-mACHR-6 antibody can be used to detect mACHR-6 polypeptide (e.g., in a cellular lysate or cell supernatant) in order to evaluate the abundance and pattern of expression of the mACHR-6 polypeptide or a fragment of an mACHR-6 polypeptide. The detection of circulating fragments of an mACHR-6 polypeptide can be used to identify mACHR-6 turnover in a subject. Anti-mACHR-6 antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

IV. Pharmaceutical Compositions

The mACHR-6 nucleic acid molecules, mACHR-6 polypeptides (particularly fragments of mACHR-6), mACHR-6 modulators, and anti-mACHR-6 antibodies (also referred to herein as “active compounds”) of the invention can be incorporated into pharmaceutical compositions suitable for administration to a subject, e.g., a human. Such compositions typically comprise the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an mACHR-6 polypeptide or anti-mACHR-6 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, polypeptides, polypeptide homologues, modulators, and antibodies described herein can be used in one or more of the following methods: a) drug screening assays; b) diagnostic assays particularly in disease identification, allelic screening and pharmocogenetic testing; c) methods of treatment; d) pharmacogenomics; and e) monitoring of effects during clinical trials. An mACHR-6 polypeptide of the invention has one or more of the activities described herein and can thus be used to, for example, modulate an acetylcholine response in an acetylcholine responsive cell, for example by binding to acetylcholine or an mACHR-6 binding partner making it unavailable for binding to the naturally present mACHR-6 polypeptide. The isolated nucleic acid molecules of the invention can be used to express mACHR-6 polypeptide (e.g., via a recombinant expression vector in a host cell or in gene therapy applications), to detect mACHR-6 mRNA (e.g., in a biological sample) or a naturally occurring or recombinantly generated genetic mutation in an mACHR-6 gene, and to modulate mACHR-6 activity, as described further below. In addition, the mACHR-6 polypeptides can be used to screen drugs or compounds which modulate mACHR-6 polypeptide activity as well as to treat disorders characterized by insufficient production of mACHR-6 polypeptide or production of mACHR-6 polypeptide forms which have decreased activity compared to wild type mACHR-6. Moreover, the anti-mACHR-6 antibodies of the invention can be used to detect and isolate an mACHR-6 polypeptide, particularly fragments of mACHR-6 present in a biological sample, and to modulate mACHR-6 polypeptide activity.

a. Drug Screening Assays:

The invention provides methods for identifying compounds or agents which can be used to treat disorders characterized by (or associated with) aberrant or abnormal mACHR-6 nucleic acid expression and/or mACHR-6 polypeptide activity. These methods are also referred to herein as drug screening assays and typically include the step of screening a candidate/test compound or agent to be an agonist or antagonist of mACHR-6, and specifically for the ability to interact with (e.g., bind to) an mACHR-6 polypeptide, to modulate the interaction of an mACHR-6 polypeptide and a target molecule, and/or to modulate mACHR-6 nucleic acid expression and/or mACHR-6 polypeptide activity. Candidate/test compounds or agents which have one or more of these abilities can be used as drugs to treat disorders characterized by aberrant or abnormal mACHR-6 nucleic acid expression and/or mACHR-6 polypeptide activity. Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); and 4) small organic and inorganic molecules (e.g., molecules obtained from combinatorial and natural product libraries).

In one embodiment, the invention provides assays for screening candidate/test compounds which interact with (e.g., bind to) mACHR-6 polypeptide. Typically, the assays are recombinant cell based or cell-free assays which include the steps of combining an mACHR-6 polypeptide or a bioactive fragment thereof, and a candidate/test compound, e.g., under conditions which allow for interaction of (e.g., binding of) the candidate/test compound to the mACHR-6 polypeptide or fragment thereof to form a complex, and detecting the formation of a complex, in which the ability of the candidate compound to interact with (e.g., bind to) the mACHR-6 polypeptide or fragment thereof is indicated by the presence of the candidate compound in the complex. Formation of complexes between the mACHR-6 polypeptide and the candidate compound can be quantitated, for example, using standard immunoassays.

In another embodiment, the invention provides screening assays to identify candidate/test compounds which modulate (e.g., stimulate or inhibit) the interaction (and most likely mACHR-6 activity as well) between an mACHR-6 polypeptide and a molecule (target molecule) with which the mACHR-6 polypeptide normally interacts. Examples of such target molecules include polypeptides in the same signaling path as the mACHR-6 polypeptide, e.g., polypeptides which may function upstream (including both stimulators and inhibitors of activity) or downstream of the mACHR-6 polypeptide in, for example, a cognitive function signaling pathway or in a pathway involving mACHR-6 activity, e.g., a G protein or other interactor involved in phosphatidylinositol turnover and/or phospholipase C activation. Typically, the assays are recombinant cell based or cell-free assays which include the steps of combining a cell expressing an mACHR-6 polypeptide, or a bioactive fragment thereof, an mACHR-6 target molecule (e.g., an mACHR-6 ligand) and a candidate/test compound, e.g., under conditions wherein but for the presence of the candidate compound, the mACHR-6 polypeptide or biologically active portion thereof interacts with (e.g., binds to) the target molecule, and detecting the formation of a complex which includes the mACHR-6 polypeptide and the target molecule or detecting the interaction/reaction of the mACHR-6 polypeptide and the target molecule. Detection of complex formation can include direct quantitation of the complex by, for example, measuring inductive effects of the mACHR-6 polypeptide. A statistically significant change, such as a decrease, in the interaction of the mACHR-6 and target molecule (e.g., in the formation of a complex between the mACHR-6 and the target molecule) in the presence of a candidate compound (relative to what is detected in the absence of the candidate compound) is indicative of a modulation (e.g., stimulation or inhibition) of the interaction between the mACHR-6 polypeptide and the target molecule. Modulation of the formation of complexes between the mACHR-6 polypeptide and the target molecule can be quantitated using, for example, an immunoassay.

To perform cell free drug screening assays, it is desirable to immobilize either mACHR-6 or its target molecule to facilitate separation of complexes from uncomplexed forms of one or both of the polypeptides, as well as to accommodate automation of the assay. Interaction (e.g., binding of) of mACHR-6 to a target molecule, in the presence and absence of a candidate compound, can be accomplished in any vessel suitable for containing the reactants. Examples of such vessels include microtitre plates, test tubes, and micro-centrifuge tubes. In one embodiment, a fusion polypeptide can be provided which adds a domain that allows the polypeptide to be bound to a matrix. For example, glutathione-S-transferase/mACHR-6 fusion polypeptides can be adsorbed onto glutathione sepharose beads (Sigma Chemical, St. Louis, Mo.) or glutathione derivatized microtitre plates, which are then combined with the cell lysates (e.g., 35S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of mACHR-6-binding polypeptide found in the bead fraction quantitated from the gel using standard electrophoretic techniques.

Other techniques for immobilizing polypeptides on matrices can also be used in the drug screening assays of the invention. For example, either mACHR-6 or its target molecule can be immobilized utilizing conjugation of biotin and streptavidin. Biotinylated mACHR-6 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with mACHR-6 but which do not interfere with binding of the polypeptide to its target molecule can be derivatized to the wells of the plate, and mACHR-6 trapped in the wells by antibody conjugation. As described above, preparations of an mACHR-6-binding polypeptide and a candidate compound are incubated in the mACHR-6-presenting wells of the plate, and the amount of complex trapped in the well can be quantitated. Methods for detecting such complexes, in addition to those described above for the GST-immobilized complexes, include immunodetection of complexes using antibodies reactive with the mACHR-6 target molecule, or which are reactive with mACHR-6 polypeptide and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant or abnormal mACHR-6 nucleic acid expression or mACHR-6 polypeptide activity. This method typically includes the step of assaying the ability of the compound or agent to modulate the expression of the mACHR-6 nucleic acid or the activity of the mACHR-6 polypeptide thereby identifying a compound for treating a disorder characterized by aberrant or abnormal mACHR-6 nucleic acid expression or mACHR-6 polypeptide activity. Disorders characterized by aberrant or abnormal mACHR-6 nucleic acid expression or mACHR-6 polypeptide activity are described herein. Methods for assaying the ability of the compound or agent to modulate the expression of the mACHR-6 nucleic acid or activity of the mACHR-6 polypeptide are typically cell-based assays. For example, cells which are sensitive to ligands which transduce signals via a pathway involving mACHR-6 can be induced to overexpress an mACHR-6 polypeptide in the presence and absence of a candidate compound. Candidate compounds which produce a statistically significant change in mACHR-6-dependent responses (either stimulation or inhibition) can be identified. In one embodiment, expression of the mACHR-6 nucleic acid or activity of an mACHR-6 polypeptide is modulated in cells and the effects of candidate compounds on the readout of interest (such as phosphatidylinositol turnover) are measured. For example, the expression of genes which are up- or down-regulated in response to an mACHR-6-dependent signal cascade can be assayed. In preferred embodiments, the regulatory regions of such genes, e.g., the 5 flanking promoter and enhancer regions, are operably linked to a detectable marker (such as luciferase) which encodes a gene product that can be readily detected. Phosphorylation of mACHR-6 or mACHR-6 target molecules can also be measured, for example, by immunoblotting.

Alternatively, modulators of mACHR-6 expression (e.g., compounds which can be used to treat a disorder characterized by aberrant or abnormal mACHR-6 nucleic acid expression or mACHR-6 polypeptide activity) can be identified in a method wherein a cell is contacted with a candidate compound and the expression of mACHR-6 mRNA or polypeptide in the cell is determined. The level of expression of mACHR-6 mRNA or polypeptide in the presence of the candidate compound is compared to the level of expression of mACHR-6 mRNA or polypeptide in the absence of the candidate compound. The candidate compound can then be identified as a modulator of mACHR-6 nucleic acid expression based on this comparison and be used to treat a disorder characterized by aberrant mACHR-6 nucleic acid expression. For example, when expression of mACHR-6 mRNA or polypeptide is greater (statistically significantly greater) in the presence of the candidate compound than in its absence, the candidate compound is identified as a stimulator of mACHR-6 nucleic acid expression. Alternatively, when mACHR-6 nucleic acid expression is less (statistically significantly less) in the presence of the candidate compound than in its absence, the candidate compound is identified as an inhibitor of mACHR-6 nucleic acid expression. The level of mACHR-6 nucleic acid expression in the cells can be determined by methods described herein for detecting mACHR-6 mRNA or polypeptide.

In yet another aspect of the invention, the mACHR-6 polypeptides, or fragments thereof, can be used as “bait proteins” in a two-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with mACHR-6 (“mACHR-6-binding proteins” or “mACHR-6-bp”) and modulate mACHR-6 polypeptide activity. Such mACHR-6-binding proteins are also likely to be involved in the propagation of signals by the mACHR-6 polypeptides as, for example, upstream or downstream elements of the mACHR-6 pathway.

The two-hybrid system is based on the modular nature of most transcription factors, which consist of separable DNA-binding and activation domains. Bartel, P. et al. “Using the Two-Hybrid System to Detect Protein-Protein Interactions” in Cellular Interactions in Development: A Practical Approach, Hartley, D. A. ed. (Oxford University Press, Oxford, 1993) pp. 153-179. Briefly, the assay utilizes two different DNA constructs. In one construct, the gene that codes for mACHR-6 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an mACHR-6-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with mACHR-6.

Modulators of mACHR-6 polypeptide activity and/or mACHR-6 nucleic acid expression identified according to these drug screening assays can be used to treat, for example, nervous system disorders, smooth muscle related disorders, cardiac muscle related disorders, and gland related disorders. These methods of treatment include the steps of administering the modulators of mACHR-6 polypeptide activity and/or nucleic acid expression, e.g., in a pharmaceutical composition as described in subsection IV above, to a subject in need of such treatment, e.g., a subject with a disorder described herein.

b. Diagnostic Assays:

The invention further provides a method for detecting the presence of mACHR-6, or fragment thereof, in a biological sample. The method involves contacting the biological sample with a compound or an agent capable of detecting mACHR-6 polypeptide or mRNA such that the presence of mACHR-6 is detected in the biological sample. A preferred agent for detecting mACHR-6 mRNA is a labeled or labelable nucleic acid probe capable of hybridizing to mACHR-6 mRNA. The nucleic acid probe can be, for example, the full-length mACHR-6 cDNA of SEQ ID NO:33, 34, or 35, or a portion thereof, such as an oligonucleotide of at least 15, 30, 50, 100, 250 or 500 nucleotides in length and sufficient to specifically hybridize under stringent conditions to mACHR-6 mRNA. A preferred agent for detecting mACHR-6 polypeptide is a labeled or labelable antibody capable of binding to mACHR-6 polypeptide. Antibodies can be polyclonal, or more preferably, monoclonal. An intact antibody, or a fragment thereof (e.g., Fab or F(ab′)2) can be used. The term “labeled or labelable”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mACHR-6 mRNA or polypeptide in a biological sample in vitro as well as in vivo. For example, in vitro techniques for detection of mACHR-6 mRNA include Northern hybridizations and in situ hybridizations. In vitro techniques for detection of mACHR-6 polypeptide include enzyme linked immunosorbent assays (ELISAs), Western blots, immunoprecipitations and immunofluorescence. Alternatively, mACHR-6 polypeptide can be detected in vivo in a subject by introducing into the subject a labeled anti-mACHR-6 antibody. For example, the antibody can be labeled with a radioactive marker whose presence and location in a subject can be detected by standard imaging techniques. Particularly useful are methods which detect the allelic variant of mACHR-6 expressed in a subject and methods which detect fragments of an mACHR-6 polypeptide in a sample.

The invention also encompasses kits for detecting the presence of mACHR-6 in a biological sample. For example, the kit can comprise a labeled or labelable compound or agent capable of detecting mACHR-6 polypeptide or mRNA in a biological sample; means for determining the amount of mACHR-6 in the sample; and means for comparing the amount of mACHR-6 in the sample with a standard. The compound or agent can be packaged in a suitable container. The kit can further comprise instructions for using the kit to detect mACHR-6 mRNA or polypeptide.

The methods of the invention can also be used to detect naturally occurring genetic mutations in an mACHR-6 gene, thereby determining if a subject with the mutated gene is at risk for a disorder characterized by aberrant or abnormal mACHR-6 nucleic acid expression or mACHR-6 polypeptide activity as described herein. In preferred embodiments, the methods include detecting, in a sample of cells from the subject, the presence or absence of a genetic mutation characterized by at least one of an alteration affecting the integrity of a gene encoding an mACHR-6 polypeptide, or the misexpression of the mACHR-6 gene. For example, such genetic mutations can be detected by ascertaining the existence of at least one of 1) a deletion of one or more nucleotides from an mACHR-6 gene; 2) an addition of one or more nucleotides to an mACHR-6 gene; 3) a substitution of one or more nucleotides of an mACHR-6 gene, 4) a chromosomal rearrangement of an mACHR-6 gene; 5) an alteration in the level of a messenger RNA transcript of an mACHR-6 gene, 6) aberrant modification of an mACHR-6 gene, such as of the methylation pattern of the genomic DNA, 7) the presence of a non-wild type splicing pattern of a messenger RNA transcript of an mACHR-6 gene, 8) a non-wild type level of an mACHR-6-polypeptide, 9) allelic loss of an mACHR-6 gene, and 10) inappropriate post-translational modification of an mACHR-6-polypeptide. As described herein, there are a large number of assay techniques known in the art which can be used for detecting mutations in an mACHR-6 gene.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the mACHR-6-gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to an mACHR-6 gene under conditions such that hybridization and amplification of the mACHR-6-gene (if present) occurs, and detecting the presence or absence of an amplification product, or detecting the size of the amplification product and comparing the length to a control sample.

In an alternative embodiment, mutations in an mACHR-6 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In yet another embodiment, any of a variety of sequencing reactions known in the art can be used to directly sequence the mACHR-6 gene and detect mutations by comparing the sequence of the sample mACHR-6 with the corresponding wild-type (control) sequence. Examples of sequencing reactions include those based on techniques developed by Maxim and Gilbert ((1977) PNAS 74:560) or Sanger ((1977) PNAS 74:5463). A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the mACHR-6 gene include methods in which protection from cleavage agents is used to detect mismatched bases in RNA/RNA or RNA/DNA duplexes (Myers et al. (1985) Science 230:1242); Cotton et al. (1988) PNAS 85:4397; Saleeba et al. (1992) Meth. Enzymol. 217:286-295), electrophoretic mobility of mutant and wild type nucleic acid is compared (Orita et al. (1989) PNAS 86:2766; Cotton (1993) Mutat Res 285:125-144; and Hayashi (1992) Genet Anal Tech Appl 9:73-79), and movement of mutant or wild-type fragments in polyacrylamide gels containing a gradient of denaturant is assayed using denaturing gradient gel electrophoresis (Myers et al (1985) Nature 313:495). Examples of other techniques for detecting point mutations include, selective oligonucleotide hybridization, selective amplification, and selective primer extension.

c. Methods of Treatment

Another aspect of the invention pertains to methods for treating a subject, e.g., a human, having a disease or disorder characterized by (or associated with) aberrant or abnormal mACHR-6 nucleic acid expression and/or mACHR-6 polypeptide activity. These methods include the step of administering an mACHR-6 modulator (agonist or antagonist) to the subject such that treatment occurs. The language “aberrant or abnormal mACHR-6 expression” refers to expression of a non-wild-type mACHR-6 polypeptide or a non-wild-type level of expression of an mACHR-6 polypeptide. Aberrant or abnormal mACHR-6 activity refers to a non-wild-type mACHR-6 activity or a non-wild-type level of mACHR-6 activity. As the mACHR-6 polypeptide is involved in a pathway involving modulation of neurotransmitter, e.g., acetylcholine, release; modulation of smooth muscle contraction; modulation of cardiac muscle contraction; and modulation of gland, e.g., exocrine gland function, aberrant or abnormal mACHR-6 activity or expression interferes with the normal neurotransmitter, e.g., acetylcholine, release; normal smooth muscle; and cardiac muscle contraction; and normal gland, e.g., exocrine gland function. Non-limiting examples of disorders or diseases characterized by or associated with abnormal or aberrant mACHR-6 activity or expression include nervous system related disorders, e.g., central nervous system related disorders. Examples of nervous system related disorders include cognitive disorders, e.g., memory and learning disorders, such as amnesia, apraxia, agnosia, amnestic dysnomia, amnestic spatial disorientation, Kluver-Bucy syndrome, Alzheimer's related memory loss (Eglen R. M. (1996) Pharmacol. and Toxicol. 78(2):59-68; Perry E. K. (1995) Brain and Cognition 28(3):240-58) and learning disability; disorders affecting consciousness, e.g., visual hallucinations, perceptual disturbances, or delerium associated with Lewy body dementia; schitzo-effective disorders (Dean B. (1996) Mol. Psychiatry 1(1):54-8), schizophrenia with mood swings (Bymaster F. P. (1997) J. Clin. Psychiatry 58 (suppl. 10):28-36; Yeomans J. S. (1995) Neuropharmacol. 12(1):3-16; Reimann D. (1994) J. Psychiatric Res. 28(3):195-210), depressive illness (primary or secondary); affective disorders (Janowsky D. S. (1994) Am. J. Med. Genetics 54(4):335-44); sleep disorders (Kimura F. (1997) J. Neurophysiol. 77(2):709-16), e.g., REM sleep abnormalities in patients suffering from, for example, depression (Riemann D. (1994) J. Psychosomatic Res. 38 Suppl. 1:15-25; Bourgin P. (1995) Neuroreport 6(3): 532-6), paradoxical sleep abnormalities (Sakai K. (1997) Eur. J. Neuroscience 9(3):415-23), sleep-wakefulness, and body temperature or respiratory depression abnormalities during sleep (Shuman S. L. (1995) Am. J. Physiol. 269(2 Pt 2):R308-17; Mallick B. N. (1997) Brain Res. 750(1-2):311-7). Other examples of nervous system related disorders include disorders affecting pain generation mechanisms, e.g., pain related to irritable bowel syndrome (Mitch C. H. (1997) J. Med. Chem. 40(4):538-46; Shannon H. E. (1997) J. Pharmac. and Exp. Therapeutics 281(2):884-94; Bouaziz H. (1995) Anesthesia and Analgesia 80(6):1140-4; or Guimaraes A. P. (1994) Brain Res. 647(2):220-30) or chest pain; movement disorders (Monassi C. R. (1997) Physiol. and Behav. 62(1):53-9), e.g., Parkinson's disease related movement disorders (Finn M. (1997) Pharmacol. Biochem. & Behavior 57(1-2):243-9; Mayorga A. J. (1997) Pharmacol. Biochem. & Behavior 56(2):273-9); eating disorders, e.g., insulin hypersecretion related obesity (Maccario M. (1997) J. Endocrinol. Invest. 20(1):8-12; Premawardhana L. D. (1994) Clin. Endocrinol. 40(5): 617-21); or drinking disorders, e.g., diabetic polydipsia (Murzi E. (1997) Brain Res. 752(1-2):184-8; Yang X. (1994) Pharmacol. Biochem. & Behavior 49(1):1-6). Yet further examples of disorders or diseases characterized by or associated with abnormal or aberrant mACHR-6 activity or expression include smooth muscle related disorders such as irritable bowel syndrome, diverticular disease, urinary incontinence, oesophageal achalasia, or chronic obstructive airways disease; heart muscle related disorders such as pathologic bradycardia or tachycardia, arrhythmia, flutter or fibrillation; or gland related disorders such as xerostomia, or diabetes mellitus. The terms “treating” or “treatment”, as used herein, refer to reduction or alleviation of at least one adverse effect or symptom of a disorder or disease, e.g., a disorder or disease characterized by or associated with abnormal or aberrant mACHR-6 polypeptide activity or mACHR-6 nucleic acid expression.

As used herein, an mACHR-6 modulator is a molecule which can modulate mACHR-6 nucleic acid expression and/or mACHR-6 polypeptide activity. For example, an mACHR-6 modulator can modulate, e.g., upregulate (activate/agonize) or down-regulate (suppress/antagonize), mACHR-6 nucleic acid expression. In another example, an mACHR-6 modulator can modulate (e.g., stimulate/agonize or inhibit/antagonize) mACHR-6 polypeptide activity. If it is desirable to treat a disorder or disease characterized by (or associated with) aberrant or abnormal (non-wild-type) mACHR-6 nucleic acid expression and/or mACHR-6 polypeptide activity by inhibiting mACHR-6 nucleic acid expression, an mACHR-6 modulator can be an antisense molecule, e.g., a ribozyme, as described herein. Examples of antisense molecules which can be used to inhibit mACHR-6 nucleic acid expression include antisense molecules which are complementary to a portion of the 5′ untranslated region of SEQ ID NO:33, 34 or 35 which also includes the start codon and antisense molecules which are complementary to a portion of the 3′ untranslated region of SEQ ID NO:33, 34, or 35. An example of an antisense molecule which is complementary to a portion of the 5′ untranslated region of SEQ ID NO:33 and which also includes the start codon is a nucleic acid molecule which includes nucleotides which are complementary to nucleotides 280 to 296 of SEQ ID NO:33. This antisense molecule has the following nucleotide sequence: 5′ CCTGCGGGGCCATGGAG 3′ (SEQ ID NO:55). An example of an antisense molecule which is complementary to a portion of the 3′ untranslated region of SEQ ID NO:33 is a nucleic acid molecule which includes nucleotides which are complementary to nucleotides 1629 to 1645 of SEQ ID NO:33. This antisense molecule has the following sequence: 5′ GTGGCCCACCAGAGCCT 3′ (SEQ ID NO:56). An additional example of an antisense molecule which is complementary to a portion of the 3′ untranslated region of SEQ ID NO:33 is a nucleic acid molecule which includes nucleotides which are complementary to nucleotides 1650 to 1666 of SEQ ID NO:33. This antisense molecule has the following sequence: 5′ CAGCCACGCCTCTCTCA 3′ (SEQ ID NO:57). An example of an antisense molecule which is complementary to a portion of the 5′ untranslated region of SEQ ID NO:34 and which also includes the start codon, is a nucleic acid molecule which includes nucleotides which are complementary to nucleotides 766 to 783 of SEQ ID NO:34. This antisense molecule has the following nucleotide sequence: 5′ GCCTGCTGGGCCATGGAG 3′ (SEQ ID NO:58). An example of an antisense molecule which is complementary to a portion of the 3′ untranslated region of SEQ ID NO:34 is a nucleic acid molecule which includes nucleotides which are complementary to nucleotides 2113 to 2128 of SEQ ID NO:34. This antisense molecule has the following sequence: 5′ TGAGCAGCTGCCCCAC 3′ (SEQ ID NO:59). An additional example of an antisense molecule which is complementary to a portion of the 3′ untranslated region of SEQ ID NO:34 is a nucleic acid molecule which includes nucleotides which are complementary to nucleotides 2133 to 2148 of SEQ ID NO:34. This antisense molecule has the following sequence: 5′ CTGAGGCCAGGCCCTT 3′ (SEQ ID NO:62).

An mACHR-6 modulator which inhibits mACHR-6 nucleic acid expression can also be a small molecule or other drug, e.g., a small molecule or drug identified using the screening assays described herein, which inhibits mACHR-6 nucleic acid expression. If it is desirable to treat a disease or disorder characterized by (or associated with) aberrant or abnormal (non-wild-type) mACHR-6 nucleic acid expression and/or mACHR-6 polypeptide activity by stimulating mACHR-6 nucleic acid expression, an mACHR-6 modulator can be, for example, a nucleic acid molecule encoding mACHR-6 (e.g., a nucleic acid molecule comprising a nucleotide sequence homologous to the nucleotide sequence of SEQ ID NO:33, 34, or 35) or a small molecule or other drug, e.g., a small molecule (peptide) or drug identified using the screening assays described herein, which stimulates mACHR-6 nucleic acid expression.

Alternatively, if it is desirable to treat a disease or disorder characterized by (or associated with) aberrant or abnormal (non-wild-type) mACHR-6 nucleic acid expression and/or mACHR-6 polypeptide activity by inhibiting mACHR-6 polypeptide activity, an mACHR-6 modulator can be an anti-mACHR-6 antibody or a small molecule or other drug, e.g., a small molecule or drug identified using the screening assays described herein, which inhibits mACHR-6 polypeptide activity. If it is desirable to treat a disease or disorder characterized by (or associated with) aberrant or abnormal (non-wild-type) mACHR-6 nucleic acid expression and/or mACHR-6 polypeptide activity by stimulating mACHR-6 polypeptide activity, an mACHR-6 modulator can be an active mACHR-6 polypeptide or portion thereof (e.g., an mACHR-6 polypeptide or portion thereof having an amino acid sequence which is homologous to the amino acid sequence of SEQ ID NO:36, 37, or 38 or a portion thereof) or a small molecule or other drug, e.g., a small molecule or drug identified using the screening assays described herein, which stimulates mACHR-6 polypeptide activity.

Other aspects of the invention pertain to methods for modulating a cell associated activity. These methods include contacting the cell with an agent (or a composition which includes an effective amount of an agent) which modulates mACHR-6 polypeptide activity or mACHR-6 nucleic acid expression such that a cell associated activity is altered relative to a cell associated activity (for example, phosphatidylinositol metabolism) of the cell in the absence of the agent. As used herein, “a cell associated activity” refers to a normal or abnormal activity or function of a cell. Examples of cell associated activities include phosphatidylinositol turnover, production or secretion of molecules, such as proteins, contraction, proliferation, migration, differentiation, and cell survival. In a preferred embodiment, the cell is neural cell of the brain, e.g., a hippocampal cell. The term “altered” as used herein refers to a change, e.g., an increase or decrease, of a cell associated activity particularly phosphatidylinositol turnover and phospholipase C activation. In one embodiment, the agent stimulates mACHR-6 polypeptide activity or mACHR-6 nucleic acid expression. Examples of such stimulatory agents include an active mACHR-6 polypeptide, a nucleic acid molecule encoding mACHR-6 that has been introduced into the cell, and a modulatory agent which stimulates mACHR-6 polypeptide activity or mACHR-6 nucleic acid expression and which is identified using the drug screening assays described herein. In another embodiment, the agent inhibits mACHR-6 polypeptide activity or mACHR-6 nucleic acid expression. Examples of such inhibitory agents include an antisense mACHR-6 nucleic acid molecule, an anti-mACHR-6 antibody, and a modulatory agent which inhibits mACHR-6 polypeptide activity or mACHR-6 nucleic acid expression and which is identified using the drug screening assays described herein. These modulatory methods can be performed in vitro (e.g., by culturing the cell with the agent) or, alternatively, in vivo (e.g., by administering the agent to a subject). In a preferred embodiment, the modulatory methods are performed in vivo, i.e., the cell is present within a subject, e.g., a mammal, e.g., a human, and the subject has a disorder or disease characterized by or associated with abnormal or aberrant mACHR-6 polypeptide activity or mACHR-6 nucleic acid expression.

A nucleic acid molecule, a polypeptide, an mACHR-6 modulator, a compound etc. used in the methods of treatment can be incorporated into an appropriate pharmaceutical composition described herein and administered to the subject through a route which allows the molecule, polypeptide, modulator, or compound etc. to perform its intended function. Examples of routes of administration are also described herein under subsection IV.

d. Pharmacogenomics

Test/candidate compounds, or modulators which have a stimulatory or inhibitory effect on mACHR-6 activity (e.g., mACHR-6 gene expression) as identified by a screening assay described herein can be administered to individuals to treat (prophylactically or therapeutically) disorders (e.g., CNS disorders) associated with aberrant mACHR-6 activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permit the selection of effective compounds (e.g., drugs) for prophylactic or therapeutic treatments based on a consideration of the individual's genotype. Such pharmacogenomics can further be used to determine appropriate dosages and therapeutic regimens. Accordingly, the activity of mACHR-6 polypeptide, expression of mACHR-6 nucleic acid, or mutation content of mACHR-6 genes in an individual can be determined to thereby select appropriate compound(s) for therapeutic or prophylactic treatment of the individual.

Pharmacogenomics deal with clinically significant hereditary variations in the response to drugs due to altered drug disposition and abnormal action in affected persons. See, e.g., Eichelbaum, M. (1996) Clin. Exp. Pharmacol. Physiol. 23(10-11):983-985 and Linder, M. W. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of mACHR-6 polypeptide, expression of mACHR-6 nucleic acid, or mutation content of mACHR-6 genes in an individual can be determined to thereby select appropriate agent(s) for therapeutic or prophylactic treatment of a subject. In addition, pharmacogenetic studies can be used to apply genotyping of polymorphic alleles encoding drug-metabolizing enzymes to the identification of a subject's drug responsiveness phenotype. This knowledge, when applied to dosing or drug selection, can avoid adverse reactions or therapeutic failure and thus enhance therapeutic or prophylactic efficiency when treating a subject with an mACHR-6 modulator, such as a modulator identified by one of the exemplary screening assays described herein.

e. Monitoring of Effects During Clinical Trials

Monitoring the influence of compounds (e.g., drugs) on the expression or activity of mACHR-6 (e.g., the ability to modulate the effects of acetylcholine on acetylcholine responsive cells) can be applied not only in basic drug screening, but also in clinical trials. For example, the effectiveness of an agent determined by a screening assay, as described herein, to increase mACHR-6 gene expression, polypeptide levels, or up-regulate mACHR-6 activity, can be monitored in clinical trails of subjects exhibiting decreased mACHR-6 gene expression, polypeptide levels, or down-regulated mACHR-6 activity. Alternatively, the effectiveness of an agent, determined by a screening assay, to decrease mACHR-6 gene expression, polypeptide levels, or down-regulate mACHR-6 activity, can be monitored in clinical trails of subjects exhibiting increased mACHR-6 gene expression, polypeptide levels, or up-regulated mACHR-6 activity. In such clinical trials, the expression or activity of mACHR-6 and, preferably, other genes which have been implicated in, for example, a nervous system related disorder can be used as a “read out” or markers of the acetylcholine responsiveness of a particular cell.

For example, and not by way of limitation, genes, including mACHR-6, which are modulated in cells by treatment with a compound (e.g., drug or small molecule) which modulates mACHR-6 activity (e.g., identified in a screening assay as described herein) can be identified. Thus, to study the effect of compounds on CNS disorders, for example, in a clinical trial, cells can be isolated and RNA prepared and analyzed for the levels of expression of mACHR-6 and other genes implicated in the disorder. The levels of gene expression (i.e., a gene expression pattern) can be quantified by Northern blot analysis or RT-PCR, as described herein, or alternatively by measuring the amount of polypeptide produced, by one of the methods described herein, or by measuring the levels of activity of mACHR-6 or other genes. In this way, the gene expression pattern can serve as a marker, indicative of the physiological response of the cells to the compound. Accordingly, this response state may be determined before, and at various points during, treatment of the individual with the compound.

In a preferred embodiment, the present invention provides a method for monitoring the effectiveness of treatment of a subject with a compound (e.g., an agonist, antagonist, peptidomimetic, polypeptide, peptide, nucleic acid, small molecule, or other drug candidate identified by the screening assays described herein) comprising the steps of (i) obtaining a pre-administration sample from a subject prior to administration of the compound; (ii) detecting the level of expression of an mACHR-6 polypeptide, mRNA, or genomic DNA in the preadministration sample; (iii) obtaining one or more post-administration samples from the subject; (iv) detecting the level of expression or activity of the mACHR-6 polypeptide, mRNA, or genomic DNA in the post-administration samples; (v) comparing the level of expression or activity of the mACHR-6 polypeptide, mRNA, or genomic DNA in the pre-administration sample with the mACHR-6 polypeptide, mRNA, or genomic DNA in the post administration sample or samples; and (vi) altering the administration of the compound to the subject accordingly. For example, increased administration of the compound may be desirable to increase the expression or activity of mACHR-6 to higher levels than detected, i.e., to increase the effectiveness of the agent. Alternatively, decreased administration of the agent may be desirable to decrease expression or activity of mACHR-6 to lower levels than detected, i.e. to decrease the effectiveness of the compound.

VI. Uses of Partial mACHR-6 Sequences

Portions or fragments of the cDNA sequences identified herein (and the corresponding complete gene sequences) can be used in numerous ways as polynucleotide reagents. For example, these sequences can be used to: (a) map their respective genes on a chromosome; and, thus, locate gene regions associated with genetic disease; (b) identify an individual from a minute biological sample (tissue typing); and (c) aid in forensic identification of a biological sample. These applications are described in the subsections below.

a. Chromosome Mapping

Once the sequence (or a portion of the sequence) of a gene has been isolated, this sequence can be used to map the location of the gene on a chromosome. This process is called chromosome mapping. Accordingly, portions or fragments of the mACHR-6, sequences, described herein, can be used to map the location of the mACHR-6 gene, respectively, on a chromosome. The mapping of the mACHR-6 sequence to chromosomes is an important first step in correlating these sequence with genes associated with disease.

Briefly, the mACHR-6 gene can be mapped to a chromosome by preparing PCR primers (preferably 15-25 bp in length) from the mACHR-6 sequence. Computer analysis of the mACHR-6, sequence can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the mACHR-6 sequence will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the mACHR-6 sequence to design oligonucleotide primers, sublocalization can be achieved with panels of fragments from specific chromosomes. Other mapping strategies which can similarly be used to map a mACHR-6 sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York, 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data (such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the mACHR-6 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

b. Tissue Typing

The mACHR-6 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the mACHR-6 sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The mACHR-6 sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NOs:33, 34, and 35, can comfortably provide positive individual identification with a panel of perhaps 10 to 1,000 primers which each yield a noncoding amplified sequence of 100 bases. If predicted coding sequences, such as those in SEQ ID NOs:39, 40, and 41, are used, a more appropriate number of primers for positive individual identification would be 500-2,000.

If a panel of reagents from mACHR-6 sequences described herein is used to generate a unique identification database for an individual, those same reagents can later be used to identify tissue from that individual. Using the unique identification database, positive identification of the individual, living or dead, can be made from extremely small tissue samples.

c. Use of Partial mACHR-6 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to noncoding regions of SEQ ID NOs:33, 34, and 35 are particularly appropriate for this use as greater numbers of polymorphisms occur in the noncoding regions, making it easier to differentiate individuals using this technique. Examples of polynucleotide reagents include the mACHR-6 sequences or portions thereof, e.g., fragments derived from the noncoding regions of SEQ ID NOs:33, 34, and 35, having a length of at least 20 bases, preferably at least 30 bases.

The mACHR-6 sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such mACHR-6 probes can be used to identify tissue by species and/or by organ type.

In a similar fashion, these reagents, e.g., mACHR-6 primers or probes can be used to screen tissue culture for contamination (i.e. screen for the presence of a mixture of different types of cells in a culture).

This invention is further illustrated by the following examples which should not be construed as limiting. The contents of all references, patent applications, patents, and published patent applications cited throughout this application are hereby incorporated by reference.

### EXAMPLES

### Example 1

### Identification of Rat and Human mACHR-6 cDNA

In this example, mACHR-6 nucleic acid molecules were identified by screening appropriate cDNA libraries. More specifically, a rat frontal cortex oligo dT-primed cDNA library was plated out and colonies picked into 96 well plates. The colonies were cultured, plasmids were prepared from each well, and the 5′ end of each insert sequenced. After automated “trimming” of non-insert sequences, the nucleotide sequences were compared against the public protein databases using the BLAST sequence comparison program (BLASTN1.3 MP, Altschul et al. (1990) J. Mol. Biol. 215:403). Upon review of the results from this sequence comparison, a single clone was identified, designated 84g5, whose highest similarity was with the rat muscarinic acetylcholine receptor M1 (MACHR M1; GenBank™ Accession Number P08482). The clone containing this sequence was recovered from the 96 well plate, plasmid was prepared using standard methods and the insert fully sequenced using standard “contigging” techniques. A repeat BLAST analysis using the entire insert sequence once again showed that the sequence in the protein database with the greatest similarity corresponded to GenBank™ Accession Number P08482. This sequence and the insert sequence were compared using the GAP program in the GCG software package using a gap weight of 5.000 and a length weight of 0.100. The results showed a 27.97% identity and 49.01% similarity between the two sequences with the insertion of 4 gaps for optimized sequence alignment. The alignment indicated that the 84g5 clone does not extend fully across the P08482 sequence, apparently missing approximately 30 amino acid residues at the N-terminal region of the molecule. A probe spanning residues 143-249 of SEQ ID NO:35 was then used to re-screen the same frontal cortex library. This resulted in the identification of the full length rat mACHR-6 sequence shown in SEQ ID NO:34. BLAST analysis of public nucleotide databases revealed no equivalent human sequences. Only a single mouse EST was identified (GenBank™ Accession Number AA118949) which is similar to the 84g5 clone between residues 1101 and 1650.

The human mACHR-6 nucleic acid molecule was identified by screening a human cerebellum cDNA library using a Nci I/Not I restriction fragment of the rat cDNA as a probe. BLAST analysis of protein and nucleic acid databases in the public domain again showed that the mACHR-6 nucleic acid molecule is most similar to mACHR M1 sequences. The alignments also revealed that mAChR-6 nucleic acid molecule encodes a full length mACHR polypeptide.

### Example 2

### Tissue Expression of the mACHR-6 Gene

Northern Analysis Using RNA from Human and Rat Tissue

Human brain multiple tissue northern (MTN) blots, human MTN I, II, and III blots, and rat MTN blots (Clontech, Palo Alto, Calif.), containing 2 g of poly A+ RNA per lane were probed with the rat mACHR-6 nucleotide sequence (Nci I/Not I restriction fragment). The filters were prehybridized in 10 ml of Express Hyb hybridization solution (Clontech, Palo Alto, Calif.) at 68° C. for 1 hour, after which 100 ng of 32P labeled probe was added. The probe was generated using the Stratagene Prime-It kit, Catalog Number 300392 (Clontech, Palo Alto, Calif.). Hybridization was allowed to proceed at 68° C. for approximately 2 hours. The filters were washed in a 0.05% SDS/2×SSC solution for 15 minutes at room temperature and then twice with a 0.1% SDS/0.1×SSC solution for 20 minutes at 50° C. and then exposed to autoradiography film overnight at −80° C. with one screen. The human tissues tested included: heart, brain (regions of the brain tested included cerebellum, corpus callosum, cerebral cortex, medulla, occipital pole, frontal lobe, temporal lobe, putamen, amygdala, caudate nucleus, hippocampus, substantia nigra, subthalamic nucleus and thalamus), placenta, lung, liver, skeletal muscle, kidney, pancreas, spleen, thymus, prostate, testis, ovary, small intestine, colon, peripheral blood leukocyte, stomach, thyroid, spinal cord, lymph node, trachea, adrenal gland and bone marrow. The rat tissues tested included: heart, brain, spleen, lung, liver, skeletal muscle, kidney, and testis.

There was a strong hybridization to human whole brain, the following human brain regions: cerebellum, corpus callosum, cerebral cortex, medulla, occipital pole, frontal lobe, temporal lobe, putamen, amygdala, caudate nucleus, hippocampus, substantia nigra, subthalamic nucleus and thalamus; and rat brain indicating that the approximately 3 kb mACHR-6 gene transcript is expressed in these tissues. There was also hybridization to human spinal cord.

In Situ Hybridization

For in situ analysis, the brain of an adult Sprague-Dawley rat was removed and frozen on dry ice. Ten-micrometer-thick coronal sections of the brain were postfixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections were rinsed in DEPC 2×SSC (1×SSC is 0.15M NaCl plus 0.015M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with 35S-radiolabeled (5×107 cpm/ml) cRNA probes encoding a 474-bp fragment of the rat gene (generated with PCR primers F, 5′-CAAGAACCCTTTAAGCCAAG (SEQ ID NO:63), and R, 5′-GAAGAAGGTAACGCTGAGGA (SEQ ID NO:64)) and a 529-bp fragment of the rat gene (generated with PCR primers F, 5′-CAGAACCCCCACCAGATGCC (SEQ ID NO:65), and R, 5′-TAGTGGCACAGTGGGTAGAG (SEQ ID NO:66)). Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1× Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2×SSC. Sections were then sequentially incubated at 37° C. in TNE (a solution containing 10 mM Tris-HCl (pH 7.6), 500 mM NaCl, and 1 mM EDTA), for 10 minutes, in TNE with 10 mg of RNase A per ml for 30 minutes, and finally in TNE for 10 minutes. Slides were then rinsed with 2×SSC at room temperature, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

Significant hybridization was seen in a number of brain regions. These included the cortex, caudate putamen, hippocampus, thalamus and cerebellum. Analysis of these regions at high magnification showed that significant labeling was seen over the cell bodies of neurons.

### Example 3

### Expression of Recombinant mACHR-6 Polypeptide in Bacterial Cells

In this example, mACHR-6 is expressed as a recombinant glutathione-S-transferase (GST) fusion polypeptide in E. coli and the fusion polypeptide is isolated and characterized. Specifically, mACHR-6 is fused to GST and this fusion polypeptide is expressed in E. coli, e.g., strain PEB199. As the human and rat mACHR-6 polypeptides are predicted to be approximately 51.3 kDa, and 51.2 kDa, respectively, and GST is predicted to be 26 kDa, the fusion polypeptides are predicted to be approximately 77.3 kDa and 77.2 kDa, respectively, in molecular weight. Expression of the GST-mACHR-6 fusion polypeptide in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant mACHR-6 Polypeptide in COS Cells

To express the mACHR-6 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire mACHR-6 polypeptide and a HA tag (Wilson et al. (1984) Cell 37:767) fused in-frame to its 3′ end of the fragment is cloned into the polylinker region of the vector, thereby placing the expression of the recombinant polypeptide under the control of the CMV promoter.

To construct the plasmid, the mACHR-6 DNA sequence is amplified by PCR using two primers. The 5′ primer contains the restriction site of interest followed by approximately twenty nucleotides of the mACHR-6 coding sequence starting from the initiation codon; the 3′ end sequence contains complementary sequences to the other restriction site of interest, a translation stop codon, the HA tag and the last 20 nucleotides of the mACHR-6 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the mACHR-6 gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the mACHR-6-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the mACHR-6 polypeptide is detected by radiolabelling (35S-methionine or 35S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with 35S-methionine (or 35S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the mACHR-6 coding sequence is cloned directly into the polylinker of the pCDNA/Amp vector using the appropriate restriction sites. The resulting plasmid is transfected into COS cells in the manner described above, and the expression of the mACHR-6 polypeptide is detected by radiolabelling and immunoprecipitation using an mACHR-6 specific monoclonal antibody.

### Example 5

### Characterization of the Human and Rat mACHR-6 Polypeptides

In this example, the amino acid sequences of the human and the rat mACHR-6 polypeptides were compared to amino acid sequences of known polypeptides and various motifs were identified.

The human mACHR-6 polypeptide, the amino acid sequence of which is shown in (SEQ ID NO:36), is a novel polypeptide which includes 445 amino acid residues. The human mACHR-6 polypeptide contains seven transmembrane domains between amino acid residues 34-59 (SEQ ID NO:42), 73-91 (SEQ ID NO:61), 109-130 (SEQ ID NO:43), 152-174 (SEQ ID NO:44), 197-219 (SEQ ID NO:45), 360-380 (SEQ ID NO:60), and 396-416 (SEQ ID NO:46). The nucleotide sequence of the human mACHR-6 was used as a database query using the BLASTN program (BLASTN1.3 MP, Altschul et al. (1990) J. Mol. Biol. 215:403). The closest hits were human, rat, mouse and pig mACHR M1 (GenBank™ Accession Numbers P11229, P08482, P12657, and P04761, respectively). The highest similarity is 32/70 amino acid identities.

The rat mACHR-6 polypeptide, the amino acid sequence of which is shown in (SEQ ID NO:37), is a novel polypeptide which includes 445 amino acid residues. The rat mACHR-6 polypeptide contains seven transmembrane domains between amino acid residues 34-59 (SEQ ID NO:47), 73-91 (SEQ ID NO:48), 109-130 (SEQ ID NO:49), 152-174 (SEQ ID NO:50), 197-219 (SEQ ID NO:51), 360-380 (SEQ ID NO:52) and 396-416 (SEQ ID NO:53), which correspond to the human mACHR-6 polypeptide transmembrane domains 1-7 (SEQ ID NOs:42-46). The nucleotide sequence of the rat mACHR-6 was used as a database query using the BLASTN program (BLASTN1.3 MP, Altschul et al. (1990) J. Mol. Biol. 215:403). The closest hits were human, rat, mouse and pig mACHR M1 (GenBank™ Accession Numbers P11229, P08482, P12657, and P04761, respectively). The highest similarity is 33/70 amino acid identities. Hydropathy plots indicated that the transmembrane domains of the rat mACHR-6 polypeptide are similar to those of the rat mACHR M1. The cysteines (residues 63 and 44 of SEQ ID NO:37) that give rise to intramolecular disulfide bonds are also conserved.

Equivalents

Those skilled in the art will recognize, or be able to ascertain using no more than routine experimentation, many equivalents to the specific embodiments of the invention described herein. Such equivalents are intended to be encompassed by the following claims

### VII. C7F2—A NOVEL POTASSIUM CHANNEL β-SUBUNIT

### Background of the Invention

The flow of potassium through the plasma membrane affects diverse biological processes including action-potential firing and control of cell volume. Potassium channels are ubiquitous integral membrane proteins serving numerous functions in excitable and nonexcitable cells (McManus, O. B., J. Bioenerg. Biomembr. 23:537-560 (1991)). Many different classes of potassium channels have evolved and have been separated into classes on the basis of their biophysical properties, physiological regulation, and pharmacology (Hille, B., Ionic Channels of Excitable Membranes, Sunderland, M. A. Sinauer (1992); Rudy, B., Neuroscience 25:729-749 (1988)). Major types include voltage-dependent, calcium-activated, and ATP-sensitive channels. Some subtypes exist within the classifications. However, certain functional features are shared among many types of potassium channels (Kukuljan et al., Am. J. Physiol. 268 (Cell Physiol. 37):C535-C556 (1995)).

Potassium channel-forming proteins can be grouped into three families that differ in the number of transmembrane segments. The largest family contains six membrane-spanning segments. Inward rectifiers comprise the second family with subunits having two transmembrane segments. The third family contains only one transmembrane segment. These channels have been studied using recombinant DNA techniques. The information has been reviewed in Kukuljan et al., cited above.

High conductance calcium-activated potassium channels are a group of proteins with a number of unique features. The channels are activated by intracellular calcium, as well as membrane depolarization. The channels display a high single-channel conductance and are highly selective for potassium. They are sensitive to specific toxins, such as charybdotoxin that binds to a receptor site located on the external vestibule of the channel and prevents potassium flow by physical occlusion of the pore.

Knaus et al. (J. Biol. Chem. 269:3921-3924 (1994)) reported on the subunit composition of the high conductance calcium-activated potassium channel from smooth muscle. This potassium channel is reported to be composed of two subunits, α and β, of 62 and 31 kilodaltons, respectively. Amino acid sequence analysis showed a high sequence homology with two cloned high conductance potassium channels from Drosophila. An antipeptide antibody directed against the amino acid sequence of one of the α-subunit fragments could also immunoprecipitate, under nondenaturing conditions, the β-subunit, demonstrating specific noncovalent association of both subunits. The results indicated that the α-subunit of this specific high conductance potassium channel is a member of a specific family of potassium channels and forms a noncovalent complex with a β-subunit. The reference reported a specific and tight interaction between the two polypeptides. The following model was proposed. The α-subunit is the central ion channel-forming element and contains the receptor for the various blocking toxins. A tetramer α-subunit is noncovalently associated with four β-subunits. The β-subunits are in close proximity (less than 12 Å) to the pore-forming and receptor carrying subunit. This high conductance potassium channel β-subunit shares characteristics with the β-subunit of rat brain sodium channels and the γ-subunit of skeletal muscle L-type calcium channels and may be analogous in structure and/or function. It is speculated that this subunit is a conserved constituent of many voltage- and calcium-dependent potassium channels.

Knaus et al. (J. Biol. Chem. 269:17274-17278 (1994)) disclosed the primary sequence and immunological characterization of the β-subunit of the high conductance calcium-activated potassium channel from smooth muscle. The amino acid sequence was used to design oligonucleotide probes with which cDNAs encoding the protein were isolated. The protein was reported to contain two hydrophobic (putative transmembrane) domains bearing little sequence homology to subunits of other known ion channels. Reports had suggested that the β-subunit plays a role in modulating the properties of the pore-forming subunit. For example, co-expression of sodium or calcium channel α- and β-subunits had been demonstrated to modulate the currents expressed from the α-subunits alone. The reference also reported small regions of homology with other β-subunits. It is reported, for example, that the β2-subunit of the rabbit cardiac calcium channel contains a stretch of eight amino acids that are 100% homologous to a region of the β-subunit of the channel under study.

McManus et al. (Neuron 14:645-650 (1995)) examined the functional contribution of the β-subunit properties of high conductance potassium channels expressed heterologously in Xenopus oocytes. The reference reported that co-expression of the bovine smooth muscle high conductance potassium channel β-subunit has dramatic effects on the properties of expressed mouse brain α-subunits. The reference noted that expression of an α-subunit alone is sufficient to generate potassium channels that are gated by voltage and intracellular calcium. Nevertheless, channels from oocytes injected with cDNAs encoding both α- and β-subunits were much more sensitive to activation voltage and calcium than channels composed of the α-subunit alone. Expression levels, single channel conductance, and ion selectivity appeared unaffected. Further, channels from oocytes expressing both subunits were sensitive to a potent agonist of native high conductance potassium channels, whereas channels composed of the α-subunit alone were insensitive. Thus, in addition to its effects on channel gating, the β-subunit conferred sensitivity to DHS-I, a potent agonist of native high conductance potassium channels. Accordingly, whereas expression of the β-subunit alone did not result in a functional potassium channel, a coexpression with the α-subunit formed channels with biophysical and pharmacological properties distinct from channels formed by the α-subunit alone. These properties more closely resemble those of native high conductance potassium channels. The report concluded that based on the effect on sensitivity of the channel to voltage and calcium conferred by the β-subunit, that the β-subunit may form part of the transduction machinery of the channel. This reference also showed that these properties could be conferred by chimeric multimers in which a β-subunit from one tissue was able to modulate the α-subunit from another tissue. The possibility was raised that regulated expression of β-subunits, as in tissue-specific or developmental-specific regulation, could constitute a mechanism for generating functional diversity among mammalian high conductance potassium channels.

Meera et al. (FEBS. Lett. 382:84-88 (1996)) disclosed the importance of calcium concentration for the functional coupling between α- and β-subunits of high conductance potassium channels. The reference pointed out that these channels are unique in that they are modulated not only by voltage, but also by calcium in the micromolar range. They referred to the β-subunit as “the regulatory subunit for the pore-forming α-subunit.” The reference demonstrated that intracellular calcium concentration controls the functional coupling between α- and β-subunits of the complex in a concentration range relevant to cellular excitation. The β-subunit used for the experiments was derived from human smooth muscle. The experiments were performed by injecting cRNA into Xenopus oocytes. Channel currents and number of channels were recorded. The results were reported as demonstrating that a minimum calcium concentration was required to switch α- and β-subunits to a functional activated mode. It was proposed that a rise in local calcium concentration would induce a conformational change in one or both of the subunits, triggering the functional coupling and causing the α-subunit to respond much more efficiently to calcium and voltage. Prior to this work, it was thought that the channels were calcium- and voltage-activated and would never open in the virtual absence of calcium. However, the report demonstrated that the channel α-subunit will open at a low calcium concentration and, in fact, becomes independent of calcium at concentrations lower than 100 nM, operating according to a purely voltage-regulated mode. Similarly, the results provided evidence for a calcium dependent mechanism that switches the α-subunit from a calcium-independent to a calcium-dependent mode and from a β-subunit-null interaction to a β-subunit-activated mode.

The β-subunits of voltage-gated potassium channels have been recently reviewed (Barry et al., Ann. Rev. Physiol. 58:363-394 (1996)).

Oberst et al. (Oncogene 14:1109-1116 (1997)) recently identified a nucleic acid sequence in quail cDNA in which the corresponding gene encodes a 200 amino acid protein with 46-48% amino acid sequence identity to regulatory β-subunits of the bovine, human, and canine high conductance calcium-activated potassium channel. Studies of gene expression in v-myc-transformed quail embryo fibroblasts led to the isolation of a clone hybridizing in the normal, but not in the transformed fibroblasts. Subsequent analysis revealed that the sequence was expressed in all normal avian fibroblasts tested, but was undetectable in a variety of cell lines transformed by a variety of oncogenes or chemical carcinogens. It was suggested that the protein encoded by this sequence is a regulatory subunit of a calcium-activated potassium channel potentially involved in the regulation of cell proliferation.

Rhodes et al. (J. Neurosci. 17:8246-8258 (1997)) examined the association and colocalization of two mammalian β-subunits with several potassium channel α-subunits in adult rat brain. The experiment showed that the two subunits associate with virtually all of the α-subunits examined. It was suggested that the differential expression and association of cytoplasmic β-subunits with pore-forming α-subunits could significantly contribute to the complexity and heterogeneity of voltage-gated potassium channels in excitable cells. The results provided a biochemical and neuroanatomical basis for the differential contribution of α and β subunits to electrophysiologically diverse neuronal potassium currents.

Ion channels are a major target for drug action and development. Accordingly, it is valuable to the field of pharmaceutical development to identify and characterize previously unknown ion channel components. The present invention advances the state of the art by providing a previously unidentified human potassium channel β-subunit and methods to utilize the potassium channel ∃-subunit for disease diagnosis and methods to modulate and regulate the function and neuronal excitability of the Slowpoke calcium-dependent potassium channel. These methods may have particular relevance for treating and diagnosing disorders involving the mammalian CNS.

### Summary of the Invention

It is a general object of the invention to modulate ion channels. Therefore, it is an object of the invention to identify novel ion channel components. It is a specific object of the invention to provide novel ion channel β-subunit polypeptides, herein referred to as C7F2 polypeptides, that are useful as reagents or targets in assays applicable to treatment and diagnosis of ion-channel-mediated disorders.

It is a further object of the invention to provide polynucleotides corresponding to the novel β-subunit polypeptides that are useful as targets and reagents in assays applicable to treatment and diagnosis of ion-channel-mediated disorders and useful for producing novel ion channel polypeptides by recombinant methods.

A further specific object of the invention is to describe the cloning and functional characterization of a novel Slo auxiliary subunit in human and mouse, ∃4, that is highly expressed in human brain. The ∃4 subunit co-immunoprecipitates with human and mouse Slo (hSlo and mSlo). The predicted ∃4 protein is structurally related to the previously cloned ∃1 and ∃2 subunits with N- and C-terminal transmembrane domains.

The novel ∃4 subunit shares 23% sequence identity with the ∃1 subunit and 32% sequence identity with the ∃2 subunit. Multiple sequence alignment identifies several conserved residues in these three proteins, including the cysteines involved in forming the disulphide-linked extracellular domain.

Human multiple tissue Northern blots (Clontech) probed with a 32P-labelled PCR fragment and quantitative PCR analysis of messenger RNA levels using Taqman™ (Perkin-Elmer) showed robust expression of ∃4 in the nervous system. Although these methods detected a faint signal in peripheral tissues, in situ hybridization demonstrated that ∃4 is restricted to the nervous system. To analyze ∃4 expression at the cellular level in situ hybridization analysis was performed in sections of brain, spinal cord, and dorsal root ganglia (DRG) from human and monkey. Sections were labeled with antisense cRNA probes to Slowpoke (Slo), ∃1, ∃2, or ∃4 subunits using standard techniques.

In the central nervous system (CNS), co-expression of ∃4 and Slo was observed in neuronal populations in the cortex, ifundibulum, hippocampal formation, thalamus, and striatum. ∃4 expression was also observed in the substantia nigra, red nucleus, pons, cerebellum, brain stem and spinal cord (including motor neuron). In contrast, ∃1 was not detected in the brain whereas ∃2 was found in several brain regions at much lower levels than ∃4. In the peripheral nervous system (PNS), ∃4, ∃2 and Slo were expressed in sensory neurons of the DRG. ∃4 is expressed in medium size neurons whereas ∃2 appears to be restricted to smaller diameter neurons. Slo expression is more widespread in DRG neurons of both small and medium size.

Taqman™ analysis was also used to determine ∃4 expression in rat tissues. In accordance with the primate expression data, ∃4 is specific to the nervous system and highest levels were observed in sympathetic neurons of the rat superior cervical ganglion.

Also, ∃4 was examined in various peripheral nerve pain models. ∃4 was up-regulated in DRG and spinal cord after chronic constriction injury of the sciatic nerve (a model of neuropathic pain) but not after intraplantar injection of complete Freund's Adjuvant (a model of inflammatory pain).

The ∃4 down-regulates Slo channel activity, by shifting its activation range to more depolarized voltages and slowing its activation kinetics. Under conditions herein described, the effects of ∃4 on Slowpoke channel properties are diametrically opposite to those of ∃1 and ∃2/3, in that channel activity is down-regulated by ∃4. Therefore, ∃4 may play a critical role in the regulation of neuronal excitability and neurotransmitter release by Slowpoke family channels.

A specific object of the invention is to identify compounds that act as agonists or antagonists and modulate the function or expression of the β-subunit.

A further specific object of the invention is to provide the compounds that modulate the expression or function of the β-subunit for treatment and diagnosis of ion-channel-related disorders.

The novel β-subunit polypeptides and polynucleotides of the invention are useful for the treatment of β-subunit-associated or related disorders, including, for example, central nervous system (CNS) disorders, cardiovascular system disorders, and musculoskeletal system disorders. β-subunit-associated or related disorders also include disorders of tissues in which the novel β-subunit C7F2 is expressed, e.g., heart, placental, lung, kidney, prostate, testicular, ovarian, spleen, small and large intestine, colon, or thymus tissues, as well as in brain tissues, including cerebellum, cerebral cortex, medulla, spinal cord, occipital lobe, frontal lobe, temporal lobe, putanem, amygdala, caudate, corpus colosum, hippocampus, substantia nigra, subthalamus and thalamus.

The invention is thus based on the identification of a novel potassium channel β-subunit.

This β-subunit is useful for modulating ion channels in view of its interaction with the pore-forming α-subunit. Accordingly, by using the β-subunit to modulate α-subunit activity, ion channel modulation is provided.

The β-subunit is also useful per se as a target or reagent for treatment and diagnosis.

The invention thus provides isolated β-subunit polypeptides including a polypeptide having the amino acid sequence shown in SEQ ID NO:68.

The invention also provides isolated β-subunit nucleic acid molecules having the sequence shown in SEQ ID NO:67 or in the deposited cDNA.

The invention also provides variant polypeptides having an amino acid sequence that is substantially homologous to the amino acid sequence shown in SEQ ID NO:67 or encoded by the deposited cDNA.

The invention also provides variant nucleic acid sequences that are substantially homologous to the nucleotide sequence shown in SEQ ID NO:67 or in the deposited cDNA.

The invention also provides fragments of the polypeptide shown in SEQ ID NO:68 and nucleotide shown in SEQ ID NO:67, as well as substantially homologous fragments of the polypeptide or nucleic acid.

The invention also provides vectors and host cells for expression of the β-subunit nucleic acid molecules and polypeptides and particularly recombinant vectors and host cells.

The invention also provides methods of making the vectors and host cells and methods for using them to produce the β-subunit nucleic acid molecules and polypeptides.

The invention also provides antibodies that selectively bind the β-subunit polypeptides and fragments.

The invention also provides methods of screening for compounds that modulate the expression or activity of the β-subunit polypeptides. Modulation can be at the level of the polypeptide β-subunit or at the level of controlling the expression of nucleic acid expressing the β-subunit polypeptide.

The invention also provides a process for modulating β-subunit expression or activity using the screened compounds, including to treat conditions related to expression or activity of the β-subunit polypeptides.

The invention also provides diagnostic assays for determining the presence, level, or activity of the β-subunit polypeptides or nucleic acid molecules in a biological sample.

The invention also provides diagnostic assays for determining the presence of a mutation in the β-subunit polypeptides or nucleic acid molecules.

### Detailed Description of the Invention

Polypeptides

The invention is based on the discovery of a novel potassium channel β-subunit. An expressed sequence tag (EST) was identified in a monkey striatum library. This EST had homology to a quail putative potassium channel β-subunit (Oberst et al., cited above) and a human calcium-activated potassium channel β-subunit (Meera et al., cited above). A human EST was identified with similarity to the 3′ end of the monkey EST. This human EST was sequenced and found to be nearly identical to the 3′ end of the monkey clone. This EST was used in a Northern blot analysis for expression in various human tissues.

The gene is expressed preferentially in brain with highest expression in the cortical regions but with expression in other regions and in the spinal cord. In the brain the following tissues showed a positive signal upon Northern blotting: cerebellum, cerebral cortex, medulla, spinal cord, occipital lobe, frontal lobe, temporal lobe, putanem, amygdala, caudate, corpus colosum, hippocampus, substantia nigra, subthalamus and thalamus. However, expression is also found in heart, kidney, placenta, lung, prostate, testes, ovary and small and large intestine. Using the sequence as a probe, a full-length human clone from fetal brain was identified and sequenced and designated C7F2.

The invention thus relates to a novel potassium channel β-subunit having the deduced amino acid sequence shown in (SEQ ID NO:68).

The “C7F2 β-subunit polypeptide” or “C7F2 β-subunit protein” refers to the polypeptide in SEQ ID NO:68 or encoded by the deposited cDNA. The term “β-subunit protein” or “β-subunit polypeptide”, however, further includes the variants described herein, as well as fragments derived from the full length C7F2 β-subunit polypeptide and variants.

The present invention thus provides an isolated or purified C7F2 potassium channel β-subunit polypeptide and variants and fragments thereof.

The C7F2 β-subunit polypeptide is a 210 residue protein exhibiting 5 structural domains. The amino terminal intracellular domain is identified to be within residues 1 to about residue 19 in SEQ ID NO:68. The first transmembrane domain is identified to be within residues from about 20 to about 40 in SEQ ID NO:68. The extracellular loop is identified to be within residues from about 41 to 167 in SEQ ID NO:68. The second transmembrane domain is identified to be within residues from about 168 to about 192 in SEQ ID NO:68. The carboxy terminal intracellular domain is identified to be within residues from about 193 to 210.

As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be considered “isolated” or “purified.”

The β-subunit polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

In one embodiment, the language “substantially free of cellular material” includes preparations of the β-subunit polypeptide having less than about 30% (by dry weight) other proteins (i.e., contaminating protein), less than about 20% other proteins, less than about 10% other proteins, or less than about 5% other proteins. When the β-subunit polypeptide is recombinantly produced, it can also be substantially free of culture medium, i.e., culture medium represents less than about 20%, less than about 10%, or less than about 5% of the volume of the protein preparation.

The language “substantially free of chemical precursors or other chemicals” includes preparations of the β-subunit polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, the β-subunit polypeptide comprises the amino acid sequence shown in SEQ ID NO:68. However, the invention also encompasses sequence variants. Variants include a substantially homologous protein encoded by the same genetic locus in an organism, i.e., an allelic variant. Variants also encompass proteins derived from other genetic loci in an organism, but having substantial homology to the C7F2 β-subunit protein of SEQ ID NO:68. Variants also include proteins substantially homologous to the C7F2 β-subunit protein but derived from another organism, i.e., an ortholog. Variants also include proteins that are substantially homologous to the C7F2 β-subunit protein that are produced by chemical synthesis. Variants also include proteins that are substantially homologous to the C7F2 β-subunit protein that are produced by recombinant methods. It is understood, however, that variants exclude any amino acid sequences disclosed prior to the invention.

As used herein, two proteins (or a region of the proteins) are substantially homologous when the amino acid sequences are at least about 55-60%, typically at least about 70-75%, more typically at least about 80-85%, and most typically at least about 90-95% or more homologous. A substantially homologous amino acid sequence, according to the present invention, will be encoded by a nucleic acid sequence hybridizing to the nucleic acid sequence, or portion thereof, of the sequence shown in SEQ ID NO:67 under stringent conditions as more fully described below.

To determine the percent homology of two amino acid sequences, or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity”. The percent homology between the two sequences is a function of the number of identical positions shared by the sequences (i.e., percent homology equals the number of identical positions/total number of positions times 100).

The invention also encompasses polypeptides having a lower degree of identity but having sufficient similarity so as to perform one or more of the same functions performed by the C7F2 β-subunit polypeptide. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

TABLE 10
Conservative Amnio Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Preferred computer program methods to determine identify and similarity between two sequences include, but are not limited to, GCG program package (Devereux, J., et al., Nucleic Acids Res. 12(1):387 (1984)), BLASTP, BLASTN, FASTA (Atschul, S. F. et al., J. Molec. Biol. 215:403 (1990)).

A variant polypeptide can differ in amino acid sequence by one or more substitutions, deletions, insertions, inversions, fusions, and truncations or a combination of any of these.

Variant polypeptides can be fully functional or can lack function in one or more activities. Thus, in the present case, variations can affect the function, for example, of one or more of the regions corresponding to ligand binding, transmembrane association, phosphorylation, and α-subunit interaction.

Fully functional variants typically contain only conservative variation of variation in non-critical residues or in non-critical regions. Functional variants can also contain substitution of similar amino acids which result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for the β-subunit polypeptide. This includes preventing immunogenicity from pharmaceutical formulations by preventing protein aggregation, for example if soluble peptides corresponding to the extracellular loop are used.

Useful variations include alteration of ligand binding characteristics. For example, one embodiment involves a variation at the binding site that results in increased or decreased extent or rate of ligand binding. A further useful variation at the same site can result in a higher or lower affinity for ligand. Useful variations also include changes that provide affinity for another ligand. Another useful variation provides for reduced or increased affinity for the α-subunit or for binding by a different α-subunit than the one with which the β-subunit is normally associated. Another useful variation provides for reduced or increased rate or extent of activation of the α-subunit. Another useful variation provides a fusion protein in which one or more segments is operatively fused to one or more segments from another β-subunit. Another useful variation provides for an increase or decrease in phosphorylation or glycosylation.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity such as ligand binding, α-subunit association or activation, or channel currents. Sites that are critical for ligand binding and α-subunit modulation can also be determined by structural analysis such as crystallization, nuclear magnetic resonance or photoaffinity labeling (Smith et al., J. Mol. Biol. 224:899-904 (1992); de Vos et al. Science 255:306-312 (1992)).

The invention also includes polypeptide fragments of the C7F2 β-subunit protein. Fragments can be derived from the amino acid sequence shown in SEQ ID NO:68. However, the invention also encompasses fragments of the variants of the β-subunit protein as described herein.

The fragments to which the invention pertains, however, are not to be construed as encompassing fragments that may be disclosed prior to the present invention.

Fragments can retain one or more of the biological activities of the protein, for example the ability to bind to an α-subunit or ligand. Biologically active fragments can comprise a domain or motif, e.g., an extracellular domain, one or more transmembrane domains, α-subunit binding domain, or intracellular domains or functional parts thereof. Such peptides can be, for example, 7, 10, 15, 20, 30, 35, 36, 37, 38, 39, 40, 50, 100 or more amino acids in length.

Possible fragments include, but are not limited to: 1) peptides comprising from about amino acid 1 to about amino acid 19 of SEQ ID NO:68; 2) peptides comprising from about amino acid 20 to about amino acid 40 of SEQ ID NO:68; 3) peptides comprising from about amino acid 41 to about amino acid 167 of SEQ ID NO:68; 4) peptides comprising from about amino acid 168 to about amino acid 192; and 5) peptides comprising from about amino acid 193 to amino acid 210, or combinations of these fragments such as two, three, or four domains. Other fragments include fragments containing the various functional sites described herein such as phosphorylation sites such as around amino acids 210, 19, 14, and 167, and glycosylation sites around amino acids 56 and 93. Fragments, for example, can extend in one or both directions from the functional site to encompass 5, 10, 15, 20, 30, 40, 50, or up to 100 amino acids. Further, fragments can include subfragments of the specific domains mentioned above, which subfragments retain the function of the domain from which they are derived. Fragments also include amino acid sequences greater than 71 amino acids. Fragments also include antigenic fragments. Further specific fragments include amino acids 1 to 29, 306 to 326, and fragments including but larger than amino acids 1-29, 30-65, 67-252, 254-305, 306-326, 330-338, 342-347, 353-361, and 366-382.

Accordingly, possible fragments include fragments defining the site of association between the β and α subunits, fragments defining a ligand binding site, fragments defining a glycosylation site, fragments defining membrane association, and fragments defining phosphorylation sites. By this is intended a discrete fragment that provides the relevant function or allows the relevant function to be identified. In a preferred embodiment, the fragment contains the site(s) of α and β subunit association.

The invention also provides fragments with immunogenic properties. These contain an epitope-bearing portion of the C7F2 β-subunit protein and variants. These epitope-bearing peptides are useful to raise antibodies that bind specifically to a β-subunit polypeptide or region or fragment. These peptides can contain at least 7, at least 14, or between at least about 15 to about 30 amino acids.

Non-limiting examples of antigenic polypeptides that can be used to generate antibodies include peptides derived from the extracellular domain.

The epitope-bearing β-subunit and polypeptides may be produced by any conventional means (Houghten, R. A., Proc. Natl. Acad. Sci. USA 82:5131-5135 (1985)). Simultaneous multiple peptide synthesis is described in U.S. Pat. No. 4,631,211.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the β-subunit fragment and an additional region fused to the carboxyl terminus of the fragment.

The invention thus provides chimeric or fusion proteins. These comprise a β-subunit protein operatively linked to a heterologous protein having an amino acid sequence not substantially homologous to the β-subunit protein. “Operatively linked” indicates that the β-subunit protein and the heterologous protein are fused in-frame. The heterologous protein can be fused to the N-terminus or C-terminus of the β-subunit protein.

In one embodiment the fusion protein does not affect β-subunit function per se. For example, the fusion protein can be a GST-fusion protein in which the β-subunit sequences are fused to the C-terminus of the GST sequences. Other types of fusion proteins include, but are not limited to, enzymatic fusion proteins, for example beta-galactosidase fusions, yeast two-hybrid GAL fusions, poly-His fusions and Ig fusions. Such fusion proteins, particularly poly-His fusions, can facilitate the purification of recombinant β-subunit protein. In certain host cells (e.g., mammalian host cells), expression and/or secretion of a protein can be increased by using a heterologous signal sequence. Therefore, in another embodiment, the fusion protein contains a heterologous signal sequence at its N-terminus.

EP-A-O 464 533 discloses fusion proteins comprising various portions of immunoglobin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition 8:52-58 (1995) and Johanson et al., The Journal of Biological Chemistry 270, 16:9459-9471 (1995). Thus, this invention also encompasses soluble fusion proteins containing a β-subunit polypeptide and various portions of the constant regions of heavy or light chains of immunoglobulins of various subclass (IgG, IgM, IgA, IgE). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence which is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A β-subunit protein-encoding nucleic acid can be cloned into such an expression vector such that the fusion moiety is linked in-frame to the β-subunit protein.

Another form of fusion protein is one that directly affects β-subunit functions. Accordingly, a β-subunit polypeptide encompassed by the present invention in which one or more of the β-subunit segments has been replaced by homologous segments from another β-subunit. Various permutations are possible. The various segments include the intracellular amino and carboxy terminal domains, the two transmembrane domains, and the extracellular loop domain. More specifically, the functional domains include the domain containing the ligand binding site, the domains containing the phosphorylation sites, and the domain containing the site that functions to bind α-subunit or modulate α-subunit activation. Any of these domains or subregions thereof containing a specific site can be replaced with the corresponding domain or subregion from another β-subunit protein, or other subunit protein that modulates α-subunit activation. Accordingly, one or more of the specific domains or functional subregions can be combined with those from another subunit that modulates an α-subunit. Thus, chimeric β-subunits can be formed in which one or more of the native domains or subregions has been replaced.

The invention also encompasses chimeric channels in which an α-subunit other than the one with which the β-subunit is naturally found is substituted. The β-subunit can therefore be tested for the ability to modulate other α-subunits. Using assays directed towards these α-subunits as end points, allows the assessment of the β-subunit function. With this type of construct, an α-subunit can be made responsive to a ligand by which it is not normally activated. Thus, by substitution of the β-subunit, a ligand binding to that β-subunit can be used to modulate the activity of the α-subunit.

The isolated β-subunit protein can be purified from cells that naturally express it, such as from brain, heart, kidney, prostate, placenta, lung, testes, ovary and intestine, purified from cells that have been altered to express it (recombinant), or synthesized using known protein synthesis methods.

In one embodiment, the protein is produced by recombinant DNA techniques. For example, a nucleic acid molecule encoding the β-subunit polypeptide is cloned into an expression vector, the expression vector introduced into a host cell and the protein expressed in the host cell. The protein can then be isolated from the cell by an appropriate purification scheme using standard protein purification techniques. Polypeptides often contain amino acids other than the 20 amino acids commonly referred to as the 20 naturally-occurring amino acids. Further, many amino acids, including the terminal amino acids, may

be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al., Meth. Enzymol. 182: 626-646 (1990) and Rattan et al., Ann. N.Y. Acad. Sci. 663:48-62 (1992).

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

Polypeptide Uses

The β-subunit polypeptides, as well as the β-subunit nucleic acid molecules, modulators of these polypeptides, and antibodies (also referred to herein as “active compounds”) of the invention are useful in the modulation, diagnosis, and treatment of β-subunit-associated or related disorders, also referred to as C7F2-associated or related disorders. Such disorders include, for example, central nervous system (CNS) disorders, cardiovascular system disorders, and musculoskeletal system disorders. CNS disorders include, but are not limited to, cognitive and neurodegenerative disorders such as Alzheimer's disease and dementias related to Alzheimer's disease (such as Pick's disease), senile dementia, Huntington's disease, amyotrophic lateral sclerosis, Parkinson's disease and other Lewy diffuse body diseases, Gilles de la Tourette's syndrome, multiple sclerosis, amyotrophic lateral sclerosis, progressive supranuclear palsy, epilepsy, and Jakob-Creutzfieldt disease autonomic function disorders such as hypertension and sleep disorders, and neuropsychiatric disorders, such as depression, schizophrenia, schizoaffective disorder, korsakoff's psychosis, learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-I), bipolar affective (mood) disorder with hypomania and major depression (BP-II), neurological disorders, e.g., migraine, and obesity. Further CNS-related disorders include, for example, those listed in the American Psychiatric Association's Diagnostic and Statistical manual of Mental Disorders (DSM), the most current version of which is incorporated herein by reference in its entirety.

β-subunit-associated or related disorders can detrimentally affect conveyance of sensory impulses from the periphery to the brain (e.g., pain disorders) and/or conductance of motor impulses from the brain to the periphery; integration of reflexes; interpretation of sensory impulses (e.g., pain); or emotional, intellectual (e.g., learning and memory), or motor processes.

Cardiovascular system disorders include, but are not limited to, arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, long-QT syndrome, congestive heart failure, sinus node disfunction, angina, heart failure, hypertension, atrial fibrillation, atrial flutter, dilated cardiomyopathy, idiopathic cardiomyopathy, myocardial infarction, coronary artery disease, coronary artery spasm, or arrhythmia. C7F2-mediated or related disorders also include disorders of the musculoskeletal system such as paralysis and muscle weakness, e.g., ataxia, myotonia, and myokymia.

β-subunit-associated or related disorders also include disorders of tissues in which C7F2 is expressed, e.g., heart, placental, lung, kidney, prostate, testicular, ovarian, spleen, small and large intestine, colon, or thymus tissues, as well as in brain tissues, including cerebellum, cerebral cortex, medulla, spinal cord, occipital lobe, frontal lobe, temporal lobe, putanem, amygdala, caudate, corpus colosum, hippocampus, substantia nigra, subthalamus and thalamus.

The β-subunit polypeptides and nucleotide sequences encoding the polypeptides find use in modulating a β-subunit function or activity. By “modulating” is intended the upregulating or downregulating of a response. That is, the β-subunit polypeptide and nucleic acid compositions of the invention affect the targeted activity in either a positive or negative fashion. The β-subunit polypeptides can be used to modulate neuronal excitability and neurotransmitter release in the Slowpoke family channels.

More specifically, Slowpoke h∃4 can be used to modulate activation kinetics of the potassium channel. h∃4 decreases Slowpoke activation rates but does not affect its deactivation rate. Likewise, ∃4 down-regulates Slowpoke channel activity. Thus, the sequences of the invention can be used to modulate Slowpoke channel activity. The sequences also find use in regulating neuronal excitability and neurotransmitter release.

The β-subunit-associated or related activities include, but are not limited to, an activity that involves a potassium channel, e.g., a potassium channel in a neuronal cell or a muscle cell, associated with receiving, conducting, and transmitting signals in, for example, the nervous system. Potassium-channel mediated activities include release of neurotransmitters, e.g., dopamine or norepinephrine, from cells, e.g., neuronal cells; modulation of resting potential of membranes, wave forms and frequencies of action potentials, and thresholds of excitation; and modulation of processes such as integration of sub-threshold synaptic responses and the conductance of back-propagating action potentials in, for example, neuronal cells or muscle cells. β-subunit-associated or related activities also include activities which involve a potassium channel in nonneuronal cells, e.g., placental, lung, kidney, prostate, testicular, ovarian, spleen, small intestine, colon, or thymus cells, such as membrane potential, cell volume, and pH regulation. β-subunit-associated or related activities include activities involved in muscle function such as maintenance of muscle membrane potential, regulation of muscle contraction and relaxation, and coordination. A preferred β-subunit activity is modulation or regulation of the pore-forming α-subunit of a potassium channel, particularly activation of the α-subunit. Moreover, the β-subunit can be used in a method to identify mammalian neurons that are most excitable.

Accordingly, in one aspect, this invention provides a method for identifying a compound suitable for treating a β-subunit-associated or related disorder by contacting a C7F2 β-subunit polypeptide, or a cell expressing a C7F2′-subunit polypeptide, with a test compound and determining whether the C7F2 β-subunit polypeptide binds to the test compound, thereby identifying a compound suitable for treating a β-subunit-associated or related disorder.

It was shown in immunoprecipitation experiments that Slowpoke ∃4 binds to hSlo. Mammalian Slowpoke ∀ subunits co-immunoprecipitate with ∃4 subunits. When HEK 293 cells were transfected with hSlo together with h∃4, and hSlo is immunoprecipitated with a specific antibody, h∃4 can be detected in the immunoprecipitate.

The β-subunit polypeptides are useful for producing antibodies specific for the C7F2 β-subunit protein, regions, or fragments.

The ∃-subunit polypeptides of the invention exhibit a unique tissue distribution, being expressed predominantly in the brain and the peripheral nervous system, and find use in modulating Slowpoke channel activity.

The actions of the ∃-subunit sequences of the invention on hSlo channel activity are unique. The sequences can be used to modulate the activation kinetics and to shift the voltage-dependence of activation to more depolarized voltages. Generally, the ∃4 sequences can be used to produce a marked down-regulation of channel activity. In particular, the β-subunit polypeptides can be used to modulate the voltage dependence of hSlo activation. h∃4 can influence the steady-state activation of hSlo. Thus, the sequences may be used to modulate the voltage-dependence of hSlo activation.

The β-subunit polypeptides can be used in a method to modulate the toxin block of Slowpoke channel alpha subunits. Generally, auxiliary subunits often alter the effects of pharmacological agents on Slowpoke channel alpha subunits. The sequences can be used to alter cellular response, particularly to decrease channel sensitivity to toxins. Thus, expression of the sequences of the invention can be used to modulate channel sensitivity, particularly sensitivity to toxins.

The β-subunit polypeptides are also useful in drug screening assays, in cell-based or cell-free systems. Cell-based systems can be native i.e., cells that normally express the β-subunit protein, as a biopsy or expanded in cell culture. In one embodiment, however, cell-based assays involve recombinant host cells expressing the β-subunit protein.

The polypeptides can be used to identify compounds that modulate β-subunit activity. Both C7F2 β-subunit protein and appropriate variants and fragments can be used in high throughput screens to assay candidate compounds for the ability to bind to the β-subunit. These compounds can be further screened against a functional β-subunit to determine the effect of the compound on the β-subunit activity. Compounds can be identified that activate (agonist) or inactivate (antagonist) the β-subunit to a desired degree.

The β-subunit polypeptides can be used to screen a compound for the ability to stimulate or inhibit interaction between the β-subunit protein and a target molecule that normally interacts with the β-subunit protein. The target can be ligand or another channel subunit with which the β-subunit protein normally interacts (for example, the α-subunit in the potassium channel). The target can be a molecule that modifies the β-subunit such as by phosphorylation, for example, casein kinase II. The assay includes the steps of combining the β-subunit protein with a candidate compound under conditions that allow the β-subunit protein or fragment to interact with the target molecule, and to detect the formation of a complex between the protein and the target or to detect the biochemical consequence of the interaction with the β-subunit protein and the target, such as ion currents or any of the associated effects of the currents, phosphorylation, change in cell volume, mutagenesis, or transformation.

The invention also encompasses chimeric channels in which a β-subunit is associated with a heterologous α-subunit. Thus, the β-subunit can be used to modulate heterologous α-subunits, as a target for drug screening and in diagnosis and treatment.

Candidate compounds include, for example, 1) small organic and inorganic molecules (e.g., molecules obtained from combinatorial and natural product libraries); 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies as well as Fab, F(ab□)2, Fab expression library fragments, and epitope-binding fragments of antibodies); and 4) peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L-configuration amino acids.

The invention provides other end points to identify compounds that modulate (stimulate or inhibit) β-subunit activity. The assays typically involve an assay of events in channeling that indicate β-subunit activity. A preferred assay involves the activation of the α-subunit.

Assays allowing the assessment of β-subunit activity are known to those of skill in the art and can be found, for example, in McManus et al. (1995), Knaus et al. (1996), Knaus et al. (1994), Meera et al., and Oberst et al., cited above.

Binding and/or modulating (activating or inhibiting) compounds can also be screened by using chimeric subunit proteins in which the ligand binding or α-subunit binding region is replaced by a heterologous region. For example, an α-subunit binding region can be used that interacts with a different α-subunit than that which is recognized by the native β-subunit. Accordingly, a different end-point assay is available. Alternatively, one or two transmembrane regions can be replaced with transmembrane portions specific to a host cell that is different from the native host cell from which the native β-subunit is derived. This allows for assays to be performed in other than the original host cell. Alternatively, the ligand binding region can be replaced by a region binding a different ligand, thus, enabling an assay for test compounds that interact with the heterologous ligand binding region but still cause channeling function, including α-subunit activation.

The β-subunit polypeptides are also useful in competition binding assays in methods designed to discover compounds that interact with the β-subunit. Thus, a compound is exposed to a β-subunit polypeptide under conditions that allow the compound to bind or to otherwise interact with the polypeptide. Competing β-subunit polypeptide is also added to the mixture. If the test compound interacts with the competing β-subunit polypeptide, it decreases the amount of complex formed or activity from the β-subunit target. This type of assay is particularly useful in cases in which compounds are sought that interact with specific regions of the β-subunit. Thus, the polypeptide that competes with the target β-subunit region is designed to contain peptide sequences corresponding to the region of interest.

The β-subunit polypeptides can be used to detect Slowpoke ∀ subunit distribution within various tissues using co-immunoprecipitation, as previously described.

A β-subunit is also useful for assessing function of a given α-subunit. Thus, alteration in channel currents, number of receptors, cell transformation, or any other biological end point can be assessed using the β-subunit of the present invention in cell-based or cell-free assays with a given α-subunit. Mutation in the α-subunit can be detected by any of the various end points. Moreover, mutations in the β-subunit that complement (i.e., correct) mutations in the α-subunit can be identified through cell-based or cell-free assays. Such assays could even be performed at the level of the organism, as with a transgenic animal (see below).

To perform cell free drug screening assays, it is desirable to immobilize either the β-subunit protein, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/flh385 fusion proteins can be adsorbed onto glutathione sepharose beads (Sigma Chemical, St. Louis, Mo.) or glutathione derivatized microtitre plates, which are then combined with the cell lysates (e.g., 35S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of β-subunit-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a β-subunit-binding protein and a candidate compound are incubated in the β-subunit protein-presenting wells and the amount of complex trapped in the well can be quantitated. Methods for detecting such complexes, in addition to those described above for the GST-immobilized complexes, include immunodetection of complexes using antibodies reactive with the β-subunit protein target molecule, or which are reactive with β-subunit protein and compete with the target molecule; as well as enzyme-linked assays which rely on detecting an enzymatic activity associated with the target molecule.

Modulators of β-subunit protein activity identified according to these drug screening assays can be used to treat a subject with a disorder mediated by the β-subunit. These methods of treatment include the steps of administering the modulators of protein activity in a pharmaceutical composition as described herein, to a subject in need of such treatment.

The β-subunit polypeptides also are useful to provide a target for diagnosing a disease or predisposition to disease mediated by the β-subunit protein. Accordingly, methods are provided for detecting the presence, or levels of, the β-subunit protein in a cell, tissue, or organism. The method involves contacting a biological sample with a compound capable of interacting with the β-subunit protein such that the interaction can be detected.

One agent for detecting β-subunit protein is an antibody capable of selectively binding to β-subunit protein. A biological sample includes tissues, cells and biological fluids isolated from a subject, as well as tissues, cells and fluids present within a subject.

The β-subunit protein also provides a target for diagnosing active disease, or predisposition to disease, in a patient having a variant β-subunit protein. Thus, β-subunit protein can be isolated from a biological sample, assayed for the presence of a genetic mutation that results in aberrant β-subunit protein. This includes amino acid substitution, deletion, insertion, rearrangement, (as the result of aberrant splicing events), and inappropriate post-translational modification. Analytic methods include altered electrophoretic mobility, altered tryptic peptide digest, altered β-subunit activity in cell-based or cell-free assay, alteration in ligand, α-subunit, or antibody-binding pattern, altered isoelectric point, direct amino acid sequencing, and any other of the known assay techniques useful for detecting mutations in a protein.

In vitro techniques for detection of β-subunit protein include enzyme linked immunosorbent assays (ELISAs), Western blots, immunoprecipitations and immunofluorescence. Alternatively, the protein can be detected in vivo in a subject by introducing into the subject a labeled anti-β-subunit antibody. For example, the antibody can be labeled with a radioactive marker whose presence and location in a subject can be detected by standard imaging techniques. Particularly useful are methods which detect the allelic variant of a β-subunit protein expressed in a subject and methods which detect fragments of a β-subunit protein in a sample.

The β-subunit polypeptides are also useful in pharmacogenomic analysis. Pharmacogenomics deal with clinically significant hereditary variations in the response to drugs due to altered drug disposition and abnormal action in affected persons. See, e.g., Eichelbaum, M. (1996) Clin. Exp. Pharmacol. Physiol. 23(10-11):983-985 and Linder, M. W. (1997) Clin. Chem. 43(2):254-266. The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes effects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer.

Accordingly, genetic polymorphism may lead to allelic protein variants of the β-subunit protein in which one or more of the β-subunit functions in one population is different from those in another population. The polypeptides thus allow a target to ascertain a genetic predisposition that can affect treatment modality. Thus, in a ligand-based treatment, for example, polymorphism may give rise to sites that are more or less active in ligand binding, and channel activation. Accordingly, ligand choice or dosage could be modified to maximize the therapeutic effect within a given population containing a polymorphism. As an alternative to genotyping, specific polymorphic polypeptides could be identified.

The β-subunit polypeptides are also useful for monitoring therapeutic effects during clinical trials and other treatment. Thus, the therapeutic effectiveness of an agent that is designed to increase or decrease gene expression, protein levels or β-subunit activity can be monitored over the course of treatment using the β-subunit polypeptides as an end-point target.

The β-subunit polypeptides are also useful for treating a β-subunit-associated disorders. Accordingly, methods for treatment include the use of soluble subunit or fragments of the β-subunit protein that compete for molecules interacting with the extracellular portions of the subunit. These β-subunits or fragments can have a higher affinity for the molecule so as to provide effective competition.

Antibodies

The invention also provides antibodies that selectively bind to the subunit protein and its variants and fragments. An antibody is considered to selectively bind, even if it also binds to other proteins that are not substantially homologous with the β-subunit protein. These other proteins share homology with a fragment or domain of the β-subunit protein. This conservation in specific regions gives rise to antibodies that bind to both proteins by virtue of the homologous sequence. In this case, it would be understood that antibody binding to the subunit protein is still selective.

Antibodies are preferably prepared from these regions or from discrete fragments in these regions. However, antibodies can be prepared from any region of the peptide as described herein.

Antibodies can be polyclonal or monoclonal. An intact antibody, or a fragment thereof (e.g. Fab or F(ab□)2) can be used.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

To generate antibodies, an isolated β-subunit polypeptide is used as an immunogen to generate antibodies using standard techniques for polyclonal and monoclonal antibody preparation. Either the full-length protein or antigenic peptide fragment can be used. A preferred fragment produces an antibody that diminishes or completely prevents association between the α and β subunits. Accordingly, a preferred antibody is one that diminishes or completely inhibits association between the two subunits. An antigenic fragment will typically comprise at least 7 contiguous amino acid residues. The antigenic peptide can comprise, however, at least 12, at least 14 amino acid residues, at least 15 amino acid residues, at least 20 amino acid residues, or at least 30 amino acid residues. In one embodiment, fragments correspond to regions that are located on the surface of the protein, e.g., hydrophilic regions.

An appropriate immunogenic preparation can be derived from native, recombinantly expressed, protein or chemically synthesized peptides.

Antibody Uses

The antibodies can be used to isolate a β-subunit protein by standard techniques, such as affinity chromatography or immunoprecipitation. The antibodies can facilitate the purification of the natural β-subunit protein from cells and recombinantly produced β-subunit protein expressed in host cells.

The antibodies are useful to detect the presence of β-subunit protein in cells or tissues to determine the pattern of expression of the β-subunit among various tissues in an organism and over the course of normal development.

The antibodies can be used to detect β-subunit protein in situ, in vitro, or in a cell lysate or supernatant in order to evaluate the abundance and pattern of expression.

The antibodies can be used to assess abnormal tissue distribution or abnormal expression during development.

Antibody detection of fragments of the full length β-subunit protein can be used to identify β-subunit turnover.

Further, the antibodies can be used to assess β-subunit expression in disease states such as in active stages of the disease or in an individual with a predisposition toward disease related to β-subunit function. When a disorder is caused by an inappropriate tissue distribution, developmental expression, or level of expression of the β-subunit protein, the antibody can be prepared against the normal β-subunit protein. If a disorder is characterized by a specific mutation in the β-subunit protein, antibodies specific for this mutant protein can be used to assay for the presence of the specific mutant β-subunit protein.

The antibodies can also be used to assess normal and aberrant subcellular localization of cells in the various tissues in an organism.

Antibodies can be developed against the whole β-subunit or portions of the β-subunit, for example, the intracellular regions, the extracellular region, the transmembrane regions, and specific functional sites such as the site of ligand binding, the site of interaction with the α-subunit, or sites that are phosphorylated, for example by casein kinase II.

The diagnostic uses can be applied, not only in genetic testing, but also in monitoring a treatment modality. Accordingly, where treatment is ultimately aimed at correcting β-subunit expression level or the presence of aberrant β-subunits and aberrant tissue distribution or developmental expression, antibodies directed against the β-subunit or relevant fragments can be used to monitor therapeutic efficacy.

Additionally, antibodies are useful in pharmacogenomic analysis. Thus, antibodies prepared against polymorphic β-subunit proteins can be used to identify individuals that require modified treatment modalities.

The antibodies are also useful as diagnostic tools as an immunological marker for aberrant β-subunit protein analyzed by electrophoretic mobility, isoelectric point, tryptic peptide digest, and other physical assays known to those in the art.

The antibodies are also useful for tissue typing. Thus, where a specific β-subunit protein has been correlated with expression in a specific tissue, antibodies that are specific for this β-subunit protein can be used to identify a tissue type.

The antibodies are also useful in forensic identification. Accordingly, where an individual has been correlated with a specific genetic polymorphism resulting in a specific polymorphic protein, an antibody specific for the polymorphic protein can be used as an aid in identification.

The antibodies are also useful for inhibiting subunit function, for example, blocking ligand binding or α-subunit binding and/or activation. Subunit function involving the extracellular loop is particularly amenable to antibody inhibition.

These uses can also be applied in a therapeutic context in which treatment involves inhibiting subunit function. Antibodies can be prepared against specific fragments containing sites required for function or against intact β-subunit associated with a cell.

The invention also encompasses kits for using antibodies to detect the presence of a β-subunit protein in a biological sample. The kit can comprise antibodies such as a labeled or labelable antibody and a compound or agent for detecting β-subunit protein in a biological sample; means for determining the amount of β-subunit protein in the sample; and means for comparing the amount of β-subunit protein in the sample with a standard. The compound or agent can be packaged in a suitable container. The kit can further comprise instructions for using the kit to detect β-subunit protein.

Polynucleotides

The nucleotide sequence in SEQ ID NO:67 was obtained by sequencing the deposited human full length cDNA. Accordingly, the sequence of the deposited clone is controlling as to any discrepancies between the two and any reference to the sequence of SEQ ID NO:67 includes reference to the sequence of the deposited cDNA.

The specifically disclosed cDNA comprises the coding region, 5′ and 3′ untranslated sequences (SEQ ID NO:67). In one embodiment, the subunit nucleic acid comprises only the coding region.

The human C7F2 β-subunit cDNA is approximately 1737 nucleotides in length and encodes a full length protein that is approximately 210 amino acid residues in length. The nucleic acid is expressed in brain, heart, kidney, placenta, lung, prostate, testes, ovary, and small and large intestine structure of the two transmembrane domains, the term “transmembrane domain” (or “region” or “segment”) refers to a structural amino acid motif which includes a hydrophobic helix that spans the plasma membrane.

The invention provides isolated polynucleotides encoding a C7F2 β-subunit protein. The term “C7F2 β-subunit polynucleotide” or “C7F2 β-subunit nucleic acid” refers to the sequence shown in SEQ ID NO:67 or in the deposited cDNA. The term “β-subunit polynucleotide” or “β-subunit nucleic acid” further includes variants and fragments of the C7F2 polynucleotide.

An “isolated” β-subunit nucleic acid is one that is separated from other nucleic acid present in the natural source of the β-subunit nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB. The important point is that the nucleic acid is isolated from flanking sequences such that it can be subjected to the specific manipulations described herein such as recombinant expression, preparation of probes and primers, and other uses specific to the β-subunit nucleic acid sequences.

Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

The β-subunit polynucleotides can encode the mature protein plus additional amino or carboxy terminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The β-subunit polynucleotides include, but are not limited to, the sequence encoding the mature polypeptide alone, the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

β-subunit polynucleotides can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

One β-subunit nucleic acid comprises the nucleotide sequence shown in SEQ ID NO:67, corresponding to human fetal brain cDNA.

The invention further provides variant subunit polynucleotides, and fragments thereof, that differ from the nucleotide sequence shown in SEQ ID NO:67 due to degeneracy of the genetic code and thus encode the same protein as that encoded by the nucleotide sequence shown in SEQ ID NO:67.

The invention also provides β-subunit nucleic acid molecules encoding the variant polypeptides described herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Variation can occur in either or both the coding and non-coding regions. The variations can produce both conservative and non-conservative amino acid substitutions.

Orthologs, homologs, and allelic variants can be identified using methods well known in the art. These variants comprise a nucleotide sequence encoding a β-subunit that is at least about 55-60%, typically at least about 70-75%, more typically at least about 80-85%, and most typically at least about 90-95% or more homologous to the nucleotide sequence shown in SEQ ID NO:67 or a fragment of this sequence. Such nucleic acid molecules can readily be identified as being able to hybridize under stringent conditions, to the nucleotide sequence shown in SEQ ID NO:67 or a fragment of the sequence. It is understood that stringent hybridization does not indicate substantial homology where it is due to general homology, such as poly A sequences, or sequences common to all or most proteins, all K+ channel β-subunits, or all channel β-subunits. Moreover, it is understood that variants do not include any of the nucleic acid sequences that may have been disclosed prior to the invention.

As used herein, the term “hybridizes under stringent conditions” is intended to describe conditions for hybridization and washing under which nucleotide sequences encoding a β-subunit at least 55-60% homologous to each other typically remain hybridized to each other. The conditions can be such that sequences at least about 65%, at least about 70%, or at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. One example of stringent hybridization conditions are hybridization in 6× sodium chloride/sodium citrate (SSC) at about 45° C., followed by one or more washes in 0.2×SSC, 0.1% SDS at 50-65° C. In one embodiment, an isolated β-subunit nucleic acid molecule that hybridizes under stringent conditions to the sequence of SEQ ID NO:67 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

The invention also provides polynucleotides that comprise a fragment of the full length β-subunit polynucleotides. The fragment can be single or double stranded and can comprise DNA or RNA. The fragment can be derived from either the coding or the non-coding sequence, e.g., transcriptional regulatory sequence.

In one embodiment, β-subunit coding region nucleic acid is at least 216 nucleotides in length and hybridizes under stringent conditions to the nucleic acid molecule comprising the nucleotide sequence of SEQ ID NO:67. Fragments also include those nucleic acid sequences encoding the specific domains described herein. Fragments also include nucleic acids encoding the entire coding sequence. Fragments also include nucleic acids encoding the mature protein. Fragments also include nucleic acid sequences encoding two or more domains. Fragments also include nucleic acid sequences corresponding to the amino acids at the specific functional sites described herein. Fragments further include nucleic acid sequences encoding a portion of the amino acid sequence described herein but further including flanking nucleotide sequences at the 5′ and/or 3′ regions. Other fragments can include subfragments of the specific domains or sites described herein. Fragments also include nucleic acid sequences corresponding to specific amino acid sequences described above or fragments thereof. In these embodiments, the nucleic acid is at least 20, 30, 40, 50, 100, 250 or 500 nucleotides in length. Nucleic acid fragments, according to the present invention, are not to be construed as encompassing those fragments that may have been disclosed prior to the invention.

However, it is understood that a β-subunit fragment includes any nucleic acid sequence that does not include the entire gene.

β-subunit nucleic acid fragments include nucleic acid molecules encoding a polypeptide comprising an amino terminal intracellular domain including amino acid residues from 1 to about 19, a polypeptide comprising the first transmembrane domain (amino acid residues from about 20 to about 40), a polypeptide comprising the extracellular loop domain (amino acid residues from about 41 to about 167), a polypeptide comprising the second transmembrane domain (amino acid residues from about 168 to about 192) and a polypeptide comprising the carboxy terminal intracellular domain (amino acid residues from about 193 to 210). Where the location of the domains have been predicted by computer analysis, one of ordinary skill would appreciate that the amino acid residues constituting these domains can vary depending on the criteria used to define the domains.

The invention also provides β-subunit nucleic acid fragments that encode epitope bearing regions of the β-subunit proteins described herein.

The isolated β-subunit polynucleotide sequences, and especially fragments, are useful as DNA probes and primers.

For example, the coding region of a β-subunit gene can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of β-subunit genes.

A probe/primer typically comprises substantially purified oligonucleotide. The oligonucleotide typically comprises a region of nucleotide sequence, as described above, that hybridizes under stringent conditions to at least about 20, typically about 25, more typically about 40, 50 or 75 consecutive nucleotides of SEQ ID NO:67 sense or anti-sense strand or other β-subunit polynucleotides. A probe further comprises a label, e.g., radioisotope, fluorescent compound, enzyme, or enzyme co-factor.

Polynucleotide Uses

The β-subunit polynucleotides are useful as a hybridization probe for cDNA and genomic DNA to isolate a full-length cDNA and genomic clones encoding the polypeptide described in SEQ ID NO:68 and to isolate cDNA and genomic clones that correspond to variants producing the same polypeptide shown in SEQ ID NO:68 or the other variants described herein. Variants can be isolated from the same tissue and organism from which the polypeptide shown in SEQ ID NO:68 was isolated, different tissues from the same organism, or from different organisms. This method is useful for isolating genes and cDNA that are developmentally controlled and therefore may be expressed in the same tissue at different points in the development of an organism.

The probe can correspond to any sequence along the entire length of the gene encoding the β-subunit. Accordingly, it could be derived from 5′ noncoding regions, the coding region, as specified above, and 3′ noncoding regions.

The nucleic acid probe can be, for example, the full-length cDNA of SEQ ID NO:68, or a fragment thereof, as described above. The probe can be an oligonucleotide of at least 20, 30, 50, 100, 250 or 500 nucleotides in length and sufficient to specifically hybridize under stringent conditions to mRNA or DNA.

Fragments of the polynucleotides described herein are also useful to synthesize larger fragments or full-length polynucleotides described herein. For example, a fragment can be hybridized to any portion of an mRNA and a larger or full-length cDNA can be produced.

The fragments are also useful to synthesize antisense molecules of desired length and sequence.

The β-subunit polynucleotides are also useful as primers for PCR to amplify any given region of a β-subunit polynucleotide.

The β-subunit polynucleotides are also useful for constructing recombinant vectors. Such vectors include expression vectors that express a portion of, or all of, the β-subunit polypeptides. Vectors also include insertion vectors, used to integrate into another polynucleotide sequence, such as into the cellular genome, to alter in situ expression of β-subunit genes and gene products. For example, an endogenous β-subunit coding sequence can be replaced via homologous recombination with all or part of the coding region containing one or more specifically introduced mutations.

The β-subunit polynucleotides are also useful as probes for determining the chromosomal positions of the β-subunit polynucleotides by means of in situ hybridization methods.

The β-subunit polynucleotide probes are also useful to determine patterns of the presence of the gene encoding the β-subunits and their variants with respect to tissue distribution, for example whether gene duplication has occurred and whether the duplication occurs in all or only a subset of tissues. The genes can be naturally occurring or can have been introduced into a cell, tissue, or organism exogenously. The β-subunit polynucleotides are also useful for designing ribozymes corresponding to all, or a part, of the mRNA produced from genes encoding the polynucleotides described herein.

The β-subunit polynucleotides are also useful for constructing host cells expressing a part, or all, of the β-subunit polynucleotides and polypeptides.

The β-subunit polynucleotides are also useful for constructing transgenic animals expressing all, or a part, of the β-subunit polynucleotides and polypeptides.

The β-subunit polynucleotides are also useful for making vectors that express part, or all, of the β-subunit polypeptides.

The β-subunit polynucleotides are also useful as hybridization probes for determining the level of β-subunit nucleic acid expression. Accordingly, the probes can be used to detect the presence of, or to determine levels of, β-subunit nucleic acid in cells, tissues, and in organisms. The nucleic acid whose level is determined can be DNA or RNA. Accordingly, probes corresponding to the polypeptides described herein can be used to assess gene copy number in a given cell, tissue, or organism. This is particularly relevant in cases in which there has been an amplification of the β-subunit genes.

Alternatively, the probe can be used in an in situ hybridization context to assess the position of extra copies of the β-subunit genes, as on extrachromosomal elements or as integrated into chromosomes in which the β-subunit gene is not normally found, for example as a homogeneously staining region.

These uses are relevant for diagnosis of disorders involving an increase or decrease in β-subunit expression relative to normal results.

In vitro techniques for detection of mRNA include Northern hybridizations and in situ hybridizations. In vitro techniques for detecting DNA includes Southern hybridizations and in situ hybridization.

Probes can be used as a part of a diagnostic test kit for identifying cells or tissues that express a β-subunit protein, such as by measuring a level of a subunit-encoding nucleic acid in a sample of cells from a subject e.g., mRNA or genomic DNA, or determining if a β-subunit gene has been mutated.

Nucleic acid expression assays are useful for drug screening to identify compounds that modulate β-subunit nucleic acid expression or activity.

The invention thus provides a method for identifying a compound that can be used to treat a disorder associated with nucleic acid expression of the β-subunit gene. The method typically includes assaying the ability of the compound to modulate the expression of the β-subunit nucleic acid and thus identifying a compound that can be used to treat a disorder characterized by undesired β-subunit nucleic acid expression.

The assays can be performed in cell-based and cell-free systems. Cell-based assays include cells naturally expressing the β-subunit nucleic acid or recombinant cells genetically engineered to express specific nucleic acid sequences.

Alternatively, candidate compounds can be assayed in vivo in patients or in transgenic animals.

The assay for β-subunit nucleic acid expression can involve direct assay of nucleic acid levels, such as mRNA levels, or on collateral compounds involved in the signal pathway (such as cyclic AMP or phosphatidylinositol turnover). Further, the expression of genes that are up- or down-regulated in response to the β-subunit protein signal pathway can also be assayed. In this embodiment the regulatory regions of these genes can be operably linked to a reporter gene such as luciferase.

Thus, modulators of β-subunit gene expression can be identified in a method wherein a cell is contacted with a candidate compound and the expression of mRNA determined. The level of expression of β-subunit mRNA in the presence of the candidate compound is compared to the level of expression of β-subunit mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Accordingly, the invention provides methods of treatment, with the nucleic acid as a target, using a compound identified through drug screening as a gene modulator to modulate β-subunit nucleic acid expression. Modulation includes both up-regulation (i.e. activation or agonization) or down-regulation (suppression or antagonization) of nucleic acid expression.

Alternatively, a modulator for β-subunit nucleic acid expression can be a small molecule or drug identified using the screening assays described herein as long as the drug or small molecule inhibits the β-subunit nucleic acid expression.

The β-subunit polynucleotides are also useful for monitoring the effectiveness of modulating compounds on the expression or activity of the β-subunit gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

The β-subunit polynucleotides are also useful in diagnostic assays for qualitative changes in β-subunit nucleic acid, and particularly in qualitative changes that lead to pathology. The polynucleotides can be used to detect mutations in β-subunit genes and gene expression products such as mRNA. The polynucleotides can be used as hybridization probes to detect naturally occurring genetic mutations in the β-subunit gene and thereby determining whether a subject with the mutation is at risk for a disorder caused by the mutation. Mutations include deletion, addition, or substitution of one or more nucleotides in the gene, chromosomal rearrangement such as inversion or transposition, modification of genomic DNA such as aberrant methylation patterns or changes in gene copy number such as amplification. Detection of a mutated form of the β-subunit gene associated with a dysfunction provides a diagnostic tool for an active disease or susceptibility to disease when the disease results from overexpression, underexpression, or altered expression of a β-subunit protein.

Individuals carrying mutations in the β-subunit gene can be detected at the nucleic acid level by a variety of techniques. Genomic DNA can be analyzed directly or can be amplified by using PCR prior to analysis. RNA or cDNA can be used in the same way.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988); and Nakazawa et al., PNAS 91:360-364 (1994)), the latter of which can be particularly useful for detecting point mutations in the gene (see Abravaya et al., Nucleic Acids Res. 23:675-682 (1995)). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

Alternatively, mutations in a β-subunit gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site.

Perfectly matched sequences can be distinguished from mismatched sequences by nuclease cleavage digestion assays or by differences in melting temperature.

Sequence changes at specific locations can also be assessed by nuclease protection assays such as RNase and S1 protection or the chemical cleavage method.

Furthermore, sequence differences between a mutant subunit gene and a wild-type gene can be determined by direct DNA sequencing. A variety of automated sequencing procedures can be utilized when performing the diagnostic assays (Biotechniques 19:448 (1995)), including sequencing by mass spectrometry (see, e.g., PCT International Publication No. WO 94/16101; Cohen et al., Adv. Chromatogr. 36:127-162 (1996); and Griffin et al., Appl. Biochem. Biotechnol. 38:147-159 (1993)).

Other methods for detecting mutations in the gene include methods in which protection from cleavage agents is used to detect mismatched bases in RNA/RNA or RNA/DNA duplexes (Myers et al., Science 230:1242 (1985); Cotton et al., PNAS 85:4397 (1988); Saleeba et al., Meth. Enzymol. 217:286-295 (1992)), electrophoretic mobility of mutant and wild type nucleic acid is compared (Orita et al., PNAS 86:2766 (1989); Cotton et al., Mutat. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (Myers et al., Nature 313:495 (1985)). Examples of other techniques for detecting point mutations include, selective oligonucleotide hybridization, selective amplification, and selective primer extension.

The β-subunit polynucleotides are also useful for testing an individual for a genotype that while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the β-subunit gene that results in altered affinity for ligand, for example, could result in an excessive or decreased drug effect with standard concentrations of ligand. Alternatively, for example, a mutation in the subunit gene that results in an altered interaction with the β-subunit could result in an increased or decreased drug effect with standard concentrations of a drug that affects this functional interaction. Accordingly, the β-subunit polynucleotides described herein can be used to assess the mutation content of the β-subunit gene in an individual in order to select an appropriate compound or dosage regimen for treatment.

Thus polynucleotides displaying genetic variations that affect treatment provide a diagnostic target that can be used to tailor treatment in an individual. Accordingly, the production of recombinant cells and animals containing these polymorphisms allow effective clinical design of treatment compounds and dosage regimens.

The β-subunit polynucleotides are also useful for chromosome identification when the sequence is identified with an individual chromosome and to a particular location on the chromosome. First, the DNA sequence is matched to the chromosome by in situ or other chromosome-specific hybridization. Sequences can also be correlated to specific chromosomes by preparing PCR primers that can be used for PCR screening of somatic cell hybrids containing individual chromosomes from the desired species. Only hybrids containing the chromosome containing the gene homologous to the primer will yield an amplified fragment. Sublocalization can be achieved using chromosomal fragments. Other strategies include prescreening with labeled flow-sorted chromosomes and preselection by hybridization to chromosome-specific libraries. Further mapping strategies include fluorescence in situ hybridization which allows hybridization with probes shorter than those traditionally used. Reagents for chromosome mapping can be used individually to mark a single chromosome or a single site on the chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

The β-subunit polynucleotides can also be used to identify individuals from small biological samples. This can be done for example using restriction fragment-length polymorphism (RFLP) to identify an individual. Thus, the polynucleotides described herein are useful as DNA markers for RFLP (See U.S. Pat. No. 5,272,057). Furthermore, the β-subunit sequence can be used to provide an alternative technique which determines the actual DNA sequence of selected fragments in the genome of an individual. Thus, the β-subunit sequences described herein can be used to prepare two PCR primers from the 5′ and 3′ ends of the sequences. These primers can then be used to amplify DNA from an individual for subsequent sequencing.

Panels of corresponding DNA sequences from individuals prepared in this manner can provide unique individual identifications, as each individual will have a unique set of such DNA sequences. It is estimated that allelic variation in humans occurs with a frequency of about once per each 500 bases. Allelic variation occurs to some degree in the coding regions of these sequences, and to a greater degree in the noncoding regions. The β-subunit sequences can be used to obtain such identification sequences from individuals and from tissue. The sequences represent unique fragments of the human genome. Each of the sequences described herein can, to some degree, be used as a standard against which DNA from an individual can be compared for identification purposes.

If a panel of reagents from the sequences is used to generate a unique identification database for an individual, those same reagents can later be used to identify tissue from that individual. Using the unique identification database, positive identification of the individual, living or dead, can be made from extremely small tissue samples.

The β-subunit polynucleotides can also be used in forensic identification procedures. PCR technology can be used to amplify DNA sequences taken from very small biological samples, such as a single hair follicle, body fluids (e.g., blood, saliva, or semen). The amplified sequence can then be compared to a standard allowing identification of the origin of the sample.

The β-subunit polynucleotides can thus be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to the noncoding region are particularly useful since greater polymorphism occurs in the noncoding regions, making it easier to differentiate individuals using this technique. Fragments are at least 12 bases.

The β-subunit polynucleotides can further be used to provide polynucleotide reagents, e.g., labeled or labelable probes which can be used in, for example, an in situ hybridization technique, to identify a specific tissue. This is useful in cases in which a forensic pathologist is presented with a tissue of unknown origin. Panels of β-subunit probes can be used to identify tissue by species and/or by organ type.

In a similar fashion, these primers and probes can be used to screen tissue culture for contamination (i.e. screen for the presence of a mixture of different types of cells in a culture).

Alternatively, the β-subunit polynucleotides can be used directly to block transcription or translation of β-subunit gene expression by means of antisense or ribozyme constructs. Thus, in a disorder characterized by abnormally high or undesirable β-subunit gene expression, nucleic acids can be directly used for treatment.

The β-subunit polynucleotides are thus useful as antisense constructs to control β-subunit gene expression in cells, tissues, and organisms. A DNA antisense polynucleotide is designed to be complementary to a region of the gene involved in transcription, preventing transcription and hence production of β-subunit protein. An antisense RNA or DNA polynucleotide would hybridize to the mRNA and thus block translation of mRNA into β-subunit protein.

Examples of antisense molecules useful to inhibit nucleic acid expression include antisense molecules complementary to a fragment of the 5′ untranslated region of SEQ ID NO:67 which also includes the start codon and antisense molecules which are complementary to a fragment of the 3′ untranslated region of SEQ ID NO:67.

Alternatively, a class of antisense molecules can be used to inactivate mRNA in order to decrease expression of β-subunit nucleic acid. Accordingly, these molecules can treat a disorder characterized by abnormal or undesired subunit nucleic acid expression. This technique involves cleavage by means of ribozymes containing nucleotide sequences complementary to one or more regions in the mRNA that attenuate the ability of the mRNA to be translated. Possible regions include coding regions and particularly coding regions corresponding to the functional activities of the β-subunit protein.

The β-subunit polynucleotides also provide vectors for gene therapy in patients containing cells that are aberrant in β-subunit gene expression. Thus, recombinant cells, which include the patient's cells that have been engineered ex vivo and returned to the patient, are introduced into an individual where the cells produce the desired β-subunit protein to treat the individual.

The invention also encompasses kits for detecting the presence of a β-subunit nucleic acid in a biological sample. For example, the kit can comprise reagents such as a labeled or labelable nucleic acid or agent capable of detecting β-subunit nucleic acid in a biological sample; means for determining the amount of β-subunit nucleic acid in the sample; and means for comparing the amount of β-subunit nucleic acid in the sample with a standard. The compound or agent can be packaged in a suitable container. The kit can further comprise instructions for using the kit to detect β-subunit mRNA or DNA.

Vectors/Host Cells

The invention also provides vectors containing the β-subunit polynucleotides and to host cells containing the β-subunit polynucleotides. As described more fully below, vectors can be used for cloning or expression but are preferably used for expression of the β-subunit. Preferably expression systems include host cells in which both the α and β subunits are expressed. The term “vector” refers to a vehicle, preferably a nucleic acid molecule, that can transport the β-subunit polynucleotides. When the vector is a nucleic acid molecule, the β-subunit polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the β-subunit polynucleotides. Alternatively, the vector may integrate into the host cell genome and produce additional copies of the β-subunit polynucleotides when the host cell replicates.

The invention provides vectors for the maintenance (cloning vectors) or vectors for expression (expression vectors) of the β-subunit polynucleotides. The vectors can function in procaryotic or eukaryotic cells or in both (shuttle vectors).

Expression vectors contain cis-acting regulatory regions that are operably linked in the vector to the β-subunit polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the β-subunit polynucleotides from the vector. Alternatively, a trans-acting factor may be supplied by the host cell. Finally, a trans-acting factor can be produced from the vector itself.

It is understood, however, that in some embodiments, transcription and/or translation of the β-subunit polynucleotides can occur in a cell-free system.

The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y. (1989).

A variety of expression vectors can be used to express a β-subunit polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g., cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y. (1989).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The β-subunit polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the β-subunit polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression can be maximized in a host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990) 119-128). Alternatively, the sequence of the polynucleotide of interest can be altered to provide preferential codon usage for a specific host cell, for example E. coli. (Wada et al., Nucleic Acids Res. 20:2111-2118 (1992)).

The β-subunit polynucleotides can also be expressed by expression vectors that are operative in yeast. Examples of vectors for expression in yeast e.g., S. cerevisiae include pYepSec1 (Baldari, et al., EMBO J. 6:229-234 (1987)), pMFa (Kurjan et al., Cell 30:933-943 (1982)), pJRY88 (Schultz et al., Gene 54:113-123 (1987)), and pYES2 (Invitrogen Corporation, San Diego, Calif.).

The β-subunit polynucleotides can also be expressed in insect cells using, for example, baculovirus expression vectors. Baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

In certain embodiments of the invention, the polynucleotides described herein are expressed in mammalian cells using mammalian expression vectors. Examples of mammalian expression vectors include pCDM8 (Seed, B. Nature 329:840 (1987)) and pMT2PC (Kaufman et al., EMBO J. 6:187-195 (1987)).

The expression vectors listed herein are provided by way of example only of the well-known vectors available to those of ordinary skill in the art that would be useful to express the β-subunit polynucleotides. The person of ordinary skill in the art would be aware of other vectors suitable for maintenance propagation or expression of the polynucleotides described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the β-subunit polynucleotides can be introduced either alone or with other polynucleotides that are not related to the β-subunit polynucleotides such as those providing trans-acting factors for expression vectors. When more than one vector is introduced into a cell, the vectors can be introduced independently, co-introduced or joined to the β-subunit polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the β-subunit polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

Uses of Vectors and Host Cells

The host cells expressing the polypeptides described herein, and particularly recombinant host cells, have a variety of uses. First, the cells are useful for producing β-subunit proteins or polypeptides that can be further purified to produce desired amounts of β-subunit protein or fragments. Thus, host cells containing expression vectors are useful for polypeptide production.

Host cells are also useful for conducting cell-based assays involving the β-subunit or β-subunit fragments. Thus, a recombinant host cell expressing a native β-subunit is useful to assay for compounds that stimulate or inhibit β-subunit function. This includes ligand binding, gene expression at the level of transcription or translation, α-subunit interaction, and ability to be phosphorylated.

Accordingly, in preferred embodiments the host cells express both the α and β subunits or relevant portions thereof. Therefore, cell-based and cell-free assays are provided in which both α and β subunits (or relevant portions thereof) provide assays useful for detection of β-subunit function. In a preferred embodiment, the invention provides a cell-based assay in which the cell expresses both the α and β subunits.

Assay end points include ligand binding, α-subunit association or activation, channel currents, phosphorylation, and conformational changes in either the α or β subunit. Interaction of the α and β subunit can be measured in assays based on dual label energy transfer, methods in which reactants are separately labeled with an energy transfer donor and acceptor, such that energy transfer results when the donor and acceptor are brought into close proximity to each other, producing a detectable lifetime change. Assay methods for detection of a complex formed between the α and β subunits include determining fluorescence emission or fluorescence quenching or other energy transfer between labels on the two subunits. One example is a fluorescein homoquenching method in which a subunit is labeled with fluorescein positioned such that when the other subunit is bound the fluorescein molecules quench one another and the fluorescence of the solution decreases. This analytical technique is well-known and within the skill of those in the art. See, for example, U.S. Pat. No. 5,631,169; U.S. Pat. No. 5,506,107; U.S. Pat. No. 5,716,784; and U.S. Pat. No. 5,763,189.

Host cells are also useful for identifying subunit mutants in which these functions are affected. If the mutants naturally occur and give rise to a pathology, host cells containing the mutations are useful to assay compounds that have a desired effect on the mutant β-subunit (for example, stimulating or inhibiting function) which may not be indicated by their effect on the native β-subunit.

Recombinant host cells are also useful for expressing the chimeric polypeptides described herein to assess compounds that activate or suppress activation by means of a heterologous intracellular or extracellular domain. Alternatively, one or more heterologous transmembrane domains can be used to assess the effect of a desired extracellular domain on any given host cell. In this embodiment, a transmembrane domain compatible with the specific host cell is used to make the chimeric polypeptide.

Further, mutant β-subunits can be designed in which one or more of the various functions is engineered to be increased or decreased (i.e., ligand binding or α-subunit activation) and used to augment or replace β-subunit proteins in an individual. Thus, host cells can provide a therapeutic benefit by replacing an aberrant β-subunit or providing an aberrant β-subunit that provides a therapeutic result. In one embodiment, the cells provide β-subunits that are abnormally active.

In another embodiment, the cells provide β-subunits that are abnormally inactive. These β-subunits can compete with endogenous β-subunits in the individual.

In another embodiment, cells expressing β-subunits that cannot be activated, are introduced into an individual in order to compete with endogenous β-subunits for ligand or α-subunit. For example, in the case in which excessive ligand is part of a treatment modality, it may be necessary to inactivate this ligand at a specific point in treatment. Providing cells that compete for the ligand, but which cannot be affected by β-subunit activation would be beneficial.

Homologously recombinant host cells can also be produced that allow the in situ alteration of endogenous β-subunit polynucleotide sequences in a host cell genome. This technology is more fully described in WO 93/09222, WO 91/12650 and U.S. Pat. No. 5,641,670. Briefly, specific polynucleotide sequences corresponding to the β-subunit polynucleotides or sequences proximal or distal to a β-subunit gene are allowed to integrate into a host cell genome by homologous recombination where expression of the gene can be affected. In one embodiment, regulatory sequences are introduced that either increase or decrease expression of an endogenous sequence. Accordingly, a β-subunit protein can be produced in a cell not normally producing it, or increased expression of β-subunit protein can result in a cell normally producing the protein at a specific level. Alternatively, the entire gene can be deleted. Still further, specific mutations can be introduced into any desired region of the gene to produce mutant β-subunit proteins. Such mutations could be introduced, for example, into the specific functional regions such as the ligand-binding site or the α-subunit interaction site.

In one embodiment, the host cell can be a fertilized oocyte or embryonic stem cell that can be used to produce a transgenic animal containing the altered β-subunit gene. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous β-subunit gene is selected (see e.g., Li, E. et al., Cell 69:915 (1992)). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a β-subunit protein and identifying and evaluating modulators of β-subunit protein activity.

Other examples of transgenic animals include non-human primates, sheep, dogs, cows, goats, chickens, and amphibians.

In one embodiment, a host cell is a fertilized oocyte or an embryonic stem cell into which β-subunit polynucleotide sequences have been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the β-subunit nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the β-subunit protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al., PNAS 89:6232-6236 (1992). Another example of a recombinase system is the FLP recombinase system of S. cerevisiae (O'Gorman et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al., Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could effect ligand binding, α-subunit activation, and ability to be phosphorylated may not be evident from in vitro cell-free or cell-based assays. Accordingly, it is useful to provide non-human transgenic animals to assay in vivo β-subunit function, including ligand and α-subunit interaction, the effect of specific mutant β-subunits on the α-subunit, channel function, and ligand interaction, and the effect of chimeric subunits or channels. It is also possible to assess the effect of null mutations, that is mutations that substantially or completely eliminate one or more β-subunit functions.

Pharmaceutical Compositions

The β-subunit nucleic acid molecules, protein (particularly fragments such as the various domains), modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL□ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a β-subunit protein or anti-β-subunit antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al., PNAS 91:3054-3057 (1994)). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

### EXAMPLES

The pore-forming α-subunit of the high conductance calcium-activated potassium channel (maxi-K) has been identified and cloned in human (called hSlo) and mouse (called mSlo). See, for example, Knaus, J. Biol. Chem. 269:3921-3924 (1994), and Butler et al., Science 261:221-224 (1993). Experiments were conducted to examine the functional role of the novel human calcium-activated potassium channel β-subunit C7F2 (SEQ ID NO:67) in the high conductance calcium-activated potassium channel maxi-K.

These experiments show that a physical interaction of C7F2 with hSlo and mSlo modifies the channel activity of maxi-K, supporting the claim that C7F2 is a functional β-subunit of maxi-K.

### Example 1

### Physical Association of C7F2 with mSLO

The open reading frame of C7F2 (nucleotides 502-1131 of SEQ ID NO:67) was cloned into the pcDNA3.11V5/His-TOPO vector (Invitrogen) to provide a V5 epitope tag. This vector and a vector containing mSlo were transiently co-transfected into HEK293 cells with lipofectamine or Fugene. mSlo was immunoprecipitated with antibodies directed against the α-subunit. The immunoprecipitates were subjected to Western blotting with monoclonal antibody directed against the V5 epitope tag to reveal the presence of the V5-tagged C7F2.

These experiments demonstrate that human C7F2 can associate with mSlo (data not shown), suggesting a physical interaction of C7F2 with the pore-forming α-subunit of maxi-K. These results confirm the claim that C7F2 is a β-subunit for maxi-K.

### Example 2

### Electrophysiological Consequences of Association of C7F2 with hSLO and mSLO on the Channel Activity of Maxi-K

The open reading frame of C7F2 was cloned into the pIRES-EGFP vector (Clontech) to express both C7F2 and green fluorescent protein (GFP) in transfected cells. This vector and vectors containing either hSlo or mSlo were transiently co-transfected into HEK293 cells with lipofectamine or Fugene. Cells were selected for recording based on the expression of GFP.

Activation and deactivation kinetics of the mouse maxi-K channel (mSlo) were dramatically different when expressed alone or when co-expressed with C7F2. These inside-out patch-clamp experiments revealed that co-expression of C7F2 with mSlo (horizontal bars labeled mSlo+C7F2) dramatically increases the time constants of activation (mSlo+C7F2 activation) and deactivation (mSlo+C7F2 deactivation) of the mouse maxi-K when compared to expression of mSlo alone (horizontal bars labeled mSlo activation and mSlo deactivation, respectfully). Similar effects were seen for the human maxi-K hSlo.

In the presence of 3 μM Ca++, C7F2 co-expression with mSlo causes a hyperpolarizing (leftward) shift of 20 mV of half-maximal channel activation, suggesting increased sensitivity of the mouse maxi-K channel to calcium ions. This is the typical behavior of the previously characterized β-subunit. However, when C7F2 is co-expressed with hSlo, there is a 20-50 mV depolarizing (rightward) shift of half-maximal channel activation, suggesting decreased sensitivity of the human maxi-K channel to calcium ions. This is a unique, novel behavior of C7F2 compared to the previously characterized β-subunit.

Functional interaction of C7F2 with mSlo and hSlo, leading to changes in activation and deactivation kinetics of the maxi-K channel along with shifts in the half-maximal channel activation in response to calcium, confirms the claim that C7F2 is a β-subunit for maxi-K.

### Example 3

### Expression Pattern on Human Slowpoke ∃4

h∃4 expression was first analyzed using multiple Northern blots and brain region Northern blots. h∃4 is expressed predominantly in human brain with a major mRNA product at 1.6 Kb and a minor one at 5 Kb, whereas only limited expression is observed in non-neural tissues. No signal could be detected in sections of these non-neural tissues by in-situ hybridization. Within the brain regions analyzed, expression is highest in cortical regions.

Tissue and regional expression of Slowpoke ∀ and ∃ was analyzed further in human and monkey by in situ hybridization. ∃4 is expressed in all layers of human cortex and no signal is detected with a sense (S) probe. At higher magnifications hybridization was observed predominantly over human cortical neurons. Film autoradiography of monkey brain sections demonstrated widespread expression of Slowpoke alpha subunit (Slo) and h∃4 particularly in cortex, basal ganglia, ifundibulum and hippocampus. Expression of h∃2/3 is less robust, and virtually no h∃1 mRNA can be detected. It is also evident from emulsion autoradiography of monkey brain sections that there is a striking overlap in expression of Slowpoke alpha subunit and h∃4 in multiple neuronal populations in cortex (CTX), dentate gyrus and CA3 regions of hippocampus (HIP) and thalamus (THL). Also, emulsion autoradiograms confirmed that there is more limited expression of h∃2/3 and no expression of h∃1 in these brain regions. ∃4 expression is also detected in spinal motor neurons, sympathetic neurons of the superior cervical ganglion and in a subpopulation of dorsal root ganglion neurons (data not shown). This extensive brain distribution of ∃4 may be contrasted with the situation in human aorta, where Slowpoke ∀ subunit and h∃1 are expressed predominantly but h∃4 mRNA cannot be detected. In addition, despite the faint signal from these tissues on Northern blots, no ∃4 expression is detected in sections of monkey heart, skeletal muscle, pancreas, liver, testes, lung or adipose tissue by in situ hybridization.

### Example 4

### Slowpoke ∃4 Binds to hSLO

An experiment was performed to determine if ∃4 can co-immunoprecipitate with Slo. When HEK 293 cells are transfected with hSlo together with h∃4, and hSlo is immunoprecipitated with a specific antibody, h∃4 can be detected in the immunoprecipitate. It is interesting that two co-immunoprecipitating protein bands are seen, one with an apparent molecular weight of (29 kD) equivalent to that predicted for the epitope-tagged h∃4, and the second several kD larger. Although the higher molecular weight band is barely detectable in the cell lysate and in h∃4 immunoprecipitate, it clearly is enriched relative to the smaller band in the hSlo immunoprecipitate. No h∃4 staining is observed in hSlo immunoprecipiates from cells in which either hSlo or h∃4 is transfected alone. A similar result is observed when the interaction between mSlo and m∃4 is analyzed. In this case the higher molecular weight m∃4 band is more evident in the lysate and m∃4 immunoprecipitate, but it too preferentially co-immunoprecipitates with mSlo. These results suggest that h∃4 and m∃4 may exist in several different post-translationally modified forms, one of which binds preferentially to Slowpoke ∀ subunits. Slowpoke-∃4 binding is also observed when the experiment is done by immunoprecipitating epitope-tagged ∃4 and probing for Slowpoke subunit with anti-Slowpoke antibodies.

### Example 5

### Slowpoke ∃4 Modulates hSlo Activation Kinetics

hSlo current was measured in inside-out membrane patches from HEK 293 cells transfected with hSlo ∀ subunit. Activation of the current in response to a depolarizing voltage step to +80 mV is much slower in cells co-transfected with h∃4. A different pattern is observed when hSlo deactivation is considered. As is evident from inspection of the current traces, h∃4 has little or no effect on the deactivation kinetics, and this is confirmed by the deactivation θ values observed.

### Example 6

### Slowpoke ∃4 Modulates the Voltage Dependance of hSLO Activation

h∃4 can influence the steady-state activation of hSlo. In cells transfected with h∃4, the voltage dependence of hSlo activation is shifted some 50 mV to the right compared with cells transfected with hSlo. This requirement for greater depolarization to activate the current is apparent at all calcium concentrations examined in the range form 0.3-3:M.

### Example 7

### Slowpoke ∃4 Modulates Toxin Block of hSLO

Because auxiliary subunits often alter the effects of pharmacological agents on Slowpoke channel ∀ subunits, the effects of h∃4 on the block of hSlo current by the scorpion venom toxins charybdotoxin and iberiotoxin were tested in the whole cellpatch configuration. As shown in in cells transfected with hSlo and control vector, the current is blocked 90% or more by 300 nM of either toxin (filled symbols). In the presence of h∃4, in contrast, no block at all is observed by 300 nM toxin and even as much as 1:M iberiotoxin is without effect.

This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. An isolated 15571, 2465, 14266, 2882, 52871, 8203 or 16852 nucleic acid molecule selected from the group consisting of:
a) a nucleic acid molecule comprising a nucleotide sequence which is at least 60% identical to the nucleotide sequence of SEQ ID NO:1, 9, 12, 14, 27, 33, 34, 35 or 67;
b) a nucleic acid molecule comprising a fragment of at least 15 nucleotides of the nucleotide sequence of SEQ ID NO:1, 9, 12, 14, 27, 33, 34, 35 or 67;
c) a nucleic acid molecule which encodes a polypeptide comprising the amino acid sequence of SEQ ID NO:2, 10, 11, 13, 28, 36, 37, 38 or 68;
d) a nucleic acid molecule which encodes a fragment of a polypeptide comprising the amino acid sequence of SEQ ID NO:2, 10, 11, 13, 28, 36, 37, 38 or 68, wherein the fragment comprises at least 15 contiguous amino acids of SEQ ID NO:2, 10, 11, 13, 28, 36, 37, 38 or 68;
e) a nucleic acid molecule which encodes a naturally occurring allelic variant of a polypeptide comprising the amino acid sequence of SEQ ID NO:2, 10, 11, 13, 28, 36, 37, 38 or 68, wherein the nucleic acid molecule hybridizes to a nucleic acid molecule comprising SEQ ID NO:1, 9, 12, 14, 27, 33, 34, 35 or 67, or a complement thereof, under stringent conditions;
f) a nucleic acid molecule comprising the nucleotide sequence of SEQ ID NO:1, 9, 12, 14, 27, 33, 34, 35 or 67, and
g) a nucleic acid molecule which encodes a polypeptide comprising the amino acid sequence of SEQ ID NO:2, 10, 11, 13, 28, 36, 37, 38 or 68.

2. The isolated nucleic acid molecule of claim 1, which is the nucleotide sequence SEQ ID NO:1, 9, 12, 14, 27, 33, 34, 35 or 67.

3. A host cell which contains the nucleic acid molecule of claim 1.

4. An isolated 15571, 2465, 14266, 2882, 52871, 8203 or 16852 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO:1, 9, 12, 14, 27, 33, 34, 35 or 67;
b) a naturally occurring allelic variant of a polypeptide comprising the amino acid sequence of SEQ ID NO:2, 10, 11, 13, 28, 36, 37, 38 or 68, wherein the polypeptide is encoded by a nucleic acid molecule which hybridizes to a nucleic acid molecule comprising SEQ ID NO:1, 9, 12, 14, 27, 33, 34, 35 or 67, or a complement thereof under stringent conditions;
c) a fragment of a polypeptide comprising the amino acid sequence of SEQ ID NO:2, 10, 11, 13, 28, 36, 37, 38 or 68, wherein the fragment comprises at least 15 contiguous amino acids of SEQ ID NO:2, 10, 11, 13, 28, 36, 37, 38 or 68; and
d) the amino acid sequence of SEQ ID NO:2,10, 11, 13, 28, 36, 37, 38 or 73.

5. An antibody which selectively binds to a polypeptide of claim 4.

6. The polypeptide of claim 4, further comprising heterologous amino acid sequences.

7. A method for producing a polypeptide selected from the group consisting of:
a) a polypeptide comprising the amino acid sequence of SEQ ID NO:2, 10, 11, 13, 28, 36, 37, 38 or 68,
b) a polypeptide comprising a fragment of the amino acid sequence of SEQ ID NO:2, 10, 11, 13, 28, 36, 37, 38 or 68, wherein the fragment comprises at least 15 contiguous amino acids of SEQ ID NO:2, 10, 11, 13, 28, 36, 37, 38 or 68;
c) a naturally occurring allelic variant of a polypeptide comprising the amino acid sequence of SEQ ID NO:2, 10, 11, 13, 28, 36, 37, 38 or 68, wherein the polypeptide is encoded by a nucleic acid molecule which hybridizes to a nucleic acid molecule comprising SEQ ID NO:1, 9, 12, 14, 27, 33, 34, 35 or 67; and
d) the amino acid sequence of SEQ ID NO:2, 10, 11, 13, 28, 36, 37, 38 or 68; comprising culturing the host cell of claim 3 under conditions in which the nucleic acid molecule is expressed.

8. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

9. A kit comprising a compound which selectively hybridizes to a nucleic acid molecule of claim 1 or binds to a polypeptide encoded by the nucleic acid molecule and instructions for use.

10. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

11. A method for modulating the activity of a polypeptide of claim 4 comprising contacting the polypeptide or a cell expressing the polypeptide with a compound which binds to the polypeptide in a sufficient concentration to modulate the activity of the polypeptide.

12. A method for identifying a compound capable of treating a disorder characterized by aberrant 15571, 2465, 14266, 2882, 52871, 8203 or 16852 activity, comprising assaying the ability of the compound to modulate 15571, 2465, 14266, 2882, 52871, 8203 or 16852 nucleic acid expression or 15571, 2465, 14266, 2882, 52871, 8203 or 16852 polypeptide activity, thereby identifying a compound capable of treating a disorder characterized by aberrant 15571, 2465, 14266, 2882, 52871, 8203 or 16852 activity.

13. A method of identifying a nucleic acid molecule associated with a disorder characterized by aberrant 15571, 2465, 14266, 2882, 52871, 8203 or 16852 activity, comprising:
a) contacting a sample from a subject with a disorder characterized by aberrant 15571, 2465, 14266, 2882, 52871, 8203 or 16852 activity, comprising nucleic acid molecules with a hybridization probe comprising at least 25 contiguous nucleotides of SEQ ID NO:1, 9, 12, 14, 27, 33, 34, 35 or 67 defined in claim 2; and
b) detecting the presence of a nucleic acid molecule in the sample that hybridizes to the probe, thereby identifying a nucleic acid molecule associated with a disorder characterized by aberrant 15571, 2465, 14266, 2882, 52871, 8203 or 16852 activity.

14. A method of identifying a polypeptide associated with a disorder characterized by aberrant 15571, 2465, 14266, 2882, 52871, 8203 or 16852 activity, comprising:
a) contacting a sample comprising polypeptides with a 15571, 2465, 14266, 2882, 52871, 8203 or 16852 polypeptide defined in claim 4; and
b) detecting the presence of a polypeptide in the sample that binds to the 15571, 2465, 14266, 2882, 52871, 8203 or 16852 binding partner, thereby identifying the polypeptide associated with a disorder characterized by aberrant 15571, 2465, 14266, 2882, 52871, 8203 or 16852 activity.

15. A method of identifying a subject having a disorder characterized by aberrant 15571, 2465, 14266, 2882, 52871, 8203 or 16852 activity, comprising:
a) contacting a sample obtained from the subject comprising nucleic acid molecules with a hybridization probe comprising at least 25 contiguous nucleotides of SEQ ID NO:1, 9, 12, 14, 27, 33, 34, 35 or 67 defined in claim 2; and
b) detecting the presence of a nucleic acid molecule in the sample that hybridizes to the probe, thereby identifying a subject having a disorder characterized by aberrant 15571, 2465, 14266, 2882, 52871, 8203 or 16852 activity.

16. A method for treating a subject having a disorder characterized by aberrant 15571, 2465, 14266, 2882, 52871, 8203 or 16852 activity, or a subject at risk of developing a disorder characterized by aberrant 15571, 2465, 14266, 2882, 52871, 8203 or 16852 activity, comprising administering to the subject a 15571, 2465, 14266, 2882, 52871, 8203 or 16852 modulator of the nucleic acid molecule defined in claim 1 or the polypeptide encoded by the nucleic acid molecule or contacting a cell with a 15571, 2465, 14266, 2882, 52871, 8203 or 16852 modulator.

17. The method of claim 16, wherein the 15571, 2465, 14266, 2882, 52871, 8203 or 16852 modulator is a small molecule; peptide; phosphopeptide; anti-15571, 2465, 14266, 2882, 52871, 8203 or 16852 antibody; a 15571, 2465, 14266, 2882, 52871, 8203 or 16852 polypeptide comprising the amino acid sequence of SEQ ID NO:2, 10, 11, 13, 28, 36, 37, 38 or 68, or a fragment thereof; a 15571, 2465, 14266, 2882, 52871, 8203 or 16852 polypeptide comprising an amino acid sequence which is at least 90 percent identical to the amino acid sequence of SEQ ID NO:2, 10, 11, 13, 28, 36, 37, 38 or 68, wherein the percent identity is calculated using the ALIGN program for comparing amino acid sequences, a PAM120 weight residue table, a gap length penalty of 12, and a gap penalty of 4; or an isolated naturally occurring allelic variant of a polypeptide consisting of the amino acid sequence of SEQ ID NO:2, 10, 11, 13, 28, 36, 37, 38 or 68, wherein the polypeptide is encoded by a nucleic acid molecule which hybridizes to a complement of a nucleic acid molecule consisting of SEQ ID NO:1, 9, 12, 14, 27, 33, 34, 35 or 67 at 6×SSC at 45° C., followed by one or more washes in 0.2×SSC, 0.1% SDS at 65° C.

18. The method of claim 16, wherein the 15571, 2465, 14266, 2882, 52871, 8203 or 16852 modulator is
a) an antisense 15571, 2465, 14266, 2882, 52871, 8203 or 16852 nucleic acid molecule;
b) is a ribozyme;
c) the nucleotide sequence of SEQ ID NO:1, 9, 12, 14, 27, 33, 34, 35 or 67 or a fragment thereof;
d) a nucleic acid molecule encoding a polypeptide comprising an amino acid sequence which is at least 90 percent identical to the amino acid sequence of SEQ ID NO:2, 10, 11, 13, 28, 36, 37, 38 or 68, wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO:2, 10, 11, 13, 28, 36, 37, 38 or 68, wherein the nucleic acid molecule which hybridizes to a complement of a nucleic acid molecule consisting of SEQ ID NO:1, 9, 12, 14, 27, 33, 34, 35 or 67 at 6×SSC at 45° C., followed by one or more washes in 0.2×SSC, 0.1% SDS at 65° C.; or
f) a gene therapy vector.
